(19) United States
(12) Patent Application Publication
(10) Pub. No.: US 2026/0256939 A1
(43) Pub. Date: Sep. 3, 2026

(54) FLUOROCAMPTOTHECIN COMPOUNDS, CONJUGATES, AND METHODS THEREOF

(71) Applicant: Aarvik Therapeutics, Inc., Hayward, CA (US)

(72) Inventors: Vasu JAMMALAMADAKA, Dublin, CA (US); Jagath Reddy JUNUTULA, Fremont, CA (US); Sunil BHAKTA, San Ramon, CA (US)

(21) Appl. No.: 19/446,754

(22) Filed: Jan. 12, 2026

Related U.S. Application Data
(63) Continuation-in-part of application No. PCT/US25/28255, filed on May 7, 2025.
(60) Provisional application No. 63/643,902, filed on May 7, 2024.
(60) Provisional application No. 63/712,308, filed on Oct. 25, 2024.
(60) Provisional application No. 63/722,061, filed on Nov. 18, 2024.

(51) Int. Cl. A61K 47/68; A61P 35/00; C07H 15/26

(52) U.S. Cl. CPC A61K 47/6889; A61K 47/68037; A61K 47/6855; A61P 35/00; C07H 15/26

(57) ABSTRACT

The present disclosure provides a compound of Formula I and Formula VII, and a process of preparing the compound of Formula I and Formula VII. The present disclosure further provides a compound of Formula IV and Formula VIII and a conjugate having Formula V and Formula IX. Further, the present disclosure provides a pharmaceutical composition comprising a compound as disclosed herein, or a conjugate as disclosed herein. Furthermore, the present disclosure provides a method for treating cancer, a method of inhibiting cell division and a use of a compound as disclosed herein, or a conjugate as disclosed herein, for the manufacture of a medicament for treating cancer.

### REFERENCE TO RELATED APPLICATIONS

This application is a continuation-in-part of International Application PCT/US2025/028255, filed May 7, 2025, which claims the benefit of the priority dates of U.S. provisional applications 63/643,902 filed May 7, 2024; 63/712,308, filed Oct. 25, 2024; and 63/722,061, filed Nov. 18, 2024, the contents of which are incorporated herein in their entirety.

### FIELD OF INVENTION

The present disclosure relates to the field of therapeutics, in particular relates to fluorocamptothecin compounds, conjugates comprising fluorocamptothecin compounds, and methods thereof.

### BACKGROUND

Antibody drug conjugates (ADCs) are an important class of oncology therapeutics that combine a tumour-targeting antibody with a cell-killing cytotoxic drug (payload). Camptothecin being a natural product inhibits topoisomerase I and has broad spectrum anti-tumor activity, several analogues of the same have been developed as payloads for ADCs. Trastuzumab deruxtecan (Enhertu™) is an ADC in which the camptothecin analogue, deruxtecan (Dxd), is conjugated to the anti-HER2 antibody trastuzumab, via a cleavable tetrapeptide-based linker, and sacituzumab govitecan (Trodelvy™) in which the camptothecin analogue, SN-38, is conjugated to the anti-Trop-2 antibody, sacituzumab, via a hydrolysable, pH-sensitive linker.

However, camptothecin being poorly soluble faces challenges in development as an effective therapeutic. In view of this, identification and development of analogues or derivatives of camptothecin with desirable therapeutic properties has been a topic of interest in the field of medical research.

In view of the above, there is a dire need in the art to develop camptothecin analogues as payloads to be used in ADCs.

### SUMMARY OF THE INVENTION

In an aspect of the present disclosure, there is provided a compound of Formula I

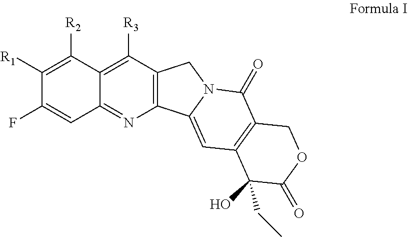

wherein, R₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy; and
R₃ is selected from hydrogen, halogen, or C₁₋₆ alkyl, wherein C₁₋₆ alkyl is optionally substituted with C₁₋₆ aminoalkyl.

In another aspect of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I

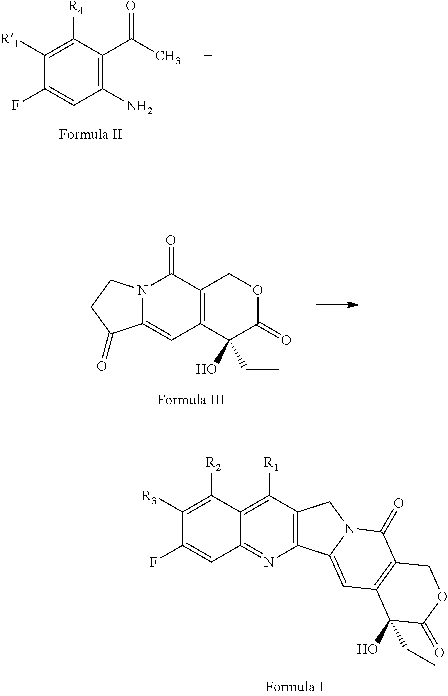

wherein, R′₁ is selected from hydrogen, hydroxy, halogen, C₁₋₆ alkyl, C₁₋₆ alkoxy, C₁₋₆ aminoalkyl, C₆₋₁₂ aryl, C₇₋₁₂ aralkyl, C₇₋₁₂ alkoxyaryl, C₈₋₁₂ alkoxyaralkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₃ is selected from hydrogen, halogen, or C₁₋₆ alkyl, wherein C₁₋₆ alkyl is optionally substituted with C₁₋₆ aminoalkyl.

In one another aspect of the present disclosure, there is provided a compound of Formula IV, its pharmaceutically acceptable salt or derivatives thereof

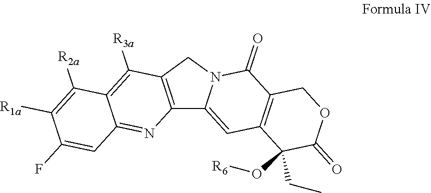

wherein, R₁ₐ is selected from hydrogen, hydroxy, halogen, C₁₋₆ alkyl, C₁₋₆ alkoxy, C₁₋₆ aminoalkyl, NHR₄ wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is a selected from hydroxy, C₁₋₆ alkoxy, C₁₋₆ aminoalkyl, and NHR₄;
R₂ₐ is selected from hydrogen, hydroxy, halogen, C₁₋₆ alkyl, C₁₋₆ alkoxy, C₁₋₆ aminoalkyl, NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M2-L-Rₓ, wherein M2 is selected from hydroxy, C₁₋₆ alkoxy, C₁₋₆ aminoalkyl, and NHR₄;
R₃ₐ is selected from hydrogen, halogen, or C₁₋₆ alkyl, wherein C₁₋₆ alkyl is optionally substituted with C₁₋₆ aminoalkyl, and -M3-L-Rₓ, wherein M3 is selected from a direct bond and C₁₋₆ alkyl substituted with C₁₋₆ aminoalkyl; and
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and wherein at least one of R₁ₐ, R₂ₐ, R₃ₐ, and R₆ comprises -L-Rₓ.

In yet another aspect of the present disclosure, there is provided a conjugate having Formula V:

T-[(W-Rm-L-D)]n Formula V
T— is a targeting moiety selected from a protein, a portion of a protein, a peptide or a nucleic acid;
—W—Rₘ— is a moiety formed by the reaction of reactive moiety Rₓ and a functional group of T;
-L-D is a moiety having Formula VI

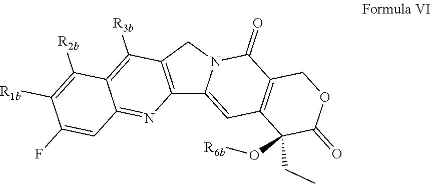

wherein, R1_{b} is selected from hydrogen, hydroxy, halogen, C₁₋₆ alkyl, C₁₋₆ alkoxy, C₁₋₆ aminoalkyl, NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is a selected from hydroxy, C₁₋₆ alkoxy, C₁₋₆ aminoalkyl, NHR₄ and —COR₅;
R2_{b} is selected from hydrogen, halogen, C₁₋₆ alkoxy, NHR₄ or -L, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxyl, and -M2-L-Rₓ, wherein M2 is selected from hydroxy, C₁₋₆ alkoxy, C₁₋₆ aminoalkyl, NHR₄, and —COR₅;
R3_{b} is selected from hydrogen, halogen, C₁₋₆ alkyl, or -L, wherein C₁₋₆ alkyl is optionally substituted with C₁₋₆ aminoalkyl and -M3-L-Rₓ, wherein M3 is selected from a direct bond and C₁₋₆ alkyl substituted with C₁₋₆ aminoalkyl;
R_{6b} is selected from hydrogen or -L;
wherein -L is a linker which is a bond or is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties;
wherein at least one of R1_{b}, R2_{b}, R3_{b}, and R_{6b} comprises -L;
m is an integer between 1 and 4; and
n is an integer between 1 and 20.

In one another aspect of the present disclosure, there is provided a compound of Formula VII, its pharmaceutically acceptable salt or derivatives thereof

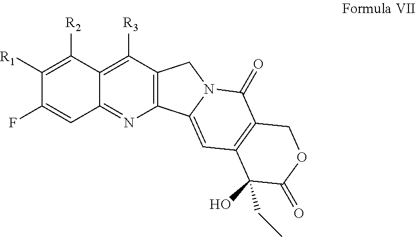

wherein, R₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is C₁₋₆ alkylamine, optionally wherein N is alkylated with C₁₋₆ alkyl;
R₃ is selected from hydrogen, halogen, or C₁₋₆ alkyl, wherein C₁₋₆ alkyl is optionally substituted with C₁₋₆ aminoalkyl.

In another aspect of the present disclosure, there is provided a process of preparing the compound of Formula VII as disclosed herein, the process comprising: reacting a compound of Formula II with a compound of Formula III in the presence of a solvent and optionally processing with a reagent to obtain the compound of Formula I

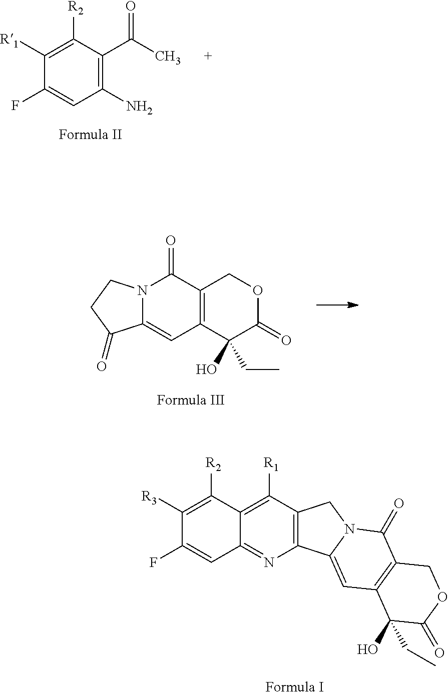

wherein, R′₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is C₁₋₆ alkylamine, optionally wherein N is alkylated with C₁₋₆ alkyl;
R₃ is selected from hydrogen, halogen, or C₁₋₆ alkyl, wherein C₁₋₆ alkyl is optionally substituted with C₁₋₆ aminoalkyl.

In one another aspect of the present disclosure, there is provided a compound of Formula VIII, its pharmaceutically acceptable salt or derivatives thereof

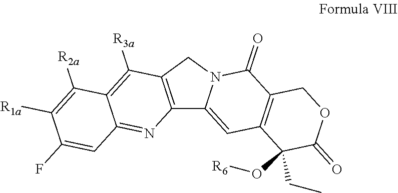

wherein, R₁ₐ is selected from hydrogen, hydroxy, halogen, C₁₋₆ alkyl, C₁₋₆ alkoxy, C₁₋₆ aminoalkyl, NHR₄ wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is a selected from hydroxy, C₁₋₆ alkoxy, C₁₋₆ aminoalkyl, and NHR₄;
R₂ₐ is selected from C₁₋₆ alkylamine, optionally wherein Nis alkylated with C₁₋₆ alkyl, and -M2-L-Rₓ, wherein M2 is selected from C₁₋₆ alkylamine, optionally wherein N is alkylated with C₁₋₆ alkyl;
R₃ₐ is selected from hydrogen, halogen, or C₁₋₆ alkyl, wherein C₁₋₆ alkyl is optionally substituted with C₁₋₆ aminoalkyl, and -M3-L-Rₓ, wherein M3 is selected from C₁₋₆ alkyl, wherein C₁₋₆ alkyl is substituted with C₁₋₆ aminoalkyl; and
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises -L-Rₓ.

In yet another aspect of the present disclosure, there is provided a conjugate having Formula IX:

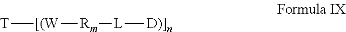

wherein:
T— is a targeting moiety selected from a protein, a portion of a protein, a peptide or a nucleic acid;
—W—Rₘ— is a moiety formed by the reaction of reactive moiety Rₓ and a functional group of T;
-L-D is a moiety having Formula VIII

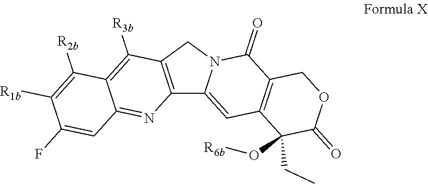

wherein, R1_{b} is selected from hydrogen, hydroxy, halogen, C₁₋₆ alkyl, C₁₋₆ alkoxy, C₁₋₆ aminoalkyl, NHR₄ wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, wherein-M is selected from, halogen, C₁₋₆ alkoxy, C₁₋₆ aminoalkyl, or NHR₄ wherein R₄ is selected from hydrogen, C₁₋₆ alkyl, C₁₋₆ haloalkyl, C₁₋₆ alkylcarboxy, or —COR₅, and -M1-L-Rₓ, wherein M1 is selected from hydroxy, C₁₋₆ alkoxy, C₁₋₆ aminoalkyl, and NHR₄.
R2_{b} is selected from C₁₋₆ alkylamine, optionally wherein N is alkylated with C₁₋₆ alkyl, and -M2-L-Rₓ, wherein-M2 is C₁₋₆ alkylamine, optionally wherein N is alkylated with C₁₋₆ alkyl;
R3_{b} is selected from hydrogen, halogen, C₁₋₆ alkyl, wherein C₁₋₆ alkyl is optionally substituted with C₁₋₆ aminoalkyl, and -M3-L-Rₓ, wherein-M3 is selected from a direct bond or C₁₋₆ alkyl is substituted with C₁₋₆ aminoalkyl; and
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and wherein at least one of R₁ₐ, R₂ₐ, R₃ₐ, and R₆ comprises -L-Rₓ.

In more aspects of the present disclosure, there is provided a pharmaceutical composition comprising a compound or a conjugate as disclosed herein, and a pharmaceutically acceptable excipient.

In more aspect of the present disclosure, there is provided a method for treating cancer, the method comprising administering to a subject suffering from cancer an effective amount of a compound or a conjugate as disclosed herein, optionally with other relevant cytotoxic or non-cytotoxic agents.

In more aspects of the present disclosure, there is provided a method of inhibiting cell division comprising contacting a cell with a compound or a conjugate as disclosed herein.

In more aspects of the present disclosure, there is provided use of a compound, or a conjugate as disclosed herein, for the manufacture of a medicament for treating cancer.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

FIG. 1A-1C show exemplary fluorocamptothecin compounds of Formula I, according to an embodiment of the present disclosure.

FIG. 2 shows a synthesis scheme 1 for AV-D021, according to an embodiment of the present disclosure.

FIG. 3 shows a synthesis scheme 2 for AV-D041, according to an embodiment of the present disclosure.

FIG. 4 shows a synthesis scheme 3 for AV-D042, according to an embodiment of the present disclosure.

FIG. 5 shows a synthesis scheme 4 for AV-D043, according to an embodiment of the present disclosure.

FIG. 6 shows a synthesis scheme 5 for AV-D046, according to an embodiment of the present disclosure.

FIG. 7 shows a synthesis scheme 6 for AV-D047, according to an embodiment of the present disclosure.

FIG. 8 shows a synthesis scheme 7 for AV-D050, according to an embodiment of the present disclosure.

FIG. 9 shows a synthesis scheme 8 for AV-D056, according to an embodiment of the present disclosure.

FIG. 10 shows a synthesis scheme 9 for AV-D061, according to an embodiment of the present disclosure.

FIG. 11 shows a synthesis scheme 10 for AV-D062, according to an embodiment of the present disclosure.

FIG. 12 shows a synthesis scheme 11 for AV-D077, according to an embodiment of the present disclosure.

FIG. 13 shows a synthesis scheme 12 for AV-D085, according to an embodiment of the present disclosure.

FIGS. 14A-I show the chemical structures of exemplary linker compounds, Linker 1, Linker 2, Linker 3, Linker 4, Linker 5, Linker 6, Linker 7, Linker 8, and Linker 9, respectively, (exemplary compounds of Formula L), with “P” representing a payload or a point of attachment, depending on context, according to an embodiment of the present disclosure. Arrows indicate possible points of attachment between reactive moiety and, e.g., a targeting polypeptide.

FIG. 15A-G depicts chemical structures of exemplary compounds of Formula IV, according to an embodiment of the present disclosure.

FIG. 16A-C depict chemical structures of linkers of this disclosure attached to payloads according to an embodiment of the present disclosure.

FIG. 17 shows a synthesis scheme 14 for AV-D038, according to an embodiment of the present disclosure.

FIG. 18A shows a synthesis scheme 15a for AV-DL039, according to an embodiment of the present disclosure.

FIG. 18B shows a synthesis scheme 15b for AV-DL039, according to an embodiment of the present disclosure.

FIG. 19 shows a synthesis scheme 16 for AV-DL051, according to an embodiment of the present disclosure.

FIG. 20 shows a synthesis scheme 17 for AV-DL055, according to an embodiment of the present disclosure.

FIG. 21 shows a synthesis scheme 18 for AV-DL094, according to an embodiment of the present disclosure.

FIG. 22 shows a synthesis scheme 19 for AV-D095, according to an embodiment of the present disclosure.

FIG. 23 shows a synthesis scheme 20 for AV-DL096, according to an embodiment of the present disclosure.

FIG. 24 shows a synthesis scheme 21 for AV-D103, according to an embodiment of the present disclosure.

FIG. 25 shows a synthesis scheme 22 for AV-DL054, according to an embodiment of the present disclosure.

FIG. 26 shows a synthesis scheme 23 for AV-D097, according to an embodiment of the present disclosure.

FIG. 27 shows a synthesis scheme 24 for AV-D104, according to an embodiment of the present disclosure.

FIG. 28 shows a synthesis scheme 25 for AV-D105, according to an embodiment of the present disclosure.

FIG. 29 shows a synthesis scheme 26 for AV-DL100, according to an embodiment of the present disclosure.

FIG. 30 shows a synthesis scheme 27 for AV-DL102, according to an embodiment of the present disclosure.

FIGS. 31A-C show exemplary fluorocamptothecin compounds of Formula VIII, namely, AV-DL160 (31A), AV-DL161 (31B), and AV-DL123 (31C).

FIG. 32 shows a synthesis scheme 29 for AV-DL111, according to an embodiment of the present disclosure.

FIG. 33 shows a synthesis scheme 30 for AV-DL112, according to an embodiment of the present disclosure.

FIG. 34 shows a synthesis scheme 31 for AV-DL117, according to an embodiment of the present disclosure.

FIGS. 35A and 35B show exemplary fluorocamptothecin compounds of Formula VII, namely, AV-D104 (35A) and AV-D105 (35B).

FIG. 36A-E depicts chemical structures of exemplary compounds of Formula VIII, namely, AV-DL100, AV-DL102, AV-DL111, AV-DL112, and AV-DL117.

FIG. 37 shows potency of various payloads against cell lines SKBR3, JIMT1, and SKOV3.

FIG. 38 shows the potency of ADCs with various linker-payloads on SKBr Cell line. All ADCs are with DAR8 conjugated to trastuzumab (P004).

FIGS. 39A-39B show that ADCs comprising trastuzumab (P004) conjugated to AV-DL055, AV-DL111 and AV-DL112 showed excellent hydrophilic properties compared to Enhertu® (fam-trastuzumab-deruxtecan-nxki)(T-DXd, P005-DXd) when analyzed by SEC (size exclusion chromatography) and HIC (hydrophobic interaction chromatography).

FIGS. 40A-40B show that ADCs comprising P004 conjugated to AV-DL055, AV-DL111 and AV-DL112 demonstrate excellent serum stability compared to Enhurtu® (T-DXd; P004-DXd) on day 0 (D0), day 3 (D3), day 7 (D7), and day 14 (D14).

FIG. 41 shows a synthesis scheme 31 for AV-DL123, according to an embodiment of the present disclosure.

FIG. 42 shows exemplary compound AV-DL125.

FIG. 43 shows a synthesis scheme 32 for AV-DL125, according to an embodiment of the present disclosure.

FIG. 44 shows exemplary compound AV-DL126.

FIG. 45 shows a synthesis scheme 33 for AV-DL126, according to an embodiment of the present disclosure.

FIG. 46 shows exemplary compound AV-DL149.

FIG. 47 shows a synthesis scheme 34 for AV-DL149, according to an embodiment of the present disclosure.

FIG. 48 shows a synthesis scheme 35 for AV-DL160, according to an embodiment of the present disclosure.

FIG. 49 shows a synthesis scheme 36 for AV-DL161, according to an embodiment of the present disclosure.

FIG. 50 shows exemplary compound AV-DL122.

FIG. 51 shows a synthesis scheme 37 for AV-DL122, according to an embodiment of the present disclosure.

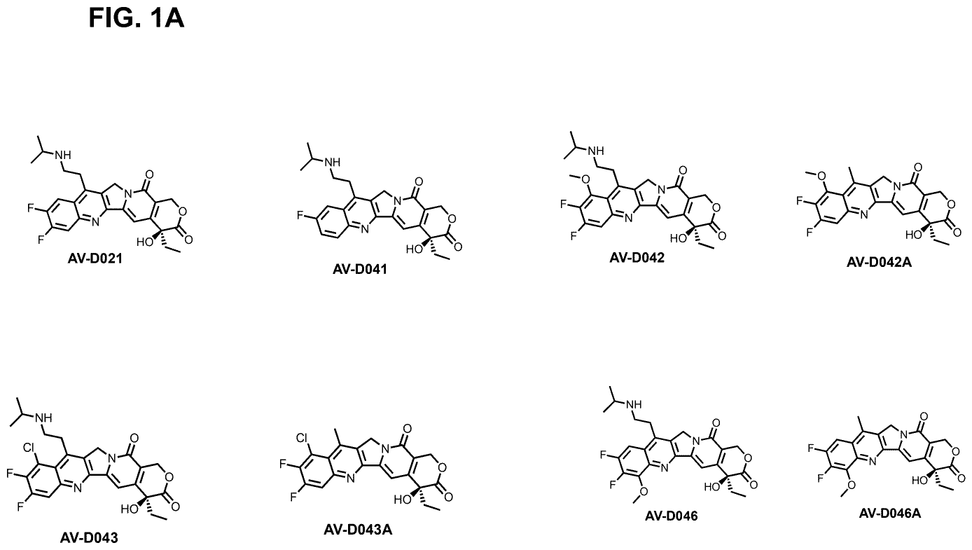

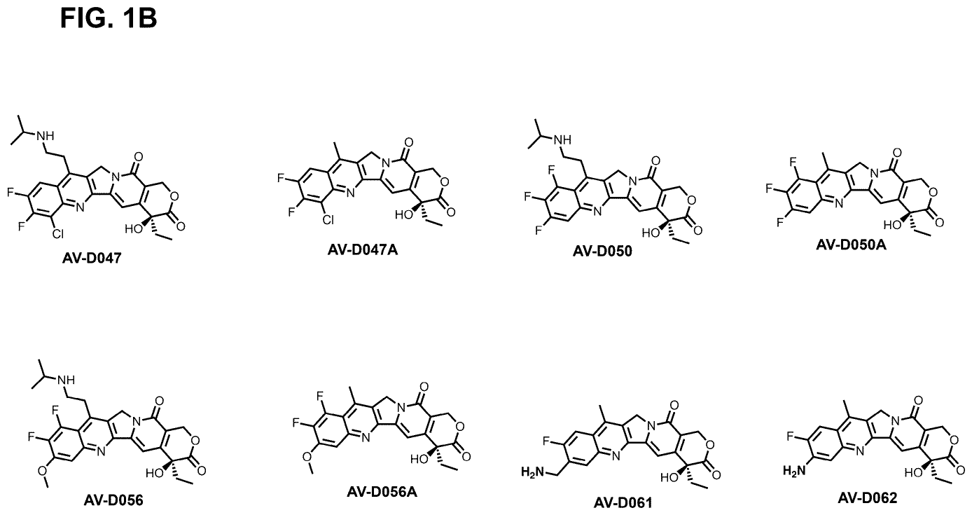

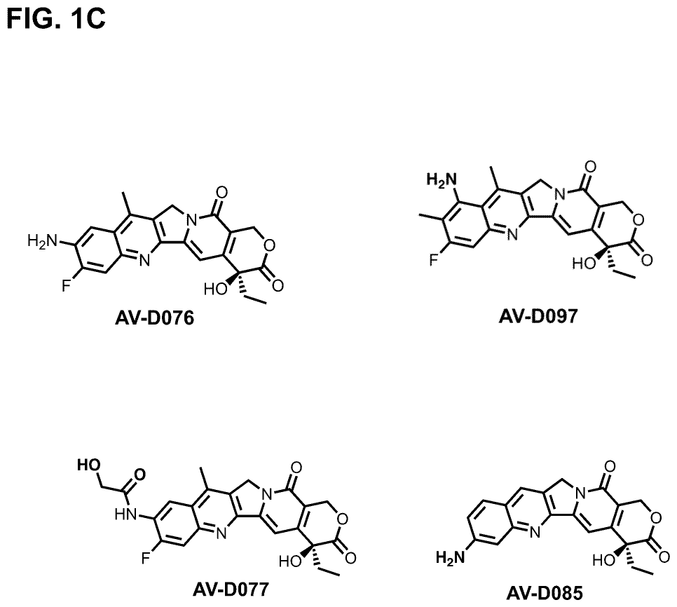

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### I. Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below. Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” used herein refers to one or more and thus includes individual components as well as mixtures/combinations.

The term “alkyl” as used herein, refers to a straight chain or branched saturated hydrocarbon group containing the specified number of carbon atoms in a range of 1 to 6. Examples of alkyl include, but are not limited to, methyl, ethyl, n-propyl, isopropyl, n-butyl, isobutyl, t-butyl, pentyl, neo-pentyl, 1-methylbutyl, 2-methylbutyl, n-hexyl, and the like. The term “alkyl” includes primary, secondary and tertiary alkyl having one to six carbon atoms.

The term “alkoxy” refers to a straight chain or branched saturated hydrocarbon group containing the specified number of carbon atoms in a range of 1 to 6, attached to an oxygen atom at the end such that the alkoxy group when attached to a moiety is linked through the oxygen atom. Examples of alkoxy include, but are not limited to, methoxy, ethoxy, propyloxy, isopropopyloxy, butoxy, sec-butoxy, isobutoxy, pentoxy, and the like.

The term “carboximidoyl” refers to the group having a carbonyl (CO) attached to amino or aminoalkyl, wherein the point of attachment of carboximidoyl could be either nitrogen or carbon.

The term “urethanyl” refers to the group —O—C(O)—N—, wherein the point of attachment of urethanyl with the rest of the molecule could be either nitrogen or oxygen.

The term “aminoalkyl,” as used herein, refers to an alkyl group as defined herein with one or more amino groups, for example, one, two or three amino groups. For example, C₁₋₆ aminoalkyl refers to an alkyl group having from 1 to 6 carbon atoms attached via amino linkage to the rest of the molecule. Preferred alkylamino groups include, without limitation, —NHCH₃, —N(CH₃)₂, —NHCH₂CH₃, —NHCH(CH₃)₂ and the like. The alkylamino group is optionally substituted.

The term “carboxy,” as used herein, refers to —C(O)O group.

The term “alkylcarboxy” refers to an alkyl group as defined herein substituted with at least one carboxy group. Examples of alkylcarboxy include methylcarboxy, ethylcarboxyl, propyl dicarboxy and the like.

The term “haloalkyl,” as used herein, refers to an alkyl group as defined herein substituted with one or more halogen atoms. Examples of haloalkyl include, but are not limited to trifluoromethyl, difluoromethyl, fluoromethyl, trichloromethyl, dichloromethyl, chloromethyl, and the like.

The terms “halogen” and “halo,” as used herein refer to fluorine (F), bromine (Br), chlorine (Cl) and iodine (I).

The term “cyano,” as used herein, refers to the group-CN.

The terms “hydroxy” and “hydroxyl,” as used herein, refer to the group —OH.

The term “oxo” refers to the ═O group.

The term “amino” refer to —NH₂ group, wherein one or both hydrogen atoms may be optionally replaced with other groups such as cyclic or acyclic hydrocarbons.

The term “alkylamine,” as used herein, refers to C₁₋₆ alkyl bearing a terminal unsubstituted or substituted amino group. Preferred alkylamine groups include, without limitation, —CH₂NH₂, —(CH₂)₂NH₂, —(CH₂)₃NH₂, —CH₂NHCH₃, —(CH₂)₂NHCH₃, —(CH₂)₃NHCH₃, —CH₂NHCH₂CH₃, —CH₂NH(CH₂)₂CH₃, —CH₂NH(CH₂)₃CH₃, and the like.

The term “cyclic” as used herein, refers to a mono- or bi- or poly-cyclic ring system containing the specified number of carbon atoms and in which one or more carbon atoms could be optionally substituted with a heteroatom, for example, O, S or N. A heterocyclyl substituent can be attached via any of its available ring atoms, for example, a ring carbon, or a ring heteroatom. Examples of cyclic groups include, cycloalkyl such as cyclopropyl, cyclobutyl, cyclopentyl, cyclohexyl and methylcyclopentane; heterocyclyl such as pyrazine, imidazole, thiazole, oxazole, triazole, and tetrazole; aryl such as benzene, anthracene, naphthalene and azulene; and heteroaryl such as pyridine, pyrrole, furan, and thiophene.

The term “aryl” as used herein, refers to a mono- or bicyclic fused or bridged hydrocarbon ring system in which at least one ring is aromatic. Examples of aryl include, but are not limited to, phenyl, naphthalenyl, 1,2,3,4-tetrahydro-naphthalenyl, 5,6,7,8-tetrahydro-naphthalenyl, indanyl, and the like.

The term “alkoxyaryl” as used herein refers to an alkoxy group as defined herein attached with an aryl group as defined herein, which may be optionally substituted.

The term “aralkyl” as used herein refers to aryl group as defined herein attached with an alkyl group as defined herein, which may be optionally substituted.

The term “alkoxyaralkyl” as used herein refers to an alkoxy group as defined herein attached with an aralkyl group as defined herein, which may be optionally substituted.

The term “heteroaryl,” or “heteroaromatic” as used herein, refers to mono- or bicyclic ring system in which at least one ring atom is a heteroatom and at least one ring is aromatic. Examples of heteroatoms include, but are not limited to, O, S and N. Examples of heteroaryl or heteroaromatic include, but are not limited to: pyridyl, benzofuranyl, pyrazinyl, pyridazinyl, pyrimidinyl, triazinyl, quinolinyl, benzoxazolyl, benzothiazolyl, isoquinolinyl, quinazolinyl, quinoxalinyl, pyrrolyl, indolyl, and the like.

Unless specifically stated as being “unsubstituted,” any alkyl, cycloalkyl, heterocycloalkyl, aryl or heteroaryl group referred to herein is understood to be “optionally substituted,” i.e. each such reference includes both unsubstituted and substituted versions of these groups. For example, reference to a “—C₁-C₆ alkyl” includes both unsubstituted-C₁-C₆ alkyl and —C₁-C₆ alkyl substituted with one or more substituents. Examples of substituents include, but are not limited to, halogen, alkoxy, carboxy, hydroxy, amino, amido, nitro, cyano, azido, alkylthio, alkyl, cycloalkyl, heterocycloalkyl, aryl or heteroaryl. In certain embodiments, each alkyl, cycloalkyl, heterocycloalkyl, aryl or heteroaryl group referred to herein is optionally substituted with one or more substituents selected from: halogen, alkoxy, carboxy, hydroxy, amino, amido, nitro, cyano, and azido.

A chemical group described herein that is “substituted” may include one substituent or a plurality of substituents up to the full valence of substitution for that group. For example, a methyl group may include 1, 2, or 3 substituents, and a phenyl group may include 1, 2, 3, 4, or 5 substituents. When a group is substituted with more than one substituent, the substituents may be the same or they may be different.

The term “amino acid” refers to an organic molecule comprising an amino group and a carboxyl group. Amino acids include the twenty naturally occurring amino acids, as well as unnatural amino acids such as alpha-aminoadipic acid, and 3-amino propionic acid.

The term “linker” refers to a moiety capable of linking two separate moieties to result in a single compound. For example, a bifunctional (or monovalent) linker, L, can link a single compound to a single site on targeting moiety whereas a multifunctional (or polyvalent) linker, L, can link more than one compound to a single site on targeting moiety. A linker that links one compound to more than one site on a targeting moiety may also be considered to be multifunctional in certain embodiments. In certain embodiments, the targeting moiety comprises an antibody. Accordingly, a linker can link a camptothecin analog of this disclosure to an antibody, to form an immunoconjugate.

The term “cleavage site” refers to the site or position of a compound or moiety where it is easily cleavable as a result of a reaction with a reagent.

The term “self-immolative group” refers to a moiety or group which undergoes cyclization, or elimination as a result of which the compound comprising the self-immolative group will attach a target site and thereby impart the desired effect. Self-immolative and self-elimination groups that find use in linkers include, for example, p-aminobenzyl (PAB) and p-aminobenzyloxycarbonyl (PABC) groups, and methylated ethylene diamine (MED). Other examples of self-immolative groups include, but are not limited to, 2-aminoimidazol-5-methanol derivatives, groups that undergo cyclization upon amide bond hydrolysis, such as substituted and unsubstituted 4-aminobutyric acid amides and 2-aminophenylpropionic acid amides. Self-immolative groups, alone or in combination are often included in peptide-based linkers but may also be included in other types of linkers.

Ther term “cytotoxic compound” as used herein refers to a compound capable of killing a cell or impairing cell function, such as disrupting cell division, inducing apoptosis, interfering with cellular metabolism, or inhibiting DNA synthesis or repair.

The term “antibody” as used herein refers to a protein or protein complex comprising a framework region from an immunoglobulin gene, which protein or protein complex binds to a target epitope. The term “antibody” includes the intact tetrameric antibody, which includes two heavy immunoglobulin chains and two light immunoglobulin chains. The term also includes any binding fragment of a tetrameric antibody. The term also includes engineered antibodies in which various immunoglobulin domains are rearranged, or are added to immunoglobulin chains, for example, to create antibodies with more than two binding sites.

The terms “subject” and “patient” as used herein refer to an animal in need of treatment. An animal in need of treatment may be a human or a non-human animal, such as a mammal, bird or fish. In certain embodiments, the subject or patient is a mammal. In some embodiments, the subject or patient is a human.

The term “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically acceptable excipient(s)/carrier(s) utilized, the route of administration, and like factors within the knowledge and expertise of the attending physician. For example, an “effective amount” of a compound when referred to in respect of the killing of cancer cells, refers to an amount of compound sufficient to produce a killing effect.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In an aspect of the present disclosure, there is provided a pharmaceutical composition comprising a compound of Formula I as disclosed herein, or a compound of Formula IV as disclosed herein, or a conjugate as disclosed herein, and a pharmaceutically acceptable excipient.

The term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free base form with a suitable acid. The pharmaceutically acceptable salt selected derived from inorganic bases such as like Li, Na, K, Ca, Mg, Fe, Cu, Zn and Mn; salts of organic bases such as N,N′-diacetylethylenediamine, glucamine, triethylamine, choline, dicyclohexylamine, benzylamine, trialkylamine, thiamine, guanidine, diethanolamine, «-phenylethylamine, piperidine, morpholine, pyridine, hydroxyethylpyrrolidine, hydroxyethylpiperidine, ammonium, substituted ammonium salts, aluminum salts and the like. Salts also include amino acid salts such as glycine, alanine, cystine, cysteine, lysine, arginine, phenylalanine, and guanidine. Salts may include acid addition salts where appropriate, which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates.

Salts and solvates having non-pharmaceutically acceptable counter-ions or associated solvents are within the scope of the present disclosure, for example, for use as intermediates in the preparation of other compounds of this disclosure, and their pharmaceutically acceptable salts. Thus, one embodiment of the disclosure embraces compounds of this disclosure and salts thereof. Compounds according to this disclosure can contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenyl acetate, propionate, butyrate, iso-butyrate, valerate, maleate, hydroxymaleate, acrylate, fumarate, malate, tartrate, citrate, salicylate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), aminobenzenesulfonate, p-toluenesulfonate (tosylate), and naphthalene-2-sulfonate.

The term “derivative” of a compound used herein refers to a product obtained from the compound either by reaction with a reagent or as addition compound. The term “derivative” also included protected version of the compound obtained by protecting one or more of the groups present in the compound.

The term “pharmaceutical composition” refers to a mixture of substances suitable for administering to an individual that includes a pharmaceutical agent. In an aspect of the present disclosure, there is provided a pharmaceutical composition comprising a compound or a conjugate as disclosed herein, and a pharmaceutically acceptable excipient.

It is to be understood that the positive recitation of a feature in one embodiment, serves as a basis for excluding the feature in an alternative embodiment. In particular, where a list of options is presented for a given embodiment or claim, it is to be understood that one or more option may be deleted from the list and the shortened list may form an alternative embodiment, whether or not such an alternative embodiment is specifically referred to.

### II. Camptothecin Compounds

There is a need in the art to develop camptothecin compounds which are more compatible for employment as payloads in ADCs. In accordance, the present disclosure provides a set of fluorocamptothecins which are capable of being used as payloads in conjugate formations.

### A. Complexes of Formula I

Accordingly, in an embodiment of the present disclosure, there is provided a compound of Formula I,

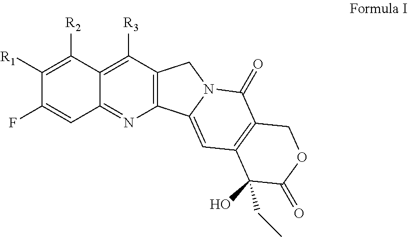

wherein, R₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₃ is selected from hydrogen, halogen, or C₁₋₆ alkyl, wherein C₁₋₆ alkyl is optionally substituted with C₁₋₆ aminoalkyl.

In an embodiment of the present disclosure, there is provided a compound of Formula I according to any previous embodiment, wherein,
R₁ is selected from hydrogen, halogen, C₁₋₃ alkyl, C₁₋₃ alkoxy, C₁₋₃ aminoalkyl, or NHR₄, wherein R₄ is selected from hydrogen, C₁₋₃ alkyl, C₁₋₃ haloalkyl, carboxy, or —COR₅; R₅ is selected from hydrogen, hydroxy, or C₁₋₃ alkyl, and wherein C₁₋₃ alkyl is optionally substituted with halogen or hydroxy;
R₂ is selected from hydrogen, halogen, C₁₋₃ alkyl, C₁₋₃ alkoxy, C₁₋₃ aminoalkyl, or NHR₄, wherein R₄ is selected from hydrogen, C₁₋₃ alkyl, C₁₋₃ haloalkyl, or —COR₅; R₅ is selected from hydrogen, hydroxy, or C₁₋₃ alkyl, and wherein C₁₋₃ alkyl is optionally substituted with halogen or hydroxy;
R₃ is selected from hydrogen, halogen or C₁₋₃ alkyl, wherein C₁₋₃ alkyl is optionally substituted with C₁₋₃ aminoalkyl.

In an embodiment of the present disclosure, there is provided a compound of Formula I according to any previous embodiment, wherein R₁ is selected from halogen, C₁₋₃ alkyl, or NHR₄, wherein R₄ is selected from hydrogen, C₁₋₃ alkyl, or —COR₅, wherein R₅ is C₁₋₂ alkyl substituted with hydroxy;
R₂ is selected from hydrogen, halogen, C₁₋₃ alkoxy or NHR₄, wherein R₄ is selected from hydrogen, C₁₋₃ alkyl, or —COR₅, wherein R₅ is C₁ alkyl substituted with hydroxy;
R₃ is selected from C₁₋₃ alkyl, wherein C₁₋₃ alkyl is optionally substituted with C₁₋₃ aminoalkyl.

In an embodiment of the present disclosure, there is provided a compound of Formula I according to any previous embodiment, wherein R₁ is selected from halogen, C₁₋₃ alkyl, or NHR₄, wherein R₄ is —COCH₂OH; R₂ is selected from hydrogen, halogen, C₁₋₂ alkoxy, or NHR₄, wherein R₄ is hydrogen or —COCH₂OH; and R₃ is selected from C₁₋₂ alkyl, or C₁₋₃ alkyl substituted with C₁₋₃ aminoalkyl.

In an embodiment of the present disclosure, there is provided a compound of Formula I according to any previous embodiment, wherein the compound is selected from the group consisting of:
a. (S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021);
b. (S)-4-ethyl-8,9-difluoro-4-hydroxy-11-(2-(isopropylamino)ethyl)-10-methoxy-1,12-dihydro-14H-pyrano[3′,4′:6,7]indolizino[1,2-b]quinoline-3,14(4H)-dione (AV-D042);
c. (S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043);
d. (S)—N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)-2-hydroxyacetamide (AV-D077); and
e. (S)-10-amino-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D097).

In an embodiment of the present disclosure, there is provided a compound of Formula I as disclosed herein, R₁ is selected from halogen, or NHR₄, wherein R₄ is —COCH₂OH; R₂ is selected from hydrogen, halogen, or C₁ alkoxy and NHCOCH₂OH; and R₃ is selected from C₁₋₂ alkyl, or —(C₁₋₃ alkyl)-NH—(C₁₋₃ alkyl).

In an embodiment of the present disclosure, there is provided a compound of Formula I as disclosed herein, wherein R₁ is selected from fluorine, chlorine, bromine, iodine, C₁₋₂ alkyl or —NHCOCH₂OH; R₂ is selected from hydrogen, halogen, NH₂, methoxy, or amino carboxy hydroxylmethyl; and R₃ is selected from methyl, or ethyl isopropylamino.

In an embodiment of the present disclosure, there is provided a compound of Formula I as disclosed herein, wherein R₁ is selected from fluorine, methyl, or —NHCOCH₂OH; R₂ is selected from hydrogen, fluorine, chlorine, methoxy or NH₂; and R₃ is selected from methyl and ethyl isopropylamino.

In an embodiment of the present disclosure, there is provided a compound of Formula I as disclosed herein, wherein, R₁ is selected from H, F, methyl, and amino carboxy hydroxylmethyl; R₂ is selected from hydrogen, halogen, methoxy; and amino carboxy hydroxylmethyl; R₃ is selected from methyl and ethyl isopropylamino.

In an embodiment of the present disclosure, there is provided a compound of Formula I as disclosed herein, wherein R₁ is selected from H, F, methyl, and amino carboxy hydroxylmethyl,

In an embodiment of the present disclosure, there is provided a compound of Formula I as disclosed herein, wherein R₂ is selected from hydrogen, halogen, NH₂, amino carboxy hydroxylmethyl and methoxy.

In an embodiment of the present disclosure, there is provided a compound of Formula I as disclosed herein, wherein R₃ is selected from methyl and ethyl isopropylamino.

In an embodiment of the present disclosure, there is provided a compound of Formula I according to any previous embodiment, wherein the compound is selected from the group consisting of:
a. (S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021);
b. (S)-4-ethyl-9-fluoro-4-hydroxy-11-(2-(isopropylamino)ethyl)-1,12-dihydro-14H-pyrano[3′,4′:6,7]indolizino[1,2-b]quinoline-3,14(4H)-dione (AV-D041);
c. (S)-4-ethyl-8,9-difluoro-4-hydroxy-10-methoxy-11-methyl-1,12-dihydro-14H-pyrano[3′,4′:6,7]indolizino[1,2-b]quinoline-3,14(4H)-dione (AV-D042A);
d. (S)-4-ethyl-8,9-difluoro-4-hydroxy-11-(2-(isopropylamino)ethyl)-10-methoxy-1,12-dihydro-14H-pyrano[3′,4′:6,7]indolizino[1,2-b]quinoline-3,14(4H)-dione (AV-D042);
e. (S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043);
f. (S)-10-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D043A);
g. (S)-4-ethyl-8,9-difluoro-4-hydroxy-7-methoxy-11-methyl-1,12-dihydro-14H-pyrano[3′,4′:6,7]indolizino[1,2-b]quinoline-3,14(4H)-dione (AV-D046A);
h. (S)-4-ethyl-8,9-difluoro-4-hydroxy-11-(2-(isopropylamino)ethyl)-7-methoxy-1,12-dihydro-14H-pyrano[3′,4′:6,7]indolizino[1,2-b]quinoline-3,14(4H)-dione (AV-D046);
i. (S)-7-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D047A);
j. (S)-7-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D047);
k. (S)-4-ethyl-8,9,10-trifluoro-4-hydroxy-11-methyl-1,12-dihydro-14H-pyrano[3′,4′:6,7]indolizino[1,2-b]quinoline-3,14(4H)-dione (AV-D050A);
l. (S)-4-ethyl-8,9,10-trifluoro-4-hydroxy-11-(2-(isopropylamino)ethyl)-1,12-dihydro-14H-pyrano[3′,4′:6,7]indolizino[1,2-b]quinoline-3,14(4H)-dione (AV-D050);
m. (S)-4-ethyl-9,10-difluoro-4-hydroxy-8-methoxy-11-methyl-1,12-dihydro-14H-pyrano[3′,4′:6,7]indolizino[1,2-b]quinoline-3,14(4H)-dione (AV-D056A);
n. (S)-4-ethyl-9,10-difluoro-4-hydroxy-11-(2-(isopropylamino)ethyl)-8-methoxy-1,12-dihydro-14H-pyrano[3′,4′:6,7]indolizino[1,2-b]quinoline-3,14(4H)-dione (AV-D056);
o. (S)-8-(aminomethyl)-4-ethyl-9-fluoro-4-hydroxy-1,12-dihydro-14H-pyrano[3′,4′:6,7]indolizino[1,2-b]quinoline-3,14(4H)-dione (AV-D061);
p. (S)-8-amino-4-ethyl-9-fluoro-4-hydroxy-1,12-dihydro-14H-pyrano[3′,4′:6,7]indolizino[1,2-b]quinoline-3,14(4H)-dione (AV-D062);
q. (S)—N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)-2-hydroxyacetamide (AV-D077);
r. (S)-8-amino-4-ethyl-4-hydroxy-1,12-dihydro-14H-pyrano[3′,4′:6,7]indolizino[1,2-b]quinoline-3,14(4H)-dione (AV-D085); and
s. (S)-10-amino-4-ethyl-8-fluoro-4-hydroxy-9,11-dimethyl-1,12-dihydro-14H-pyrano[3′,4′:6,7]indolizino[1,2-b]quinoline-3, 14 (4H)-dione (AV-D097).

### B. Complexes of Formula VII

In an embodiment of the present disclosure, there is provided a compound of Formula VII

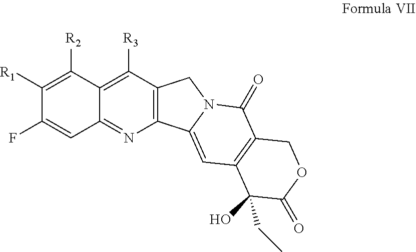

wherein, R₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is C₁₋₆ alkylamine, optionally wherein N is alkylated with C₁₋₆ alkyl;
R₃ is selected from hydrogen, halogen, or C₁₋₆ alkyl, wherein C₁₋₆ alkyl is optionally substituted with C₁₋₆ aminoalkyl.

In an embodiment of the present disclosure, there is provided a compound of Formula VII according to any previous embodiment, wherein,
R₁ is C₁₋₃ alkyl;
R₂ is propylamine, optionally wherein N is alkylated with C₁₋₃ alkyl;
R₃ is C₁₋₃ alkyl.

In an embodiment of the present disclosure, there is provided a compound of Formula VII according to any previous embodiment, wherein
R₁ is methyl;
R₂ is propylamine, optionally wherein Nis alkylated with C₁₋₃ alkyl;
R₃ is methyl.

In an embodiment of the present disclosure, there is provided a compound of Formula VII according to any previous embodiment, wherein the compound is selected from the group consisting of:
(S)-10-(3-aminopropyl)-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D104); and
(S)-4-ethyl-8-fluoro-4-hydroxy-9,11-dimethyl-10-(3-(methylamino)propyl)-1,12-dihydro-14H-pyrano[3′,4′:6,7]indolizino[1,2-b]quinoline-3,14(4H)-dione (AV-D105).

### III. Linker-Camptothecin Compounds

In an embodiment of the present disclosure, there is provided a compound of Formula IV or Formula VII as disclosed herein, or otherwise, wherein the compound is selected from the group consisting of:

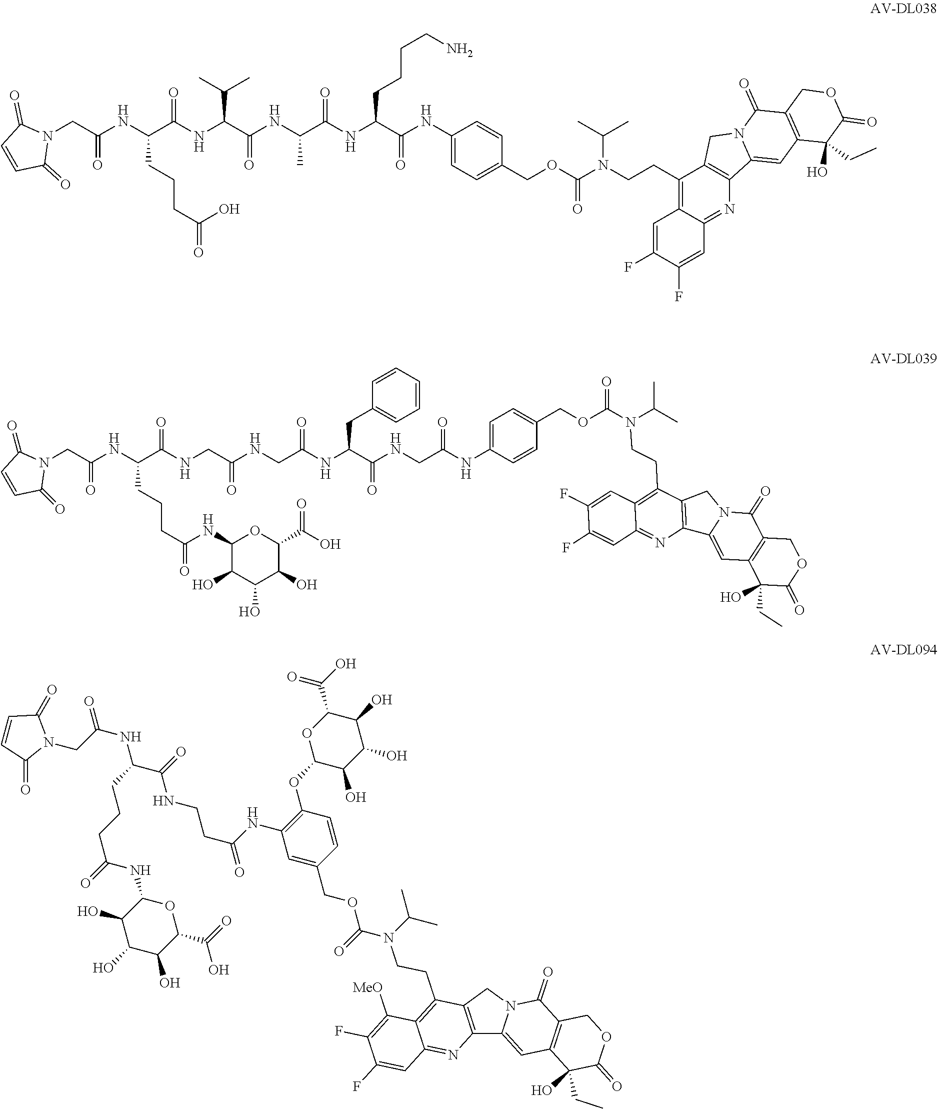

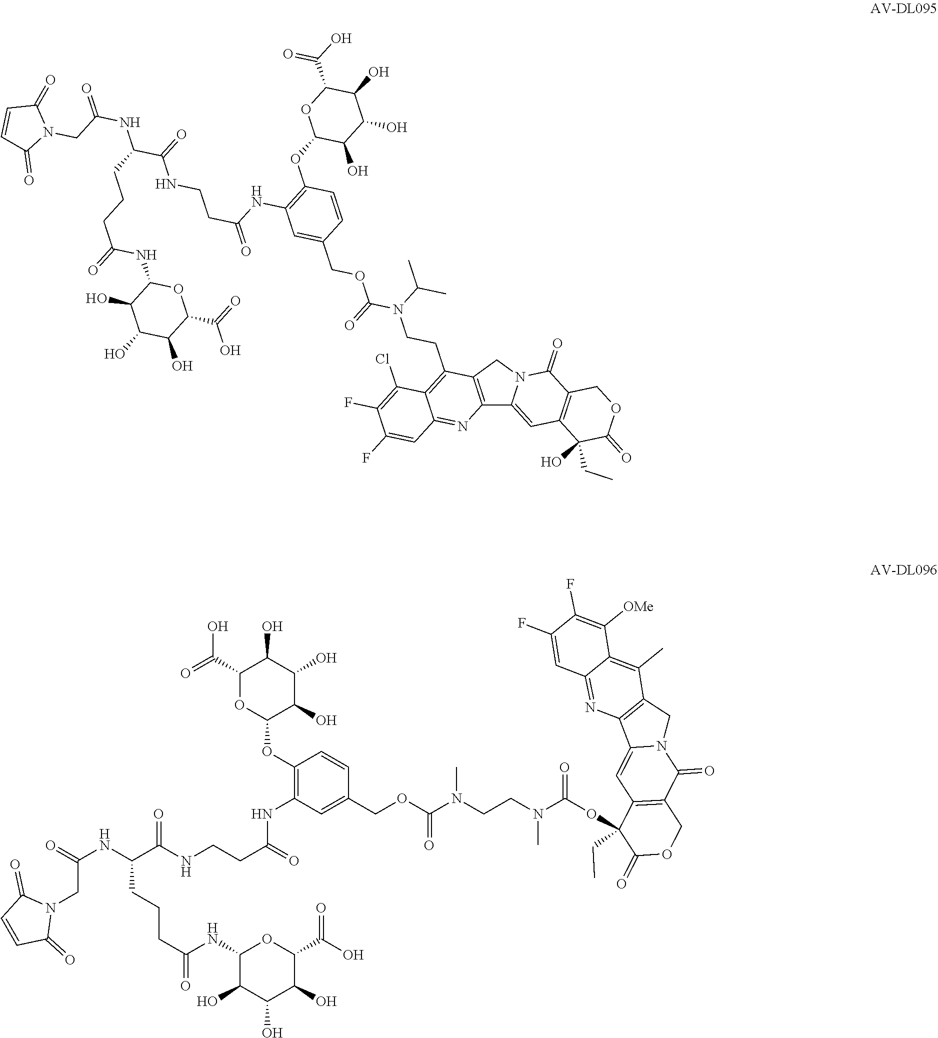 [SMILES: CC[C@@]1(O)C(=O)OCc2c1cc1n(c2=O)Cc2c-1nc1cc(F)c(F)c(Cl)c1c2CCN(C(=O)OCc1ccc(O[C@@H]2O[C@H](C(=O)O)[C@@H](O)[C@H](O)[C@H]2O)c(NC(=O)CCNC(=O)[C@H](CCCC(=O)N[C@@H]2O[C@H](C(=O)O)[C@@H](O)[C@H](O)[C@H]2O)NC(=O)CN2C(=O)C=CC2=O)c1)C(C)C ; CC[C@@]1(OC(=O)N(C)CCN(C)C(=O)OCc2ccc(O[C@@H]3O[C@H](C(=O)O)[C@@H](O)[C@H](O)[C@H]3O)c(NC(=O)CCNC(=O)[C@H](CCCC(=O)N[C@@H]3O[C@H](C(=O)O)[C@@H](O)[C@H](O)[C@H]3O)NC(=O)CN3C(=O)C=CC3=O)c2)C(=O)OCc2c1cc1n(c2=O)Cc2c-1nc1cc(F)c(F)c(OC)c1c2C]

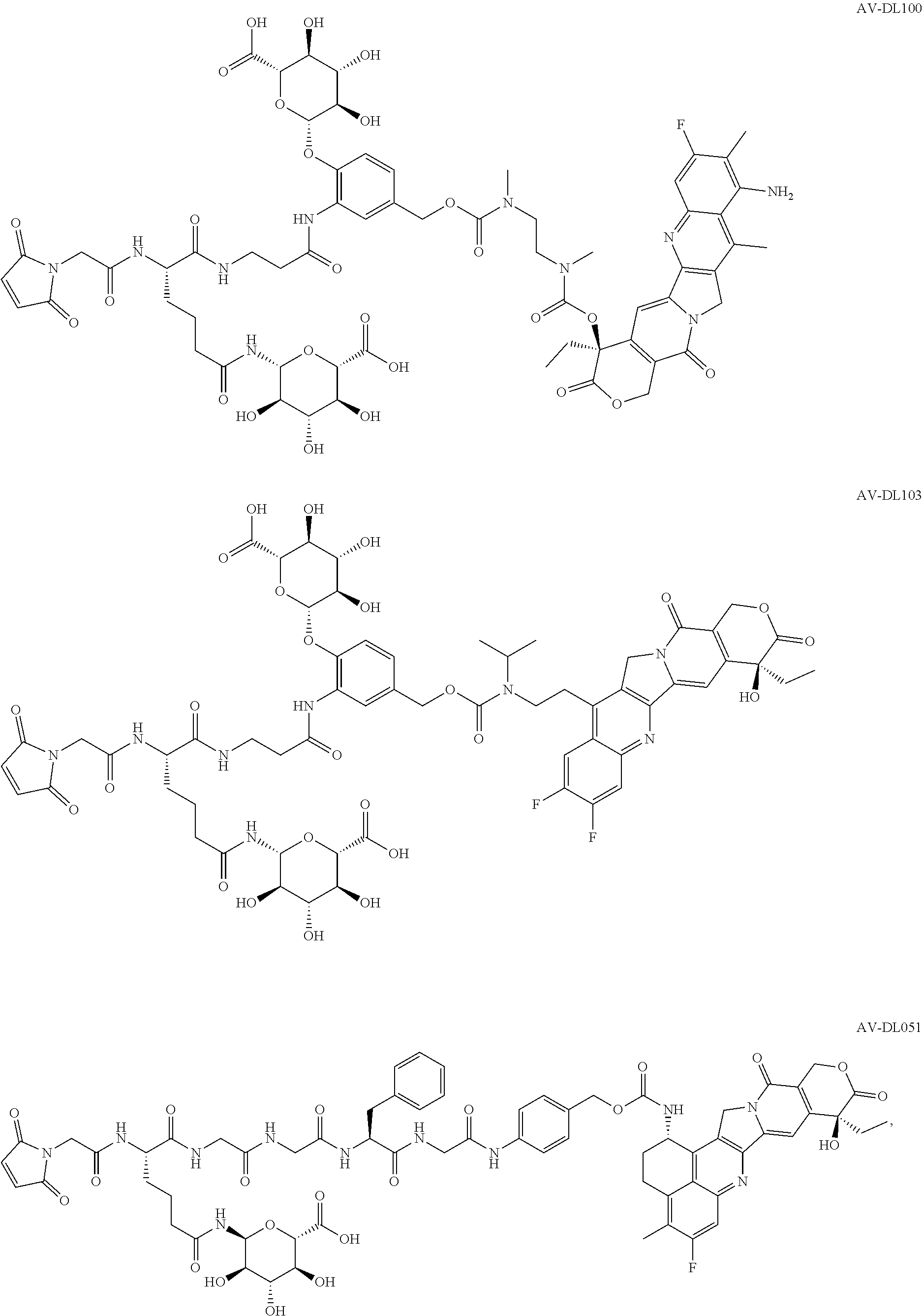

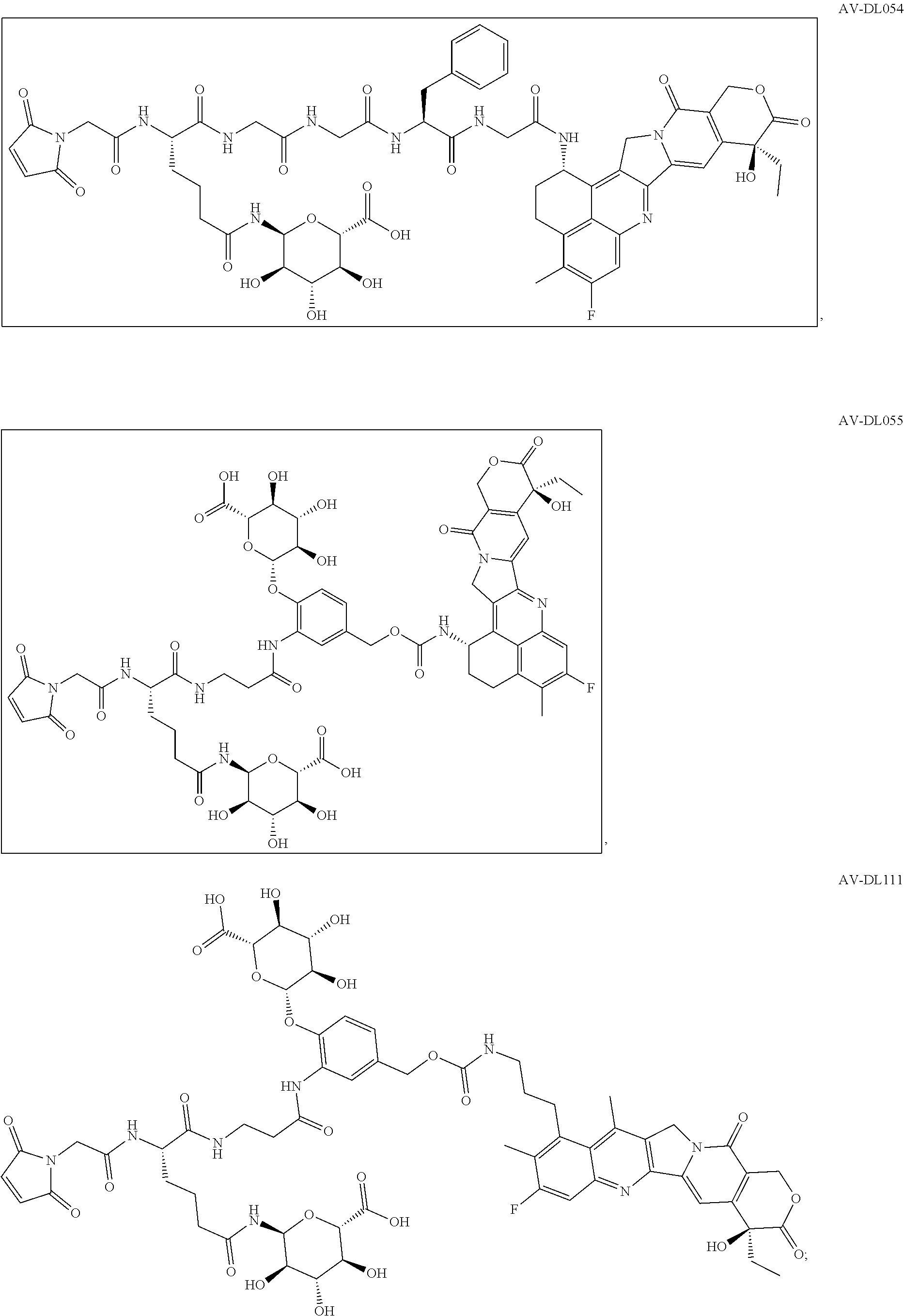

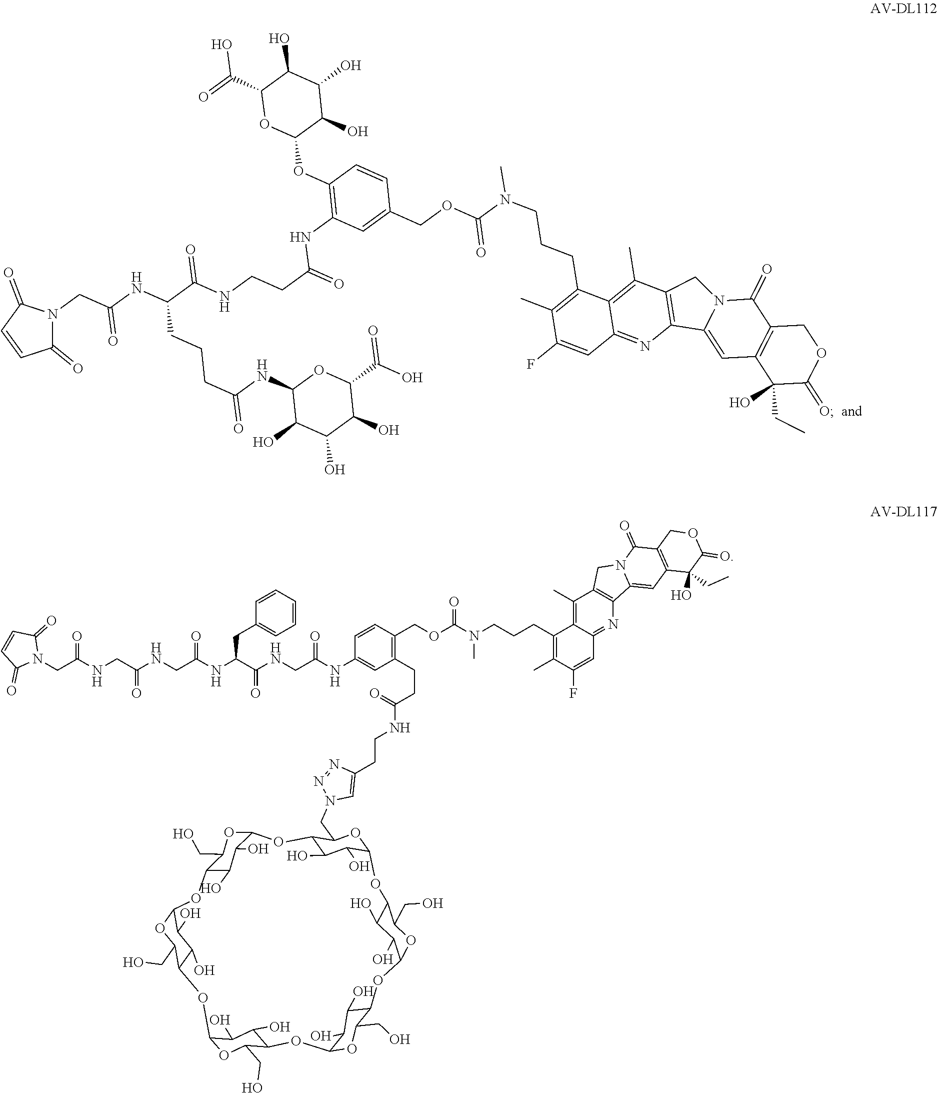

### IV. Processes for Preparing Compounds

In an embodiment of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I

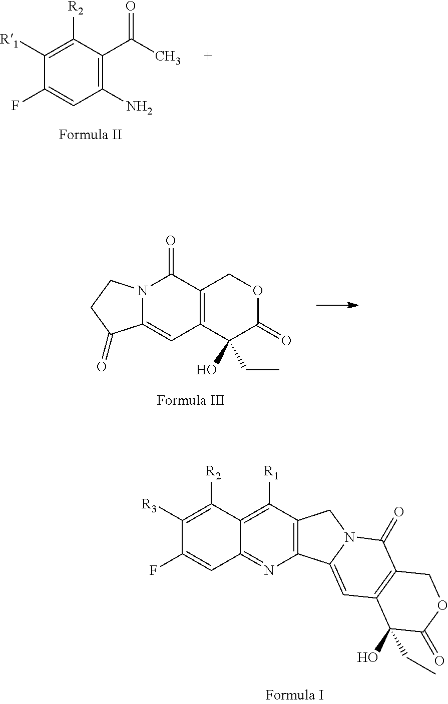

wherein, R′₁ is selected from hydrogen, hydroxy, halogen, C₁₋₆ alkyl, C₁₋₆ alkoxy, C₁₋₆ aminoalkyl, C₆₋₁₂ aryl, C₇₋₁₂ aralkyl, C₇₋₁₂ alkoxyaryl, C₈₋₁₂ alkoxyaralkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or — COR₅; R₅ is selected from hydrogen, hydroxy, or C₁₋₆ alkyl, and wherein C₁₋₆ alkyl is optionally substituted with halogen or hydroxy;
R₃ is selected from hydrogen, halogen, or C₁₋₆ alkyl, wherein C₁₋₆ alkyl is optionally substituted with C₁₋₆ aminoalkyl.

In an embodiment of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I, wherein R₁ is selected from hydrogen, halogen, C₁₋₃ alkyl, C₁₋₃ alkoxy, C₁₋₃ aminoalkyl, or NHR₄, wherein R₄ is selected from hydrogen, C₁₋₃ alkyl, C₁₋₃ haloalkyl, carboxy, or —COR₅; R₅ is selected from hydrogen, hydroxy, or C₁₋₃ alkyl, and wherein C₁₋₃ alkyl is optionally substituted with halogen or hydroxy; R₂ is selected from hydrogen, halogen, C₁₋₃ alkyl, C₁₋₃ alkoxy, C₁₋₃ aminoalkyl, or NHR₄, wherein R₄ is selected from hydrogen, C₁₋₃ alkyl, C₁₋₃ haloalkyl, or —COR₅; R₅ is selected from hydrogen, hydroxy, or C₁₋₃ alkyl, and wherein C₁₋₃ alkyl is optionally substituted with halogen or hydroxy; and R₃ is selected from hydrogen, halogen or C₁₋₃ alkyl, wherein C₁₋₃ alkyl is optionally substituted with C₁₋₃ aminoalkyl.

In an embodiment of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I, wherein R₁ is selected from halogen, C₁₋₃ alkyl, or NHR₄, wherein R₄ is selected from hydrogen, C₁₋₃ alkyl, or —COR₅, wherein R₅ is C₁₋₂ alkyl substituted with hydroxy; R₂ is selected from hydrogen, halogen, C₁₋₃ alkoxy or NHR₄, wherein R₄ is selected from H, C₁₋₃ alkyl, or —COR₅, wherein R₅ is C₁ alkyl substituted with hydroxy; and R₃ is selected from C₁₋₃ alkyl, wherein C₁₋₃ alkyl is optionally substituted with C₁₋₃ aminoalkyl.

In an embodiment of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I, wherein R₁ is selected from halogen, C₁₋₃ alkyl, or NHR₄, wherein R₄ is —COCH₂OH; R₂ is selected from hydrogen, halogen, C₁₋₂ alkoxy, or NHR₄, wherein R₄ is H or —COCH₂OH; and R₃ is selected from C₁₋₂ alkyl, or C₁₋₃ alkyl substituted with C₁₋₃ aminoalkyl.

In an embodiment of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I, wherein R₁ is selected from fluorine, chlorine, bromine, iodine, C₁₋₂ alkyl or —NHCOCH₂OH; R₂ is selected from hydrogen, halogen, NH₂, methoxy, or amino carboxy hydroxylmethyl; and R₃ is selected from methyl, or ethyl isopropylamino.

In an embodiment of the present disclosure, there is provided a process of preparing the compound of Formula I as disclosed herein, the process comprising: reacting a compound of Formula II with a compound of Formula III in the presence of a solvent and optionally processing with a reagent to obtain the compound of Formula I, wherein R₁ is selected from fluorine, methyl, or —NHCOCH₂OH; R₂ is selected from hydrogen, fluorine, chlorine, methoxy or NH₂; and R₃ is selected from methyl and ethyl isopropylamino.

In an embodiment of the present disclosure, there is provided a process of preparing the compound of Formula VII as disclosed herein, the process comprising: reacting a compound of Formula II with a compound of Formula III in the presence of a solvent and optionally processing with a reagent to obtain the compound of Formula VII

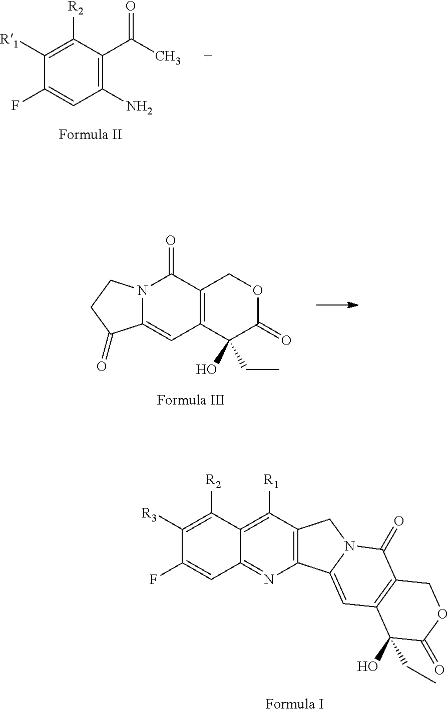

wherein, R′₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₁ is selected from hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy;
R₂ is C₁₋₆ alkylamine, optionally wherein N is alkylated with C₁₋₆ alkyl;
R₃ is selected from hydrogen, halogen, or C₁₋₆ alkyl, wherein C₁₋₆ alkyl is optionally substituted with C₁₋₆ aminoalkyl.

In an embodiment of the present disclosure, there is provided a process of preparing the compound of Formula VII as disclosed herein, the process comprising: reacting a compound of Formula II with a compound of Formula III in the presence of a solvent and optionally processing with a reagent to obtain the compound of Formula VII, wherein the compound of Formula VII is any embodiment of Formula VII disclosed herein.

In an embodiment of the present disclosure, there is provided a process of preparing the compound of Formula I or Formula VII as disclosed herein, wherein the solvent is selected from dimethyl sulphoxide, toluene, dichloromethane, dimethylformamide, methanol, or combinations thereof. In another embodiment of the present disclosure, the solvent is dimethyl sulphoxide. In yet another embodiment of the present disclosure, the solvent is toluene. In still another embodiment of the present disclosure, the solvent is dichloromethane. In more embodiments of the present disclosure, the solvent is dimethylformamide. In yet another embodiment of the present disclosure, the solvent is methanol.

In an embodiment of the present disclosure, there is provided a process of preparing a compound of Formula I or Formula VII according to any previous embodiment, wherein the reagent is selected from isopropyl amine, trifluoro acetic acid, 2-(benzyloxy) acetic acid, ethylenediamine chloride, or combinations thereof.

### V. Linkers

In certain embodiments, the cytotoxic compounds of this disclosure are attached to a linker, typically comprising a reactive moiety, or, through a linker, to a targeting moiety, such as an antibody.

In certain embodiments, linker “L” has 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties.

### In Certain Embodiments, Linker “L” has Formula L Given as,

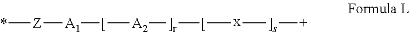

wherein:
* represents a binding site on a first compound, e.g., a reactive moiety or a targeting moiety;
—Z— is a linking group selected from C₁₋₁₀ acyl, propionyl, butyroyl, and carbonyl;
A1 and A2 are independently amino acid moieties, wherein A₁-[A₂], forms a cleavage site. Amino acids include, without limitation, alanine, lysine alpha-aminoadipic acid, valine and 3-amino propionic acid;
r is 1, 2, 3, 4 or 5;
X is self-immolative group;
s is 0, 1 or 2; and
+ represents a binding site on a second compound, such as a fluorocamptothecin compound disclosed herein.

Accordingly, when “*” is a reactive moiety, Rₓ, it is capable of reacting with the target group or groups on targeting moiety T. The group that attaches to the second compound can be a functional group capable of reacting with a target group on the second compound, e.g., on the fluorocamptothecin analogue. Suitable functional groups are known in the art and include those described, for example, in Bioconjugate Techniques (G. T. Hermanson, 2013, Academic Press). Self-immolative groups are typically attached to an amino or hydroxyl group on the compound, at R₁, R₂, R₃ or R₆.

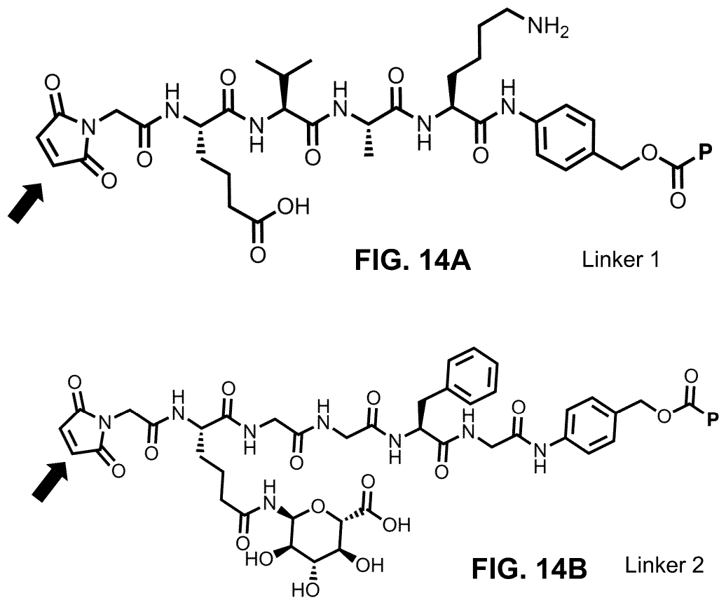

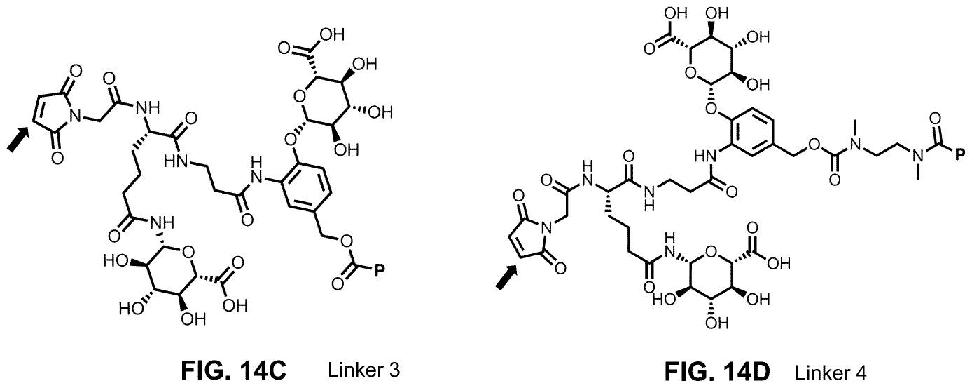

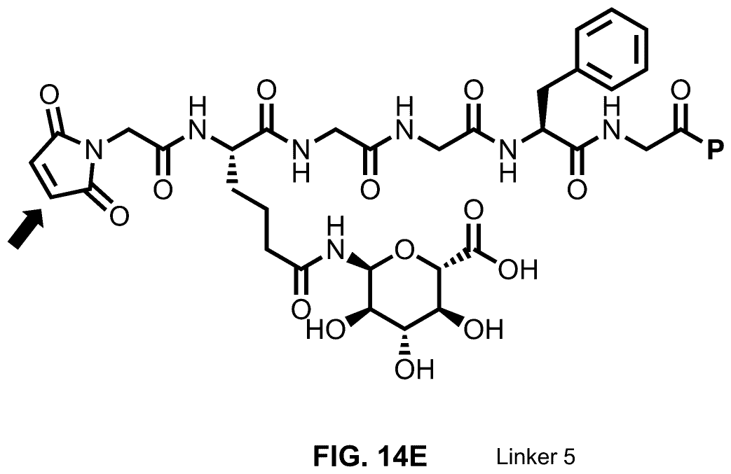

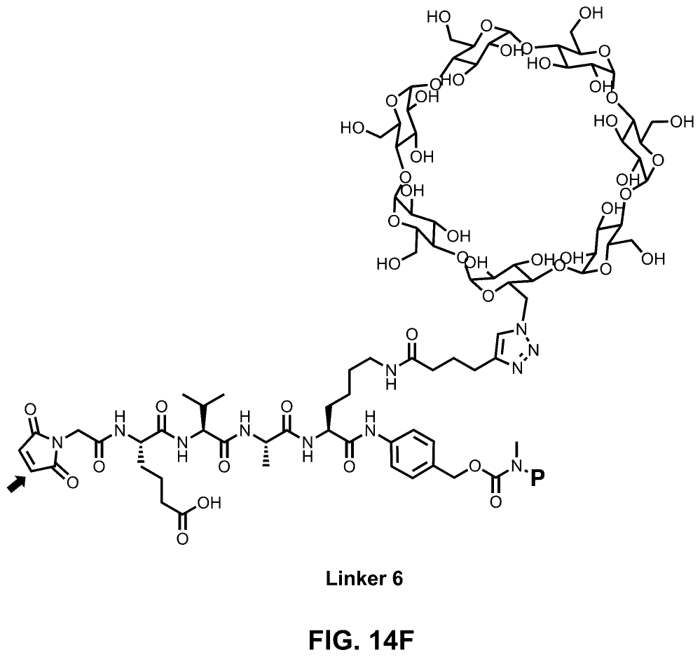

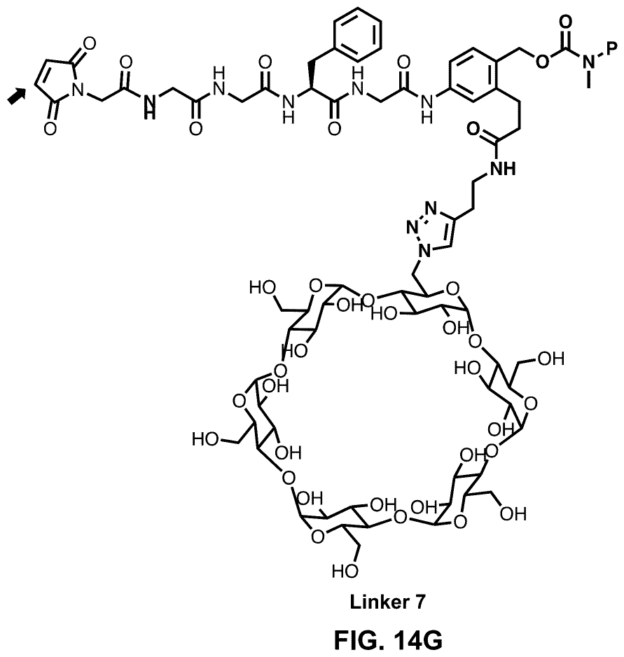

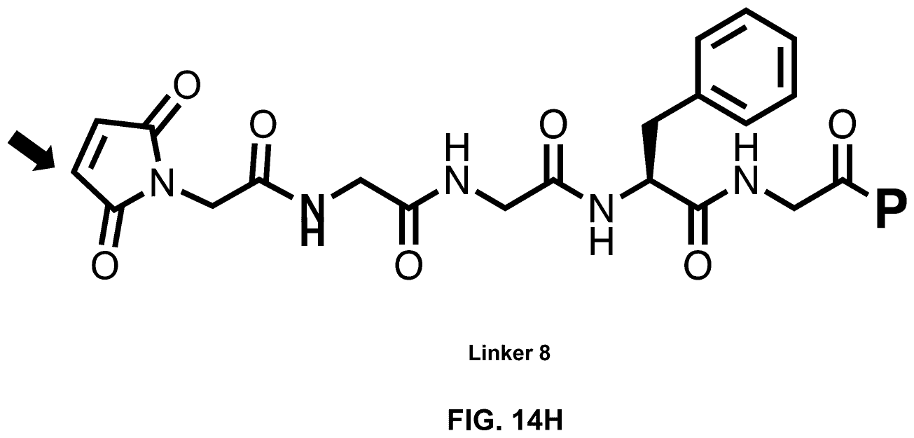

Exemplary linkers include Linker 1, Linker 2, Linker 3, Linker 4, Linker 5, Linker 6, Linker 7, Linker 8, and Linker 9, as depicted in FIGS. 14A-14I. In these structures, the arrow depicts a point of possible attachment to a targeting moiety. Depending on context, P is either (1) a compound to which the linker is attached, such as a toxic payload, e.g., a fluorocamptothecin compound of this disclosure, or (2) a binding site on the attached compound (as per “+” in Formula L, above). Accordingly, in the compounds of Formulae IV, V, VIII and IX, “P” represents the point of attachment to the fluorocamptothecin molecule. These compounds comprise moieties R₁ₐ, R₂ₐ, R₃ₐ, and R₆. R₁ₐ, R₂ₐ, and R₃ₐ can be -M-L-Rₓ (e.g., -M1-L-Rₓ, -M2-L-Rₓ, -M3-L-Rₓ, etc.) or -M-L- (e.g., -M1-L-, -M2-L-, -M3-L-, etc.). R₆ can be -L-Rₓ or -L-(attached through oxygen). Accordingly, in these contexts, P represents the point of attachment to the “M” moiety within the R groups, R₁ₐ, R₂ₐ, and R₃ₐ, or, in the case of R₆, the point of attachment to the oxygen atom. As used herein, this can be referred to simply as, “wherein P is the point of attachment.”

### VI. Drug-Linker Complexes

### a. Complexes of Formula IV

In an embodiment of the present disclosure, there is provided a compound of Formula IV, its a pharmaceutically acceptable salt or derivative thereof:

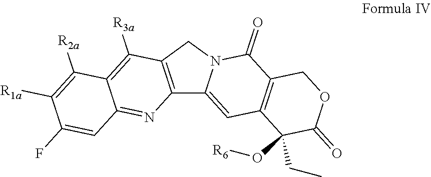

wherein, R₁ₐ is selected from hydrogen, hydroxy, halogen, C₁₋₆ alkyl, C₁₋₆ alkoxy, C₁₋₆ aminoalkyl, NHR₄ wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is a selected from hydroxy, C₁₋₆ alkoxy, C₁₋₆ aminoalkyl, and NHR₄;
R₂ₐ is selected from hydrogen, hydroxy, halogen, C₁₋₆ alkyl, C₁₋₆ alkoxy, C₁₋₆ aminoalkyl, NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M2-L-Rₓ, wherein M2 is selected from hydroxy, C₁₋₆ alkoxy, C₁₋₆ aminoalkyl, and NHR₄;
R₃ₐ is selected from hydrogen, halogen, or C₁₋₆ alkyl, wherein C₁₋₆ alkyl is optionally substituted with C₁₋₆ aminoalkyl, and -M3-L-Rₓ, wherein M3 is selected from a direct bond and C₁₋₆ alkyl substituted with C₁₋₆ aminoalkyl; and
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises -L-Rₓ. In certain embodiments, the -L-Rₓ; is attached through the R₃ₐ group.

In another embodiment of the present disclosure, there is provided a compound of Formula IV according to any previous embodiment, wherein
R₁ is selected from hydrogen, halogen, C₁₋₃ alkyl, C₁₋₃ alkoxy, C₁₋₃ aminoalkyl, NHR₄, wherein R₄ is selected from hydrogen, C₁₋₃ alkyl, C₁₋₃ haloalkyl, carboxy, or —COR₅; R₅ is selected from hydrogen, hydroxy, or C₁₋₃ alkyl, and wherein C₁₋₃ alkyl is optionally substituted with halogen or hydroxy, and -M4-L-Rₓ, wherein M4 is a selected from C₁₋₃ alkoxy, C₁₋₃ aminoalkyl, and NHR₄;
R₂ is selected from hydrogen, halogen, C₁₋₃ alkyl, C₁₋₃ alkoxy, C₁₋₃ aminoalkyl, NHR₄, wherein R₄ is selected from hydrogen, C₁₋₃ alkyl, C₁₋₃ haloalkyl, or —COR₅; R₅ is selected from hydrogen, hydroxy, or C₁₋₃ alkyl, and wherein C₁₋₃ alkyl is optionally substituted with halogen or hydroxy, and -M5-L-Rₓ, wherein M5 is a selected from C₁₋₃ alkoxy, C₁₋₃ aminoalkyl, and NHR₄;
R₃ is selected from hydrogen, halogen, C₁₋₃ alkyl, wherein C₁₋₃ alkyl is optionally substituted with C₁₋₃ aminoalkyl, and -M6-L-Rₓ, wherein M6 is a selected from a direct bond and C₁₋₃ alkyl substituted with C₁₋₃ aminoalkyl.
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises L-Rₓ.

In yet another embodiment of the present disclosure, there is provided a compound of Formula IV as disclosed herein, wherein
R₁ₐ is selected from halogen, C₁₋₃ alkyl, NHR₄, wherein R₄ is selected from hydrogen, C₁₋₃ alkyl, or —COR₅, wherein R₅ is C₁₋₂ alkyl substituted with hydroxy, and M7-L-Rₓ, wherein M7 is NHR₄;
R₂ₐ is selected from hydrogen, halogen, C₁₋₃ alkoxy, NHR₄, wherein R₄ is selected from hydrogen, C₁₋₃ alkyl, —COR₅, wherein R₅ is C₁ alkyl substituted with hydroxy, and M8-L-Rₓ, wherein M8 is selected from C₁₋₃ alkoxy, and NHR₄;
R₃ₐ is selected from C₁₋₃ alkyl wherein C₁₋₃ alkyl is optionally substituted with C₁₋₃ aminoalkyl, and M9-L-Rₓ, wherein M9 is selected from and C₁₋₃ alkyl substituted with C₁₋₃ aminoalkyl;
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ is L-Rₓ.

In still another embodiment of the present disclosure, there is provided a compound of Formula IV as disclosed herein, wherein
R₁ₐ is selected from halogen, C₁₋₃ alkyl, NHR₄, wherein R₄ is selected from hydrogen, C₁₋₃ alkyl, or —COR₅, wherein R₅ is C₁₋₂ alkyl substituted with hydroxy, and M7-L-Rₓ, wherein M10 is NHR₄;
R₂ₐ is selected from hydrogen, halogen, C₁₋₃ alkoxy, NHR₄, wherein R₄ is selected from hydrogen, C₁₋₃ alkyl, —COR₅, wherein R₅ is C₁ alkyl substituted with hydroxy, and M11-L-Rₓ, wherein M8 is selected from C₁₋₃ alkoxy, and NHR₄;
R₃ₐ is selected from C₁₋₃ alkyl wherein C₁₋₃ alkyl is optionally substituted with C₁₋₃ aminoalkyl, and M12-L-Rₓ, wherein M9 is selected from and C₁₋₃ alkyl substituted with C₁₋₃ aminoalkyl;
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and wherein at least one of R₁ₐ, R₂ₐ, R₃ₐ, and R₆ is L-Rₓ.

In further embodiments of the present disclosure, there is provided a compound of Formula IV as disclosed herein, wherein
R₁ₐ is selected from halogen, C₁₋₃ alkyl, NHR₄, wherein R₄ is —COCH₂OH, and M13-L-Rₓ, wherein M13 is NHR₄;
R₂ₐ is selected from hydrogen, halogen, C₁₋₂ alkoxy, NHR₄, wherein R₄ is hydrogen or —COCH₂OH, and M14-L-Rₓ, wherein M14 is selected from C₁₋₂ alkoxy, and NHR₄;
R₃ₐ is selected from C₁₋₂ alkyl, C₁₋₃ alkyl substituted with C₁₋₃ aminoalkyl, and M15-L-Rₓ, wherein M15 is selected from C₁₋₃ alkoxy, and NHR₄;
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises L-Rₓ.

In further embodiments of the present disclosure, there is provided a compound of Formula IV as disclosed herein, wherein
R₁ₐ is selected from fluorine, chlorine, bromine, iodine, C₁₋₂ alkyl or —NHCOCH₂OH and -M13-L-Rₓ, wherein M13 is-NHCOCH₂OH;
R₂ₐ is selected from hydrogen, halogen, NH₂, methoxy, amino carboxy hydroxylmethyl or M16-L-Rₓ, wherein M16 is selected from NH₂, methoxy, and amino carboxy hydroxylmethyl;
R₃ₐ is selected from methyl, or ethyl isopropylamino or M17-L-Rₓ wherein M17 is ethyl isopropylamino; and
R₆ is selected from hydrogen or -L-Rₓ,
wherein -L-Rₓ is a linker L attached to a reactive moiety Rₓ,
wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises L-Rₓ.

In more embodiments of the present disclosure, there is provided a compound of Formula IV as disclosed herein, wherein
R₁ₐ is selected from fluorine, methyl, —NHCOCH₂OH or M18-L-Rₓ, wherein M18 is-NHCOCH₂OH;
R₂ₐ is selected from hydrogen, fluorine, chlorine, methoxy, NH₂ or M19-L-Rₓ, wherein M19 is NH₂;
R₃ₐ is selected from methyl, ethyl isopropylamino, or M20-L-Rₓ, wherein M20 is ethyl isopropylamino; and
R₆ is selected from hydrogen or -L-Rₓ,
wherein -L-Rₓ is a linker L attached to a reactive moiety Rₓ,
wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises -L-Rₓ.

According to Formula I, chemical moieties at positions R₁, R₂ and R₃ can be selected from a variety of organic chemical groups. So, for example, according to Formula IV, position R₁, can be substituted with hydrogen, hydroxy, halogen, C₁₋₆ alkyl, C₁₋₆ alkoxy, C₁₋₆ aminoalkyl, or NHR₄, wherein R₄ is selected from hydrogen, C₁₋₆ alkyl, C₁₋₆ haloalkyl, C₁₋₆ alkylcarboxy, or —COR ₅; R₅ is selected from hydrogen, hydroxy, or C₁₋₆ alkyl, and wherein C₁₋₆ alkyl is optionally substituted with halogen or hydroxy.

A. Complexes with Formula VIII

In an embodiment of the present disclosure, there is provided a compound of Formula VIII, its a pharmaceutically acceptable salt or derivative thereof

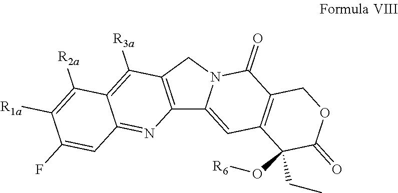

wherein, R₁ₐ is selected from hydrogen, hydroxy, halogen, C₁₋₆ alkyl, C₁₋₆ alkoxy, C₁₋₆ aminoalkyl, or NHR₄ wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is a selected from hydroxy, C₁₋₆ alkoxy, C₁₋₆ aminoalkyl, and NHR₄;
R₂ₐ is selected from C₁₋₆ alkylamine, optionally wherein Nis alkylated with C₁₋₆ alkyl, and -M2-L-Rₓ, wherein M2 is selected from C₁₋₆ alkylamine, optionally wherein N is alkylated with C₁₋₆ alkyl;
R₃ₐ is selected from hydrogen, halogen, or C₁₋₆ alkyl, wherein C₁₋₆ alkyl is optionally substituted with C₁₋₆ aminoalkyl, and -M3-L-Rₓ, wherein M3 is selected from or C₁₋₆ alkyl, wherein C₁₋₆ alkyl is substituted with C₁₋₆ aminoalkyl; and
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises -L-Rₓ. In certain embodiments, -L-Rₓ is attached through the R₂ₐ group.

In an embodiment of the present disclosure, there is provided a compound of Formula VIII according to any previous embodiment, wherein R₁ₐ is selected from halogen, C₁₋₃ alkyl and -L-Rₓ;
R₂ₐ is selected from propylamine, optionally wherein N is alkylated with C₁₋₃ alkyl, and -M5-L-Rₓ, wherein M5 is selected from propylamine, optionally wherein N is alkylated with —C₁₋₃ alkyl;
R₃ₐ is selected from C₁₋₃ alkyl and -L-Rₓ;
R₆ is selected from hydrogen or -L-Rₓ.

In an embodiment of the present disclosure, there is provided a compound of Formula VIII according to any previous embodiment, wherein
R₁ₐ is methyl or -L-Rₓ;
R₂ₐ is selected from propylamine and N-methyl propylamine, and -M8-L-Rₓ, wherein M8 is selected from propylamine or N-methyl propylamine;
R₃ₐ is methyl or -L-Rₓ;
R₆ is selected from hydrogen or -L-Rₓ.

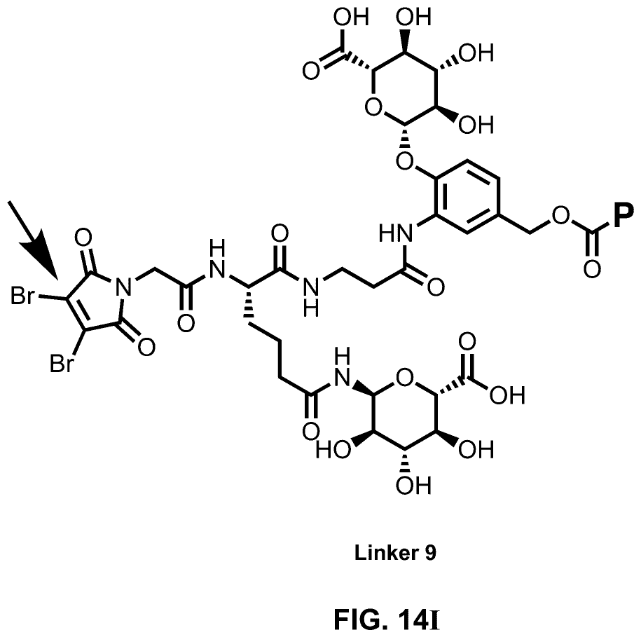

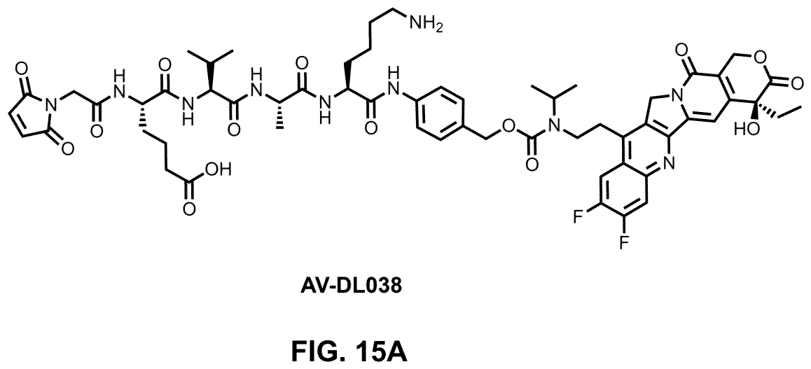

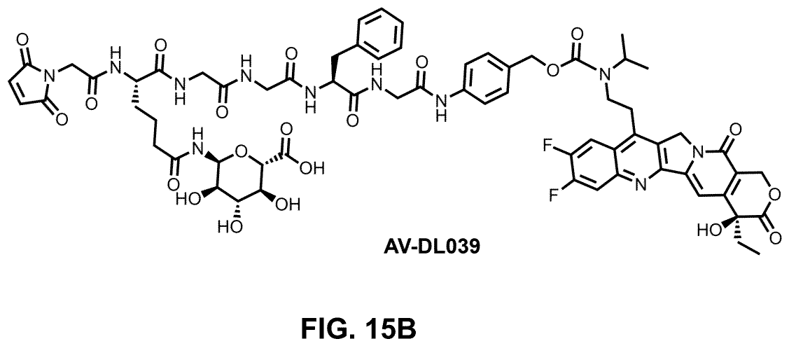

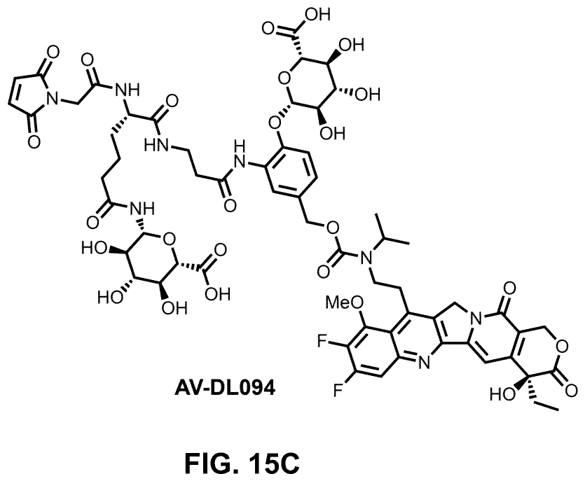

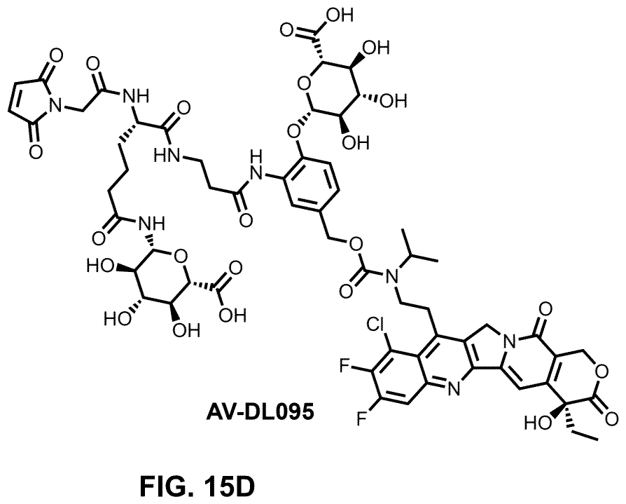

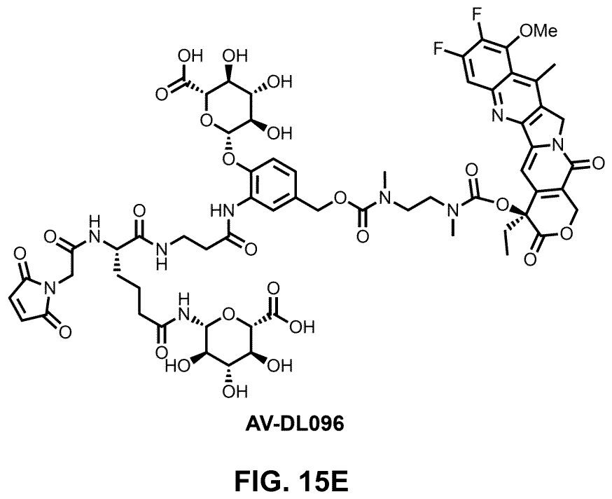

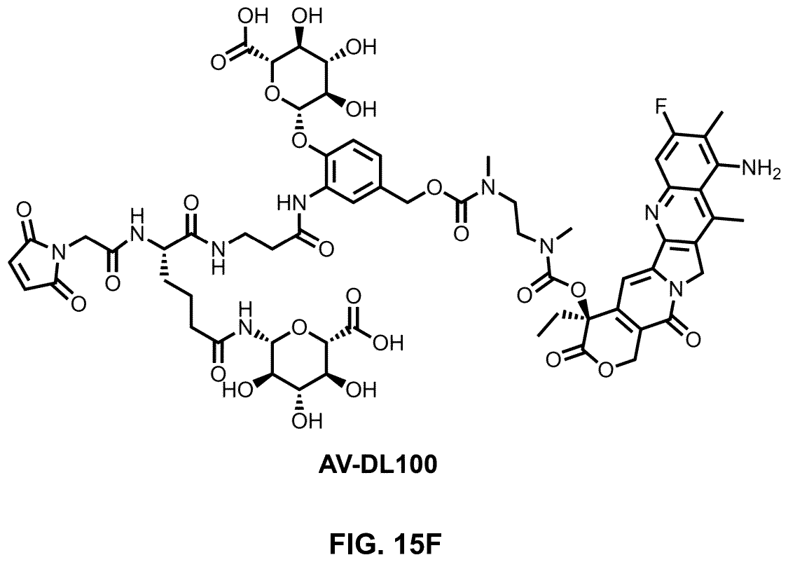

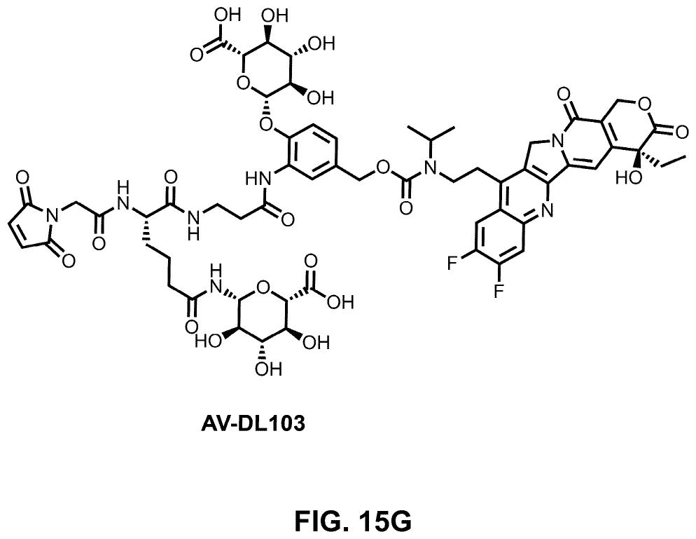

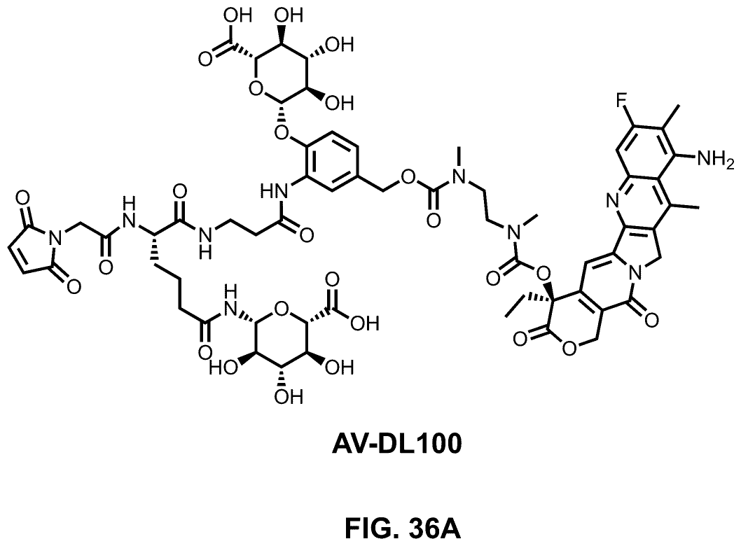

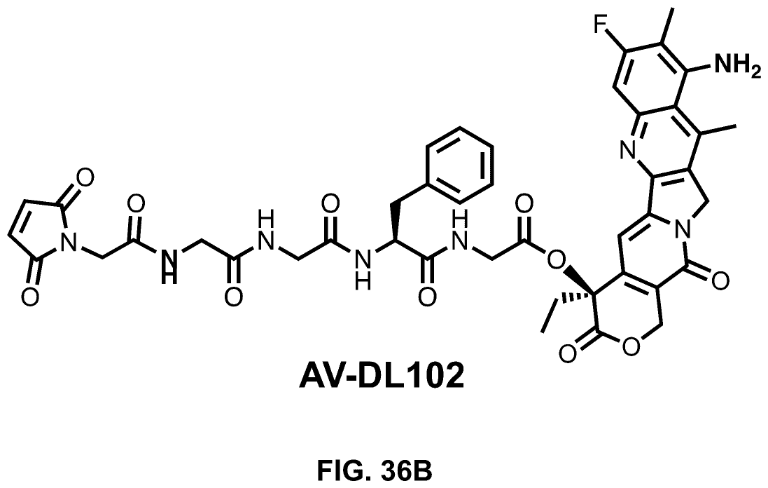

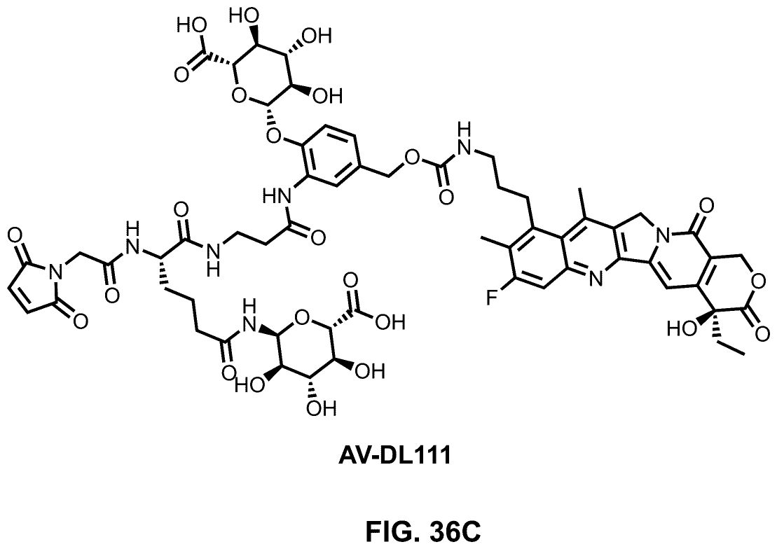

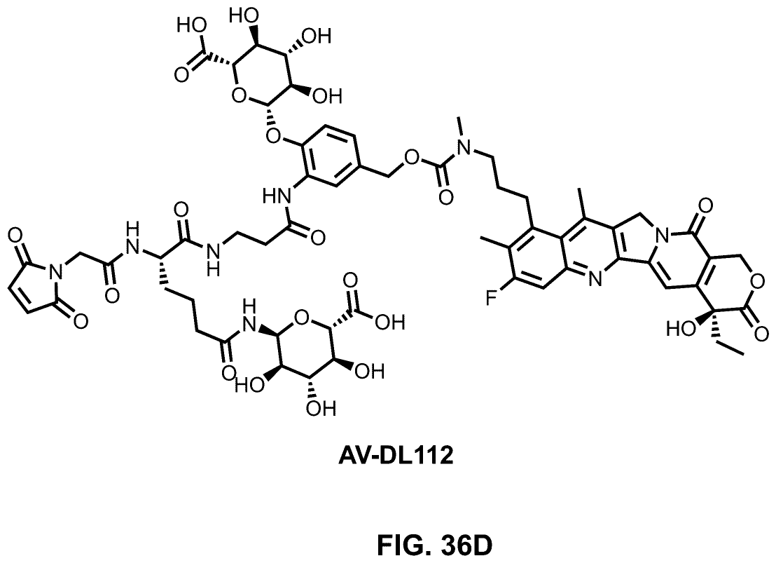

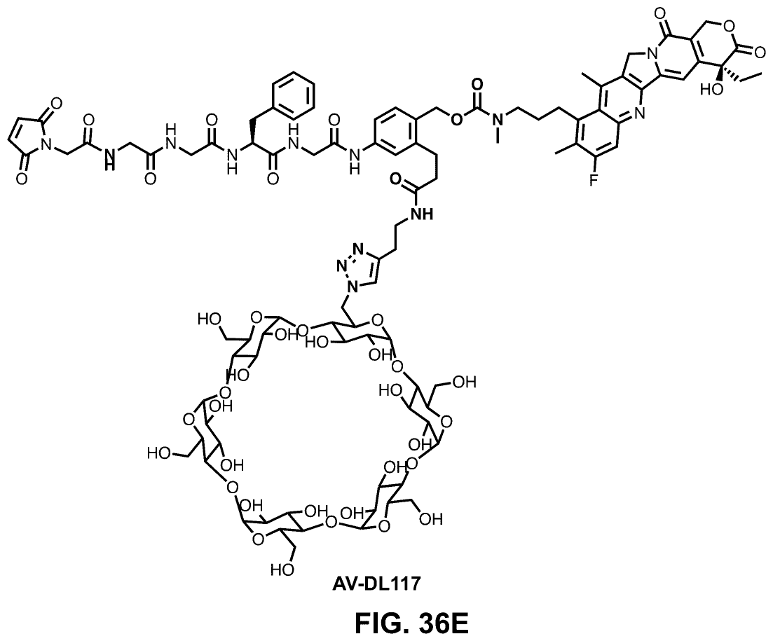

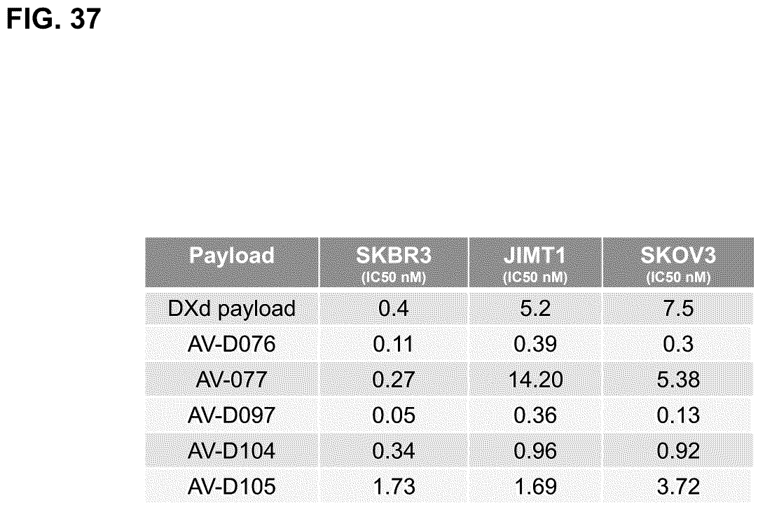

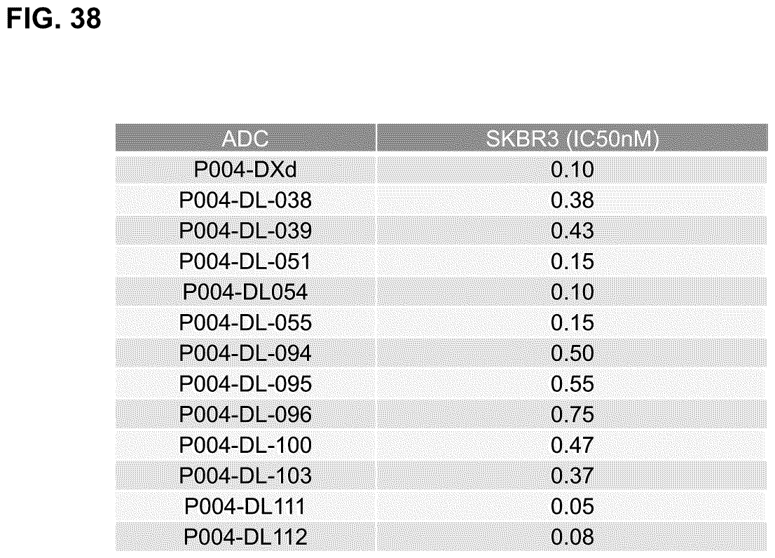

In Formula IV, Formula V, Formula VIII and Formula IX, Linker-L is attached to a camptothecin compound as disclosed herein via any suitable attachment site on the camptothecin compound. In some embodiments, this is through a moiety of any of R₁, R₂, R₃, R₄, R₅ or through the oxygen attached to R₆. For example, a hydrogen atom of a hydroxyl, thiol, primary amine, secondary amine or tertiary amine of the camptothecin compound is replaced with a bond to -L or a tertiary amine of the camptothecin compound is quaternized to form a bond to -L. Thus, the site of attachment can be at the positions of R₁, R₂, R₃, or R₆. Accordingly, in Formula IV, Formula V, Formula VIII and Formula IX, reference to a substituent “M-L-Rₓ” or “M-L” means a bond of -L-Rₓ or -L-through the selected moieties as suitable. It is understood that such linkage may involve replacement of a hydrogen atom, so, for example, reference to M being “hydroxy” means the bond of oxygen to hydrogen is replaced with a bond of oxygen to -L, and reference to M being “amino” means one of the bonds of nitrogen to hydrogen is replaced with a bond of nitrogen to -L, and so forth. Examples of such compounds include those depicted in FIG. 15, such as AV-DL038, AV-DL039, AV-DL094, AV-DL095, AV-DL096, AV-DL100, and AV-DL103, and FIGS. 36C and 36D, such as AV-DL111 and AV-DL112, AV-DL122, AV-DL123, AV-DL 160 and AV-DL161.

In an embodiment of the present disclosure, there is provided a compound of Formula IV or Formula VIII as disclosed herein, wherein-Rₓ is selected from succinimide, maleimide, iodoacetamide, azide, thiazole, pyrridopyridazine, bisulfone, isothiocyanate, succinimidyl ester, sulfonyl halide, carboxylic acids, sulfosuccinimidyl ester, 4-sulfotetrafluorophenyl ester, tetrafluorophenyl ester, sulfodichlorophenol ester, hydrazine, semihydrazide, carbohydrazide, hydroxylamine, bis-sulfone, phosphine, and alkyne.

B. Complexes with Linkers 1-9

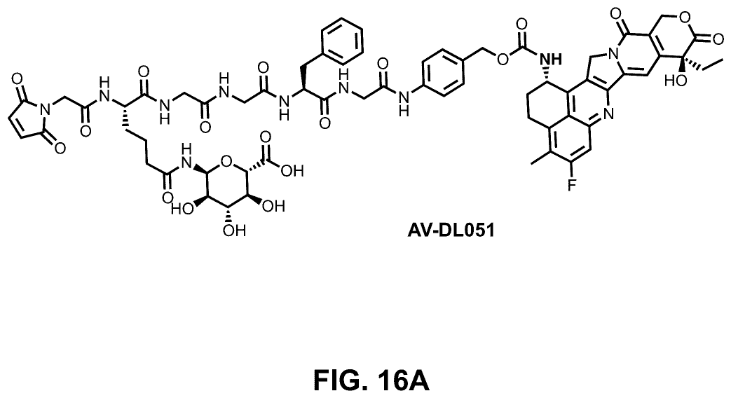

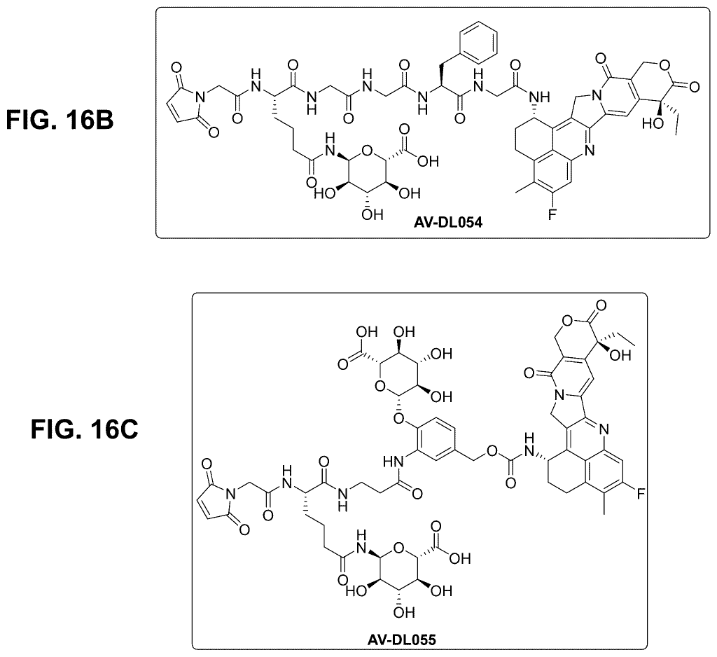

Other cytotoxic compounds comprising the linkers of this disclosure are set forth in FIGS. 16A-16C and 36A, 36B and 36E. They include AV-DL051, AV-DL054, AV-DL055, AV-DL100, AV-DL102, AV-DL117, AV-DL125, AV-DL126, and AV-DL149.

AV-DL055 is the product of a bond between Linker 3, herein, and exatecan (4-methyl-5-fluoro substituted hexacyclic camptothecin analog).

### VII. Targeting Conjugates and Immunoconjugates

a. Conjugates of Formula V

In another embodiment of the present disclosure, there is provided a conjugate having Formula V:

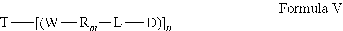

wherein T— is a targeting moiety selected from a protein, a portion of a protein, a peptide or a nucleic acid; —W—Rₘ— is a moiety formed by the reaction of reactive moiety Rₓ and a functional group of T; -L-D is a moiety having Formula VI

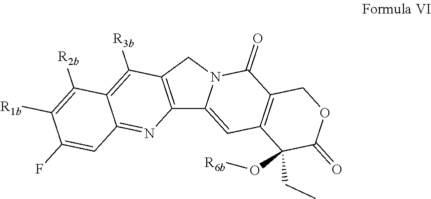

wherein, R1_{b} is selected from hydrogen, hydroxy, halogen, C₁₋₆ alkyl, C₁₋₆ alkoxy, C₁₋₆ aminoalkyl, NHR₄, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is a selected from hydroxy, C₁₋₆ alkoxy, C₁₋₆ aminoalkyl, NHR₄ and —COR₅;
R2_{b} is selected from hydrogen, halogen, C₁₋₆ alkoxy, NHR₄ or -L, wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxyl, and -M2-L-Rₓ, wherein M2 is selected from hydroxy, C₁₋₆ alkoxy, C₁₋₆ aminoalkyl, NHR₄;
R3_{b} is selected from hydrogen, halogen, C₁₋₆ alkyl, wherein C₁₋₆ alkyl is optionally substituted with C₁₋₆ aminoalkyl, and -M3-L-Rₓ, wherein M3 is selected from and C₁₋₆ alkyl substituted with C₁₋₆ aminoalkyl;
R_{6b} is selected from hydrogen or -L;
-L is a linker which is a bond or is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties;
wherein at least one of R1_{b}, R2_{b}, R3_{b}, and R_{6b} comprises -L;
m is an integer between 1 and 4; and
n is an integer between 1 and 20.

In yet another embodiment of the present disclosure, there is provided a conjugate having Formula V, wherein -L-D is a compound of Formula VI as disclosed herein, wherein
R1_{b} is selected from hydrogen, halogen, C₁₋₃ alkyl, C₁₋₃ alkoxy, C₁₋₃ aminoalkyl, NHR₄, or -L, wherein R₄ is selected from hydrogen, C₁₋₃ alkyl, C₁₋₃ haloalkyl, carboxy, or —COR₅; R₅ is selected from hydrogen, hydroxy, or C₁₋₃ alkyl, and wherein C₁₋₃ alkyl is optionally substituted with halogen or hydroxy, and -M4-L-Rₓ, wherein M4 is selected from C₁₋₃ alkoxy, C₁₋₃ aminoalkyl, and NHR₄;
R2_{b} is selected from hydrogen, halogen, C₁₋₃ alkyl, C₁₋₃ alkoxy, C₁₋₃ aminoalkyl, NHR₄, wherein R₄ is selected from hydrogen, C₁₋₃ alkyl, C₁₋₃ haloalkyl, or —COR₅; R₅ is selected from hydrogen, hydroxy, or C₁₋₃ alkyl, and wherein C₁₋₃ alkyl is optionally substituted with halogen or hydroxy, and -M5-L-Rₓ, wherein M5 is selected from C₁₋₃ alkoxy, C₁₋₃ aminoalkyl, and NHR₄;
R3_{b} is selected from hydrogen, halogen, C₁₋₃ alkyl wherein C₁₋₃ alkyl is optionally substituted with C₁₋₃ aminoalkyl, and -M6-L-Rₓ, wherein M6 is selected from C₁₋₃ alkyl is substituted with C₁₋₃ aminoalkyl;
R_{6b} is selected from hydrogen or -L;
-L is a linker which is a bond or is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and
wherein at least one of R1_{b}, R2_{b}, R3_{b}, and R6_{b} comprises -L;
m is an integer between 1 and 4; and
n is an integer between 1 and 20.

In yet another embodiment of the present disclosure, there is provided a conjugate having Formula V as disclosed herein, wherein -L-D is a compound of Formula VI, wherein
R1_{b} is selected from halogen, C₁₋₃ alkyl, NHR₄, wherein R₄ is selected from hydrogen, C₁₋₃ alkyl, or —COR₅, wherein R₅ is C₁₋₂ alkyl substituted with hydroxy, and -M7-L-Rₓ, wherein M7 is NHR₄;
R2_{b} is selected from hydrogen, halogen, C₁₋₃ alkoxy, NHR₄; wherein R₄ is selected from hydrogen, C₁₋₃ alkyl, or —COR₅, wherein R₅ is C₁ alkyl substituted with hydroxy, and -M8-L-Rₓ, wherein M8 is selected from C₁₋₃ alkoxy, and NHR₄;
R3_{b} is selected from C₁₋₃ alkyl or -L, wherein C₁₋₃ alkyl is optionally substituted with C₁₋₃ aminoalkyl, and -M9-L-Rₓ, wherein M9 is selected from C₁₋₃ alkyl is substituted with C₁₋₃ aminoalkyl;
R_{6b} is selected from hydrogen or -L;
-L is a linker which is a bond or is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and wherein at least one of R1_{b}, R2_{b}, R3_{b}, and R6_{b} comprises L;
m is an integer between 1 and 4; and
n is an integer between 1 and 20.

In still another embodiment of the present disclosure, there is provided a conjugate having Formula V as disclosed herein, wherein -L-D is a compound of Formula VI as disclosed herein, wherein
R1_{b} is selected from halogen, C₁₋₃ alkyl, NHR₄, wherein R₄ is —COCH₂OH, and -M10-L-Rₓ, wherein M10 is NHR₄;
R2_{b} is selected from hydrogen, halogen, C₁₋₂ alkoxy, NHR₄, wherein R₄ is hydrogen or —COCH₂OH, and -M11-L-Rₓ, wherein M11 is selected from C₁₋₂ alkoxy and NHR₄; and
R3_{b} is selected from C₁₋₂ alkyl, C₁₋₃ alkyl substituted with C₁₋₃ aminoalkyl or -L, and -M12-L-Rₓ, wherein M12 is selected from C₁₋₂ alkoxy, and NHR₄;
R_{6b} is selected from hydrogen or -L;
-L is a linker which is a bond or is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and
wherein at least one of R1_{b}, R2_{b}, R3_{b}, and R_{6b} comprises -L.

In further embodiments of the present disclosure, there is provided a conjugate having Formula V as disclosed herein, wherein -L-D is a compound of Formula VI, wherein
R1_{b} is selected from fluorine, chlorine, bromine, iodine, C₁₋₂ alkyl or —NHCOCH₂OH and -M13-L-Rₓ, wherein M13 is-NHCOCH₂OH;
R2_{b} is selected from hydrogen, halogen, NH₂, methoxy, amino carboxy hydroxylmethyl and -M13-L-Rₓ, wherein M13 is selected from methoxy, and amino carboxy hydroxylmethyl;
R3_{b} is selected from methyl, or ethyl isopropylamino and -M14-L-Rₓ, wherein M14 is isopropylamino; and
R_{6b} is selected from hydrogen or -L,
wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and
wherein at least one of R1_{b}, R2_{b}, R3_{b}, and R_{6b} comprises -L.

### A. Conjugates of Formula IX

In another embodiment of the present disclosure, there is provided a conjugate having Formula IX:

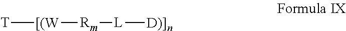

wherein:
T— is a targeting moiety selected from a protein, a portion of a protein, a peptide or a nucleic acid;
—W—Rₘ— is a moiety formed by the reaction of reactive moiety Rₓ and a functional group of T;
-L-D is a moiety having Formula X

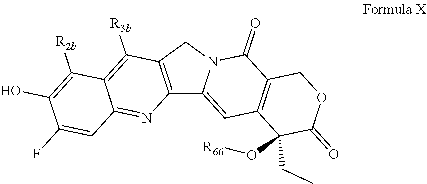

wherein, R1_{b} is selected from hydrogen, hydroxy, halogen, C₁₋₆ alkyl, C₁₋₆ alkoxy, C₁₋₆ aminoalkyl, NHR₄ wherein R₄ is selected from hydrogen, C₁₋₆ alkyl, C₁₋₆ haloalkyl, C₁₋₆ alkylcarboxy, or —COR₅; R₅ is selected from hydrogen, hydroxy, or C₁₋₆ alkyl, and wherein C₁₋₆ alkyl is optionally substituted with halogen or hydroxy, and -M1-L-Rₓ, wherein M1 is selected from hydroxy, C₁₋₆ alkoxy, C₁₋₆ aminoalkyl, and NHR₄.
R2_{b} is selected from C₁₋₆ alkylamine, optionally wherein Nis alkylated with C₁₋₆ alkyl, and -M2-L-Rₓ, wherein-M2 is C₁₋₆ alkylamine, optionally wherein Nis alkylated with C₁₋₆ alkylamine;
R3_{b} is selected from hydrogen, halogen, C₁₋₆ alkyl, wherein C₁₋₆ alkyl is optionally substituted with C₁₋₆ aminoalkyl, and -M3-L-Rₓ, wherein-M3 is selected from C₁₋₆ alkyl substituted with C₁₋₆ aminoalkyl; and
R₆ is selected from hydrogen or -L-Rₓ;
wherein -L-Rₓ is a linker L attached to a reactive moiety Rₓ, wherein -L is a moiety having 1-200 nonhydrogen atoms selected from C, N, O, S, or halogen, and optionally incorporates ether, oxo, carboximidoyl, urethanyl, branched, cyclic, unsaturated, heterocyclic, aromatic or heteroaromatic moieties; and Rₓ is a reactive moiety, and
wherein at least one of R₁ₐ, R₂ₐ, R₃ₐ, and R₆ comprises -L-Rₓ.

In yet another embodiment of the present disclosure, there is provided a conjugate having Formula IX as disclosed herein, wherein -L-D is a compound of Formula X, wherein
R1_{b} is selected from C₁₋₃ alkyl, and -L-Rₓ;
R2_{b} is selected from propylamine, optionally substituted with C₁₋₃ alkyl, and M5-L-Rₓ, wherein M5 is selected from and propylamine, optionally substituted with C₁₋₃ alkyl;
R3_{b} is selected from C₁₋₃ alkyl, and -L-Rₓ;
R₆ is selected from hydrogen, and -L-Rₓ.

In yet another embodiment of the present disclosure, there is provided a conjugate having Formula IX as disclosed herein, wherein -L-D is a compound of Formula X, wherein
R1_{b} is selected from methyl, and -L-Rₓ;
R2_{b} is selected from propylamine and N-methyl propylamine, and -M8-L-Rₓ, wherein-M8 is selected from and propylamine, optionally substituted with C₁₋₃ alkyl;
R3_{b} is selected from methyl, and -L-Rₓ;
R₆ is selected from hydrogen or -L-Rₓ.
   B. Conjugates with AV-DL055

In another embodiment of the present disclosure, there is provided a conjugate having a targeting moiety attached to AV-DL055. Such conjugates are described by Formula XI:

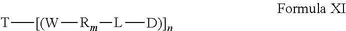

wherein:
T— is a targeting moiety selected from a protein, a portion of a protein, a peptide or a nucleic acid;
—W—Rₘ— is a moiety formed by the reaction of maleimide and a functional group of T;
-L-D is a moiety having Formula XII:

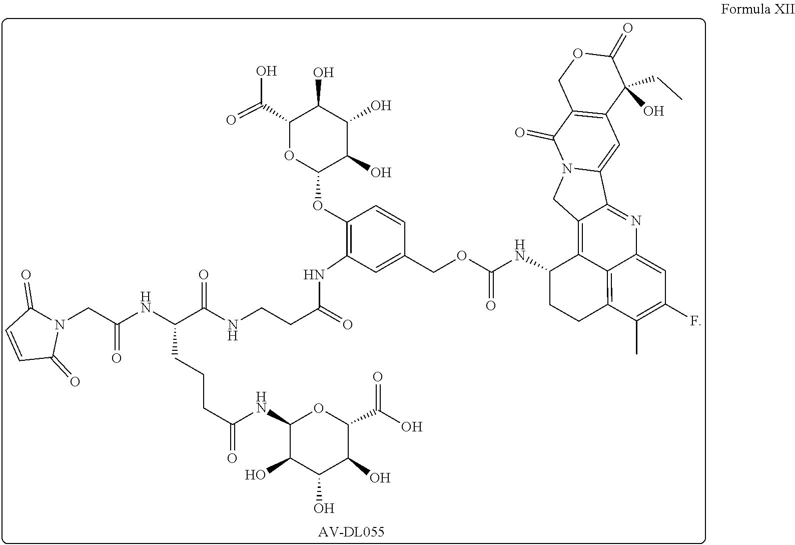 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCNC(=O)[C@H](CCCC(=O)N[C@H]2O[C@H](C(=O)O)[C@@H](O)[C@H](O)[C@H]2O)NC(=O)CN2C(=O)C=CC2=O)c1)CC3]

In an embodiment of the present disclosure, there is provided a conjugate having Formula V, Formula IX or Formula XI as disclosed herein, wherein the targeting moiety, T, comprises an antibody.

In an embodiment of the present disclosure, there is provided a conjugate having Formula V, Formula IX or Formula XI as disclosed herein, wherein the targeting moiety, T, comprises an antibody, and the antibody binds to a cancer cell.

In an embodiment of the present disclosure, there is provided a conjugate having Formula V, Formula IX or Formula XI as disclosed herein, wherein the targeting moiety, T, comprises an antibody, and the antibody binds to a cancer cell which is affected with a cancer selected from adenocarcinoma of the lung, squamous carcinoma of the lung, bladder cancer, blastoma, breast cancer, cancer of the peritoneum, cervical cancer, colon cancer, colorectal cancer, endometrial or uterine carcinoma, esophageal cancer, gastric cancer, glioblastoma, head & neck cancer, hepatic carcinoma, hepatocellular cancer, hepatoma, renal cancer, leukemia, liver cancer, low-grade gliomas (LGG), lung cancer including small-cell lung cancer, lymphoma, mesothelioma, melanoma, multiple myeloma, non-small cell lung cancer (NSCLC), ovarian cancer, pancreatic cancer, prostate cancer, rectal cancer, salivary gland carcinoma, sarcoma, squamous cell cancer, stomach cancer, thyroid cancer, and vulval cancer.

In an embodiment of the present disclosure, there is provided a conjugate having Formula V, Formula IX or Formula XI as disclosed herein, wherein the targeting moiety, T, comprises an antibody which binds to an antigen selected from 0772P (CA125, MUC16), 5T4, ADAM9, AG-7, Alkaline phosphatase (ALPP), ASCT2, ASLG659, Axl, BAFF-R (B cell-activating factor receptor), BLyS receptor 3, BR3, BMPR1B (bone morphogenetic protein receptor-type IB), Brevican, B7-H3, B7-H4, BCMA, C4.4a, CA6, CA9, CanAg, CCR7, CD19, CD20, CD21 (CR2 (Complement receptor 2), CD22 (B-cell receptor CD22-B isoform), CD30, CD33, CD352, CD37, CD38, CD44v6, CD46, CD47, CD48, CD51, CD56, CD70, CD71, CD72 (B-cell differentiation antigen CD72, Lyb-2), CD74, CD79a, CD79b, CD123, CD138, CD142, CD166, CD205, CD228, CD352, CDH6, CEACAM5, CEACAM6, cKIT, CLDN18.2, CLDN6, CLDN9, CLL-1, c-MET, CRIPTO (EGF-CFC), CXCR5 (Burkitt's lymphoma receptor 1), EGFR, EGFRviii, Cripto, CSP-1, DLK-1, DLL3, DPEP3, DR5, EFNA4, ENPP3, EphA2, EphA5, EphB2R, ETBR (Endothelin type B receptor), E16 (LAT1, SLC7A₅), FAP, FcRH1 (Fc receptor-like protein 1), FcRH2, FCRH5, FGFR2, FGFR3, FGFR1-IIIc, FGFR2-IIIc, FGFR3-IIIc, Fibronectin extra-domain B, FLT3, Folate Receptor Alpha, GCC, GD3, Globo H, GPC3, GEDA, GFRA1, gpNMB, GPR20, GPRC5D, HER-2, HER-3, HLA-DOB, IGF-1R, IL1RAP, IL20Rα, IRTA2 (Immunoglobulin superfamily receptor translocation associated 2), Integrin beta-6, KAAG-1, LAMP-1, Lewis Y antigen, LIV-1, LRRC15, Ly6E, LY64 (Lymphocyte antigen 64), MDP, MPF (megakaryocyte potentiating factor), Mesothelin (MSLN), MT1-MMP (MMP14), MUC-1, MUC18, NaPi2b, Napi3b, NCA, Nectin-4, NOTCH3, P2X (Purinergic receptor P2X ligand-gated ion channel 5), P-Cadherin, PDGFR, PD-L1, Prolactin Receptor (PRLR), PSCA, PSCA homolog, PSMA, PTK7, RET, RNF43, ROR1, ROR2, Sema 5b, SEZ6, SLAMF7, SLC44A4, SLITRK6, STEAP-1, STEAP2, STn, TAA, TIM-1, Tissue factor, TNF-alpha, TENB2, TrpM4, and TROP-2. In certain embodiments, the antibody binds to an antigen selected from: ALPV/ALPPL2; CCR2; CD25; CDH17; TAG-72; and TM4SF1.

In an embodiment of the present disclosure, there is provided a conjugate of Formula V, Formula IX or Formula XI according to any previous embodiment, wherein the antibody is multispecific, e.g., bispecific, trispecific or tetraspecific.

In an embodiment of the present disclosure, there is provided a conjugate of Formula V, Formula IX or Formula XI according to any previous embodiment, wherein each antibody binding site binds an antigen as disclosed herein.

### VIII. Pharmaceutical Compositions

Further provided herein is a composition comprising an immunoconjugate as described herein, optionally with a suitable diluent, e.g., a pharmaceutically acceptable carrier.

In one embodiment, a pharmaceutical composition comprises an immunoconjugate as described herein formulated in a pharmaceutically acceptable carrier. The composition is typically formulated for intravenous injection. Accordingly, the carrier can be an aqueous carrier, such as sterilized water or saline solution. The composition can further comprise ingredients to stabilize the antibody molecules. These can include, for example, sugars and polyols, which assist in freeze-drying. Mannitol also can be used as a tonicity adjuster and bulking agent in freeze-dried formulations. Surfactants, such as polysorbate 20 or polysorbate 80 are also commonly used in monoclonal antibody formulations. Buffers, such as phosphate, histidine and citrate, are frequently used in such formulations.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

The term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt a (HBSS) and multiple electrolyte solutions such as PlasmaLyte ATM (Baxter).

Suitable diluents for polypeptides, including antibodies and/or cells include but are not limited to saline solutions, pH buffered solutions and glycerol solutions or other solutions suitable for freezing polypeptides and/or cells.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Exemplary injection solutions and suspensions may be prepared from sterile powders, granules, tablets, or concentrated solutions or suspensions. The composition may be supplied, for example, but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1 (2,3-dioleyloxy)propyl) N,N,N-trimethylammonium chloride (DOTMA), dioleoylphosphatidyl-ethanolamine (DOPE), and liposomes. Such compositions should contain a therapeutically effective amount of the compound, optionally with a suitable amount of carrier so as to provide the form for direct administration to the patient.

The composition may be in the form of a pharmaceutically acceptable salt which includes, without limitation, those formed by hydrochloric, phosphoric, acetic, oxalic, and tartaric acids, etc. with free amino groups, and those formed by sodium, potassium, ammonium, calcium, ferric hydroxides, isopropylamine, triethylamine, and 2-ethylamino ethanol, etc. with free carboxyl groups.

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable label (if present). One of skill in the art will recognize that the dose can be modified depending on the above factors and/or based on therapeutic progress. The term “dosage form” refers to the particular format of the pharmaceutical, and depends on the route of administration. For example, a dosage form can be in a liquid, e.g., a saline solution for injection.

### IX. Methods of Use

Provided herein are methods of treating a disease in a subject that comprises administering to the subject an effective amount of a compound or an immunoconjugate of this disclosure. An “effective amount” is an amount sufficient for a compound or an immunoconjugate to accomplish a stated purpose relative to the absence of the compound. The stated purpose may be, for example, killing pathological cells, either in vitro or in vivo, or producing a therapeutic benefit to a subject. An example of an “effective amount” is an amount sufficient to contribute to the treatment, prevention, or reduction of a symptom or symptoms of a disease, which could also be referred to as a “therapeutically effective amount.” A “reduction” of, or “reducing” a symptom means elimination of, or decreasing of the severity or frequency of, the symptom. A “prophylactically effective amount” of a substance (e.g., an antibody) is an amount of that substance, when administered to a subject, will prevent or delay the onset or reoccurrence of a disease, pathology or condition, or its symptoms, or reduce the likelihood of such. An effective amount does not necessarily occur by administration of one dose, and may occur only after administration of a series of doses. Thus, an effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any ADC described herein, the therapeutically effective amount can be initially estimated or determined using cell culture assays. Target concentrations will be those concentrations of ADC that are capable of a desired result (e.g., slowing cell growth), as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Cancers that can be treated include, without limitation, leukemias, carcinomas, sarcomas, adenocarcinomas, lymphomas, solid and lymphoid cancers. Examples of different types of cancer include, but are not limited to, lung cancer (e.g., non-small cell lung cancer or NSCLC), breast cancer, ovarian cancer, prostate cancer, colorectal cancer, bladder cancer, leukemia, liver cancer (i.e., hepatocarcinoma), renal cancer (i.e., renal cell carcinoma), thyroid cancer, pancreatic cancer, uterine cancer, cervical cancer, testicular cancer, esophageal cancer, stomach (gastric) cancer, kidney cancer, cancer of the central nervous system, skin cancer, glioblastoma, melanoma, low-grade gliomas (LGG), mesothelioma, melanoma, multiple myeloma, and head and neck cancer.

### X. Routes of Administration

Pharmaceutical compositions comprising antibodies of this disclosure can be administered to a subject by any suitable route of administration.

Intravenous administration involves delivering the pharmaceutical composition directly into the bloodstream which facilitates rapid systemic distribution and enables immediate therapeutic action. Intravenous administration can comprise administration of a bolus or can comprise a slow infusion depending on the desired pharmacokinetic profile in the therapeutic indication.

Intraperitoneal administration involves introducing the pharmaceutical composition into the peritoneal cavity. This mode of administration is especially useful for diseases localized within the abdominal region, as it offers direct access to the site of pathology.

The pharmaceutical composition can be administered subcutaneously, that is into the fatty tissue underneath the skin.

In intramuscular administration, a pharmaceutical composition is injected into muscle tissue. In certain cancers, the pharmaceutical composition can be administered directly into the tumor.

For diseases of the central nervous system the composition can be administered intrathecally or intraventricularly, e.g., into the spinal canal or the brain ventricles.

### XI. Treatment Regimen

The treatment regimen for cancers using the compounds or ADCs of this disclosure is customized to the cancer in the patient. However, exemplary regimens for existing treatments include the following. Between 1 mg and 12 mg of ADC per kilogram of body weight is administered as a dose. After an initial dose, the drug can be administered about every week for about every three weeks for five or more cycles, e.g., until remission, sufficient reduction of symptoms, complete reduction of symptoms, or until side effects or toxicity become unmanageable or the drug ceases to have therapeutic benefit.

### XII. Methods of Killing Cells

The immunoconjugates of this disclosure are useful for killing cells or labelling cells, either in vitro or in vivo. The methods involve contacting the cells with an immunoconjugate of this disclosure (e.g., by introducing said immunoconjugate into a subject in need thereof) and allowing the antibodies to bind to the target cells. Effectiveness of killing cells can be a function of ability to kill target cells, but not non-target cells in a mixed population of cells. Ability to kill cells also can be compared with that of a control, e.g., a control antibody, wherein greater ability of the test antibody compared with the control antibody to kill cells indicates that the test antibody is effective in killing those cells.

Contacting cells from cancer cell lines with the compositions disclosed herein, in vitro, is useful for determining toxicity of the composition on the cells. In an embodiment of the present disclosure, there is provided a conjugate having Formula V as disclosed herein, wherein W is a moiety of a natural or unnatural amino acid residue or a carbohydrate of the targeting moiety T.

In an embodiment of the present disclosure, there is provided a pharmaceutical composition comprising a compound of Formula I as disclosed herein, or a compound of Formula IV as disclosed herein, or a conjugate as disclosed herein, and a pharmaceutically acceptable excipient.

In an embodiment of the present disclosure, there is provided a pharmaceutical composition as disclosed herein, wherein the composition is in the form of a tablet, capsule, liquid, or solution.

In an embodiment of the present disclosure, there is provided a method for treating cancer, the method comprising administering to a subject suffering from cancer an effective amount of a compound as disclosed herein, or a conjugate as disclosed herein, optionally with other relevant cytotoxic or non-cytotoxic agents.

In an embodiment of the present disclosure, there is provided a method of inhibiting cell division, the method comprising: contacting a cell with a compound as disclosed herein, or a conjugate as disclosed herein.

In an embodiment of the present disclosure, there is provided a use of a compound as disclosed herein, or a conjugate as disclosed herein, for the manufacture of a medicament for treating cancer.

In an embodiment of the present disclosure, there is provided a use of a compound as disclosed herein, or a conjugate as disclosed herein, for the treatment of cancer.

Although the subject matter has been described in considerable detail with reference to certain examples and implementations thereof, other implementations are possible.

### EXAMPLES

The disclosure will now be illustrated with following examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### PREPARATION OF COMPOUNDS OF THIS DISCLOSURE

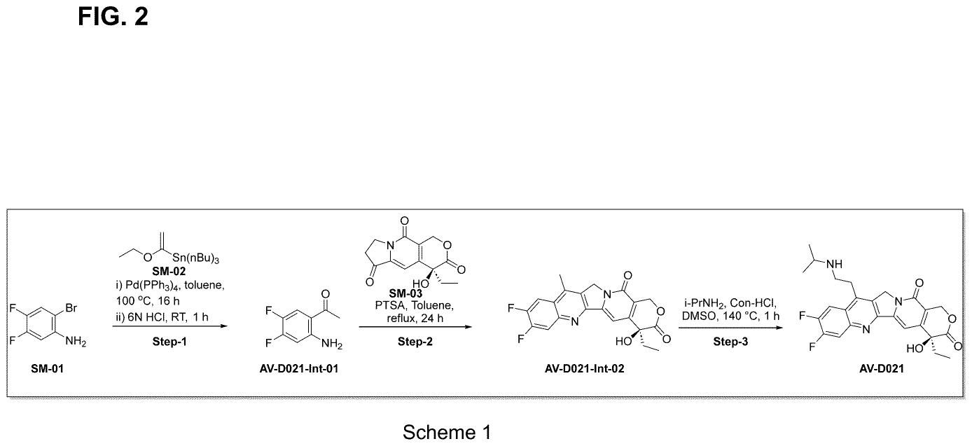

### I. Preparation of(S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021) (Scheme 1, FIG. 2)

The following example provides a synthetic reaction for the preparation of the compound(S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021) as Scheme 1 provided in FIG. 2.

### a. Synthesis of 1-(2-amino-4,5-difluorophenyl) ethan-1-one (AV-D021-Int-01; Formula II)

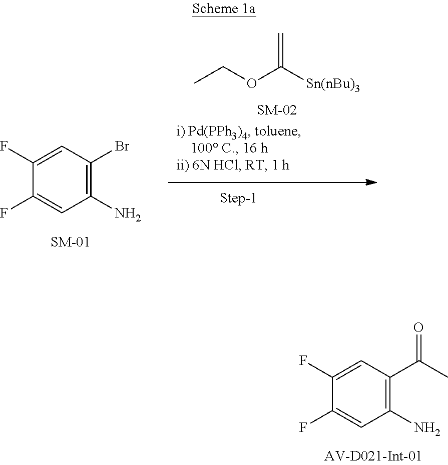

To a stirred solution of 2-bromo-4,5-difluoroaniline (SM-01, 2 g, 9.61 mmol) and tributyl(1-ethoxyvinyl) stannane (SM-02, 4.1 g, 11.53 mmol) in toluene (20 mL), Pd(PPh₃)₄ was added at room temperature. The reaction was stirred at 100° C. for 16 h. The reaction was monitored by thin layer chromatography (TLC). The reaction was cooled to 25° C., treated with 6N HCl (10 mL) and stirred at same temperature for 1 h. The reaction was diluted with water (20 mL) and extracted with EtOAc (ethyl acetate, 2× 10 mL). The combined organic layer was washed with brine (20 mL), dried over anhydrous Na₂SO₄, filtered, and concentrated under reduced pressure to obtain crude compound. The crude compound was purified by silica gel column chromatography (SiO₂, 100-200 mesh, 30% EtOAc/hexane) to afford 1-(2-amino-4,5-difluorophenyl) ethan-1-one (AV-D021-Int-01, Formula II, 700 mg, 4.04 mmol, 42% yield) as a colourless liquid. The reaction is as provided above in Scheme 1a.

MS (ESI): 171.60 [M+H]⁺; ¹H NMR (400 MHz, DMSO-d₆): δ 7.49 (dd, J=8.8, 11.2 Hz, 1H), 6.41 (dd, J=10.8, 12.0 Hz, 1H), 6.29 (bs, 2H), 2.52 (s, 3H).

### b. Synthesis of(S)-4-ethyl-8,9-difluoro-4-hydroxy-11-methyl-1,12-dihydro-14H-pyrano[3′,4′:6,7]indolizino[1,2-b]quinoline-3,14(4H)-dione (AV-D021-Int-02)

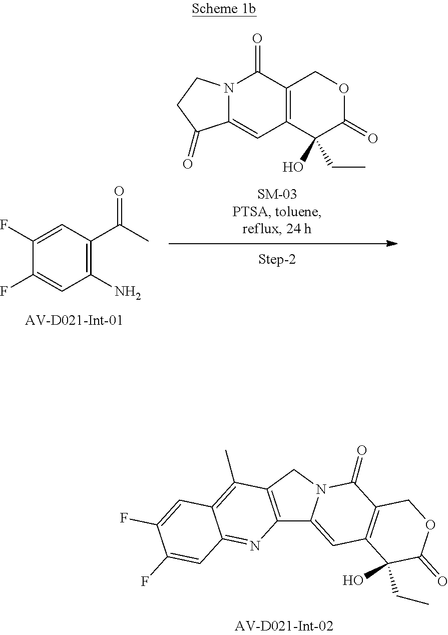 [SMILES: CC[C@@]1(O)C(=O)OCc2c1cc1n(c2=O)CCC1=O ; CC(=O)c1cc(F)c(F)cc1N ; "SM-03PTSA, toluene,reflux, 24 h" ; CC[C@@]1(O)C(=O)OCc2c1cc1n(c2=O)Cc2c-1nc1cc(F)c(F)cc1c2C]

To a stirred solution of 1-(2-amino-4,5-difluorophenyl) ethan-1-one (AV-D021-Int-01, Formula II, 250 mg, 0.95 mmol), and(S)-4-ethyl-4-hydroxy-7,8-dihydro-1H-pyrano[3,4-f]indolizine-3,6,10 (4H)-trione (SM-03, Formula III, 296 mg, 1.71 mmol) in toluene (solvent, 5 mL), p-toluenesulfonic acid (PTSA, 18 mg, 0.095 mmol) was added at room temperature and the obtained reaction mixture was heated at 110° C. for 24 h. The reaction was monitored by TLC. The resulting reaction mixture was concentrated under reduced pressure to get crude product. The crude product was further purified by RP Biotage to get(S)-4-ethyl-8,9-difluoro-4-hydroxy-11-methyl-1,12-dihydro-14H-pyrano[3′,4′:6,7]indolizino[1,2-b]quinoline-3, 14 (4H)-dione (AV-D021-Int-02, 250 mg, 0.628 mmol, 65% yield) as an off-white solid. The reaction is as provided above in Scheme 1b.

MS (ESI): 397.0 [M−H]⁻; ¹H NMR (400 MHz, DMSO-d₆): δ 8.29 (dd, J=8.8, 12.0 Hz, 1H), 8.17 (dd, J=8.0, 11.6 Hz, 1H), 7.30, (s, 1H), 5.43 (s, 2H), 5.26 (s, 2H), 2.80 (s, 3H), 1.92-1.81 (m, 2H), 0.88 (t, J=7.2 Hz, 3H), —OH peak was not observed.

### c. Synthesis of(S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021)

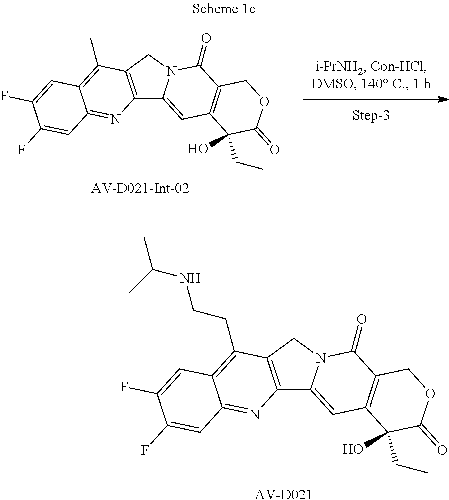

To a stirred solution of(S)-4-ethyl-8,9-difluoro-4-hydroxy-11-methyl-1,12-dihydro-14H-pyrano[3′,4′:6,7]indolizino[1,2-b]quinoline-3,14(4H)-dione (AV-D021-Int-02, 150 mg, 0.37 mmol), and isopropylamine (0.2 mL, 2.24 mmol) in 5 mL of dimethyl sulfoxide, concentrated hydrochloric acid (0.4 mL) was added and the resulting mixture was heated at 140° C. for 2 hour. The obtained reaction mixture was lyophilised with MeCN/H₂O to get brown gum which was purified by Reverse Phase High-Performance Liquid Chromatography (RP prep HPLC) to obtain (S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021, 8.5 mg, 0.018 mmol, 5% yield). The reaction is as provided above in Scheme 1c.

MS (ESI): 468.0 [M−H]⁻; ¹H NMR (400 MHz, DMSO-d₆): δ 8.39 (dd, J=3.2, 12.0 Hz, 1H), 8.21 (dd, J=3.6, 11.6, Hz, 1H), 7.33 (s, 1H), 6.54 (brs, 1H), 5.44 (s, 2H), 5.37 (s, 2H), 3.32-3.26 (m, 2H), 2.96-2.93 (m, 2H), 2.90-2.85 (m, 1H), 1.92-1.81 (m, 2H), 1.00 (d, J=6.0 Hz, 6H), 0.87 (t, J=7.2 Hz, 3H), —NH proton was not observed.

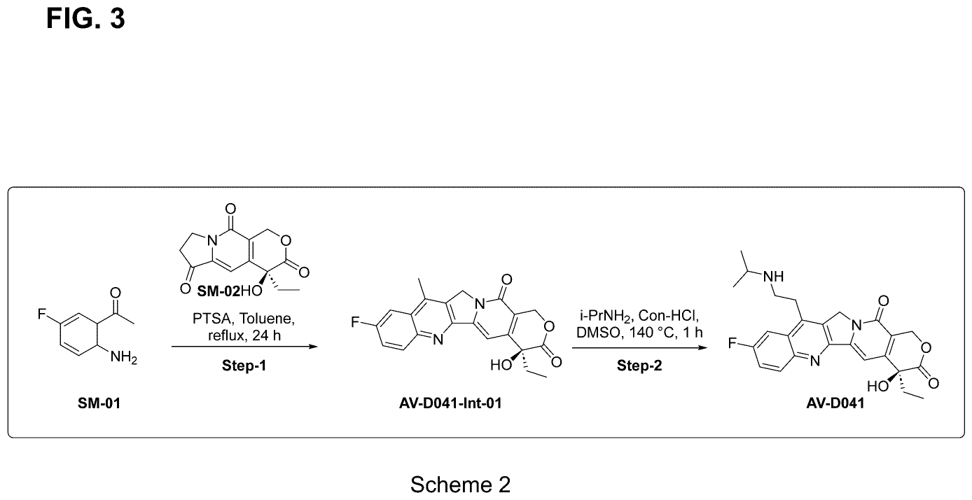

### II. Preparation of(S)-4-ethyl-9-fluoro-4-hydroxy-11-(2-(isopropylamino)ethyl)-1,12-dihydro-14H-pyrano[3′,4′:6,7]indolizino[1,2-b]quinoline-3,14(4H)-dione (AV-D041) (Scheme 2, FIG. 3)

The following example provides a synthetic procedure for the preparation of the compounds(S)-4-ethyl-9-fluoro-4-hydroxy-11-(2-(isopropylamino)ethyl)-1,12-dihydro-14H-pyrano[3′,4′:6,7]indolizino[1,2-b]quinoline-3,14(4H)-dione (AV-D041). The FIG. 3 depicts Scheme 2, the schematic representation of synthesis reaction of the above-mentioned compound.

### a. Synthesis of(S)-4-ethyl-9-fluoro-4-hydroxy-11-methyl-1,12-dihydro-14H-pyrano[3′,4′:6,7]indolizino[1,2-b]quinoline-3,14(4H)-dione (Int-01)

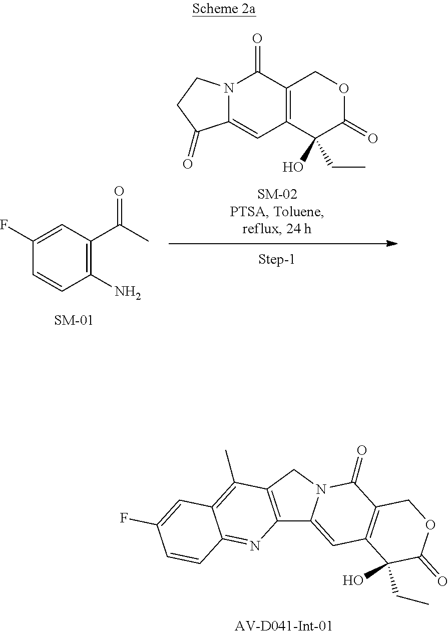 [SMILES: CC[C@@]1(O)C(=O)OCc2c1cc1n(c2=O)CCC1=O ; CC(=O)c1cc(F)ccc1N ; "SM-02PTSA, Toluene,reflux, 24 h" ; CC[C@@]1(O)C(=O)OCc2c1cc1n(c2=O)Cc2c-1nc1ccc(F)cc1c2C]

To a stirred solution of 1-(2-amino-5-fluorophenyl) ethan-1-one (SM-01, 350 mg, 1.33 mmol), and(S)-4-ethyl-4-hydroxy-7,8-dihydro-1H-pyrano[3,4-f]indolizine-3,6,10 (4H)-trione (SM-02, 366 mg, 2.39 mmol) in toluene (5 mL) was added p-toluenesulfonic acid (25 mg, 0.13 mmol) at room temperature and the reaction was heated at 110° C. for 24 h. The reaction was monitored by TLC. The resulting reaction mixture was concentrated under reduced pressure to get crude product. The crude product was triturated with diethyl ether to get(S)-4-ethyl-9-fluoro-4-hydroxy-11-methyl-1,12-dihydro-14H-pyrano[3′,4′:6,7indolizino[1,2-b]quinoline-3,14(4H)-dione (AV-D041-Int-01, 500 mg, 1.31 mmol, 98% yield).

MS (ESI): 381.03 [M+H]⁺; ¹H NMR (400 MHz, DMSO-d₆): δ 8.25-8.21 (m, 1H), 8.04 (dd, J=2.8, 10.4 Hz, 1H), 7.81-7.76 (m, 1H), 7.32, (s, 1H), 6.55-6.44 (m, 1H), 5.43 (s, 2H), 5.30 (s, 2H), 2.76 (s, 3H), 1.92-1.81 (m, 2H), 0.88 (t, J=7.2 Hz, 3H).

### b. Synthesis of(S)-4-ethyl-9-fluoro-4-hydroxy-11-(2-(isopropylamino)ethyl)-1,12-dihydro-14H-pyrano[3′,4′:6,7]indolizino[1,2-b]quinoline-3,14(4H)-dione (AV-D041)

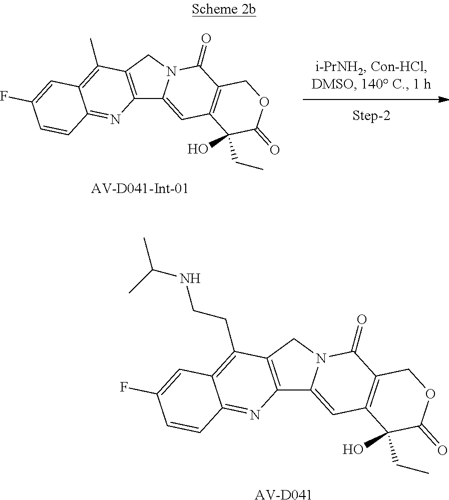

To a stirred solution of(S)-4-ethyl-9-fluoro-4-hydroxy-11-methyl-1,12-dihydro-14H-pyrano[3′,4′:6,7]indolizino[1,2-b]quinoline-3,14(4H)-dione (AV-D041-Int-01, 500 mg, 1.31 mmol), and isopropylamine (0.6 mL, 7.84 mmol) in dimethyl sulfoxide (5 ml) was added concentrated hydrochloric acid (0.5 mL) and the mixture was heated at 140° C. for 2 h. The reaction mixture was lyophilized with MeCN/H₂O to obtained brown gum which was purified by RP prep HPLC to get ((S)-4-ethyl-9-fluoro-4-hydroxy-11-(2-(isopropylamino)ethyl)-1,12-dihydro-14H-pyrano[3′,4′:6,7]indolizino[1,2-b]quinoline-3,14(4H)-dione (2.7 mg, 0.0059 mmol, 1% yield) as an off-white solid

MS (ESI): 450.0 [M−H]⁺; ¹H NMR (400 MHz, DMSO-d₆): δ 8.28-8.26 (m, 1H), 8.10 (dd, J=2.8, 10.8 Hz, 1H) 7.83-7.78 (m, 1H), 7.33 (s, 1H), 6.54 (brs, 1H), 5.44 (s, 2H), 5.39 (s, 2H), 3.38-3.36 (m, 2H), 3.04-3.01 (m, 2H), 3.54 (m, 1H), 1.91-1.83 (m, 2H), 1.07 (d, J=6.4 Hz, 6H), 0.87 (t, J=7.2 Hz, 3H); —NH peak was not observed.

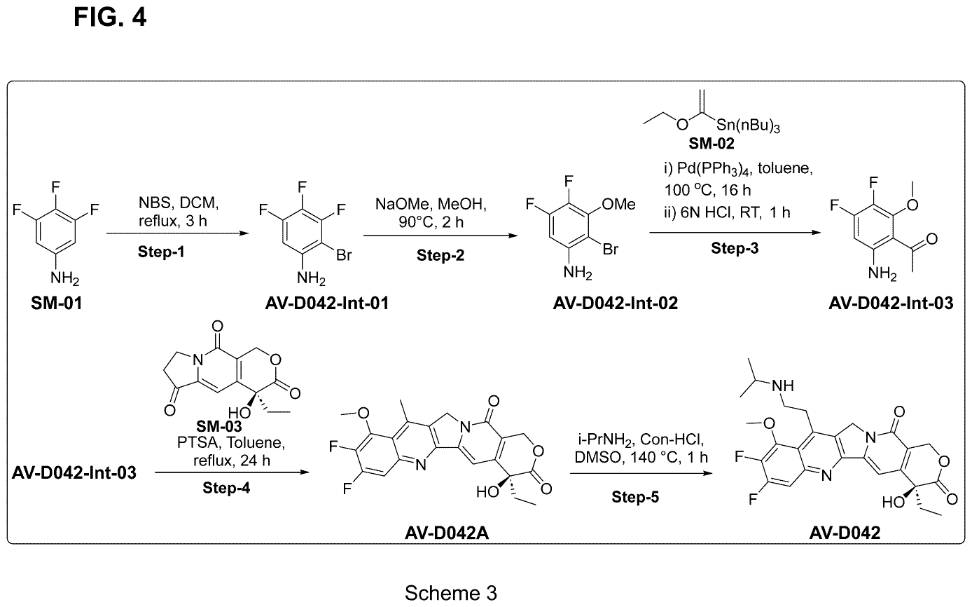

### III. Preparation of(S)-4-ethyl-8,9-difluoro-4-hydroxy-11-(2-(isopropylamino)ethyl)-10-methoxy-1,12-dihydro-14H-pyrano[3′,4′:6,7]indolizino[1,2-b]quinoline-3,14(4H)-dione (AV-D042)(Scheme 3, FIG. 4)

The following example provides a synthetic procedure for the preparation of the compounds(S)-4-ethyl-8,9-difluoro-4-hydroxy-11-(2-(isopropylamino)ethyl)-10-methoxy-1,12 -dihydro-14H-pyrano[3′,4′:6,7]indolizino[1,2-b]quinoline-3,14(4H)-dione (AV-D042). The FIG. 4 depicts the schematic representation of synthesis reaction of the above-mentioned compound.

### a. Synthesis of 2-bromo-3,4,5-trifluoroaniline (AV-D042-Int-01)

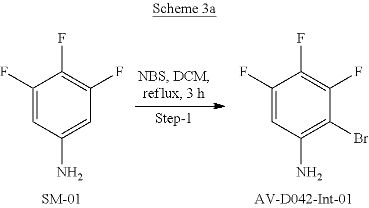 [SMILES: Nc1cc(F)c(F)c(F)c1 ; Nc1cc(F)c(F)c(F)c1Br]

To a stirred solution of 3,4,5-trifluoroaniline (SM-01, 3 g, 20.4 mmol) in dichloromethane (DCM, 60 mL), N-bromosuccinimide (NBS, 3.6 g, 20.4 mmol) was added. The reaction was stirred at room temperature for 3 h. The reaction mixture was passed through by celite bed and washed with DCM (50 mL). The filtrate was evaporated under reduced pressure and residue was purified by silica gel column chromatography eluted with 20% hexanes in ethyl acetate to obtained 2-bromo-3,4,5-trifluoroaniline (AV-D042-Int-01, 2.6 g, 12.05 mmol, 56% yield) as a brown semi solid. The schematic representation of the reaction is shown in Scheme 3a. ¹H NMR (400 MHz, DMSO-d₆): δ 6.63 (ddd, J=2.4, 7.2, 9.6 Hz, 1H), 5.80 (bs, 2H).

### b. Synthesis of 2-bromo-4,5-difluoro-3-methoxyaniline (AV-D042-Int-02)

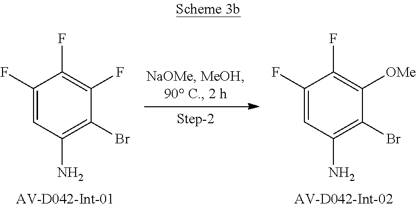 [SMILES: Nc1cc(F)c(F)c(F)c1Br ; COc1c(F)c(F)cc(N)c1Br]

To a stirred solution of 2-bromo-3,4,5-trifluoroaniline (AV-D042-Int-01, 1 g, 4.4 mmol) in MeOH (5 mL), 30% NaOMe in methanol (7.2 g, 38.4 mmol) was added at room temperature. The reaction was stirred at 90° C. for 2 h. The solvent was evaporated under reduced pressure and residue was purified by reverse phase GRACE (column 30 g, C18) eluted with 60% MeCN in water to obtain 2-bromo-4,5-difluoro-3-methoxyaniline (AV-D042-Int-02, 900 mg, 3.8 mmol, 85% yield) as a brown liquid. The schematic representation of the reaction is shown in Scheme 3b.

¹H NMR (400 MHz, DMSO-d₆): δ 6.55 (dd, J=7.2, 12.8 Hz, 1H), 5.51 (brs, 2H).3.86 (d, J=1.2, 3H).

### c. Synthesis of 1-(6-amino-3,4-difluoro-2-methoxyphenyl) ethan-1-one (AV-D042-Int-02; Formula II)

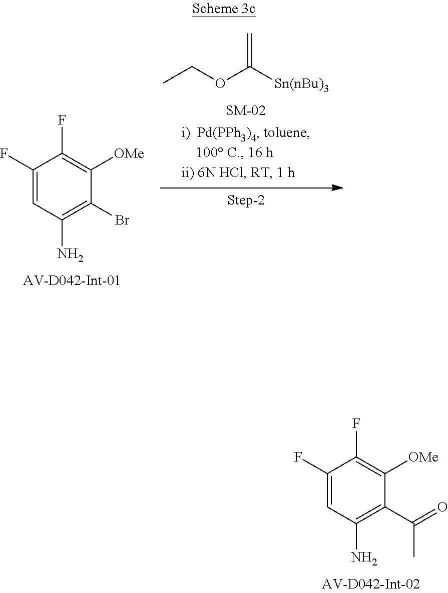

To a stirred solution of 2-bromo-4,5-difluoro-3-methoxyaniline (AV-D042-Int-02, Formula II, 500 mg, 2.11 mmol) in toluene (10 mL), tributyl(1-ethoxyvinyl) tin (917 mg, 2.54 mmol), and Pd(PPh₃)₄ (24 mg, 0.21 mmol) were added under nitrogen. The reaction was stirred at 100° C. for 16 h. The reaction was passed through celite bed and washed with 10% methanol in DCM (50 mL). The filtrate was evaporated under reduced pressure and residue was purified by silica gel column chromatography eluted with 30% hexanes in ethyl acetate to obtain to get 1-(6-amino-3,4-difluoro-2-methoxyphenyl) ethan-1-one (AV-D042-Int-02, 30 mg, 0.158 mmol, 7.5% yield) as a pale-yellow liquid. The schematic representation of the reaction is shown in Scheme 3c.

¹H NMR (400 MHz, DMSO-d₆): δ 6.86 (brs, 2H), 6.43 (dd, J=6.4, 12.8 Hz, 1H), 3.94 (d, J=2.4 Hz, 3H), 2.46 (s, 3H).

### d. Synthesis of(S)-4-ethyl-8,9-difluoro-4-hydroxy-10-methoxy-11-methyl-1,12-dihydro-14H-pyrano[3′,4′:6,7]indolizino[1,2-b]quinoline-3,14(4H)-dione (AV-D042A)

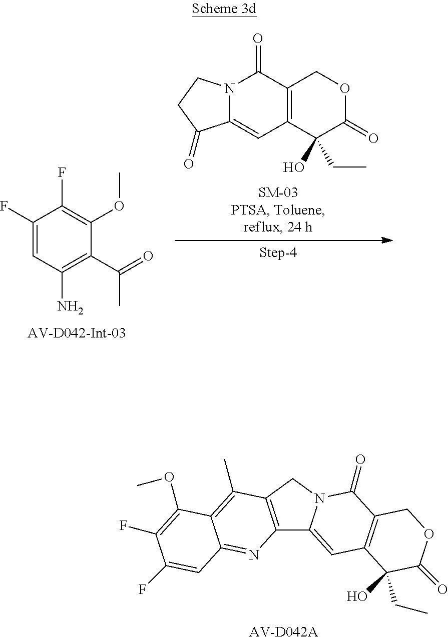

To a stirred solution of 1-(6-amino-3,4-difluoro-2-methoxyphenyl) ethan-1-one (AV-D042-Int-03, Formula II, 30 mg, 1.49 mmol), and(S)-4-ethyl-4-hydroxy-7,8-dihydro-1H-pyrano[3,4-f]indolizine-3,6,10 (4H)-trione (SM-03, Formula III, 31 m g, 1.19 mmol) in toluene (1 mL), PTSA (2.8 mg, 0.14 mmol) was added and the reaction was heated at 110° C. for 16 h. The reaction was passed through celite bed and washed with 10% methanol in DCM (50 mL). The filtrate was evaporated under reduced pressure and residue was purified by reverse phase prep HPLC to obtain(S)-4-ethyl-8,9-difluoro-4-hydroxy-10-methoxy-11-methyl-1,12-dihydro-14H-pyrano[3′,4′:6,7]indolizino[1,2-b]quinoline-3,14(4H)-dione (AV-D042A, 2.5 mg, 0.0058 mmol, 5% yield). The schematic representation of the reaction is shown in Scheme 3d.

MS (ESI): 429.15 [M+H]⁺; ¹H NMR (400 MHz, DMSO-d₆): δ 7.96 (dd, J=3.6, 7.6 Hz, 1H), 7.29 (s, 1H), 6.52 (s, 1H), 5.43 (s, 2H), 5.30 (s, 2H), 4.09 (d, J=1.6 Hz, 3H), 2.92 (s, 3H), 1.88-1.84 (m, 2H), 0.87 (t, J=7.2 Hz, 3H).

### e. Synthesis of(S)-4-ethyl-8,9-difluoro-4-hydroxy-11-(2-(isopropylamino)ethyl)-10-methoxy-1,12-dihydro-14H-pyrano[3′,4′:6,7]indolizino[1,2-b]quinoline-3,14(4H)-dione (AV-D042)

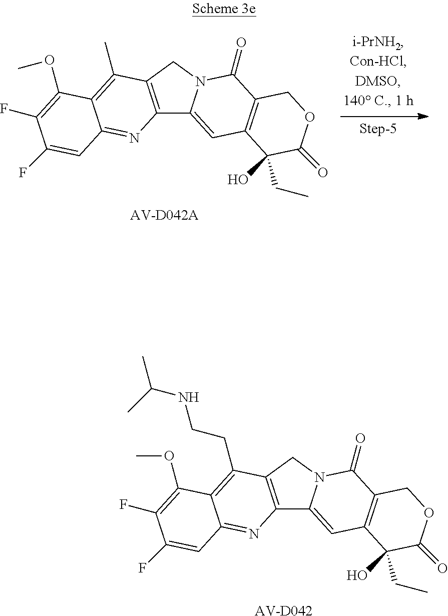

To a stirred solution of(S)-4-ethyl-8,9-difluoro-4-hydroxy-10-methoxy-11-methyl-1,12-dihydro-14H-pyrano[3′,4′:6,7]indolizino[1,2-b]quinoline-3,14(4H)-dione (AV-D042A, 30 mg, 0.07 mmol), and isopropylamine (0.03 mL, 0.41 mmol) in dimethyl sulfoxide (1 mL) was added concentrated hydrochloric acid (0.01 mL) and the mixture was heated at 140° C. for 1 h. The reaction was lyophilized with MeCN/H₂O to obtain brown gum which was purified by reverse phase prep HPLC to obtain(S)-4-ethyl-8,9-difluoro-4-hydroxy-11-(2-(isopropylamino)ethyl)-10-methoxy-1,12-dihydro-14H-pyrano[3′,4′:6,7]indolizino[1,2-b]quinoline-3,14(4H)-dione (AV-D042, 2.0 mg, 0.004 mmol, 5.7% yield). The schematic representation of the reaction is shown in Scheme 3e.

MS (ESI): 500.37 [M+H]⁺: ¹H NMR (400 MHz, DMSO-d₆): δ 8.29 (s, 1H), 7.95 (dd, J=8.0, 11.2 Hz, 1H), 7.30 (s, 1H), 6.53 (s, 1H), 5.44 (s, 2H), 5.37 (s, 2H), 4.17 (s, 3H), 3.48 (m, 2H), 3.93 (d, J=6.8 Hz, 2H), 2.89-2.86 (m, 1H), 1.90-1.84 (m, 2H), 1.00 (d, J=6.4 Hz, 6H), 0.87 (t, J=7.2 Hz, 3H).

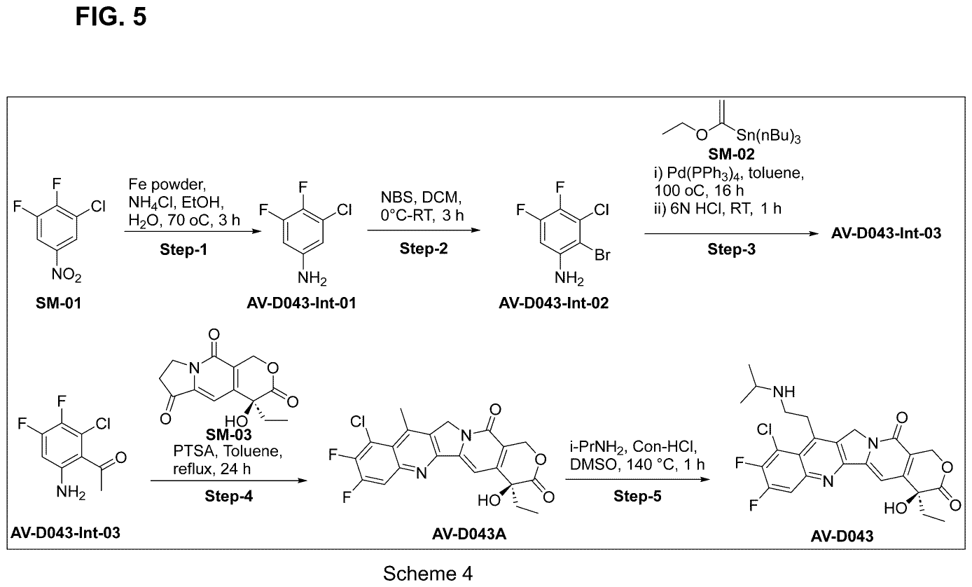

### IV. Preparation of(S)-10-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D043A) and(S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043)(Scheme 4, FIG. 5)

The following example provides synthetic procedure for the preparation of the compounds(S)-10-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D043A) and(S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043). The FIG. 5 depicts the schematic representation of synthesis reaction of the above-mentioned compounds.

### a. Synthesis of 3-chloro-4,5-difluoroaniline (AV-D043-Int-01)

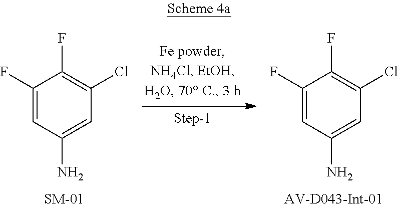 [SMILES: Nc1cc(F)c(F)c(Cl)c1 ; Nc1cc(F)c(F)c(Cl)c1]

To a stirred solution of 1-chloro-2,3-difluoro-5-nitrobenzene (SM-01, 2 g, 10.33 mmol) and Fe powder (5.7 g, 103 mmol) in ethanol:H₂O (8:2, 40 mL) was added NH₄Cl (5.4 g, 103 mmol) at room temperature. The reaction mixture was stirred at 70° C. for 3 h. The reaction was monitored by TLC. The reaction was cooled to 25° C. and then filtered through celite pad. The obtained filtrate was diluted with water (20 mL) and extracted with EtOAc (2×100 mL). The combined organic layer was washed with brine (50 mL), dried over anhydrous Na₂SO₄, filtered, and concentrated under reduced pressure to obtain 3-chloro-4,5-difluoroaniline (AV-D043-Int-01, 1.7 g, 10.4 mmol, quantitative yield) as a brown solid. The schematic representation of the reaction is shown in Scheme 4a.

MS (ESI): 164.19 [M+H]⁺; ¹H NMR (400 MHz, DMSO-d₆): δ 6.51-6.45 (m, 2H), 5.53 (brs, 2H).

### b. Synthesis of 2-bromo-3-chloro-4,5-difluoroaniline (AV-D043-Int-02)

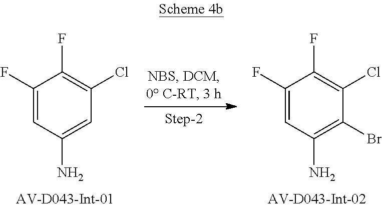 [SMILES: Nc1cc(F)c(F)c(Cl)c1 ; Nc1cc(F)c(F)c(Cl)c1Br]

To a stirred solution of 3-chloro-4,5-difluoroaniline (AV-D043-Int-01, 1.7 g, 10.36 mmol) in DCM (20 mL) was added NBS (1.8 g, 10.36 mmol) at room temperature. The reaction mixture was stirred at RT for 3 h. The reaction was monitored by TLC. The reaction was diluted with water (20 mL) and extracted with EtOAc (2×100 mL). The combined organic layer was washed with brine (50 mL), dried over anhydrous Na₂SO₄, filtered and concentrated under reduced pressure to obtain crude compound. The crude compound was purified by silica gel column chromatography (SiO₂, 100-200 mesh, 20% EtOAc/hexane) to afford 2-bromo-3-chloro-4,5-difluoroaniline (AV-D043-Int-02, 1.9 g, 7.88 mmol, 76% yield) as a colourless semi solid. The schematic representation of the reaction is shown in Scheme 4b.

MS (ESI): 242.07 [M+H]⁺; ¹H NMR (400 MHz, DMSO-d₆): δ 6.80 (dd, J=7.2, 12.8 Hz, 1H), 5.79 (brs, 2H).

### c. Synthesis of 1-(6-amino-2-chloro-3,4-difluorophenyl) ethan-1-one (AV-D043-Int-03; Formula II)

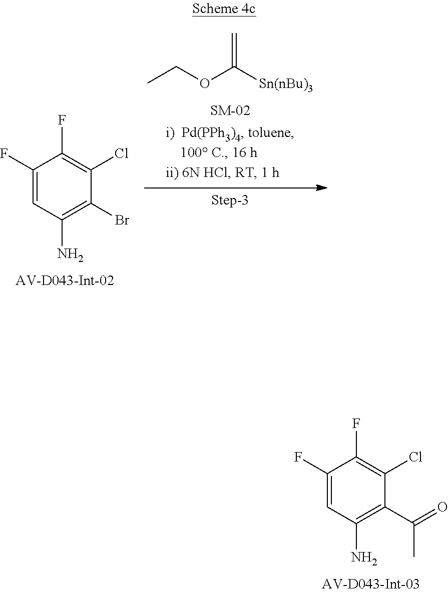

To a stirred solution of 2-bromo-3-chloro-4,5-difluoroaniline (AV-D043-Int-02, 900 mg, 3.75 mmol) and tributyl(1-ethoxyvinyl) stannane (SM-02, 1.62 g, 4.5 mmol) in toluene (20 mL), Pd(PPh₃)₄ (43 mg, 0.037 mmol) was added at room temperature. The reaction mixture was stirred at 100° C. for 16 h. The reaction was monitored by TLC. The reaction was cooled to 25° C. and then treated with 6N HCl (10 mL) and stirred at same temperature for 1 h. The reaction was diluted with water (20 mL) and extracted with EtOAc (2× 50 mL). The combined organic layer was washed with brine (50 mL), dried over anhydrous Na₂SO₄, filtered and concentrated under reduced pressure to obtain crude compound. The crude compound was purified by silica gel column chromatography (SiO₂, 100-200 mesh, 30% EtOAc/hexane) to afford 1-(6-amino-2-chloro-3,4-difluorophenyl) ethan-1-one (AV-D043-Int-03, 300 mg, 0.95 mmol, 39% yield) as a pale-yellow liquid. The schematic representation of the reaction is shown in Scheme 4c.

¹H NMR (400 MHz, DMSO-d₆): δ 6.61 (dd, J=6.8, 12.8 Hz, 1H), 5.28 (brs, 2H), 2.50 (s, 3H).

### d. Synthesis of(S)-10-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D043A)

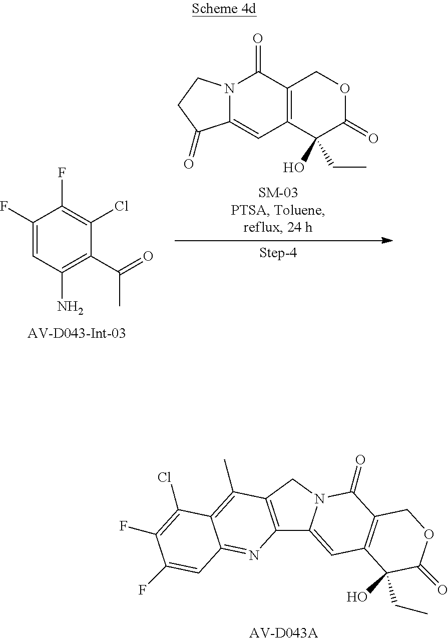

To a stirred solution of 1-(6-amino-2-chloro-3,4-difluorophenyl) ethan-1-one (AV-D043-Int-03, Formula II, 300 mg, 0.95 mmol), and(S)-4-ethyl-4-hydroxy-7,8-dihydro-1H-pyrano[3,4-f]indolizine-3,6, 10 (4H)-trione (SM-03, Formula III, 207 mg, 1.17 mmol) in toluene (solvent, 5 mL), p-toluenesulfonic acid (27 mg, 0.14 mmol) was added at room temperature and the reaction mixture was heated at 110° C. for 24 h. The reaction was monitored by TLC. The resulting reaction mixture was concentrated under reduced pressure to get crude product. The crude product was further purified by RP Biotage to get(S)-10-chloro-4-ethyl-8,9-difluoro-4-hydroxy-11-methyl-1,12-dihydro-14H-pyrano[3′,4′:6,7]indolizino[1,2-b]quinoline-3, 14 (4H)-dione (AV-D043A 200 mg, 0.46 mmol, 40% yield), as a light brown solid. The schematic representation of the reaction is shown in Scheme 4d.

MS (ESI): 433.23 [M+H]⁺; ¹H NMR (400 MHz, DMSO-d₆): δ 8.26 (dd, J=8.4, 10.8 Hz, 1H), 7.31 (s, 1H), 6.55 (s, 1H), 5.44 (s, 2H), 5.31 (s, 2H), 3.03 (s, 3H), 1.88-1.84 (m, 2H), 0.87 (t, J=7.2 Hz, 3H).

### e. Synthesis of(S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043)

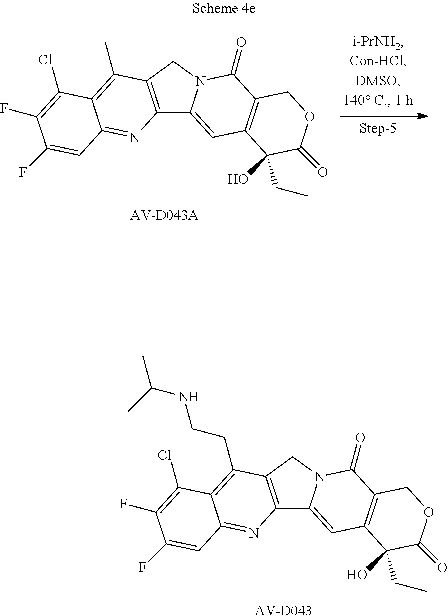

To a stirred solution of(S)-10-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D043A, 200 mg, 0.462 mmol), and isopropylamine (reagent, 0.2 mL, 2.7 mmol) in 6 mL of dimethyl sulfoxide, concentrated hydrochloric acid (0.4 mL) was added and the mixture was heated for 2 hour at 140° C. The reaction mixture was lyophilised to get brown gum which was purified by RP Prep HPLC to obtain(S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043, 4.9 mg, 0.097 mmol, 2% yield) as a pale yellow solid. The schematic representation of the reaction is shown in Scheme 4e.

MS (ESI): 504.16 [M+H]⁺; ¹H NMR (400 MHz, DMSO-d₆): δ 8.32 (dd, J=2.4, 12.0 Hz, 1H), 7.33 (s, 1H), 6.58 (brs, 1H), 5.45 (s, 2H), 5.42 (s, 2H), 3.72-3.70 (m, 2H), 3.11-3.07 (m, 3H), 1.88-1.85 (m, 2H), 1.08 (d, J=6.4 Hz, 6H), 0.87 (t, J=7.2 Hz, 3H); —NH proton was not observed.

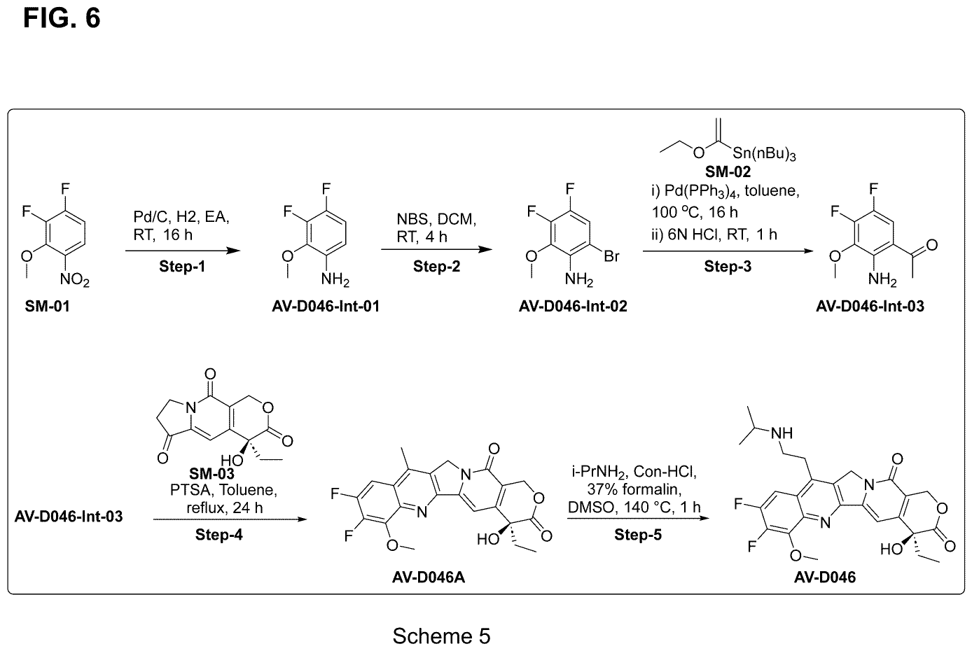

### V. Preparation of(S)-4-ethyl-8,9-difluoro-4-hydroxy-7-methoxy-11-methyl-1,12-dihydro-14H-pyrano[3′,4′:6,7]indolizino[1,2-b]quinoline-3,14(4H)-dione (AV-D046A) and(S)-4-ethyl-8,9-difluoro-4-hydroxy-11-(2-(isopropylamino)ethyl)-7-methoxy-1,12-dihydro-14H-pyrano[3′,4′:6,7]indolizino[1,2-b]quinoline-3,14(4H)-dione (AV-D046)(Scheme 5, FIG. 6)

The following example provides synthetic procedure for the preparation of the compounds(S)-4-ethyl-8,9-difluoro-4-hydroxy-7-methoxy-11-methyl-1,12-dihydro-14H-pyrano[3′,4′:6,7]indolizino[1,2-b]quinoline-3,14(4H)-dione (AV-D046A) and(S)-4-ethyl-8,9-difluoro-4-hydroxy-11-(2-(isopropylamino)ethyl)-7-methoxy-1,12-dihydro-14H-pyrano[3′,4′:6,7]indolizino[1,2-b]quinoline-3,14(4H)-dione (AV-D046). The FIG. 6 depicts the schematic representation of synthesis reaction of the above-mentioned compounds.

### a. Synthesis of 3,4-difluoro-2-methoxyaniline (AV-D046-Int-01)

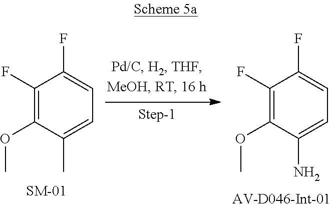

To a stirred solution of 1,2-difluoro-3-methoxy-4-nitrobenzene (SM-01, 5 g, 26.46 mmol) in THF (50 mL) and methanol (50 mL), was added 10% w/w Pd/C (1.5 g). The mixture was stirred at room temperature for 16 h under hydrogen balloon pressure. The reaction mixture was filter through celite pad and washed with THF (50 mL). The filtrate was evaporated was under reduced pressure and dried to afford 3,4-difluoro-2-methoxyaniline (AV-D046-Int-01, 4 g, 25.16 mmol, 95%) as a brown gum.

MS (ESI): 160.2 [M+H]⁺; ¹H NMR (400 MHz, DMSO-d₆): δ 6.87-6.80 (m, 1H), 6.45-6.40 (m, 1H), 5.02 (bs, 2H), 3.78 (s, 3H).

### b. Synthesis of 6-bromo-3,4-difluoro-2-methoxyaniline (AV-D046-Int-02)

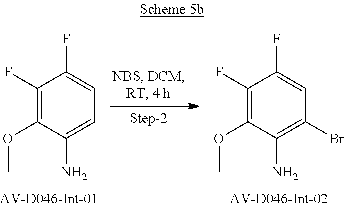

To a stirred solution of 3,4-difluoro-2-methoxyaniline (AV-D046-Int-01, 4 g, 25.16 mmol) in DCM (80 mL) was added NBS (1.5 g, 25.16 mmol). The reaction was stirred at room temperature for 4 h. The reaction mixture was diluted with water (20 mL) and extracted with ethylacetate (100 mL), organic layer was washed with brine solution (25 mL), dried over Na₂SO₄, filtered and evaporated under reduced pressure. The residue was then purified by column chromatography (100-200 silica gel mesh) eluted with 25% ethylacetate in hexanes to obtain 6-bromo-3,4-difluoro-2-methoxyaniline (AV-D046-Int-02, 3 g, 12.61 mmol, 50%) as a light pink gum.

MS (ESI): 237.98. [M+H]⁺; ¹H NMR (400 MHz, DMSO-d₆): δ 7.34 (dd, J=7.6, 10.0 Hz, 1H), 5.14 (brs, 2H), 3.83 (d, J=1.2 Hz, 3H).

### c. Synthesis of 1-(2-amino-4,5-difluoro-3-methoxyphenyl) ethan-1-one (AV-D046-Int-03)

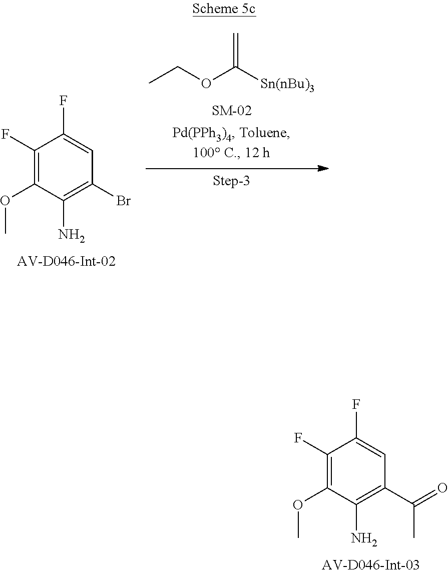

To a stirred solution of 6-bromo-3,4-difluoro-2-methoxyaniline (AV-D046-Int-02, 3 g, 12.61 mmol) in toluene (30 mL) was added tributyl-(1-ethoxyvinyl) tin (5.5 g, 15.12 mmol), Pd(PPh₃)₄ (146 mg, 0.126 mmol) under nitrogen. The reaction was stirred at 100° C. for 12 h. The reaction mixture was diluted with water (20 mL) and extracted with ethylacetate (100 mL). The organic layer was separated, washed with brine (25 mL), dried over Na₂SO₄, filtered and evaporated under reduced pressure. The residue was purified by column chromatography (100-200 silica gel mesh) eluted with 30% ethylacetate in hexanes to obtain 1-(2-amino-4,5-difluoro-3-methoxyphenyl) ethan-1-one (AV-D046-Int-03, 1.6 g, 6.08 mmol, 63%) as a light brown solid. MS (ESI): 201.89 [M+H]⁺; ¹H NMR (400 MHz, DMSO-d₆): δ 7.62 (dd, J=8.8, 12.4 Hz, 1H), 7.11 (brs, 2H), 3.84 (d, J=1.2 Hz, 3H), 2.50-2.49 (m, 3H).

### d. Synthesis of(S)-4-ethyl-8,9-difluoro-4-hydroxy-7-methoxy-11-methyl-1,12-dihydro-14H-pyrano[3′,4′:6,7]indolizino[1,2-b]quinoline-3,14(4H)-dione (AV-D046A)

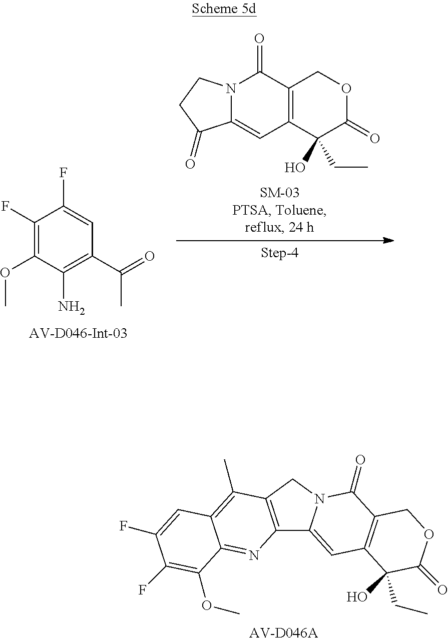

To a stirred solution of 1-(2-amino-4,5-difluoro-3-methoxyphenyl) ethan-1-one (AV-D046-Int-03, 0.4 g, 1.53 mmol), and(S)-4-ethyl-4-hydroxy-7,8-dihydro-1H-pyrano[3,4-f]indolizine-3,6,10 (4H)-trione (SM-03, 0.55 g, 2.74 mmol) in 4 mL of toluene was added PTSA (0.029 g, 0.15 mmol) and the mixture was heated at 120° C. for 24 h. The reaction mixture was diluted with water (20 mL) and extracted with 10% methanol in dichloromethane (100 mL). The organic layer was washed with brine (25 mL), dried over Na₂SO₄, filtered and evaporated under reduced pressure. The residue was purified by column chromatography (100-200 silica gel mesh) eluted with 10% methanol in dichloromethane to obtain(S)-4-ethyl-8,9-difluoro-4-hydroxy-7-methoxy-11-methyl-1,12-dihydro-14H-pyrano[3′,4′:6,7]indolizino[1,2-b]quinoline-3,14(4H)-dione (AV-D046A, 350 mg, 0.817 mmol, 29% yield) as a light brown solid.

MS (ESI): 429.27 [M+H]⁺; ¹H NMR (400 MHz, DMSO-d₆): δ 8.01 (dd, J=8.0, 12.0 Hz, 1H), 7.33 (s, 1H), 6.55 (s, 1H), 5.43 (s, 2H), 5.28 (s, 2H), 4.26 (s, 3H), 2.73 (s, 3H), 1.91-1.84 (m, 2H), 0.88 (t, J=7.2 Hz, 3H).

### e. Synthesis of(S)-4-ethyl-8,9-difluoro-4-hydroxy-11-(2-(isopropylamino)ethyl)-7-methoxy-1,12-dihydro-14H-pyrano[3′,4′:6,7]indolizino[1,2-b]quinoline-3,14(4H)-dione (AV-D046)

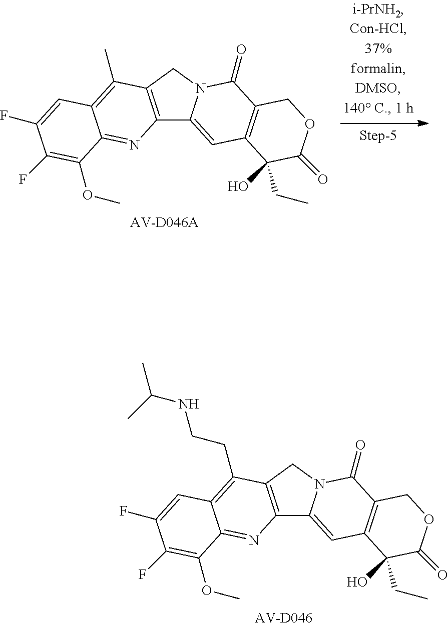

To a stirred solution of(S)-4-ethyl-8,9-difluoro-4-hydroxy-7-methoxy-11-methyl-1,12-dihydro-14H-pyrano[3′,4′:6,7]indolizino[1,2-b]quinoline-3,14(4H)-dione (AV-D046A, 150 mg, 0.36 mmol) and isopropylamine (0.2 mL, 2.11 mmol) in dimethyl sulfoxide (3 mL) was added concentrated hydrochloric acid (0.2 mL) and 37% formalin in water (0.1 mL), and the reaction was heated for 1 hour at 140° C. The reaction mixture was lyophilised with MeCN/H₂O (1:1, 2 mL) and the residue was purified by reverse phase Prep HPLC to obtained (S)-4-ethyl-8,9-difluoro-4-hydroxy-11-(2-(isopropylamino)ethyl)-7-methoxy-1,12-dihydro-14H-pyrano[3′,4′:6,7]indolizino[1,2-b]quinoline-3,14(4H)-dione (AV-D046, 2.8 mg, 0.0056 mmol, 1.6% yield), as an off white solid.

MS (ESI): 498.0 [M−H]⁻, ¹H NMR (400 MHz, DMSO-d₆): δ 8.42 (brs, 1H), 8.04 (dd, J=8.0, 11.6 Hz, 1H), 7.37 (s, 1H), 6.62 (s, 1H), 5.46 (s, 2H), 5.42 (s, 2H), 4.27 (s, 3H), 3.51-3.29 (m, 3H), 3.23-3.21 (m, 1H), 2.55-2.50 (m, 1H), 1.91-1.86 (m, 2H), 1.23 (d, J=6.0 Hz, 6H), 0.87 (t, J=4.8 Hz, 3H).

Method Details: Column: Shim-pack C18 (250*20.0 mm) 5μ Mobile Phase A: 0.1% TFA in water Mobile Phase B: 0.1% TFA in methanol Gradient Time: 0/20,5/50,26/50,26.1/98 at 12 mL/min Solubility: MeCN+THF+H₂O

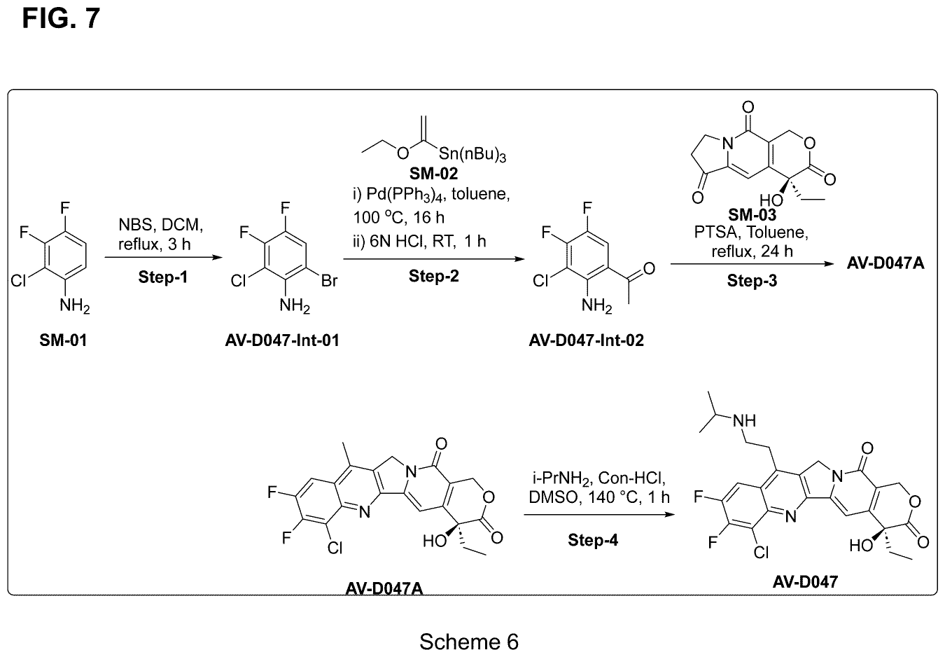

### VI. Preparation of(S)-7-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D047A) and(S)-7-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D047)(Scheme 6, FIG. 7)

The following example provides synthetic procedure for the preparation of the compounds(S)-7-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D047A) and(S)-7-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D047). The FIG. 7 depicts the schematic representation of synthesis reaction of the above-mentioned compounds.

### a. Synthesis of 6-bromo-2-chloro-3,4-difluoroaniline (AV-D047-Int-01)

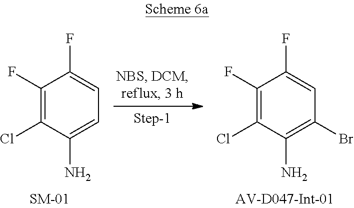 [SMILES: Nc1ccc(F)c(F)c1Cl ; Nc1c(Br)cc(F)c(F)c1Cl]

To a stirred solution of 2-chloro-3,4-difluoroaniline (SM-01, 2 g, 12.26 mmol) in DCM (20 mL), was added NBS (2.1 g, 12.26 mmol) and stirred at room temperature for 3 h. The reaction mixture was diluted with water (30 mL) and extracted with ethylacetate (50 mL). The organic layer was washed with brine (30 mL), dried over Na₂SO₄, filtered and evaporated under reduced pressure to get the crude product which was purified by column chromatography (100-200 silica gel mesh) eluted with 10% ethylacetate in n-hexanes to obtained to 6-bromo-2-chloro-3,4-difluoroaniline (AV-D047-Int-01, 2.8 g, 12.44 mmol, 94%) as a light yellow liquid. ¹H NMR (400 MHz, DMSO-d₆): δ 7.68 (dd, J=7.6, 10 Hz, 1H), 5.62 (brs, 2H).

### A. Synthesis of 1-(2-amino-3-chloro-4,5-difluorophenyl) ethan-1-one (AV-D047-Int-02)

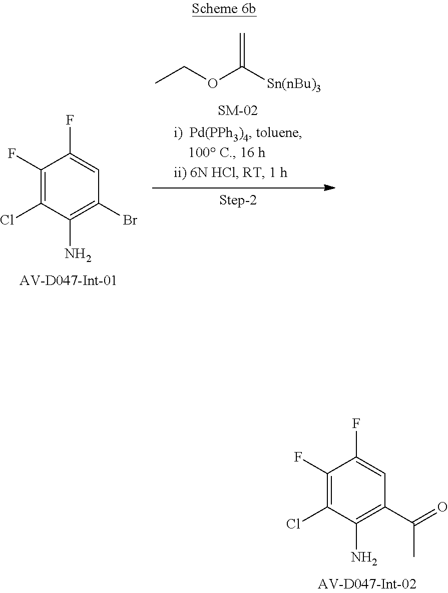

To a stirred solution of 6-bromo-2-chloro-3,4-difluoroaniline (AV-D047-Int-01, 500 mg, 2.08 mmol) in toluene (5 mL) was added tributyl-(1-ethoxyvinyl) tin (902 mg, 2.499 mmol) and Pd(PPh₃)₄ (24 mg, 0.0208 mmol) under nitrogen. The reaction was stirred at 100° C. for 16 h. The reaction was diluted with water (10 mL) and extracted with ethylacetate (30 mL). The organic layer was washed with brine (25 mL), dried over Na₂SO₄, filtered and evaporated under reduced pressure to get the crude product which was purified by column chromatography (100-200 silica gel mesh) eluted with 20% ethylacetate in hexanes to obtain 1-(2-amino-3-chloro-4,5-difluorophenyl) ethan-1-one (AV-D047-Int-02, 400 mg, 1.95 mmol, 94% yield) as a pale yellow solid. ¹H NMR (400 MHz, DMSO-d₆): δ 7.98-7.93 (m, 1H), 7.46 (brs, 2H), 2.57 (s, 3H).

### B. Synthesis of(S)-7-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D047A)

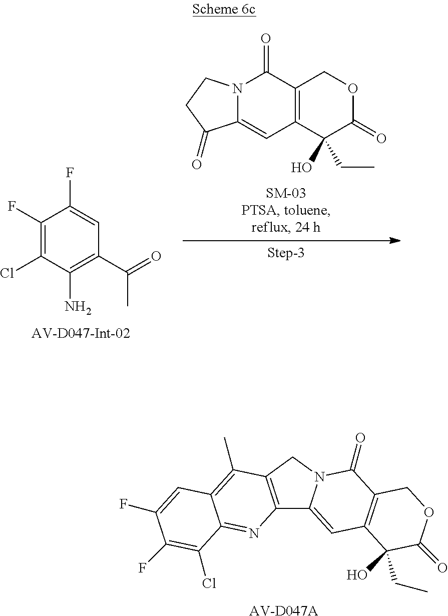

To a stirred solution of 1-(2-amino-3-chloro-4,5-difluorophenyl) ethan-1-one (AV-D047-Int-02, 400 mg, 1.95 mmol), and(S)-4-ethyl-4-hydroxy-7,8-dihydro-1H-pyrano[3,4 -f]indolizine-3,6,10 (4H)-trione (SM-03, 205 mg, 1.56 mmol) in toluene (5 mL) was added PTSA (18 mg, 0.19 mmol) and the mixture was heated at 110° C. for 24 h. The reaction mixture was diluted with water (20 mL) and extracted with 10% methanol in dichloromethane (50 mL). The organic layer was washed with brine solution (25 mL), dried over Na₂SO₄, filtered and evaporated under reduced pressure to obtain(S)-7-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D047A, 300 mg, 0.692 mmol, 88% crude yield) as a light brown solid.

MS (ESI): 433.28 [M+H]⁺; ¹H NMR (400 MHz, DMSO-d₆): δ 8.34 (dd, J=8.8, 12.0 Hz, 1H), 7.35 (s, 1H), 6.59 (s, 1H), 5.44 (s, 2H), 5.31 (s, 2H), 2.78 (s, 3H), 1.89-1.62 (m, 2H), 0.89 (t, J=7.2 Hz, 3H).

### C. Synthesis of(S)-7-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D047)

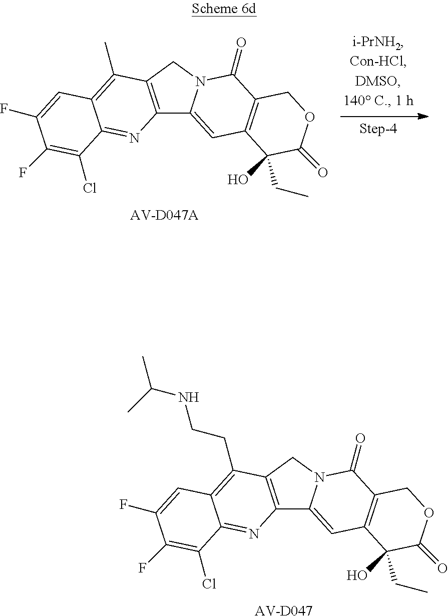

To a stirred solution of(S)-7-chloro-4-ethyl-8,9-difluoro-4-hydroxy-11-methyl-1,12-dihydro-14H-pyrano[3′,4′:6,7]indolizino[1,2-b]quinoline-3,14(4H)-dione (AV-D047A, 25 mg, 0.041 mmol), and isopropylamine (0.2 mL, 2.11 mmol) in dimethyl sulfoxide (3 mL) was added concentrated hydrochloric acid (0.2 mL) and the mixture was heated at 140° C. for 1 h. The reaction was lyophilised with MeCN/H₂O and residue was purified by RP prep HPLC to obtain (S)-7-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D047, 1.8 mg, 0.0035 mmol, 6% yield) as an off white solid.

MS (ESI): 504.16 [M+H]⁺; ¹H NMR (400 MHz, DMSO-d₆): δ 8.44 (dd, J=8.4, 11.6 Hz, 1H), 8.27 (s, 1H), 7.36 (s, 1H), 6.60 (s, 1H), 5.45 (s, 2H), 5.40 (s, 2H), 2.89 (t, J=6.8 Hz, 2H), 2.80-2.74 (m, 2H), 1.93-1.84 (m, 3H), 1.23 (d, J=6.4 Hz, 6H), 0.88 (t, J=4.8 Hz, 3H).

Method Details: Column: X-BRIDGE C18 (250*21.2 mm) 5μ: Mobile Phase A: 0.1% FA IN WATER; B: ACN: Gradient Time: 0/18,1/18,20/45,20.1/98: 10 mL/Min Solubility: ACN+H₂O.

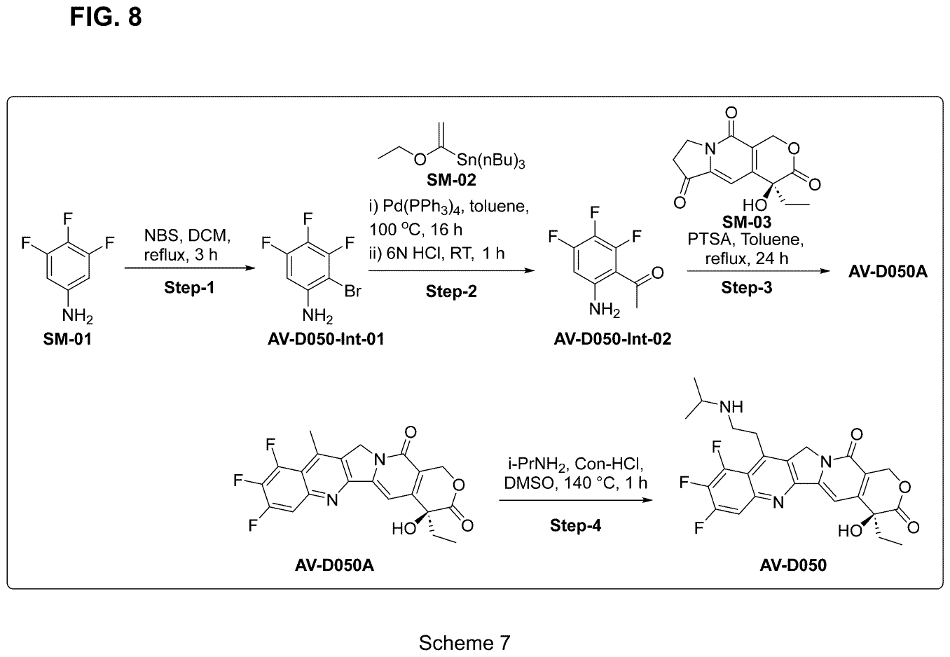

### VII. Preparation of(S)-4-ethyl-8,9,10-trifluoro-4-hydroxy-11-methyl-1,12-dihydro-14H-pyrano[3′,4′:6,7]indolizino[1,2-b]quinoline-3,14(4H)-dione (AV-D050A) and(S)-4-ethyl-8,9,10-trifluoro-4-hydroxy-11-(2-(isopropylamino)ethyl)-1,12-dihydro-14H-pyrano[3′,4′:6,7]indolizino[1,2-b]quinoline-3,14(4H)-dione (AV-D050)(Scheme 7, FIG. 8)

The following example provides a synthetic procedure for the preparation of the compounds(S)-4-ethyl-8,9,10-trifluoro-4-hydroxy-11-methyl-1,12-dihydro-14H-pyrano[3′,4′:6,7]indolizino[1,2-b]quinoline-3,14(4H)-dione (AV-D050A) and(S)-4-ethyl-8,9,10-trifluoro-4-hydroxy-11-(2-(isopropylamino)ethyl)-1,12-dihydro-14H-pyrano[3′,4′:6,7]indolizino[1,2-b]quinoline-3,14(4H)-dione (AV-D050). The FIG. 8 depicts the schematic representation of synthesis reaction of the above-mentioned compounds.

### a. Synthesis of 2-bromo-3,4,5-trifluoroaniline (AV-D050-Int-01)

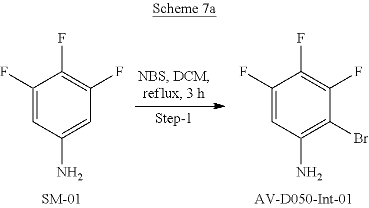 [SMILES: Nc1cc(F)c(F)c(F)c1 ; Nc1cc(F)c(F)c(F)c1Br]

To a stirred solution of 3,4,5-trifluoroaniline (SM-01, 1.7 g, 10.36 mmol) in DCM (20 mL) NBS (1.8 g, 10.36 mmol) was added at room temperature and then reflux for 3 h. The reaction was monitored by TLC. The reaction was diluted with water (20 mL) and extracted with EtOAc (2×50 mL). The combined organic layer was washed with brine (50 mL), dried over anhydrous Na₂SO₄, filtered and concentrated under reduced pressure to obtain crude compound. The crude compound was purified by column chromatography (SiO₂, 100-200 mesh, 15% EtOAc/hexane) to afford 2-bromo-3,4,5-trifluoroaniline (AV-D050-Int-01, 1.9 g, 8.4 mmol, 81% yield) as a colourless semi solid. The schematic representation of the reaction is shown in Scheme 7a.

MS (ESI): 242.07 [M+H]⁺; ¹H NMR (400 MHz, DMSO-d₆): δ 6.80 (dd, J=7.2, 12.8 Hz, 1H), 5.79 (brs, 2H).

### b. Synthesis of 1-(6-amino-2,3,4-trifluorophenyl) ethan-1-one (AV-D050-Int-02)

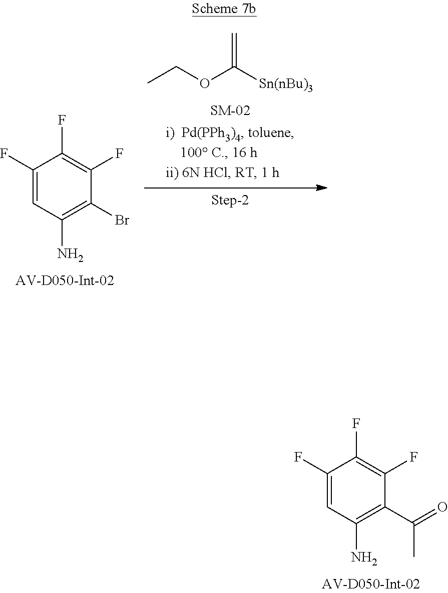

To a stirred solution of 2-bromo-3,4,5-trifluoroaniline (AV-D050-Int-01, 2 g, 8.92 mmol) and tributyl(1-ethoxyvinyl) stannane (SM-02, 3.8 g, 10.7 mmol) in toluene (20 mL) Pd(PPh₃)₄ (103 mg, 0.089 mmol) was added at room temperature. The reaction mixture was stirred at 100° C. for 16 h. The reaction was monitored by TLC. The reaction was cooled to 25° C., treated with 6N HCl (20 mL) and stirred at same temperature for 1 h. The reaction was diluted with water (20 mL) and extracted with EtOAc (2×100 mL). The combined organic layer was washed with brine (50 mL), dried over anhydrous Na₂SO₄, filtered and concentrated under reduced pressure to obtain crude compound. The crude compound was purified by silica gel column chromatography (SiO₂, 100-200 mesh, 15% EtOAc/hexane) to afford 1-(6-amino-2,3,4-trifluorophenyl) ethan-1-one (AV-D050-Int-02, 1.8 g, 9.5 mmol, quantitative yield) as a pale-yellow semi solid. The schematic representation of the reaction is shown in Scheme 7b.

¹H NMR (400 MHz, DMSO-d₆): δ 7.43 (brs, 2H), 6.61-6.54 (m, 1H), 2.49 (s, 3H).

### c. Synthesis of(S)-4-ethyl-8,9,10-trifluoro-4-hydroxy-11-methyl-1,12-dihydro-14H-pyrano[3′,4′:6,7]indolizino[1,2-b]quinoline-3,14(4H)-dione (AV-D050A)

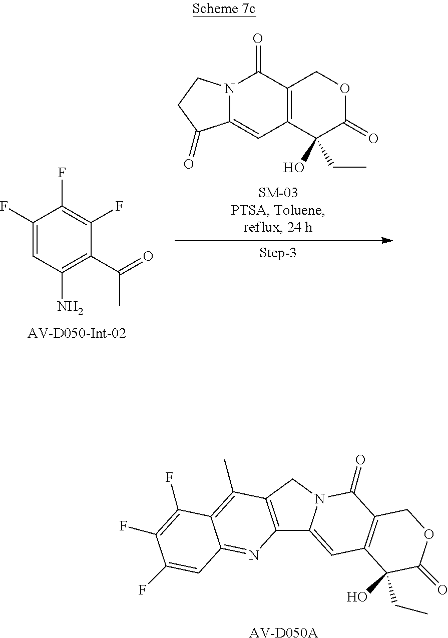

To a stirred solution of 1-(6-amino-2,3,4-trifluorophenyl) ethan-1-one (AV-D050-Int-02, Formula II, 800 mg, 3.04 mmol), and(S)-4-ethyl-4-hydroxy-7,8-dihydro-1H-pyrano[3,4 -f]indolizine-3,6,10 (4H)-trione (SM-03, Formula III, 1 g, 5.47 mmol) in toluene (10 mL) p-toluenesulfonic acid (57 mg, 0.34 mmol) was added at room temperature and the reaction mixture was heated at 110° C. for 16 h. The reaction was monitored by TLC. The resulting reaction mixture was concentrated under reduced pressure to get crude product. The crude product was further purified by RP Biotage to get(S)-4-ethyl-8,9,10-trifluoro-4-hydroxy-11-methyl-1,12-dihydro-14H-pyrano[3′,4′:6,7]indolizino[1,2-b]quinoline-3,14(4H)-dione (AV-D050A, 1 g, 2.4 mmol, 63% yield) as a brown solid. The schematic representation of the reaction is shown in Scheme 7c.

MS (ESI): 415.0 [M−H]⁻; ¹H NMR (400 MHz, DMSO-d₆): δ 8.14-8.10 (m, 1H), 7.32 (s, 1H), 6.54 (s, 1H), 5.44 (s, 2H), 5.32 (s, 2H), 3.88 (d, J=6.0 Hz, 3H), 1.89-1.82 (m, 2H), 0.87 (t, J=7.2 Hz, 3H).

### d. Synthesis of(S)-4-ethyl-8,9,10-trifluoro-4-hydroxy-11-(2-(isopropylamino)ethyl)-1,12-dihydro-14H-pyrano[3′,4′:6,7]indolizino[1,2-b]quinoline-3,14(4H)-dione (AV-D050)

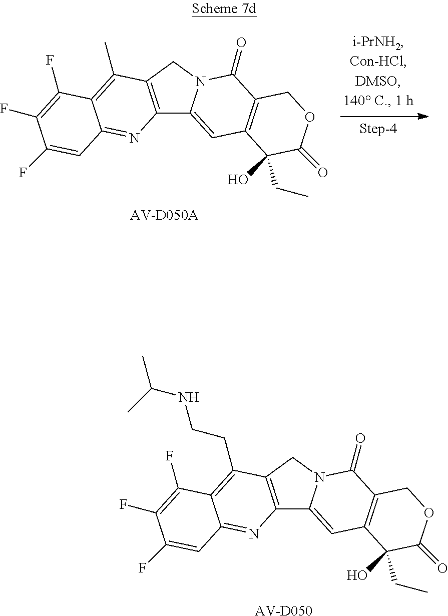

To a solution of(S)-4-ethyl-8,9,10-trifluoro-4-hydroxy-11-methyl-1,12-dihydro-14H-pyrano[3′,4′:6,7]indolizino[1,2-b]quinoline-3,14(4H)-dione (AV-D050A, 500 mg, 1.2 mmol), and isopropylamine (0.6 mL, 7.15 mmol) in 5 mL of dimethyl sulfoxide, concentrated hydrochloric acid (0.8 mL) was added and the mixture was heated for 2 hours at 140° C. The reaction mixture was lyophilised in MeCN/H₂O to get brown gum which was purified by RP Prep HPLC to obtain (S)-4-ethyl-8,9,10-trifluoro-4-hydroxy-11-(2-(isopropylamino)ethyl)-1,12-dihydro-14H-pyrano[3′,4′:6,7]indolizino[1,2-b]quinoline-3,14(4H)-dione (AV-D050, 6.5 mg, 0.013 mmol, 1% yield) as an off white solid. The schematic representation of the reaction is shown in Scheme 7d.

MS (ESI): 486.0 [M−H]⁻; ¹H NMR (400 MHz, DMSO-d₆): δ 8.23-8.19 (m, 1H), 7.36 (s, 1H), 6.58 (s, 1H), 5.46 (s, 2H), 5.43 (s, 2H), 3.52 (brs, 2H), 3.39-3.31 (m, 3H), 1.92-1.82 (m, 2H), 1.24 (d, J=6.0 Hz, 6H), 0.87 (t, J=7.2 Hz, 3H); —NH proton was not observed. Method Details: Column: GEMINI C18 (250*21.2 mm) 5μ: Mobile Phase A: 0.1% TFA IN WATER; B: ACN: Gradient Time: 0/10,20/45,24/45,24.1/98: 10 mL/Min Solubility: ACN+H₂O

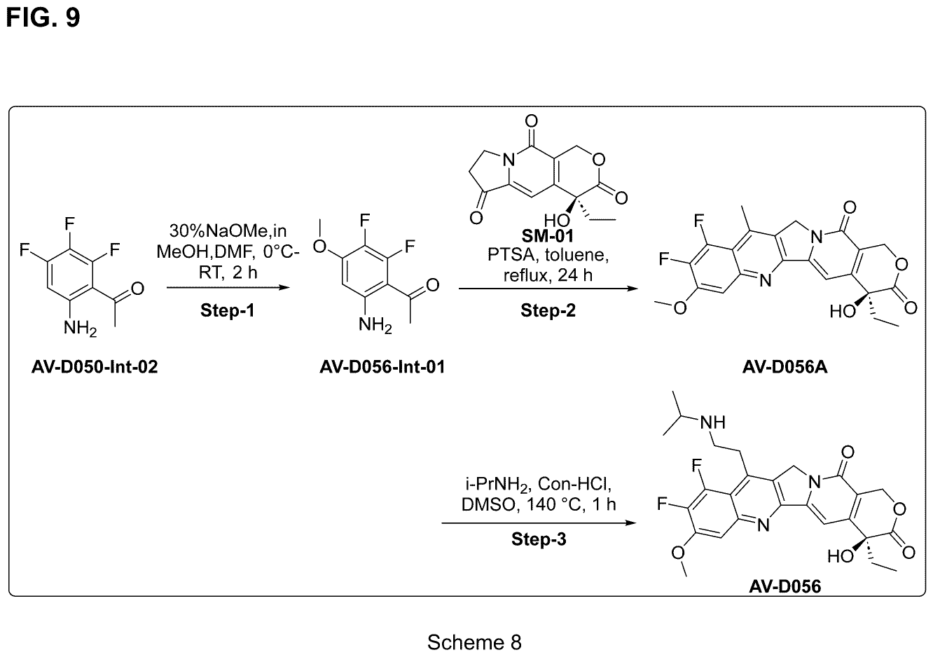

### VIII. Preparation of(S)-4-ethyl-9,10-difluoro-4-hydroxy-8-methoxy-11-methyl-1,12-dihydro-14H-pyrano[3′,4′:6,7]indolizino[1,2-b]quinoline-3,14(4H)-dione (AV-D056A) and (S)-4-ethyl-9,10-difluoro-4-hydroxy-11-(2-(isopropylamino)ethyl)-8-methoxy-1,12-dihydro-14H-pyrano[3′,4′:6,7]indolizino[1,2-b]quinoline-3,14(4H)-dione (AV-D056)(Scheme 8, FIG. 9)

The following example provides a synthetic procedure for the preparation of the compounds(S)-4-ethyl-9,10-difluoro-4-hydroxy-8-methoxy-11-methyl-1,12-dihydro-14H-pyrano[3′,4′:6,7]indolizino[1,2-b]quinoline-3,14(4H)-dione (AV-D056A) and(S)-4-ethyl-9,10-difluoro-4-hydroxy-11-(2-(isopropylamino)ethyl)-8-methoxy-1,12-dihydro-14H-pyrano[3′,4′:6,7]indolizino[1,2-b]quinoline-3,14(4H)-dione (AV-D056). The FIG. 9 depicts the schematic representation of synthesis reaction of the above-mentioned compounds.

### a. Synthesis of 1-(6-amino-2,3-difluoro-4-methoxyphenyl) ethan-1-one (AV-D056-Int-01)

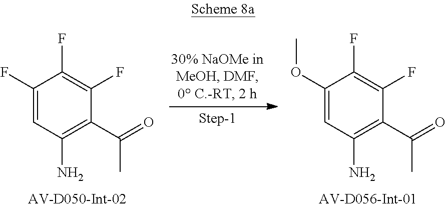

To a stirred solution of 1-(6-amino-2,3,4-trifluorophenyl) ethan-1-one (AV-D050-Int-02, 1 g, 6.8 mmol) in DMF (20 mL) was added 30% NaOMe in MeOH (2 mL, 34 mmol) at 0° C. and the reaction mixture was stirred at RT for 2 h. The reaction was monitored by TLC. The reaction was diluted with water (20 mL) and extracted with EtOAc (2×100 mL). The combined organic layer was washed with brine (50 mL), dried over anhydrous Na₂SO₄, filtered and concentrated under reduced pressure to obtain crude compound. The crude compound was purified by column chromatography (SiO₂, 100-200 mesh, 5% EtOAc/hexane) to afford 1-(6-amino-2,3-difluoro-4-methoxyphenyl) ethan-1-one (AV-D056-Int-01, 300 mg, 1.49 mmol, 31% yield) as a colourless semi solid.

¹H NMR (400 MHz, DMSO-d₆): δ 7.41 (brs, 2H), 6.27 (dd, J=2.0, 6.8 Hz, 1H), 3.83 (s, 3H), 2.44 (d, J=8.4 Hz, 3H).

### b. Synthesis of(S)-4-ethyl-9,10-difluoro-4-hydroxy-8-methoxy-11-methyl-1,12-dihydro-14H-pyrano[3′,4′:6,7]indolizino[1,2-b]quinoline-3,14(4H)-dione (AV-D056A)

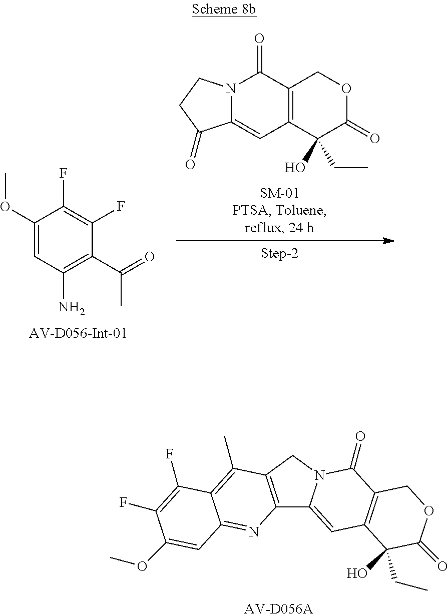

To a stirred solution of 1-(6-amino-2,3-difluoro-4-methoxyphenyl) ethan-1-one (AV-D056-Int-01, 180 mg, 0.89 mmol), and(S)-4-ethyl-4-hydroxy-7,8-dihydro-1H-pyrano[3,4-f]indolizine-3,6,10 (4H)-trione (SM-01, 188 mg, 0.71 mmol) in toluene (10 mL) was added p-toluenesulfonic acid (17 mg, 0.89 mmol) at room temperature and the reaction mixture was heated at 110° C. for 16 h. The reaction monitored by TLC. The resulting reaction mixture was concentrated under reduced pressure to get the crude product. It was purified by RP Prep HPLC and the fractions were lyophilized to obtain(S)-4-ethyl-9,10-difluoro-4-hydroxy-8-methoxy-11-methyl-1,12-dihydro-14H-pyrano[3′,4′:6,7]indolizino[1,2-b]quinoline-3,14(4H)-dione (AV-D056A, 7.5 g, 0.017 mmol, 2.2% yield), as a pale yellow solid.

MS (ESI): 428.75 [M+H]⁺; ¹H NMR (400 MHz, DMSO-d₆): δ 7.64-7.62 (m, 1H), 7.31 (s, 1H), 6.52 (s, 1H), 5.43 (s, 2H), 5.23 (s, 2H), 4.07 (s, 3H), 2.83 (d, J=6.0 Hz, 3H), 1.92-1.81 (m, 2H), 0.87 (t, J=7.2 Hz, 3H).

### c. Synthesis of(S)-4-ethyl-9,10-difluoro-4-hydroxy-11-(2-(isopropylamino)ethyl)-8-methoxy-1,12-dihydro-14H-pyrano[3′,4′:6,7]indolizino[1,2-b]quinoline-3,14(4H)-dione (AV-D056)

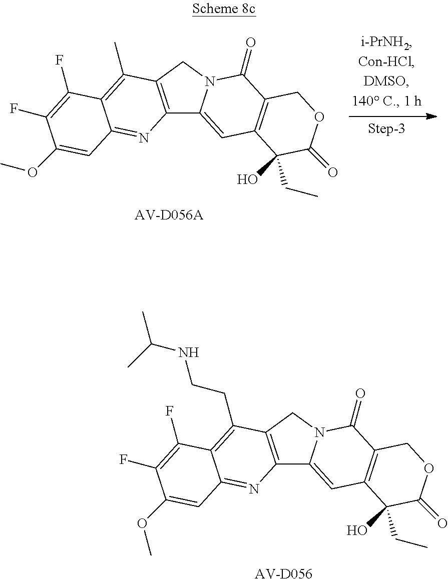

To a stirred solution of(S)-4-ethyl-9,10-difluoro-4-hydroxy-8-methoxy-11-methyl-1,12-dihydro-14H-pyrano[3′,4′:6,7]indolizino[1,2-b]quinoline-3,14(4H)-dione (AV-D056A, 180 mg, 0.42 mmol), and isopropylamine (0.2 mL, 2.5 mmol) in dimethyl sulfoxide (3 mL) was added concentrated hydrochloric acid (0.3 mL) and the mixture was heated for 2 h at 140° C. The reaction mixture was lyophilised with MeCN/H₂O to get brown gum which was purified by RP Prep HPLC and the fractions were lyophilized to obtain(S)-4-ethyl-9,10-difluoro-4-hydroxy-11-(2-(isopropylamino)ethyl)-8-methoxy-1,12-dihydro-14H-pyrano[3′,4′:6,7]indolizino[1,2-b]quinoline-3,14(4H)-dione (AV-D056, 3.5 mg, 0.007 mmol, 1.6% yield) as an off white solid.

MS (ESI): 497.92 [M−H]⁻; ¹H NMR (400 MHz, DMSO-d₆): δ 8.56 (brs, 1H), 7.74 (d, J=6.8 Hz, 1H), 7.34 (s, 1H), 6.57 (s, 1H), 5.45 (s, 2H), 5.40 (s, 2H), 4.10 (s, 3H), 3.54-3.47 (m, 2H), 3.40-3.30 (m, 2H), 1.92-1.81 (m, 2H), 1.24 (d, J=6.4 Hz, 6H), 0.87 (t, J=7.2 Hz, 3H); —NH proton was not observed.

Method Details: Column: Shim-pack C18 (250*20.0 mm) 5μ: Mobile Phase A: 0.1% TFA IN WATER; B: ACN: Gradient Time: 0/10, 12/42,20/42,20.1/98: 15 mL/Min Solubility: ACN+H₂O+THF

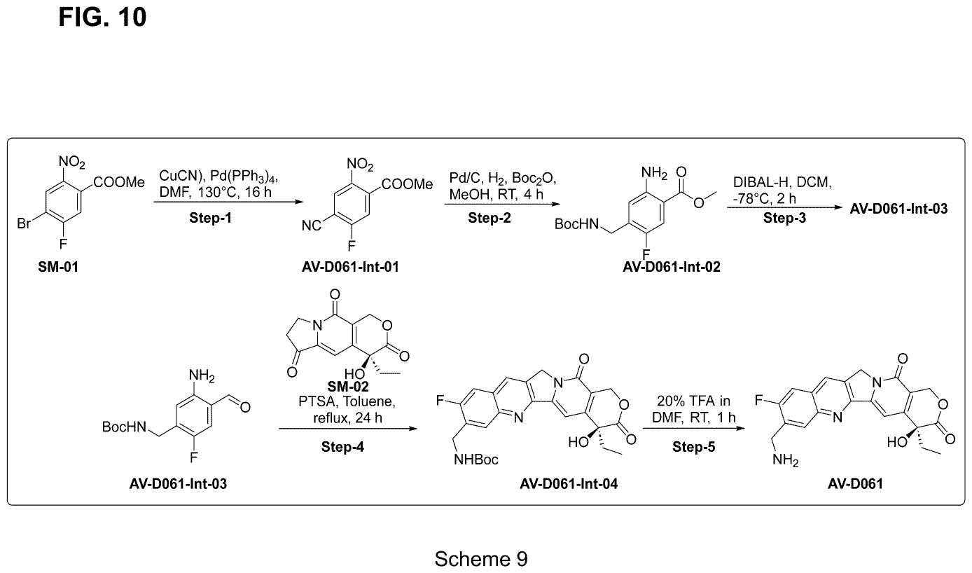

### IX. Synthetic scheme for(S)-8-(aminomethyl)-4-ethyl-9-fluoro-4-hydroxy-1,12-dihydro-14H-pyrano[3′,4′:6,7]indolizino[1,2-b]quinoline-3,14(4H)-dione (AV-D061)(Scheme 9, FIG. 10)

The following example provides a synthetic procedure for the preparation of the compound(S)-8-(aminomethyl)-4-ethyl-9-fluoro-4-hydroxy-1,12-dihydro-14H-pyrano[3′,4′:6,7]indolizino[1,2-b]quinoline-3,14(4H)-dione (AV-D061). The FIG. 10 depicts the schematic representation of synthesis reaction of the above-mentioned compound.

### a. Synthesis of methyl 4-cyano-5-fluoro-2-nitrobenzoate (AV-D061-Int-01)

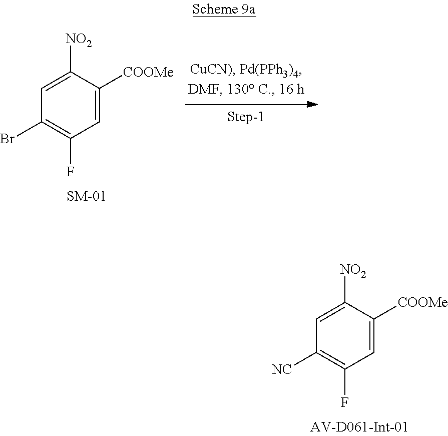 [SMILES: COC(=O)c1cc(F)c(Br)cc1[N+](=O)[O-] ; COC(=O)c1cc(F)c(C#N)cc1[N+](=O)[O-]]

To a stirred solution of methyl 4-bromo-5-fluoro-2-nitrobenzoate (SM-01, 2 g, 17.2 mmol) in DMF (20 mL) was added CuCN (1.3 g, 14.38 mmol) and Pd(PPh₃)₄ (207 mg, 0.18 mmol) at room temperature. The reaction was stirred at 130° C. for 16 h. The reaction was monitored by TLC. The reaction was passed through celite bed and washed with ethyl acetate (50 mL). The filtrate was extracted with ethyl acetate (3×150 mL). The combined organic layer was washed with brine (100 mL), dried over anhydrous Na₂SO₄, filtered and concentrated under reduced pressure. The residue was purified by silica gel column chromatography eluted with 5% ethyl acetate in hexanes to obtain methyl 4-cyano-5-fluoro-2-nitrobenzoate (AV-D061-Int-01, 410 mg, 1.8 mmol, 12% yield) as a pale yellow solid.

¹H NMR (400 MHz, DMSO-d₆): δ 8.92 (d, J=6.4, Hz, 1H), 8.13 (d, J=8.8, Hz, 1H), 3.90 (s, 3H).

### b. Synthesis of methyl 2-amino-4-(((tert-butoxycarbonyl)amino)methyl)-5-fluorobenzoate (AV-D061-Int-02)

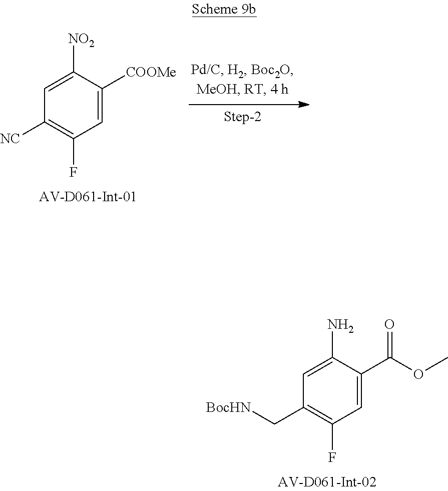 [SMILES: COC(=O)c1cc(F)c(C#N)cc1[N+](=O)[O-] ; COC(=O)c1cc(F)c(CNC(=O)OC(C)(C)C)cc1N]

To a stirred solution of methyl 4-cyano-5-fluoro-2-nitrobenzoate (AV-D061-Int-01, 600 mg, 2.67 mmol) in MeOH (26 mL) was added 10% Pd/C (180 mg, 30% w/w) and Boc₂O (0.9 mL, 4.1 mmol) at RT and stirred at same temperature for 4 h. The reaction was monitored by TLC. The resulting reaction mixture was filtered through celite pad, the filtrate was concentrated under reduced pressure to get methyl 2-amino-4-(((tert-butoxycarbonyl)amino)methyl)-5-fluorobenzoate (AV-D061-Int-02, 670 mg, 1.68 mmol, 44% yield) as a pale yellow solid. This was used as such for the next step.

MS (ESI): 299.11 [M+H]⁺.

### c. Synthesis of tert-butyl (5-amino-2-fluoro-4-formylbenzyl)carbamate (AV-D061-Int-03)

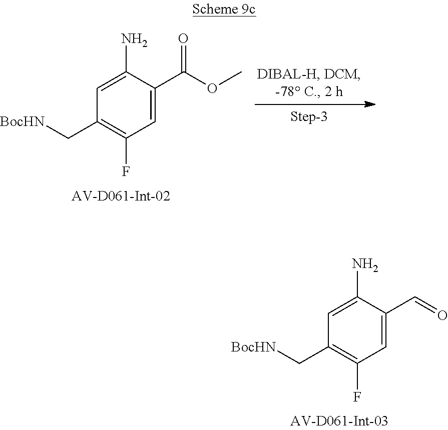 [SMILES: COC(=O)c1cc(F)c(CNC(=O)OC(C)(C)C)cc1N ; CC(C)(C)OC(=O)NCc1cc(N)c(C=O)cc1F]

To a stirred solution of methyl 2-amino-4-(((tert-butoxycarbonyl)amino)methyl)-5-fluorobenzoate (AV-D061-Int-02, 275 mg, 0.922 mmol) in dry THF (2 mL) was added DIBAL-H (1.5 M in THF, 3 mL, 4.6 mmol) at −78° C. The reaction was stirred at −78° C. for 1 h. The reaction was quenched with sodium potassium tartrate solution and extracted with ethyl acetate. The solvent was evaporated under reduced pressure to obtain tert-butyl (5-amino-2-fluoro-4-formylbenzyl)carbamate (AV-D061-Int-03, 2.2 mg) as a pale yellow semi solid, The crude was used as such for the next step.

MS (ESI): 212.58 [M+H−56]⁺.

### d. Synthesis of tert-butyl(S)-((4-ethyl-9-fluoro-4-hydroxy-3,14-dioxo-3,4,12,14-tetrahydro-1H-pyrano[3′,4′:6,7]indolizino[1,2-b]quinolin-8-yl)methyl)carbamate (AV-D061-Int-04)

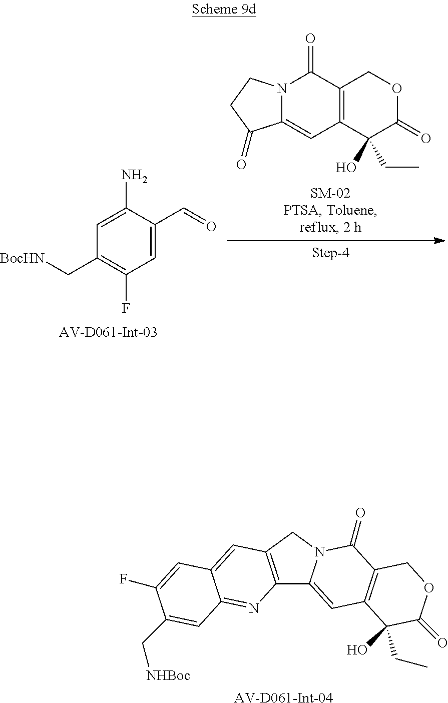 [SMILES: CC[C@@]1(O)C(=O)OCc2c1cc1n(c2=O)CCC1=O ; CC(C)(C)OC(=O)NCc1cc(N)c(C=O)cc1F ; CC[C@@]1(O)C(=O)OCc2c1cc1n(c2=O)Cc2cc3cc(F)c(CNC(=O)OC(C)(C)C)cc3nc2-1 ; "AV-D061-Int-04"]

To a stirred solution of tert-butyl (5-amino-2-fluoro-4-formylbenzyl)carbamate (AV-D061-Int-03, 400 mg, 1.49 mmol), and(S)-4-ethyl-4-hydroxy-7,8-dihydro-1H-pyrano[3,4-f]indolizine-3,6,10 (4H)-trione (78 mg, 0.29 mmol) in toluene (4 mL) was added PTSA (28 mg, 0.14 mmol) and the reaction was heated at 110° C. for 2 h. The reaction was passed through celite bed and washed with 10% methanol in DCM (20 mL). The filtrate was collected in RBF, solvent was evaporated under reduced pressure, and residue was purified by reverse phase Biotage to obtain tert-butyl(S)-((4-ethyl-9-fluoro-4-hydroxy-3,14-dioxo-3,4,12,14-tetrahydro-1H-pyrano[3′,4′:6,7]indolizino[1,2-b]quinolin-8-yl)methyl)carbamate (AV-D061-Int-04, 50 mg, 0.1 mmol, 7% yield).

MS (ESI): 496.21 [M+H]⁺.

### e. Synthesis of(S)-8-(aminomethyl)-4-ethyl-9-fluoro-4-hydroxy-1,12-dihydro-14H-pyrano[3′,4′:6,7]indolizino[1,2-b]quinoline-3,14(4H)-dione (AV-D061)

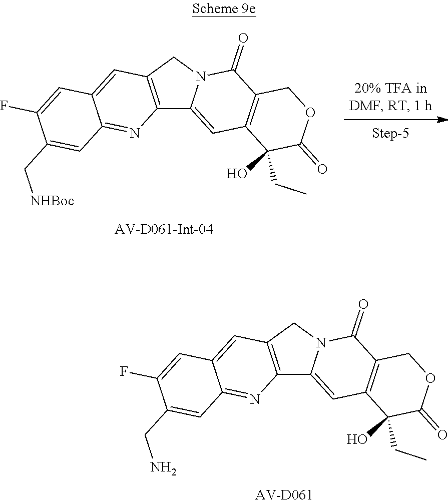 [SMILES: CC[C@@]1(O)C(=O)OCc2c1cc1n(c2=O)Cc2cc3cc(F)c(CNC(=O)OC(C)(C)C)cc3nc2-1 ; CC[C@@]1(O)C(=O)OCc2c1cc1n(c2=O)Cc2cc3cc(F)c(CN)cc3nc2-1 ; "AV-D061"]

To a stirred solution of tert-butyl(S)-((4-ethyl-9-fluoro-4-hydroxy-3,14-dioxo-3,4,12,14-tetrahydro-1H-pyrano[3′,4′:6,7]indolizino[1,2-b]quinolin-8-yl)methyl)carbamate (AV-D061-Int-04, 50 mg, 0.1 mmol) in DCM (1 mL) was added 40% TFA in DCM (1 mL) and the mixture was stirred at room temperature for 1 h. The volatiles were evaporated under reduced pressure and the residue was purified by reverse phase prep HPLC to obtain(S)-8-(aminomethyl)-4-ethyl-9-fluoro-4-hydroxy-1,12-dihydro-14H-pyrano[3′,4′:6,7]indolizino[1,2-b]quinoline-3,14(4H)-dione (AV-D061, 1.5 mg, 0.004 mmol, 4% yield) as an off-white solid.

PREP-HPLC Conditions: GEMINI C18 (250*21.2*) 5μ A: 0.1% TFA IN WATER; B: A CN:H₂O (80:20): 0/5,16/35,16.1/98@12 ML/MIN; diluent: ACN+H₂O.

MS (ESI): 396.11 [M+H]⁺: ¹H NMR (400 MHz, DMSO-d₆): δ 8.66 (s, 1H), 8.32 (d, J=7.6 Hz, 1H), 7.97 (d, J=10.8 Hz, 1H), 7.32 (s, 1H), 6.55 (s, 1H), 5.43 (s, 2H), 5.30 (s, 2H), 4.12 (s, 2H), 1.91-1.85 (m, 2H), 0.88 (t, J=7.2 Hz, 3H); —NH₂ peak was not observed.

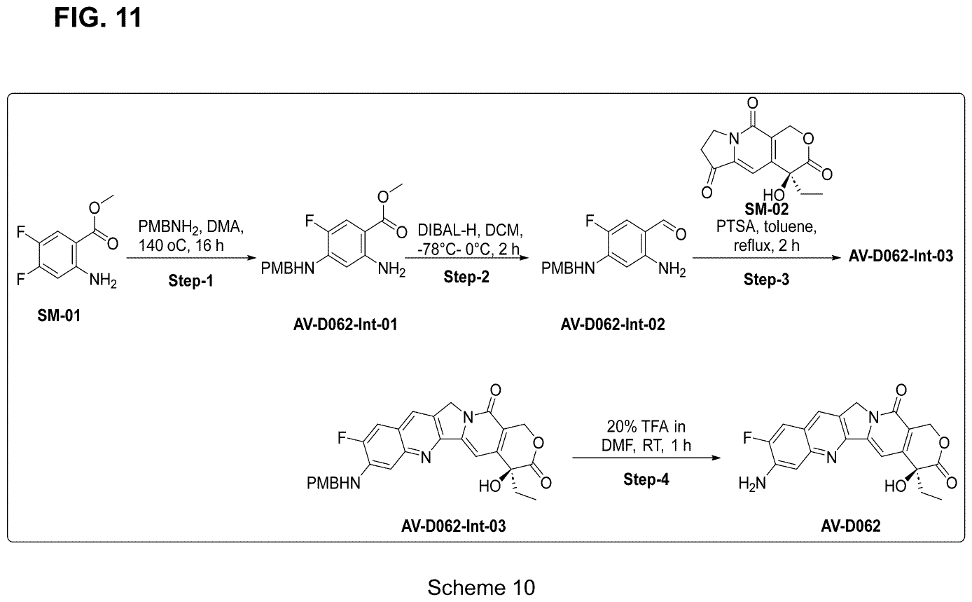

### X. Synthetic scheme for(S)-8-amino-4-ethyl-9-fluoro-4-hydroxy-1,12-dihydro-14H-pyrano[3′,4′:6,7]indolizino[1,2-b]quinoline-3,14(4H)-dione (AV-D062)(Scheme 10, FIG. 11)

The following example provides a synthetic procedure for the preparation of the compounds(S)-8-amino-4-ethyl-9-fluoro-4-hydroxy-1,12-dihydro-14H-pyrano[3′,4′:6,7]indolizino[1,2-b]quinoline-3,14(4H)-dione (AV-D062). The FIG. 11 depicts the schematic representation of synthesis reaction of the above-mentioned compounds.

### a. Synthesis of methyl 2-amino-5-fluoro-4-((4-methoxybenzyl)amino)benzoate (AV-D062-Int-01)

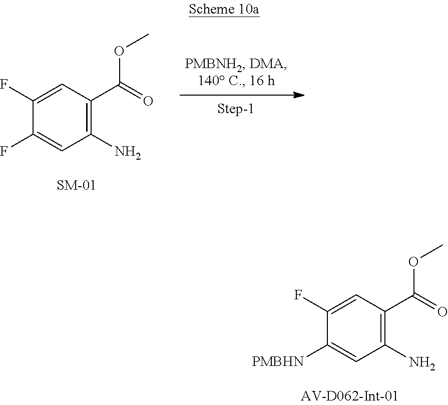

To a stirred solution of methyl 2-amino-4,5-difluorobenzoate (SM-01, 3 g, 16.04 mmol) in DMA (30 mL) was added 4-methoxybenzylamine (2.6 g, 19.2 mmol) at 0° C. and the reaction mixture was heated at 140° C. for 16 h. The reaction was monitored by TLC. The reaction was cooled to room temperature, diluted with water (20 mL) and extracted with EtOAc (2×150 mL). The combined organic layer was washed with brine (100 mL), dried over anhydrous Na₂SO₄, filtered and concentrated under reduced pressure to obtain crude compound. The crude compound was purified by column chromatography (SiO₂, 100-200 mesh, 20% EtOAc/hexane) to afford methyl 2-amino-5-fluoro-4-((4-methoxybenzyl)amino)benzoate (AV-D062-Int-01, 2 g, 7.27 mmol, 45% yield) as an off white solid.

MS (ESI): 304.980 [M+H]⁺.

### b. Synthesis of 2-amino-5-fluoro-4-((4-methoxybenzyl)amino)benzaldehyde (AV-D062-Int-02)

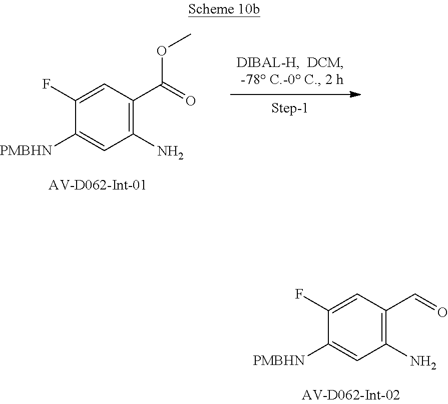

To a solution of methyl 2-amino-5-fluoro-4-((4-methoxybenzyl)amino)benzoate (AV-D062-Int-01, 500 mg, 1.64 mmol) in THF (5 mL) was added 1.5 M DIBAL-H in toluene (5.48 mmol) at −78° C. and the reaction mixture was stirred at same temperature for 2 h. The reaction was monitored by TLC. The reaction was quenched with sodium potassium tartrate and extracted with EtOAc (2×100 mL). The combined organic layer was washed with brine (50 mL), dried over anhydrous Na₂SO₄, filtered and concentrated under reduced pressure to obtain 2-amino-5-fluoro-4-((4-methoxybenzyl)amino)benzaldehyde (AV-D062-Int-02, 500 mg, 1.82 mmol, quantitative yield) as a pale yellow semi solid. The crude was used as such for the next step. MS (ESI): 275.0 [M+H]⁺.

### c. Synthesis of(S)-4-ethyl-9-fluoro-4-hydroxy-8-((4-methoxybenzyl)amino)-1,12-dihydro-14H-pyrano[3′,4′:6,7]indolizino[1,2-b]quinoline-3,14(4H)-dione (AV-D062-Int-03)

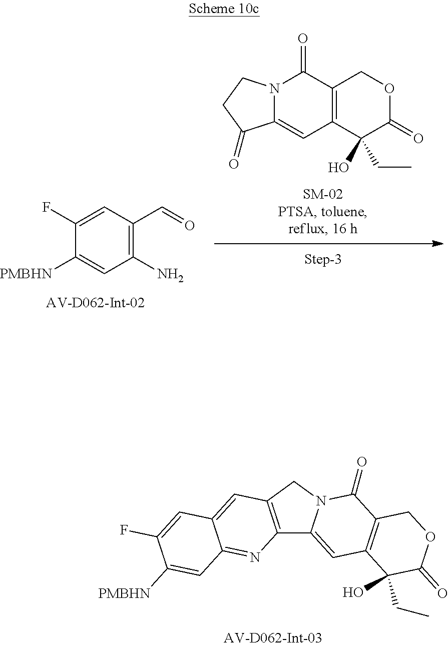

To a stirred solution of 2-amino-5-fluoro-4-((4-methoxybenzyl)amino)benzaldehyde (AV-D062-Int-02, 500 mg, 1.82 mmol), and(S)-4-ethyl-4-hydroxy-7,8-dihydro-1H-pyrano[3,4-f]indolizine-3,6,10 (4H)-trione (SM-02, 95 mg, 0.36 mmol) in toluene (3 mL) was added p-toluene sulfonic acid (34 mg, 0.18 mmol) at room temperature and the reaction mixture was heated at 110° C. for 16 h. The reaction was monitored by TLC. The resulting reaction mixture was concentrated under reduced pressure to get the crude product. This was purified by RP Prep HPLC to obtain(S)-4-ethyl-9-fluoro-4-hydroxy-8-((4-methoxybenzyl)amino)-1,12-dihydro-14H-pyrano[3′,4′:6,7]indolizino[1,2-b]quinoline-3,14(4H)-dione (AV-D062-Int-03, 200 mg, 0.399 mmol, 18% yield) as a yellow solid.

MS (ESI): 502.1 [M+H]⁺.

### d. Synthesis of(S)-8-amino-4-ethyl-9-fluoro-4-hydroxy-1,12-dihydro-14H-pyrano[3′,4′:6,7]indolizino[1,2-b]quinoline-3,14(4H)-dione (AV-D062)

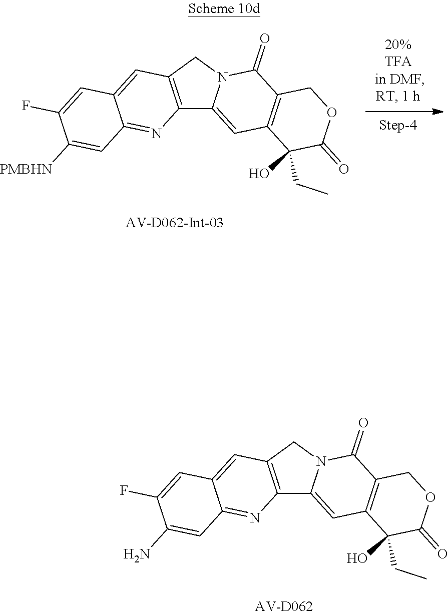

To a(S)-4-ethyl-9-fluoro-4-hydroxy-8-((4-methoxybenzyl)amino)-1,12-dihydro-14H-pyrano[3′,4′:6,7]indolizino[1,2-b]quinoline-3,14(4H)-dione (AV-D062-Int-03, 200 mg, 0.399 mmol) was added 50% TFA in DCM was added at 0° C. and the mixture was stirred at RT. The reaction monitored by TLC. The resulting reaction mixture was concentrated under reduced pressure to get the crude. The was purified by RP Prep HPLC to obtain(S)-8-amino-4-ethyl-9-fluoro-4-hydroxy-1,12-dihydro-14H-pyrano[3′,4′:6,7]indolizino[1,2-b]quinoline-3,14(4H)-dione (AV-D062, 7.3 mg, 0.019 mmol, 5% yield) as a light orange solid.

MS (ESI): 382.17 [M+H]⁺; ¹H NMR (400 MHz, DMSO-d₆): δ 8.38 (s, 1H), 7.75 (d, J=12 Hz, 1H), 7.26-7.24 (m, 2H), 6.50 (brs, 1H), 6.16 (brs, 2H), 5.41 (s, 2H), 5.17 (s, 2H), 1.90-1.80 (m, 2H), 0.87 (t, J=7.2 Hz, 3H).

Method Details: Column: GEMINI C18 (250*21.2 mm) 5μ: Mobile Phase A: 0.1% TFA in Water; B: ACN: Gradient Time: 0/30,10/50, 13/50,13.1/98:14 mL/Min Solubility: ACN+H₂O+THF

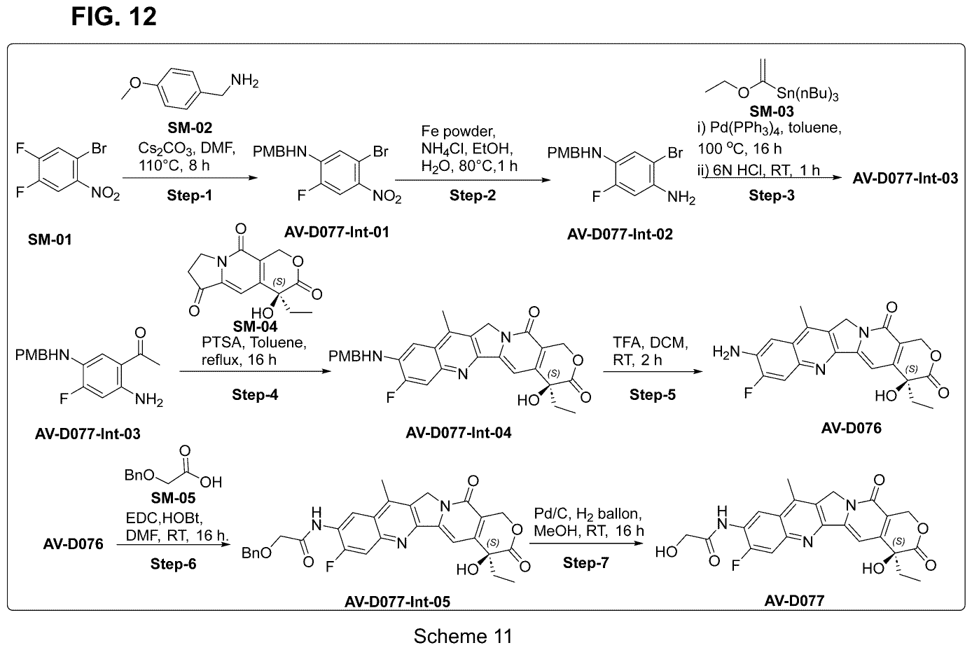

### XI. Preparation of(S)-9-amino-4-ethyl-8-fluoro-4-hydroxy-11-methyl-1,12-dihydro-14H-pyrano[3′,4′:6,7]indolizino[1,2-b]quinoline-3,14(4H)-dione (AV-D076) and(S)—N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)-2-hydroxyacetamide (AV-D077)(Scheme 11, FIG. 12)

The following example provides synthetic procedure for the preparation of the compounds(S)-9-amino-4-ethyl-8-fluoro-4-hydroxy-11-methyl-1,12-dihydro-14H-pyrano[3′,4′:6,7]indolizino[1,2-b]quinoline-3,14(4H)-dione (AV-D076) and(S)—N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)-2-hydroxyacetamide (AV-D077). The FIG. 12 depicts the schematic representation of synthesis reaction of the above-mentioned compounds.

### a. Synthesis of 5-bromo-2-fluoro-N-(4-methoxybenzyl)-4-nitroaniline (AV-D077-Int-01)

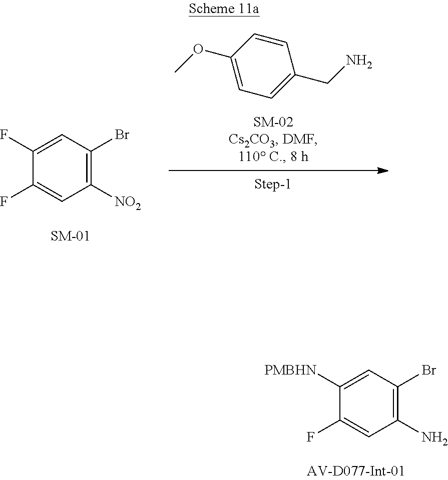

To a stirred solution of 1-bromo-4,5-difluoro-2-nitrobenzene (SM-01, 5 g, 21.09 mmol) and (4-methoxyphenyl)methylamine (SM-02, 3.3 mL, 25 mmol) in dimethyl formamide (DMF, 50 mL), Cs₂CO₃ (20.5 g, 63 mmol) was added at room temperature and the reaction mixture was stirred at 110° C. for 8 h. The reaction was monitored by TLC. The reaction was cooled to 25° C., diluted with water (200 mL) and extracted with EtOAc (2×250 mL). The combined organic layer was washed with brine (200 mL), dried over anhydrous Na₂SO₄, filtered and concentrated under reduced pressure to obtain the crude product. The crude product was purified by silica gel column chromatography (eluted with 8% ethyl acetate in hexanes) to get 5-bromo-2-fluoro-N-(4-methoxybenzyl)-4-nitroaniline (AV-D077-Int-01, 6 g, 16.9 mmol, 80% yield) as a pink solid. The schematic representation of the reaction is shown in Scheme 11a.

MS (ESI): 354.84 [M+H]⁺; ¹H NMR (400 MHz, DMSO-d₆): δ 7.98 (d, J=12 Hz, 1H), 7.81 (brt, 1H), 7.28 (d, J=8.4 Hz, 2H), 6.96-6.88 (m, 3H), 4.42 (d, J=6.4 Hz, 2H), 3.71 (s, 3H).

### b. Synthesis of 5-bromo-2-fluoro-N1-(4-methoxybenzyl)benzene-1,4-diamine (AV-D077-Int-02)

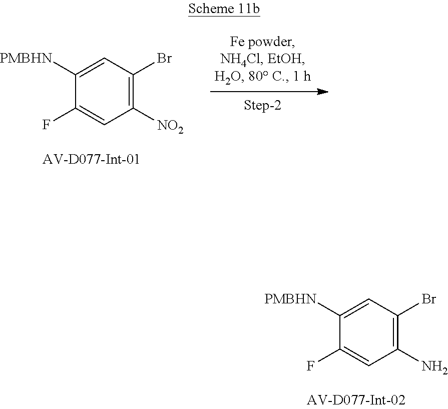

To a stirred solution of 5-bromo-2-fluoro-N-(4-methoxybenzyl)-4-nitroaniline (AV-D077-Int-01, 1 g, 2.82 mmol) and Fe powder (777 mg, 14.2 mmol) in ethanol:H₂O (8:2, 14 mL), NH₄Cl (749 mg, 14.1 mmol) was added at room temperature and stirred at 70° C. for 1 h. The reaction was monitored by TLC. The reaction was cooled to 25° C. and filtered through celite pad. The filtrate was diluted with water (20 mL) and extracted with EtOAc (2×50 mL). The combined organic layer was washed with brine (50 mL), dried over anhydrous Na₂SO₄, filtered and concentrated under reduced pressure to obtain 5-bromo-2-fluoro-N1-(4-methoxybenzyl)benzene-1,4-diamine (AV-D077-Int-02, 900 mg, 2.77 mmol, 98% yield) as a brown solid. The schematic representation of the reaction is shown in Scheme 11b.

MS (ESI): 326.91 [M+2H+1]⁺; ¹H NMR (400 MHz, DMSO-d₆): δ 7.25 (d, J=8.4 Hz, 2H), 6.88-6.84 (m, 2H), 6.61-6.57 (m, 2H), 5.41 (t, J=5.2 Hz, 1H), 4.66 (s, 2H), 4.13 (d, J=6.4 Hz, 2H), 3.71 (s, 3H).

### c. Synthesis of 1-(2-amino-4-fluoro-5-((4-methoxybenzyl)amino)phenyl) ethan-1-one (AV-D077-Int-03)

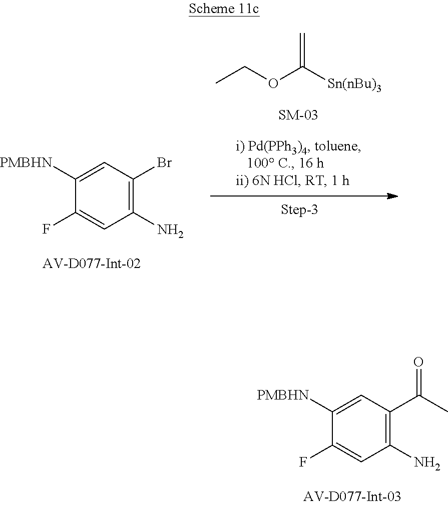

To a stirred solution of 5-bromo-2-fluoro-N1-(4-methoxybenzyl)benzene-1,4-diamine (AV-D077-Int-02, 2.8 g, 8.64 mmol) and tributyl(1-ethoxyvinyl) stannane (SM-02, 6.2 g, 17.8 mmol) in toluene (30 mL), Pd(PPh₃)₄ (449 mg, 0.216 mmol) was added at room temperature. The reaction mixture was stirred at 100° C. for 16 h. The reaction was monitored by TLC. The reaction was cooled to 25° C., treated with 6N HCl (25 mL) and stirred at same temperature for 1 h. The reaction was diluted with water (100 mL) and extracted with EtOAc (2×150 mL). The combined organic layer was washed with brine (100 mL), dried over anhydrous Na₂SO₄, filtered, and concentrated under reduced pressure to obtain crude compound. The crude compound was purified by silica gel column chromatography (SiO₂, 100-200 mesh, 30% EtOAc/hexane) to afford 1-(2-amino-4-fluoro-5-((4-methoxybenzyl)amino)phenyl) ethan-1-one (AV-D077-Int-03, 1.0 g, 3.5 mmol, 25% yield) as a pale-yellow gum. The schematic representation of the reaction is shown in Scheme 11c.

MS (ESI): 289.12 [M+H]⁺; ¹H NMR (400 MHz, DMSO-d₆): δ 7.32 (d, J=8.4 Hz, 2H), 6.90-6.86 (m, 3H), 6.76 (brs, 2H), 6.48 (d, J=8.4 Hz, 1H), 5.37 (t, J=5.6 Hz, 1H), 4.18 (d, J=6.4 Hz, 2H), 3.71 (s, 3H), 2.30 (s, 3H).

### d. Synthesis of(S)-4-ethyl-8-fluoro-4-hydroxy-9-((4-methoxybenzyl)amino)-11-methyl-1,12-dihydro-14H-pyrano[3′,4′:6,7]indolizino[1,2-b]quinoline-3,14(4H)-dione (AV-D077-Int-04)

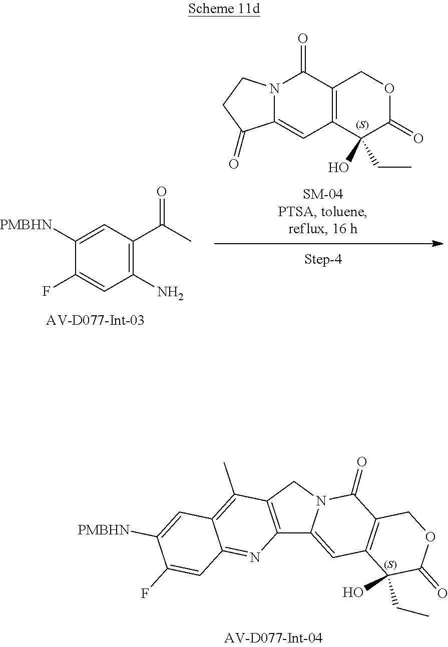

To a stirred solution of 1-(2-amino-4-fluoro-5-((4-methoxybenzyl)amino)phenyl) ethan-1-one (AV-D077-Int-03, Formula II, 500 mg, 1.74 mmol), and(S)-4-ethyl-4-hydroxy-7,8-dihydro-1H-pyrano[3,4-f]indolizine-3,6,10 (4H)-trione (SM-04, Formula III, 365 mg, 1.38 mmol) in toluene (solvent, 5 mL), p-toluenesulfonic acid (29 mg, 0.17 mmol) was added at room temperature and the reaction mixture was heated at 110° C. for 16 h. The reaction was monitored by TLC. The resulting reaction mixture was concentrated under reduced pressure to get crude product. The crude product was further purified by RP Biotage to get(S)-4-ethyl-8-fluoro-4-hydroxy-9-((4-methoxybenzyl)amino)-11-methyl-1,12-dihydro-14H-pyrano[3′,4′:6,7]indolizino[1,2-b]quinoline-3,14(4H)-dione (AV-D077-Int-04, 500 mg, 0.46 mmol, 56% yield) as a light brown solid. The schematic representation of the reaction is shown in Scheme 11d.

MS (ESI): 516.25 [M+H]⁺.

### e. Synthesis of(S)-9-amino-4-ethyl-8-fluoro-4-hydroxy-11-methyl-1,12-dihydro-14H-pyrano[3′,4′:6,7]indolizino[1,2-b]quinoline-3,14(4H)-dione (AV-D076)

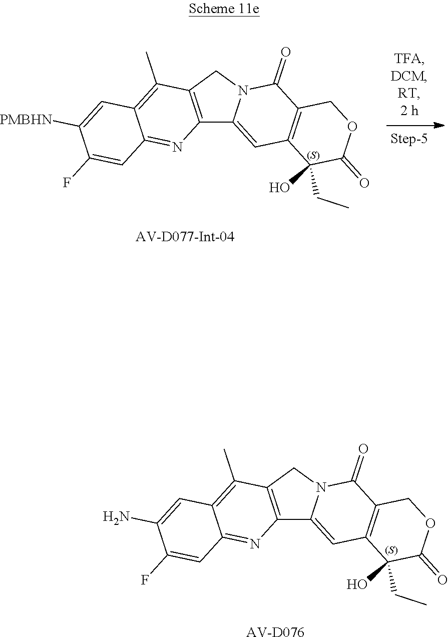

To a stirred solution of(S)-4-ethyl-8-fluoro-4-hydroxy-9-((4-methoxybenzyl)amino)-11-methyl-1,12-dihydro-14H-pyrano[3′,4′:6,7]indolizino[1,2-b]quinoline-3,14(4H)-dione (AV-D077-Int-04, 300 mg, 0.582 mmol) in DCM (1.5 mL) was added TFA (1.5 mL) at room temperature and the reaction mixture was stirred at same temperature for 2 h. The reaction was monitored by TLC. The resulting reaction mixture was concentrated under reduced pressure to get crude product (250 mg) and the 50 mg of crude product was purified by RP Biotage to obtain (S)-9-amino-4-ethyl-8-fluoro-4-hydroxy-11-methyl-1,12-dihydro-14H-pyrano[3′,4′:6,7]indolizino[1,2-b]quinoline-3,14(4H)-dione (AV-D076, 1.2 mg, 0.003 mmol) as an off white solid. The schematic representation of the reaction is shown in Scheme 11e.

MS (ESI): 396.17 [M+H]⁺; ¹H NMR (400 MHz, DMSO-d₆): δ 7.74 (d, J=13.6 Hz, 1H), 7.25 (d, J=9.6 Hz, 1H), 7.19 (s, 1H), 6.47 (s, 1H), 6.10 (s, 2H), 5.41 (s, 2H), 5.22 (s, 2H), 2.61 (s, 3H), 1.90-1.80 (m, 2H), 0.87 (t, J=7.2 Hz, 3H).

### f. Synthesis of(S)-2-(benzyloxy)-N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)acetamide (AV-D077-Int-05)

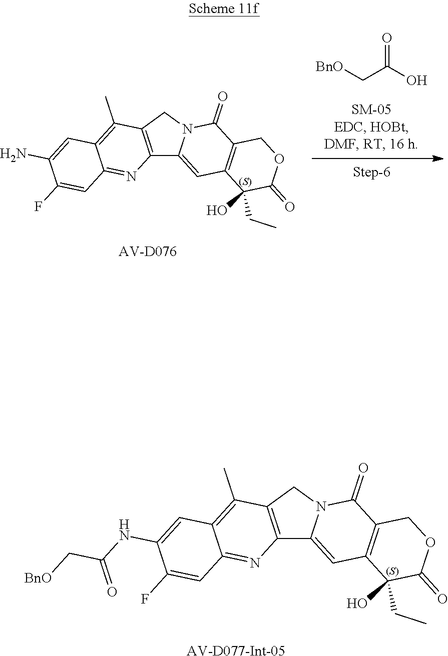

To a stirred solution of(S)-9-amino-4-ethyl-8-fluoro-4-hydroxy-11-methyl-1,12-dihydro-14H-pyrano[3′,4′:6,7]indolizino[1,2-b]quinoline-3,14(4H)-dione (AV-D076, 100 mg, 0.602 mmol), and 2-(benzyloxy) acetic acid (SM-05, 238 mg, 0.602 mmol) in DMF (1 mL) were added HOBt (121 mg, 0.903 mmol), ECD·HCl (172 mg, 0.902 mmol) and the mixture was stirred at room temperature for 16 h. The reaction mixture was diluted with water (4 mL) and extracted with ethylacetate (10 mL). The organic layer was washed with brine solution (20 mL), dried over Na₂SO₄, filtered and solvent was evaporated by reduced pressure to obtain(S)-2-(benzyloxy)-N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)acetamide (AV-D077-Int-05, 100 mg, 0.184 mmol) as a brown gum. The crude was used as such for the next step. The schematic representation of the reaction is shown in Scheme 11f.

MS (ESI): 544.13 [M+H]⁺.

### g. Synthesis of(S)—N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)-2-hydroxyacetamide (AV-D077)

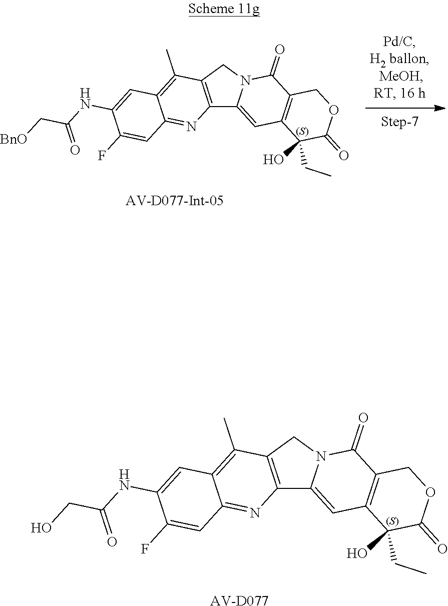

To a stirred solution of(S)-2-(benzyloxy)-N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)acetamide (AV-D077-Int-05, 30 mg, 0.055 mmol) in MeOH (3 mL) was added 10% w/w Pd/C (9 mg). The reaction was stirred at room temperature for 16 h under hydrogen balloon pressure. The reaction mixture was filter through celite pad, washed with THF (50 mL) and evaporated under reduced pressure to get the crude product which was purified by RP Prep HPLC to afford(S)—N-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl)-2-hydroxyacetamide (AV-D077, 1 mg, 0.002 mmol, 4% yield) as a light brown solid. The schematic representation of the reaction is shown in Scheme 12g.

MS (ESI): 452.05 [M−H]⁻: ¹H NMR (400 MHz, DMSO-d₆): δ 9.72 (brs, 1H), 8.89 (d, J=8.4 Hz, 1H), 8.04 (d, J=12.0 Hz, 1H), 7.30 (s, 1H), 6.53 (brs, 1H), 5.99 (t, J=6.0 Hz, 1H), 5.43 (s, 2H), 5.30 (s, 2H), 4.14 (d, J=6.0 Hz, 2H), 2.74 (s, 3H), 1.90-1.81 (m, 2H), 0.87 (t, J=7.2 Hz, 3H).

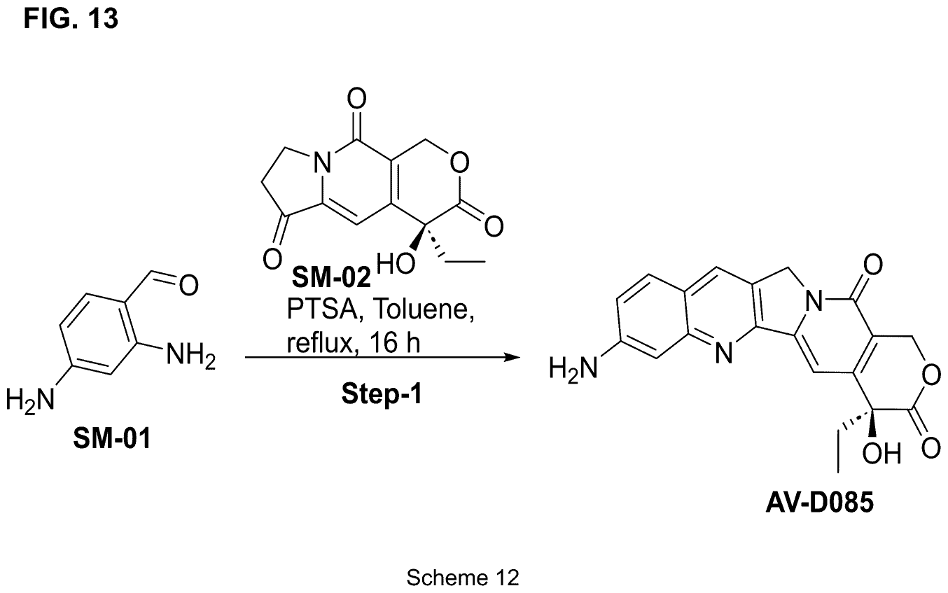

### XII. Preparation of(S)-8-amino-4-ethyl-4-hydroxy-1,12-dihydro-14H-pyrano[3′,4′:6,7]indolizino[1,2-b]quinoline-3,14(4H)-dione (AV-D085)(Scheme 12, FIG. 13)

The following example provides synthetic procedure for the preparation of the compound ((S)-8-amino-4-ethyl-4-hydroxy-1,12-dihydro-14H-pyrano[3′,4′:6,7]indolizino[1,2-b]quinoline-3,14(4H)-dione (AV-D085). The FIG. 13 depicts the schematic representation of synthesis reaction of the above-mentioned compound.

To a stirred solution of 2,4-diaminobenzaldehyde (SM-01, 250 mg, 1.83 mmol), and (S)-4-ethyl-4-hydroxy-7,8-dihydro-1H-pyrano[3,4-f]indolizine-3,6,10 (4H)-trione (SM-02, 483 mg, 1.83 mmol) in toluene (2.5 mL, 10 vol) was added PTSA (34 m g, 0.183 mmol) and the reaction was heated at 110° C. for 16 h. The reaction was passed through celite bed and washed with 10% methanol in DCM (50 mL). The filtrate was evaporated under reduced pressure and purified by RP prep HPLC to obtain(S)-8-amino-4-ethyl-4-hydroxy-1,12-dihydro-14H-pyrano[3′,4′:6,7]indolizino[1,2-b]quinoline-3,14(4H)-dione (AV-D085, 2.9 mg, 0.008 mmol, 1% yield).

MS (ESI): 364.12 [M+H]⁺, ¹H NMR (400 MHz, DMSO-d₆): δ 8.39 (s, 1H), 7.78 (d, J=8.8 Hz, 1H), 7.28 (s, 1H), 7.11 (dd, J=2.0, 8.8 Hz, 1H), 7.03 (d, J=4.0 Hz, 1H), 6.54 (brs, 1H), 6.11 (brs, 2H), 5.41 (s, 2H), 5.16 (s, 2H), 1.90-1.80 (m, 2H), 0.87 (t, J=6.8 Hz, 3H).

PREP-HPLC Conditions: GEMINI C18 (250*21.2*) 5μ: A: 0.1% TFA IN WATER; B: A CN:H₂O (80:20): 0/12,15/50, 15.1/98@14 ML/MIN; diluent: ACN+H₂O+THF

### XIII Preparation of Linker (Compound of Formula L)

### a. 2,5-dioxopyrrolidin-1-yl(((9H-fluoren-9-yl)methoxy)carbonyl)glycylglycinate (Linker a)

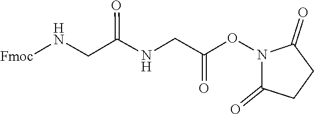

The compound was prepared according to the procedure described in Chinese Patent Publication No. CN105218644.

### b. (((9H-fluoren-9-yl)methoxy)carbonyl)glycylglycyl-L-phenylalanine (Fmoc-GGF-OH; Linker b)

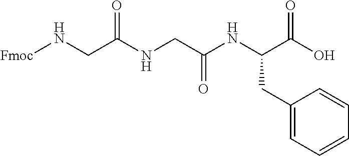

N,N-Diisopropylethylamine (DIPEA, 1.51 mL) was added to L-phenylalanine (965 mg) in acetonitrile (10 mL) and dimethyl formamide (0.5 mL) and then Linker a (1.3 g) was added. After 1 h the reaction was concentrated to dryness. Flash purification was accomplished as described in General Procedure 9, eluting with a 10 to 50% CH₃CN/H₂O+0.1% TFA gradient to provide the Linker b compound as a white solid (430 mg, 30% yield).

LC/MS: Calc'd m/z=501.2 for C₂₈H₇₁N₃O₆S, found [M+H]⁺=502.4.

¹H NMR (300 MHz, DMSO) δ 8.16 (d, J=8.1 Hz, 1H), 8.04 (t, J=5.8 Hz, 1H), 7.90 (d, J=7.5 Hz, 2H), 7.72 (d, J=7.4 Hz, 2H), 7.59 (t, J=6.0 Hz, 1H), 7.54-7.39 (m, 2H), 7.33 (t, J=7.6 Hz, 2H), 7.28-7.13 (m, 5H), 4.44 (td, J=8.5, 5.1 Hz, 1H), 4.33-4.13 (m, 3H), 3.83-3.59 (m, 4H), 3.06 (dd, J=13.7, 5.1 Hz, 1H), 2.88 (dd, J=13.8, 9.0 Hz, 1H).

### c. 2,3,5,6-tetrafluorophenyl 3-(2-(2-(2-(2,5-dioxo-2,5-dihydro-1H-pyrrol-1-yl) ethoxy) ethoxy)ethoxy)propanoate (MT-OTfp; Linker c)

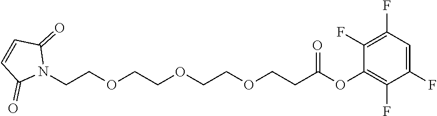 [SMILES: O=C(CCOCCOCCOCCN1C(=O)C=CC1=O)Oc1c(F)c(F)cc(F)c1F]

The Linker c compound was prepared according to the procedure described in International (PCT) Publication No. WO 2017/054080.

### d. (3-(2-(2-(2-(2,5-dioxo-2,5-dihydro-1H-pyrrol-1-yl) ethoxy) ethoxy) ethoxy) propanoyl)glycylglycyl-L-phenylalanine (Linker d)

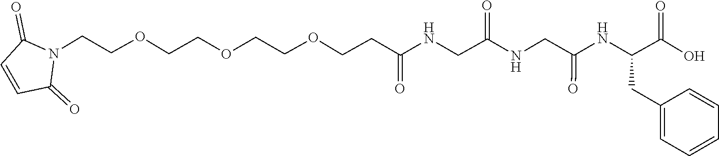 [SMILES: O=C(CCOCCOCCOCCN1C(=O)C=CC1=O)NCC(=O)NCC(=O)N[C@@H](Cc1ccccc1)C(=O)O]

To a solution of Linker c (1.61 g, 3.58 mmol) in DMF (35 mL) was added Gly-Gly-Phe (1 g, 3.58 mmol) as a single portion followed by iPr₂NEt (1.25 mL, 7.2 mmol). This solution was stirred at room temperature for 1 h, then evaporated to dryness. Purification was accomplished as described in General Procedure 9 using a 30 g C18 flash column and eluting with a 10 to 90% CH₃CN/H₂O+0.1% TFA gradient to provide the Linker d compound as a white solid (400 mg, 20% yield).

LC/MS: Calc'd m/z=562.6 for C26H₃4N4O10, found [M−H]−=561.5.

¹H NMR (300 MHz, CDCl3) δ 7.60 (t, J=5.6 Hz, 2H), 7.41 (d, J=7.7 Hz, 1H), 7.32-7.07 (m, 5H), 6.70 (s, 2H), 6.33-6.07 (m, 3H), 4.72 (td, J=7.6, 5.3 Hz, 1H), 4.12-3.78 (m, 4H), 3.72 (ddd, J=15.2, 6.9, 4.8 Hz, 5H), 3.60 (dd, J=11.6, 6.1 Hz, 10H), 3.12 (ddd, J=48.2, 14.0, 6.5 Hz, 2H), 2.52 (d, J=11.7 Hz, 2H).

### e. (S)-11-benzyl-1-(9H-fluoren-9-yl)-3,6,9,12,15-pentaoxo-2-oxa-4,7,10,13,16-pentaazaheptadecan-17-yl Acetate (Linker e)

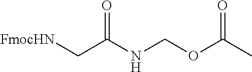

The Linker e compound was prepared according to the procedure described in US Patent Publication No. US 2017/021031.

### f. (S)-11-benzyl-1-(9H-fluoren-9-yl)-3,6,9,12,15-pentaoxo-2-oxa-4,7,10,13,16-pentaazaheptadecan-17-yl Acetate (Linker f)

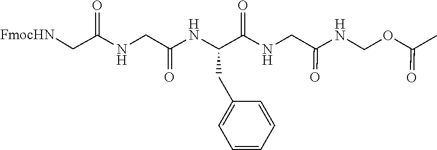

The Linker f was prepared according to the procedure described in US Patent Publication No. US 2017/021031 using Fmoc-GGFGG-OH as the starting peptide.

Other linkers linker g, linker h, linker i, and linker j are provided in FIG. 14A-D. These linkers were prepared and used for the preparation of compounds of Formula IV and conjugate molecules.

### Preparation of Compounds of Formula IV

The compounds of Formula IV were prepared using the compounds of Formula I and linker molecules as provided in examples 1 and 2 respectively. The compounds of Formula IV were prepared for the purpose of present disclosure: AV-DL038, AV-DL039, AV-DL094, AV-DL 095, AV-DL096, AV-DL100, and AV-DL103. The structures of the obtained compounds of Formula IV are shown in FIG. 15(A)-(G).

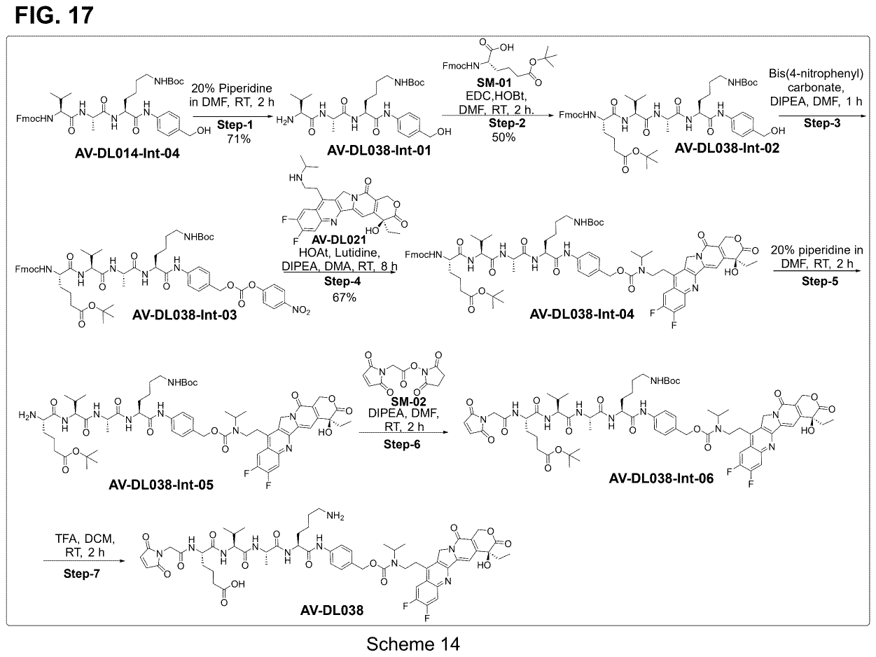

### XIV. Synthetic scheme for(S)-6-(((S)-1-(((S)-1-(((S)-6-amino-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxohexan-2-yl)amino)-1-oxopropan-2-yl)amino)-3-methyl-1-oxobutan-2-yl)amino)-5-(2-(2,5-dioxo-2,5-dihydro-1H-pyrrol-1-yl)acetamido)-6-oxohexanoic Acid (AV-DL038)(Scheme 14, FIG. 17)

### a. Synthesis of tert-butyl ((S)-5-((S)-2-((S)-2-amino-3-methylbutanamido)propanamido)-6-((4-(hydroxymethyl)phenyl)amino)-6-oxohexyl)carbamate (AV-DL038-Int-01)

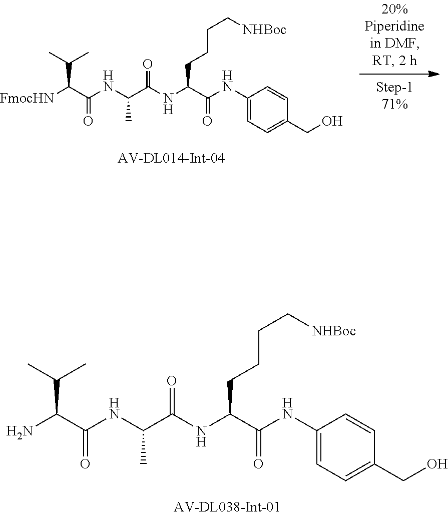

To a stirred solution of (9H-fluoren-9-yl)methyl((10S,13S,16S)-10-((4-(hydroxymethyl)phenyl)carbamoyl)-2,2,13,17-tetramethyl-4,12,15-trioxo-3-oxa-5,11,14-triazaoctadecan-16-yl)carbamate (AV-DL014-Int-04, 3 g, 4.04 mmol) in DMF (30 mL) was added piperdine (1.9 mL, 18.18 mmol) at 0° C. and stirred at RT for 2 h. The reaction was monitored by TLC. The reaction mixture was concentrated under reduced pressure to get crude product. The crude product was triturated with diethyl ether (3×50 mL) and dried to get tert-butyl ((S)-5-((S)-2-((S)-2-amino-3-methylbutanamido)propanamido)-6-((4-(hydroxymethyl)phenyl)amino)-6-oxohexyl)carbamate (AV-DL038-Int-01, 1.5 g, 2.88 mmol, 71% yield) as a light brown solid.

MS (ESI): 522.43 [M+H]⁺.

### b. Synthesis of tert-butyl (10S,13S,16S,19S)-19-((((9H-fluoren-9-yl)methoxy)carbonyl)amino)-10-((4-(hydroxymethyl)phenyl)carbamoyl)-16-isopropyl-2,2,13-trimethyl-4,12,15,18-tetraoxo-3-oxa-5,11,14,17-tetraazatricosan-23-oate (AV-DL038-Int-02)

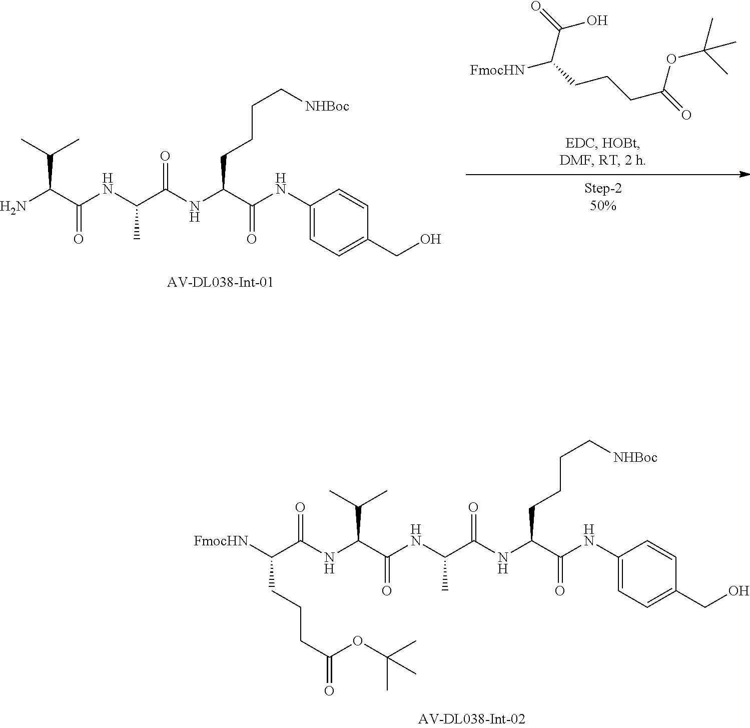

To a stirred solution of tert-butyl ((S)-5-((S)-2-((S)-2-amino-3-methylbutanamido)propanamido)-6-((4-(hydroxymethyl)phenyl)amino)-6-oxohexyl)carbamate (AV-DL038-Int-01, 1.5 g, 2.88 mmol) and Fmoc-Adi(tBu)-OH (890 mg, 2.02 mmol) in DMF (165 mL) was added EDC (560 mg, 2.88 mmol), HOBt (390 mg, 2.88 mmol) at 0° C. and stirred at RT for 2 h. The reaction was monitored by LCMS. The resulting reaction mixture was concentrated under reduced pressure to get crude product. The crude product was purified with reverse phase Biotage (column 60 g, C18) eluted with 40% MeCN in water and fractions were lyophilized to get tert-butyl (10S,13S,16S,19S)-19-((((9H-fluoren-9-yl)methoxy)carbonyl)amino)-16-isopropyl-2,2,13-trimethyl-10-((4-((((4-nitrophenoxy)carbonyl)oxy)methyl)phenyl)carbamoyl)-4,12,15,18-tetraoxo-3-oxa-5,11,14,17-tetraazatricosan-23-oate (AV-DL038-Int-02, 1.35 g, 1.433 mmol, 50% yield) as a light brown solid.

MS (ESI): 944.09 [M+H]⁺.

### c. Synthesis of tert-butyl (10S,13S,16S,19S)-19-((((9H-fluoren-9-yl)methoxy)carbonyl)amino)-16-isopropyl-2,2,13-trimethyl-10-((4-((((4-nitrophenoxy)carbonyl)oxy)methyl)phenyl)carbamoyl)-4,12,15,18-tetraoxo-3-oxa-5,11,14,17-tetraazatricosan-23-oate (AV-DL038-Int-03)

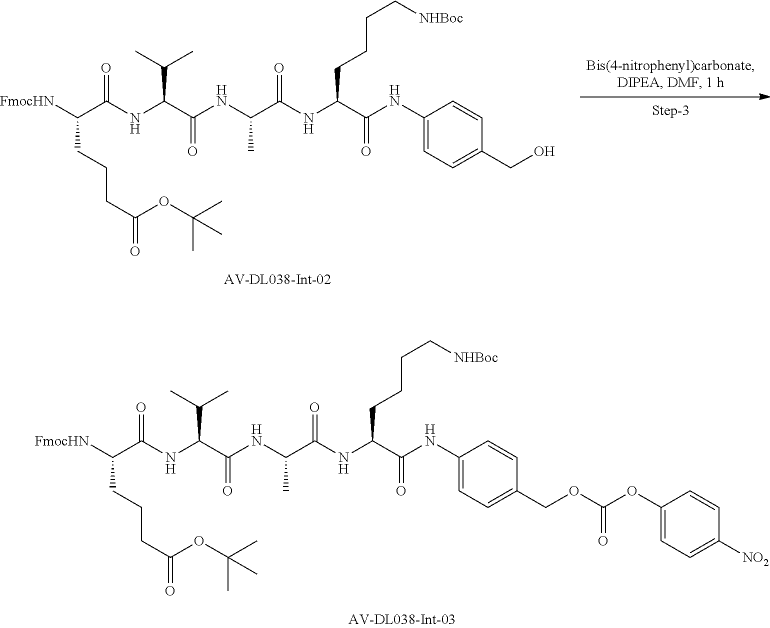

To a stirred solution of tert-butyl (10S,13S,16S,19S)-19-((((9H-fluoren-9-yl)methoxy)carbonyl)amino)-10-((4-(hydroxymethyl)phenyl)carbamoyl)-16-isopropyl-2,2,13-trimethyl-4,12,15,18-tetraoxo-3-oxa-5,11,14,17-tetraazatricosan-23-oate (AV-DL038-Int-02, 750 mg, 0.797 mmol) in DMF (10 mL) was added bis(4-nitrophenyl)carbonate (727 mg, 2.389 mmol), DIPEA (0.42 mL, 2.389 mmol) at 0° C. and stirred at RT for 1 h. The reaction was monitored by TLC. The reaction mixture was concentrated under reduced pressure to get crude product. The crude product was triturated with diethyl ether (3×50 mL) and dried to get tert-butyl (10S,13S,16S,19S)-19-((((9H-fluoren-9-yl)methoxy)carbonyl)amino)-16-isopropyl-2,2,13-trimethyl-10-((4-((((4-nitrophenoxy)carbonyl)oxy)methyl)phenyl)carbamoyl)-4,12,15,18-tetraoxo-3-oxa-5,11,14,17-tetraazatricosan-23-oate (AV-DL038-Int-03, 1 g, 0.903 mmol, quantitative yield) as a pale yellow solid.

MS (ESI): 1108.38 [M+H]⁺.

### d. Synthesis of tert-butyl (10S,13S,16S,19S)-19-((((9H-fluoren-9-yl)methoxy)carbonyl)amino)-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-04)

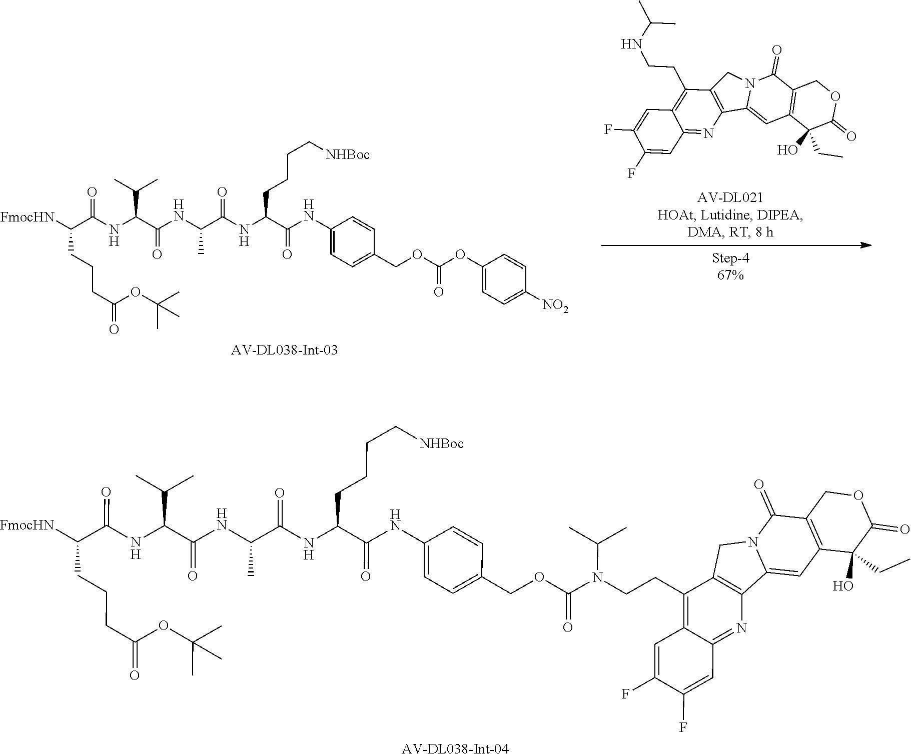

To a stirred solution of tert-butyl (10S,13S,16S,19S)-19-((((9H-fluoren-9-yl)methoxy)carbonyl)amino)-16-isopropyl-2,2,13-trimethyl-10-((4-((((4-nitrophenoxy)carbonyl)oxy)methyl)phenyl)carbamoyl)-4,12,15,18-tetraoxo-3-oxa-5,11,14,17-tetraazatricosan-23-oate (AV-DL038-Int-03, 200 mg, 0.18 mmol), (S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-DL021, 85 mg, 0.18 mmol), and 2,6-lutidine (0.14 mL, 0.91 mmol) in DMF (2 mL, 10 vol) was added HOPO (101 mg, 0.91 mmol) at 0° C. and stirred at RT for 8 h. The reaction was monitored by TLC. The resulting reaction mixture was concentrated under reduced pressure to get crude product. The crude product was further purified by reverse phase Biotage (column 30 g, C18) eluted with 30% MeCN in water to get tert-butyl (10S,13S,16S,19S)-19-((((9H-fluoren-9-yl)methoxy)carbonyl)amino)-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-04, 175 mg, 0.122 mmol, 67% yield) as an off white solid.

MS (ESI): 1439.98 [M+H]⁺.

### e. Synthesis of tert-butyl (10S,13S,16S,19S)-19-amino-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-05)

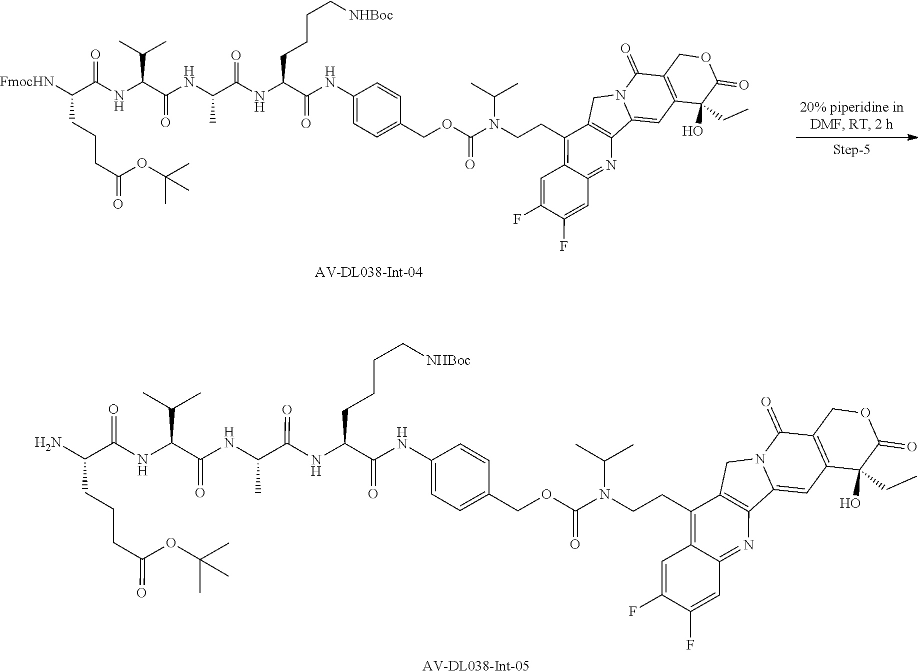

To a stirred solution tert-butyl (10S,13S,16S,19S)-19-((((9H-fluoren-9-yl)methoxy)carbonyl)amino)-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-04, 175 mg, 0.122 mmol) in DMF (1 mL) was added piperdine (0.06 mL, 0.549 mmol) at 0° C. and stirred at RT for 2 h. The reaction was monitored by TLC. The reaction mixture was concentrated under reduced pressure to get crude product. The crude product was triturated with diethyl ether (3×10 mL) and dried to get tert-butyl (10S,13S,16S,19S)-19-amino-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-05, 150 mg, 0.124 mmol, quantitative yield) as a light brown solid.

MS (ESI): 1215.88 [M+H]⁺.

### f. Synthesis of tert-butyl (10S,13S,16S,19S)-19-(2-(2,5-dioxo-2,5-dihydro-1H-pyrrol-1-yl)acetamido)-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-06)

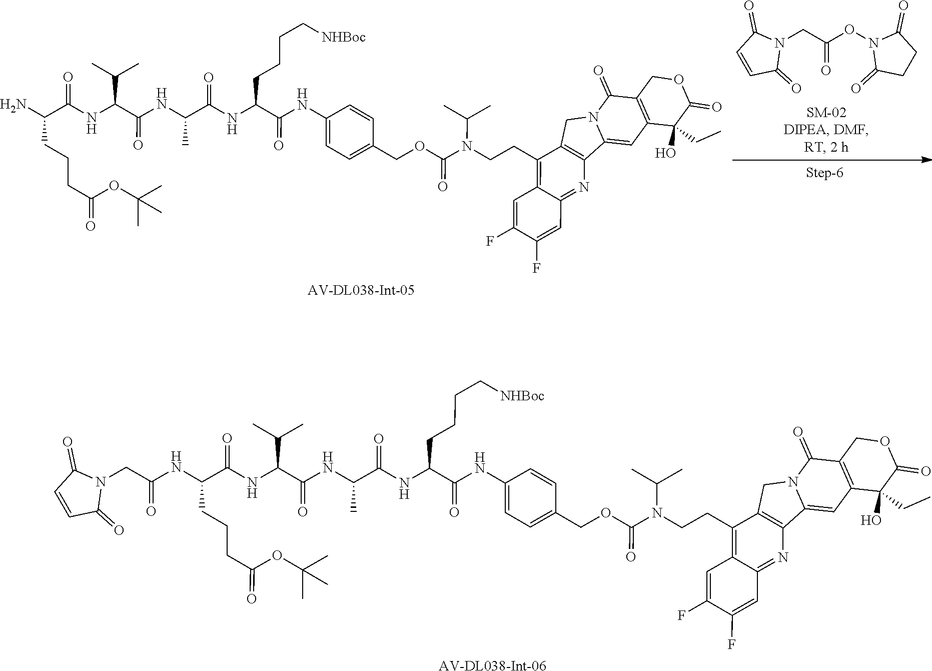 [SMILES: CC[C@@]1(O)C(=O)OCc2c1cc1n(c2=O)Cc2c-1nc1cc(F)c(F)cc1c2CCN(C(=O)OCc1ccc(NC(=O)[C@H](CCCCNC(=O)OC(C)(C)C)NC(=O)[C@H](C)NC(=O)[C@@H](NC(=O)[C@@H](N)CCCC(=O)OC(C)(C)C)C(C)C)cc1)C(C)C ; O=C(CN1C(=O)C=CC1=O)ON1C(=O)CCC1=O ; "AV-DL038-Int-05" ; CC[C@@]1(O)C(=O)OCc2c1cc1n(c2=O)Cc2c-1nc1cc(F)c(F)cc1c2CCN(C(=O)OCc1ccc(NC(=O)[C@H](CCCCNC(=O)OC(C)(C)C)NC(=O)[C@H](C)NC(=O)[C@@H](NC(=O)[C@H](CCCC(=O)OC(C)(C)C)NC(=O)CN2C(=O)C=CC2=O)C(C)C)cc1)C(C)C ; "AV-DL038-Int-06"]

To a stirred solution of tert-butyl (10S,13S,16S,19S)-19-amino-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-05, 150 mg, 0.124 mmol) and 2,5-dioxopyrrolidin-1-yl 2-(2,5-dioxo-2,5-dihydro-1H-pyrrol-1-yl)acetate (32 mg, 0.124 mmol) in DMF (1.5 mL) and stirred at RT for 2 h. The reaction was monitored by LCMS. The resulting reaction mixture was concentrated under reduced pressure to get semi pure tert-butyl (10S,13S,16S,19S)-19-(2-(2,5-dioxo-2,5-dihydro-1H-pyrrol-1-yl)acetamido)-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-06, 175 mg, 0.166 mmol, quantitative yield) as a light brown solid. This was used as such for the next step without further purification.

MS (ESI): 1353.71 [M+H]⁺.

### g. Synthesis of (2S,3S,4S,5R,6S)-6-(4-((10S,11R,14R,15R)-15-((S)-1-((5S,8S,11S,12R)-1-(4-((S)-6-amino-2-((S)-2-((S)-5-carboxy-2-(3-(2,5-dioxo-2,5-dihydro-1H-pyrrol-1-yl)propanamido)pentanamido)-3-methylbutanamido)hexanamido)phenyl)-11-((S)-sec-butyl)-5,8-diisopropyl-12-methoxy-4,10-dimethyl-3,6,9-trioxo-2-oxa-4,7,10-triazatetradecan-14-oyl)pyrrolidin-2-yl)-4,7,11,14-tetramethyl-3,8,13-trioxo-10-phenyl-2,9,16-trioxa-4,7,12-triazaheptadecyl)phenoxy)-3,4,5-trihydroxytetrahydro-2H-pyran-2-carboxylic Acid (AV-DL038)

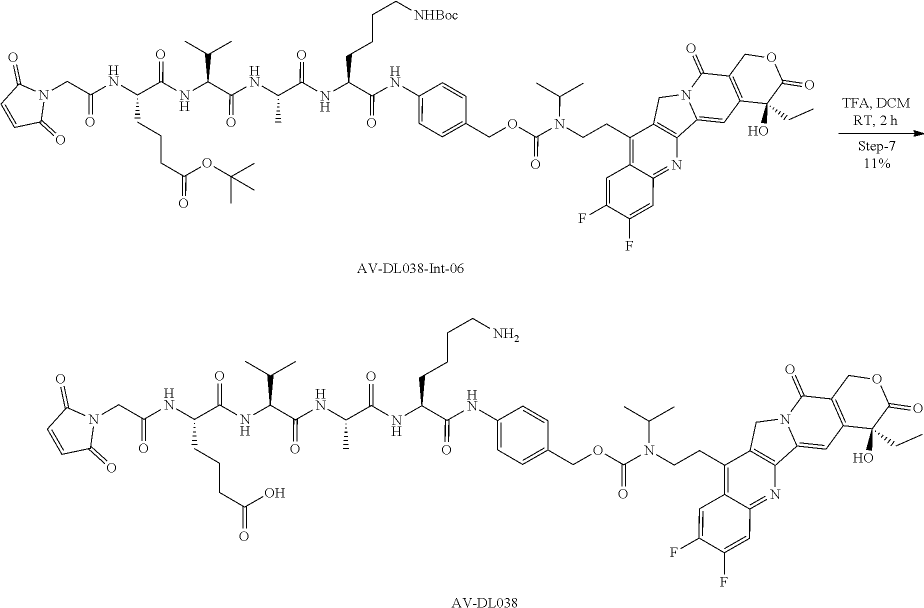 [SMILES: CC[C@@]1(O)C(=O)OCc2c1cc1n(c2=O)Cc2c-1nc1cc(F)c(F)cc1c2CCN(C(=O)OCc1ccc(NC(=O)[C@H](CCCCNC(=O)OC(C)(C)C)NC(=O)[C@H](C)NC(=O)[C@@H](NC(=O)[C@H](CCCC(=O)OC(C)(C)C)NC(=O)CN2C(=O)C=CC2=O)C(C)C)cc1)C(C)C ; "AV-DL038-Int-06" ; CC[C@@]1(O)C(=O)OCc2c1cc1n(c2=O)Cc2c-1nc1cc(F)c(F)cc1c2CCN(C(=O)OCc1ccc(NC(=O)[C@H](CCCCN)NC(=O)[C@H](C)NC(=O)[C@@H](NC(=O)[C@H](CCCC(=O)O)NC(=O)CN2C(=O)C=CC2=O)C(C)C)cc1)C(C)C ; "AV-DL038"]

A solution of tert-butyl (10S,13S,16S,19S)-19-(2-(2,5-dioxo-2,5-dihydro-1H-pyrrol-1-yl)acetamido)-10-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)carbamoyl)-16-isopropyl-2,2,13-trimethyl-4,12,15,18-tetraoxo-3-oxa-5,11,14,17-tetraazatricosan-23-oate (AV-DL038-Int-06, 225 mg, 0.166 mmol) in 20% TFA in DCM (4.5 mL) was stirred at RT for 2 h. The reaction was monitored by LCMS. The resulting reaction mixture was concentrated under reduced pressure to get crude product. The crude product was further purified by RP Prep-HPLC to get (2S,3S,4S,5R,6S)-6-(4-((10S,11R,14R,15R)-15-((S)-1-((5S,8S,11S,12R)-1-(4-((S)-6-amino-2-((S)-2-((S)-5-carboxy-2-(3-(2,5-dioxo-2,5-dihydro-1H-pyrrol-1-yl)propanamido)pentanamido)-3-methylbutanamido)hexanamido)phenyl)-11-((S)-sec-butyl)-5,8-diisopropyl-12-methoxy-4,10-dimethyl-3,6,9-trioxo-2-oxa-4,7,10-triazatetradecan-14-oyl)pyrrolidin-2-yl)-4,7,11,14-tetramethyl-3,8,13-trioxo-10-phenyl-2,9,16-trioxa-4,7,12-triazaheptadecyl)phenoxy)-3,4,5-trihydroxytetrahydro-2H-pyran-2-carboxylic acid (AV-DL038, 22.4 mg, 0.0187 mmol, 11% yield) as an off white solid.

PREP-HPLC Conditions: X-Bridge C18 (250*21.2 mm) 5μ, 250×19.1 mm×5 μm A: A: 0.1% TFA IN WATER; B: A CN:H₂O (80:20): 0/20, 16/60, 20/60, 20.1/98@14 ML/MIN; diluent: MeCN, Water.

MS (ESI): 1197.58 [M+H]⁺; ¹H NMR (400 MHz, DMSO-d₆): δ 12.01 (brs, 1H), 10.05 (brs, 1H), 8.35 (d, J=8.0 Hz, 1H), 8.01 (d, J=6.8 Hz, 2H), 7.34 (d, J=8.4 Hz, 1H), 7.64-7.60 (m, 5H), 7.36-7.33 (m, 3H), 7.11 (s, 2H), 6.54 (s, 1H), 5.49-5.38 (m, 4H), 5.42-5.36 (m, 2H), 5.13 (s, 2H), 4.41-4.32 (m, 3H), 4.28-4.13 (m, 2H), 4.07 (s, 2H), 3.44-3.40 (m, 4H), 2.78-2.74 (m, 2H), 2.18 (t, J=6.8 Hz, 2H), 1.89-1.85 (m, 3H), 1.75-1.48 (m, 7H), 1.46-1.38 (m, 2H), 1.20 (d, J=6.8 Hz, 3H), 1.11 (d, J=6.4 Hz, 6H), 0.89-0.80 (m, 9H).

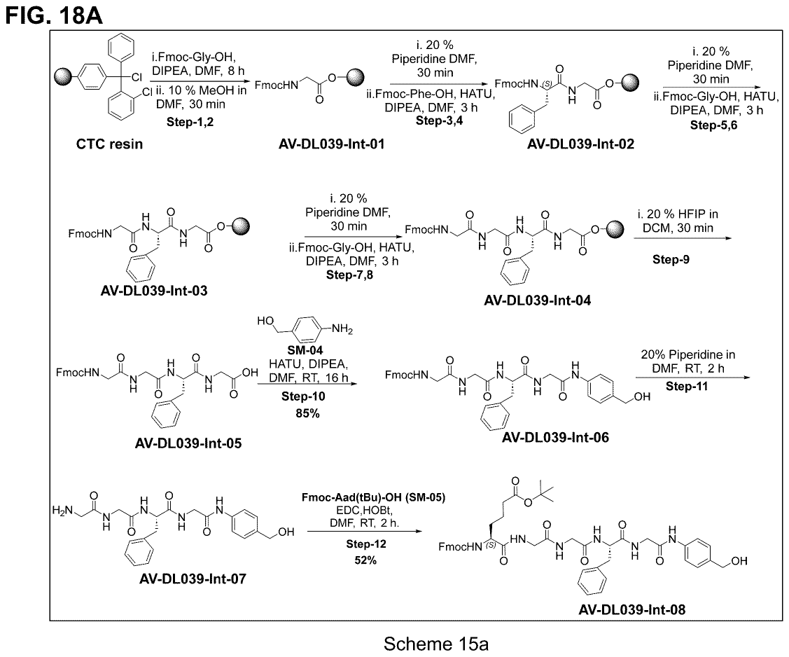

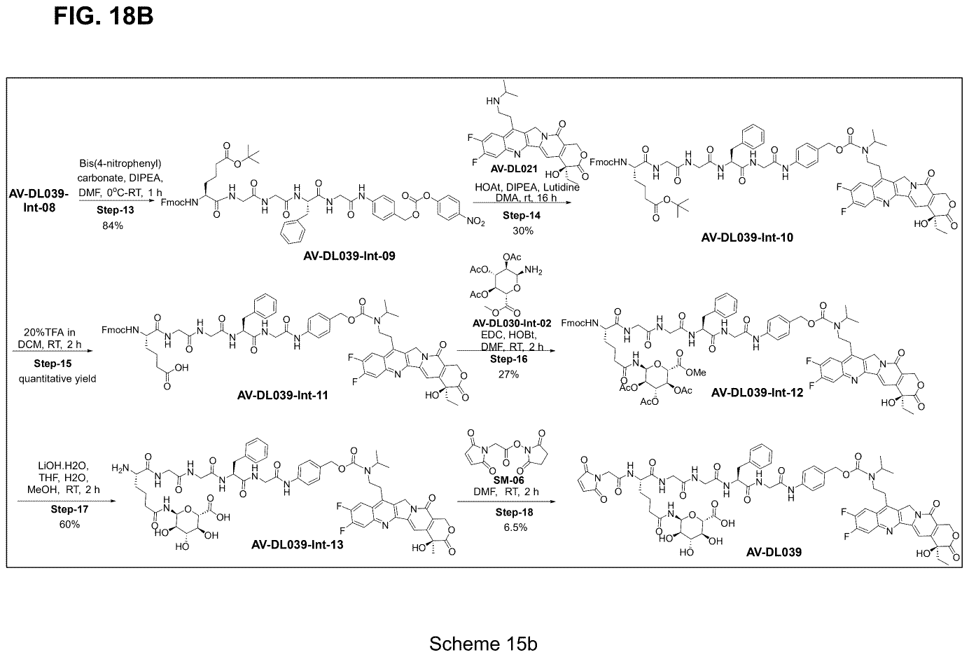

### XV. Synthetic Scheme for (2S,3S,4S,5R,6S)-6-((5S,14S)-5-benzyl-14-(2-(2,5-dioxo-2,5-dihydro-1H-pyrrol-1-yl)acetamido)-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL039)(Scheme 15a and Scheme 15b, FIGS. 18A and 18B)

### a. Synthesis of (((9H-fluoren-9-yl)methoxy)carbonyl)glycylglycyl-L-phenylalanylglycine (AV-DL039-Int-05)(Scheme 15a)

### General Procedure for Solid Phase Peptide Synthesis

### First Amino Acid Loading:

Amino acid (3 eq) and diisopropyl-ethylamine (DIPEA)(6 eq) were dissolved in DMF (10 vol), the solution was mixed with Pre-Swelled 2-CTC (1.0 g, 1.0 mmol/g) resin in peptide flask and then placed on shaker for 8 h and the resin was then washed with DMF and DCM (3 times, 10 Vol).

Capping: To the resin 10% MeOH in DMF (10 vol), DIPEA (6 eq) were added and agitated for 30 minutes. Subsequently, the resin was washed with DMF and DCM (3 times, 10 Vol).

### Fmoc Deprotection:

The peptidyl-resin was treated twice with 20% piperidine in DMF (10 vol) for 15 minutes (2 times). The resin was washed 4 times using DMF and DCM (3 times, 10 Vol).

### Kaiser Test: +Ve (Blue Colour)

### AA Coupling:

Fmoc-protected amino acid (1.5 eq), (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium)(HATU, 1.5 eq) and diisopropyl-ethylamine (DIPEA)(3 eq) were dissolved in DMF (10 vol). The solution was mixed with the resin-bound peptide in peptide flask and then placed on shaker for 3 h and the resin was then washed with DMF and DCM (3 times, 10 Vol).

### Kaiser Test: −Ve (Off-White Colour)

### Resin Cleavage:

### b. Synthesis of (((9H-fluoren-9-yl)methoxy)carbonyl)glycylglycyl-L-phenylalanylglycine (AV-DL039-Int-05)

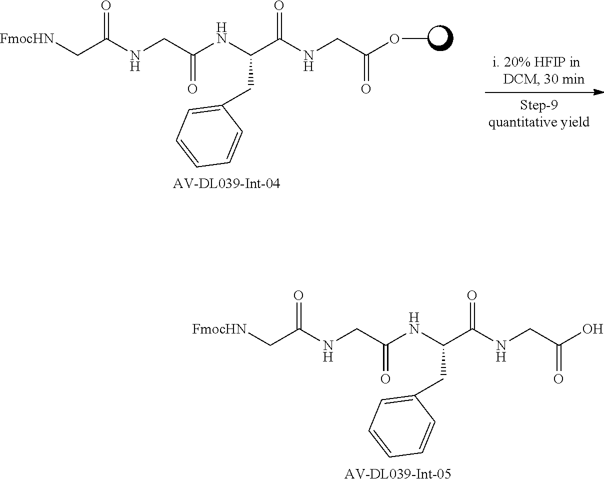

The pre-swelled peptidyl resin (AV-DL039-Int-04, 8.8 g, 1.0 mmol/g) was treated with 30% HFIP in DCM (88 mL, 10 vol) and agitated for 15 minutes. The reaction was filtered and the filtrate was concentrated under vacuum. The same procedure was repeated again to get (((9H-fluoren-9-yl)methoxy)carbonyl)glycylglycyl-L-phenylalanylglycine (AV-DL039-Int-05, 5 g, 8.9 mmol, quantitative yield) as an off white solid.

MS (ESI): 559.0 [M+H]⁺.

### c. Synthesis of (9H-fluoren-9-yl)methyl(S)-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)carbamate (AV-DL039-Int-06)

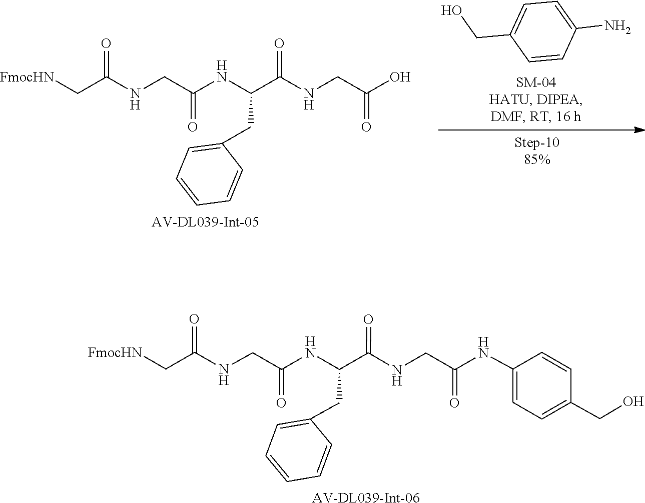

To a stirred solution of (((9H-fluoren-9-yl)methoxy)carbonyl)glycylglycyl-L-phenylalanylglycine (AV-DL039-Int-05, 3 g, 5.37 mmol) in DMF (30 mL) was added HATU (3.06 g, 8.06 mmol), DIPEA (2.8 mL, 16.13 mmol) and (4-aminophenyl) methanol (662 mg, 5.37 mmol) at 0° C., and the reaction was allowed to warm gradually to RT and stirred for 16 h. The reaction was monitored by TLC. The resulting reaction mixture was concentrated under reduced pressure to get crude product. The crude product was purified by reverse phase Biotage (column 60 g, C18) eluted with 41% MeCN in water and fractions were lyophilized to get (9H-fluoren-9-yl)methyl(S)-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)carbamate (AV-DL039-Int-06, 3 g, 4.5 mmol, 85% yield) as a light brown solid.

MS (ESI): 664.30 [M+H]⁺.

### d. Synthesis of(S)-2-(2-(2-aminoacetamido)acetamido)-N-(2-((4-(hydroxymethyl)phenyl)amino)-2-oxoethyl)-3-phenylpropanamide (AV-DL039-Int-07)

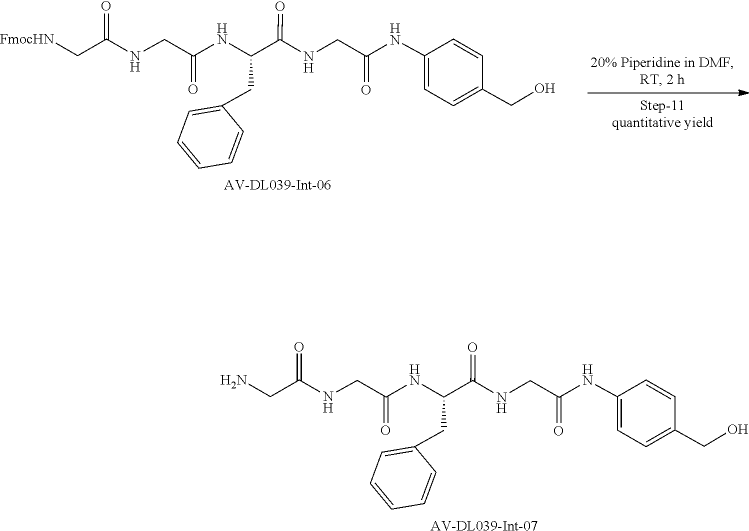

To a stirred solution of tert-butyl(S)-5-((S)-2-((((9H-fluoren-9-yl)methoxy)carbonyl)amino)-6-((tert-butoxycarbonyl)amino)hexanamido)-6-(((S)-1-(((S)-1-((4 -(hydroxymethyl)phenyl)amino)-1-oxopropan-2-yl)amino)-3-methyl-1-oxobutan-2-yl)amino)-6-oxohexanoate (AV-DL039-Int-06, 3 g, 4.52 mmol) in DMF (20 mL) was added piperdine (2 mL, 20.34 mmol) at 0° C. and stirred at RT for 2 h. The reaction was monitored by TLC. The reaction mixture was concentrated under reduced pressure to get crude product. The crude product was triturated with diethyl ether (3×100 mL) and dried to get(S)-2-(2-(2-aminoacetamido)acetamido)-N-(2-((4-(hydroxymethyl)phenyl)amino)-2-oxoethyl)-3-phenylpropanamide (AV-DL039-Int-07, 2 g, 2.32 mmol, quantitative yield) as a pale yellow solid.

MS (ESI): 442.12 [M+H]⁺.

### e. Synthesis of tert-butyl (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oate (AV-DL039-Int-08)

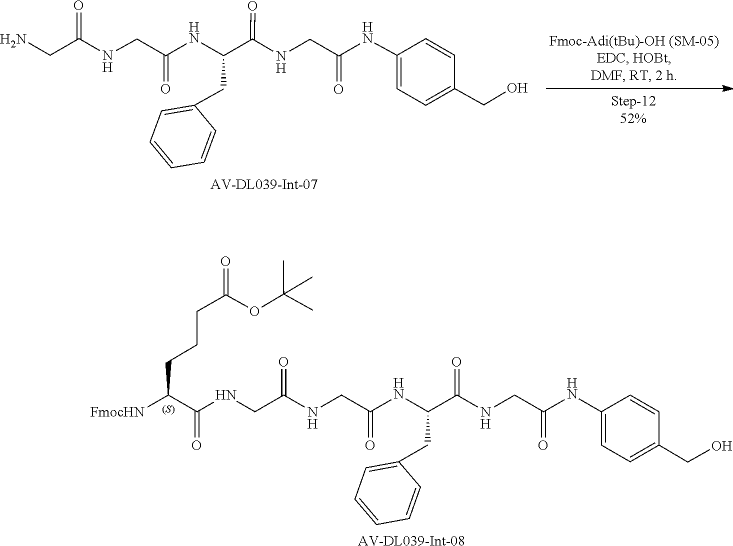

To a stirred solution of(S)-2-(2-(2-aminoacetamido)acetamido)-N-(2-((4-(hydroxymethyl)phenyl)amino)-2-oxoethyl)-3-phenylpropanamide (AV-DL039-Int-07, 2 g, 4.53 mmol) and Fmoc-Adi(tBu)-OH (1.99 g, 4.53 mmol) in DMF (30 mL) was added EDC (1.7 g, 8.96 mmol) and HOBt (1.21 g, 8.96 mmol) at 0° C. and stirred at RT for 2 h. The reaction was monitored by LCMS. The resulting reaction mixture was concentrated under reduced pressure to get crude product. The crude product was purified by reverse phase Biotage (column 60 g, C18) eluted with 41% MeCN in water and fractions were lyophilized to get tert-butyl (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oate (AV-DL039-Int-08, 1 g, 1.16 mmol, 52% yield) as an off white solid.

MS (ESI): 864.04 [M+H]⁺.

### f. Synthesis of tert-butyl (5S,14S)-14-((((9H-fluoren-9-yl)methoxy)carbonyl)amino)-5-benzyl-1-((4-((((4-nitrophenoxy)carbonyl)oxy)methyl)phenyl)amino)-1,4,7,10,13-pentaoxo-3,6,9,12-tetraazaoctadecan-18-oate (AV-DL039-Int-09)

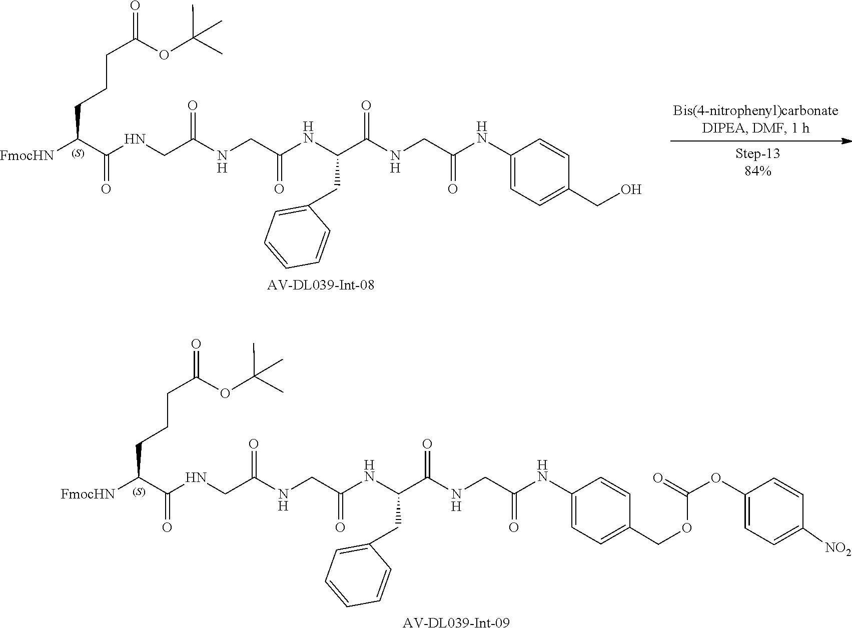

To a stirred solution of tert-butyl (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oate (AV-DL039-Int-08, 1 g, 1.16 mmol), bis(4-nitrophenyl)carbonate (1 g, 3.47 mmol) in DMF (10 mL), was added DIPEA (0.56 mL, 3.47 mmol) at 0° C. and stirred at RT for 1 h. The reaction was monitored by TLC. The solvent was removed under vacuum and triturated with diethyl ether, dried to get tert-butyl (5S,14S)-14-((((9H-fluoren-9-yl)methoxy)carbonyl)amino)-5-benzyl-1-((4-((((4-nitrophenoxy)carbonyl)oxy)methyl)phenyl)amino)-1,4,7,10,13-pentaoxo-3,6,9,12-tetraazaoctadecan-18-oate (AV-DL039-Int-09, 1 g, 0.973 mmol, 84% yield) as a pale yellow solid. This was used as such for the next step.

MS (ESI): 1028.27 [M+H]⁺.

### g. Synthesis of tert-butyl (5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-oate (AV-DL039-Int-10)

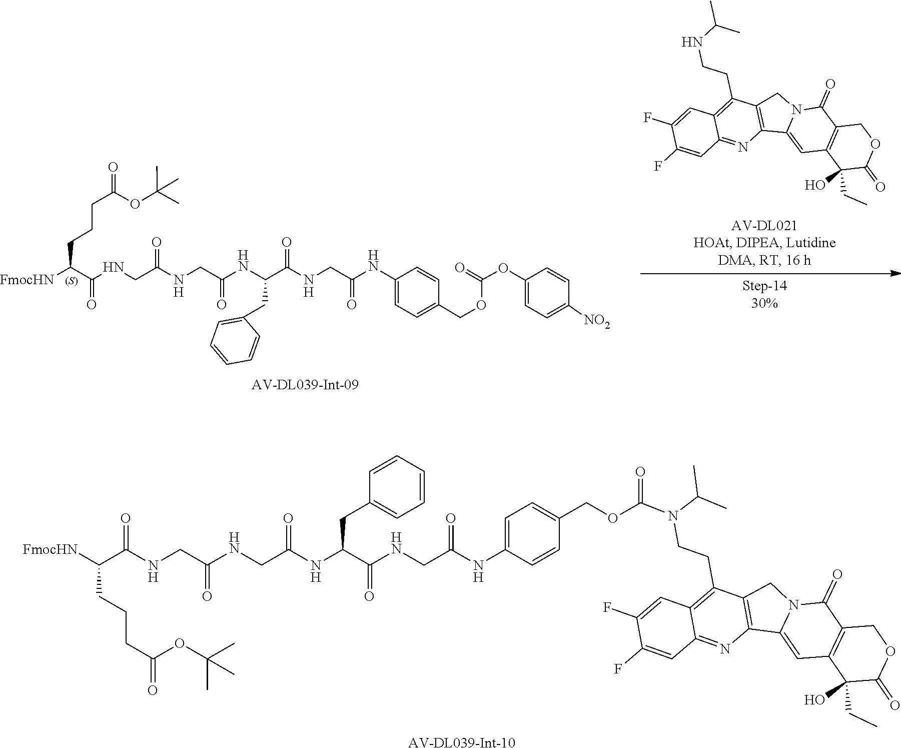

To a stirred solution of tert-butyl (5S,14S)-14-((((9H-fluoren-9-yl)methoxy)carbonyl)amino)-5-benzyl-1-((4-((((4-nitrophenoxy)carbonyl)oxy)methyl)phenyl)amino)-1,4,7,10,13-pentaoxo-3,6,9,12-tetraazaoctadecan-18-oate (AV-DL039-Int-09, 750 mg, 0.730 mmol), (S)-4-ethyl-8,9-difluoro-4 -hydroxy-11-(2-(isopropylamino)ethyl)-1,12-dihydro-14H-pyrano[3′,4′:6,7]indolizino[1,2-b]quinoline-3,14(4H)-dione (AV-DL021, 340 mg, 0.73 mmol), and 2,6-lutidine (0.43 mL, 2.92 mmol) in DMA (10 mL, 10 vol) was added HOAt in DMA (1 mL, 1.095 mmol) and DIPEA (0.37 mL, 2.92 mmol) at 0° C., and stirred at RT for 16 h. The reaction was monitored by LCMS. The resulting reaction mixture was concentrated under reduced pressure to get crude product. The crude product was further purified by reverse phase GRACE (column 60 g, C18) eluted with 58% MeCN in water to get tert-butyl (5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-oate (AV-DL039-Int-10, 300 mg, 0.22 mmol, 30% yield) as an off white solid.

MS (ESI): 1359.02 [M+H]⁺.

### h. Synthesis of (5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-oic Acid (AV-DL039-Int-11)

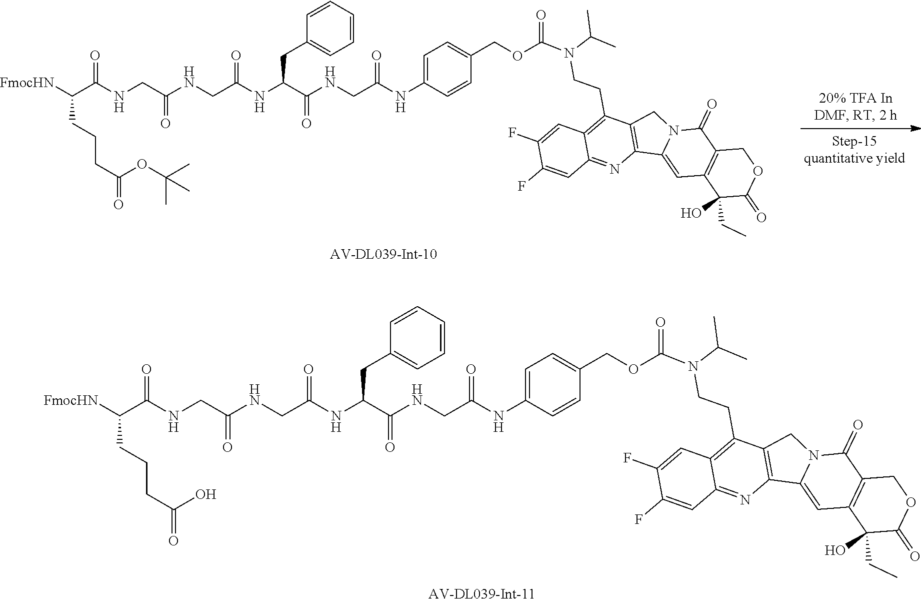

A solution of tert-butyl (5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-oate (AV-DL039-Int-10, 300 mg, 0.22 mmol) in 20% TFA in DCM (10 mL) was stirred at RT for 2 h. The reaction was monitored by LCMS. The resulting reaction mixture was concentrated under reduced pressure to get (5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-oic acid (AV-DL039-Int-11, 300 mg, 0.23 mmol, quantitative yield) as an off white solid.

MS (ESI): 1303.14 [M+H]⁺.

### i. Synthesis of (2S,3R,4S,5S,6S)-2-((5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL039-Int-12)

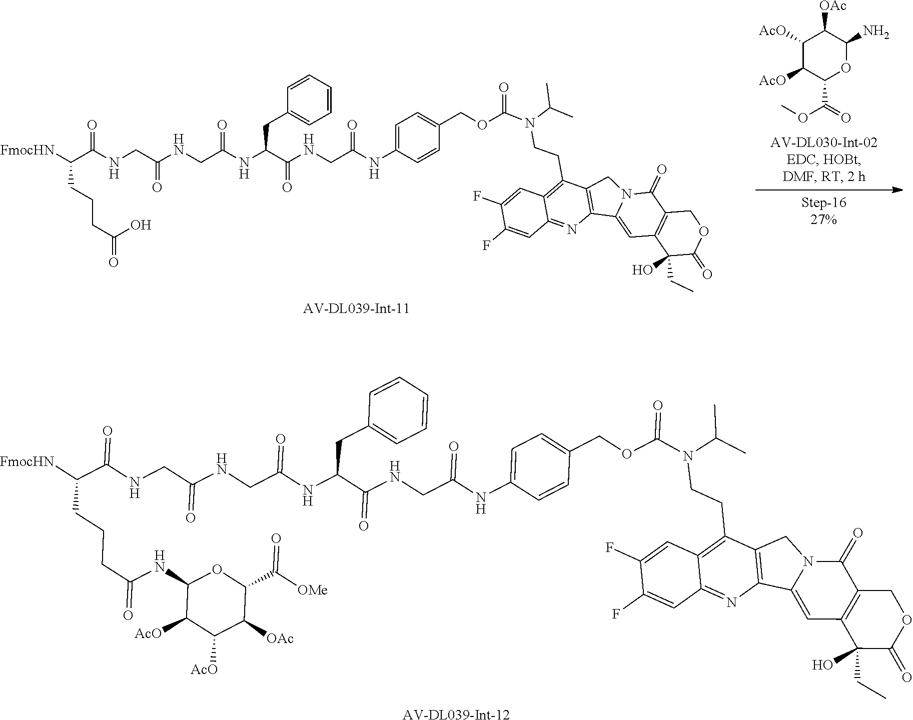

To a stirred solution of (5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-oic acid (AV-DL039-Int-11, 300 mg, 0.23 mmol), EDC·HCl (87 mg, 0.46 mmol), HOBt (63 mg, 0.46 mmol) and DIPEA (0.19 mL, 1.07 mmol) in DMF was added (2S,3R,4S,5S,6S)-2-amino-6-(methoxycarbonyl)tetrahydro-2H-pyran-3,4,5-triyl triacetate (AV-DL030-Int-02, 230 mg, 0.691 mmol) at 0° C. and stirred at RT for 2 h. The reaction was monitored by LCMS. The resulting reaction mixture was concentrated under reduced pressure to get crude product. The crude product was purified with reverse phase Biotage (column 30 g, C18) eluted with 29% MeCN in water and fractions were lyophilized to get (2S,3R,4S,5S,6S)-2-((5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL039-Int-12, 100 mg, 0.062 mmol, 27% yield) as an off white solid.

MS (ESI): 1618.29 [M+H]⁺.

### j. Synthesis of (2S,3S,4S,5R,6S)-6-((5S,14S)-14-amino-5-benzyl-1-((4-((((2-((S)-8,9-difluoro-4-hydroxy-4-methyl-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic Acid (AV-DL039-Int-13)

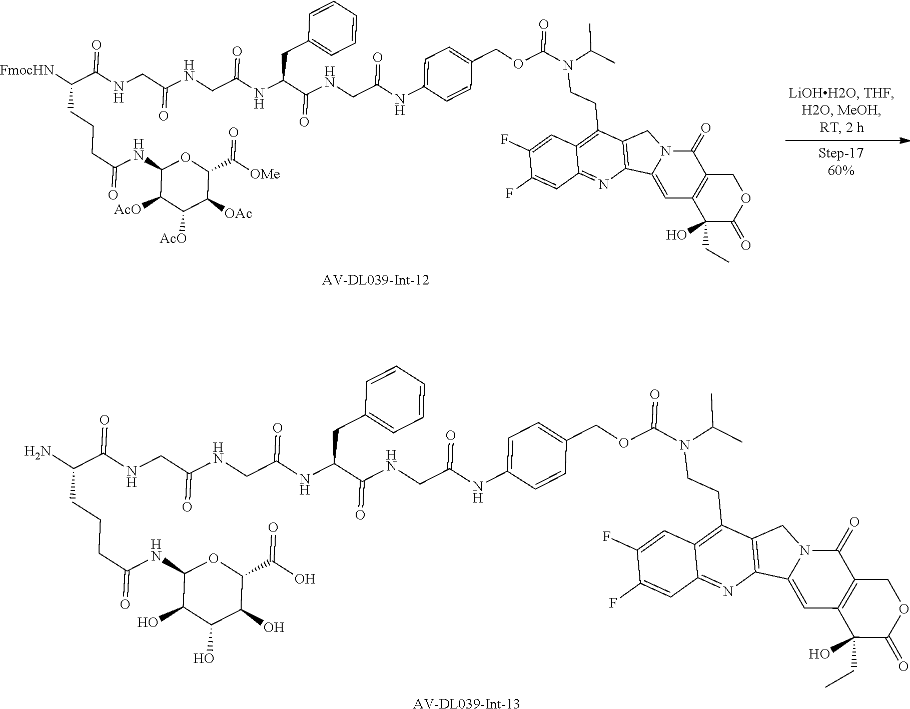

To a stirred solution of (2S,3R,4S,5S,6S)-2-((5S,14S)-14-((((9H-fluoren-9-yl)methoxy)carbonyl)amino)-5-benzyl-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL039-Int-12, 130 mg, 0.080 mmol) in THF:H₂O:MeOH (1:1:1)(3 mL) was added LiOH·H₂O (20 mg, 0.48 mmol) at 0° C. and stirred at RT for 2 h. The reaction was monitored by LCMS. The reaction mixture was concentrated under reduced pressure to get crude product. The crude product was further purified by reverse phase Biotage (column 12 g, C18) eluted with 15% MeCN in water to get (2S,3S,4S,5R,6S)-6-((5S,14S)-14-amino-5-benzyl-1-((4-((((2-((S)-8,9-difluoro-4-hydroxy-4-methyl-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL039-Int-13, 60 mg, 0.048 mmol, 60% yield) as an off white solid.

MS (ESI): 1255.56 [M+H]⁺.

### k. Synthesis of (2S,3S,4S,5R,6S)-6-((5S,14S)-5-benzyl-14-(2-(2,5-dioxo-2,5-dihydro-1H-pyrrol-1-yl)acetamido)-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL039)

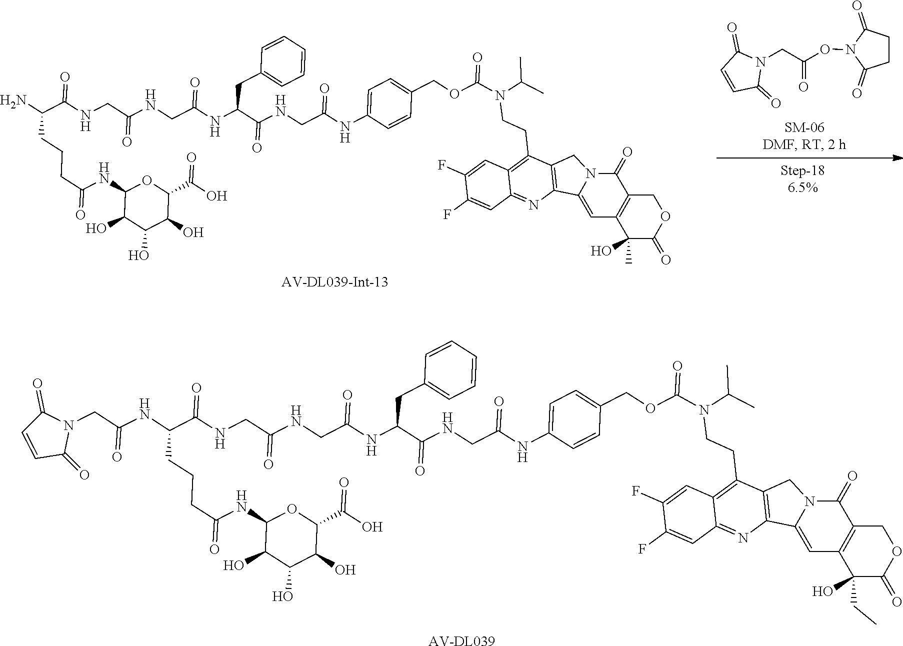 [SMILES: CC(C)N(CCc1c2c(nc3cc(F)c(F)cc13)-c1cc3c(c(=O)n1C2)COC(=O)[C@@]3(C)O)C(=O)OCc1ccc(NC(=O)CNC(=O)[C@H](Cc2ccccc2)NC(=O)CNC(=O)CNC(=O)[C@@H](N)CCCC(=O)N[C@H]2O[C@H](C(=O)O)[C@@H](O)[C@H](O)[C@H]2O)cc1 ; O=C(CN1C(=O)C=CC1=O)ON1C(=O)CCC1=O ; "AV-DL039-Int-13" ; CC[C@@]1(O)C(=O)OCc2c1cc1n(c2=O)Cc2c-1nc1cc(F)c(F)cc1c2CCN(C(=O)OCc1ccc(NC(=O)CNC(=O)[C@H](Cc2ccccc2)NC(=O)CNC(=O)CNC(=O)[C@H](CCCC(=O)N[C@H]2O[C@H](C(=O)O)[C@@H](O)[C@H](O)[C@H]2O)NC(=O)CN2C(=O)C=CC2=O)cc1)C(C)C ; "AV-DL039"]

To a stirred solution of (2S,3S,4S,5R,6S)-6-((5S,14S)-14-amino-5-benzyl-1-((4-((((2-((S)-8,9-difluoro-4-hydroxy-4-methyl-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL039-Int-13, 60 mg, 0.047 mmol) in DMF (2 mL) was added 2,5-dioxopyrrolidin-1-yl 2-(2,5-dioxo-2,5-dihydro-1H-pyrrol-1-yl)acetate (SM-06, 24 mg, 0.095 mmol) at 0° C. and stirred at RT for 1 h. The reaction was monitored by LCMS. The resulting reaction mixture was concentrated under reduced pressure to get crude product. The crude product was further purified by RP Prep-HPLC and fractions were lyophilized to get (2S,3S,4S,5R,6S)-6-((5S,14S)-5-benzyl-14-(2-(2,5-dioxo-2,5-dihydro-1H-pyrrol-1-yl)acetamido)-1-((4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL039, 4.3 mg, 0.0031 mmol, 6.5% yield) as an off white solid.

PREP-HPLC Conditions: Shim-pack C18 (250*20.0 mm) 5μ: 0.1% TFA in water B: Acetonitrile: 0/40, 1/40, 15/52,22/52,22.1/98; diluent: MeCN, Water.

MS (ESI): 1392.15 [M+H]⁺: ¹H NMR (400 MHz, DMSO-d₆): δ 12.78 (brs, 1H), 9.89 (s, 1H), 8.40-8.35 (m, 3H), 8.30-8.11 (m, 3H), 8.06-7.99 (m, 1H), 7.65-7.55 (m, 2H), 7.41-7.30 (m, 4H), 7.28-7.15 (m, 6H), 7.05 (s, 2H), 6.54 (s, 1H), 5.44 (s, 2H), 5.40-5.30 (m, 2H), 5.14-5.10 (m, 2H), 4.78-4.71 (m, 1H), 4.59-4.50 (m, 1H), 4.30-4.05 (m, 4H), 3.91-3.85 (m, 2H), 3.80-3.55 (m, 5H), 3.50-3.40 (m, 5H), 3.26-3.05 (m, 4H), 2.89-2.79 (m, 1H), 2.20-2.05 (m, 2H), 1.91-1.80 (m, 2H), 1.70-1.40 (m, 4H), 1.30-1.10 (m, 7H), 0.87 (t, J=7.6 Hz, 3H).

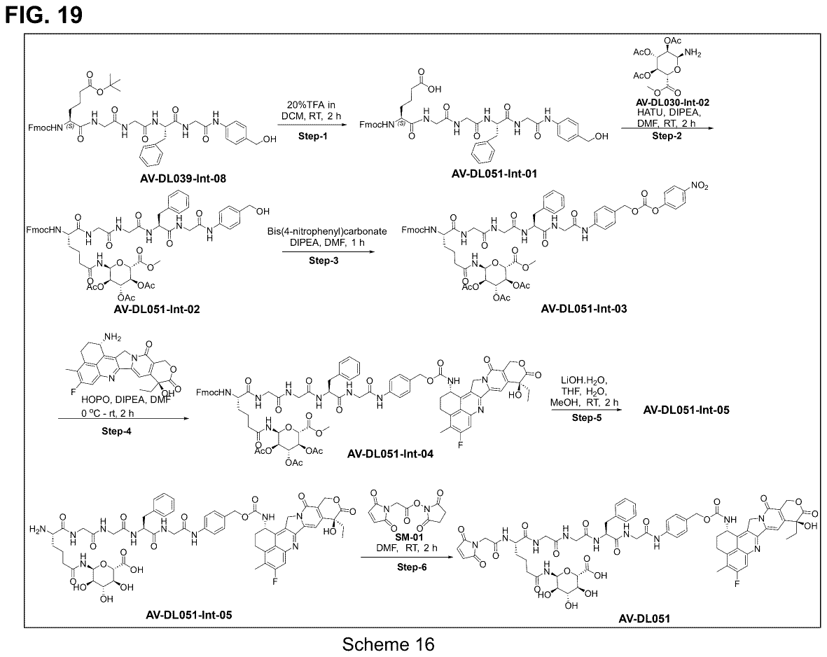

### XVI. Synthetic scheme for (2S,3S,4S,5R,6S)-6-((5S,14S)-5-benzyl-14-(2-(2,5-dioxo-2,5-dihydro-1H-pyrrol-1-yl)acetamido)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic Acid (AV-DL051)(Scheme 16, FIG. 19)

### a. Synthesis of (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oic Acid (AV-DL051-Int-01)

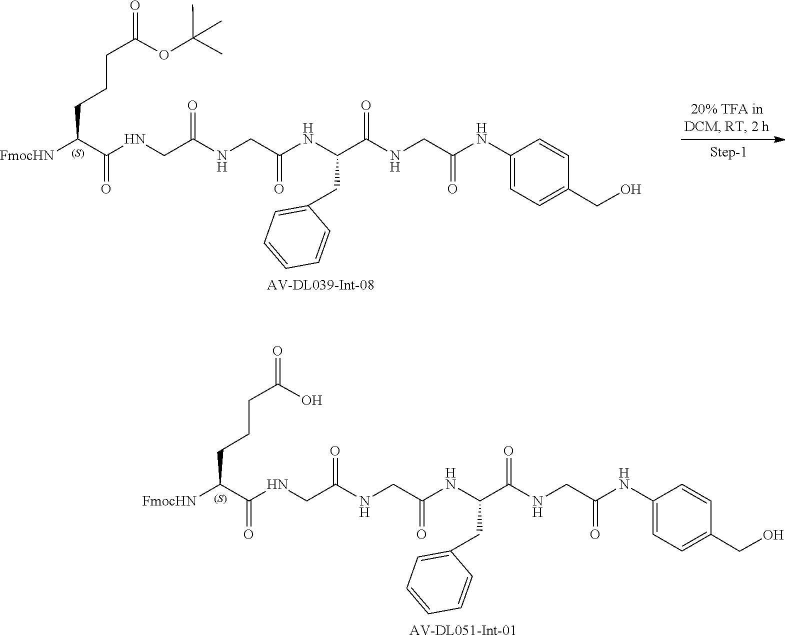

To a stirred solution of tert-butyl (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oate (AV-DL039-Int-08, 300 mg, 0.376 mmol) in 20% TFA in DCM (5 mL) was stirred at RT for 2 h. The reaction was monitored by LCMS. The resulting reaction mixture was concentrated under reduced pressure to get (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oic acid (AV-DL051-Int-01, 300 mg, 0.37 mmol, quantitative yield) as an off white solid.

MS (ESI): 807.42 [M+H]⁺.

### b. Synthesis of (2S,3R,4S,5S,6S)-2-((5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl Triacetate (AV-DL051-Int-02)

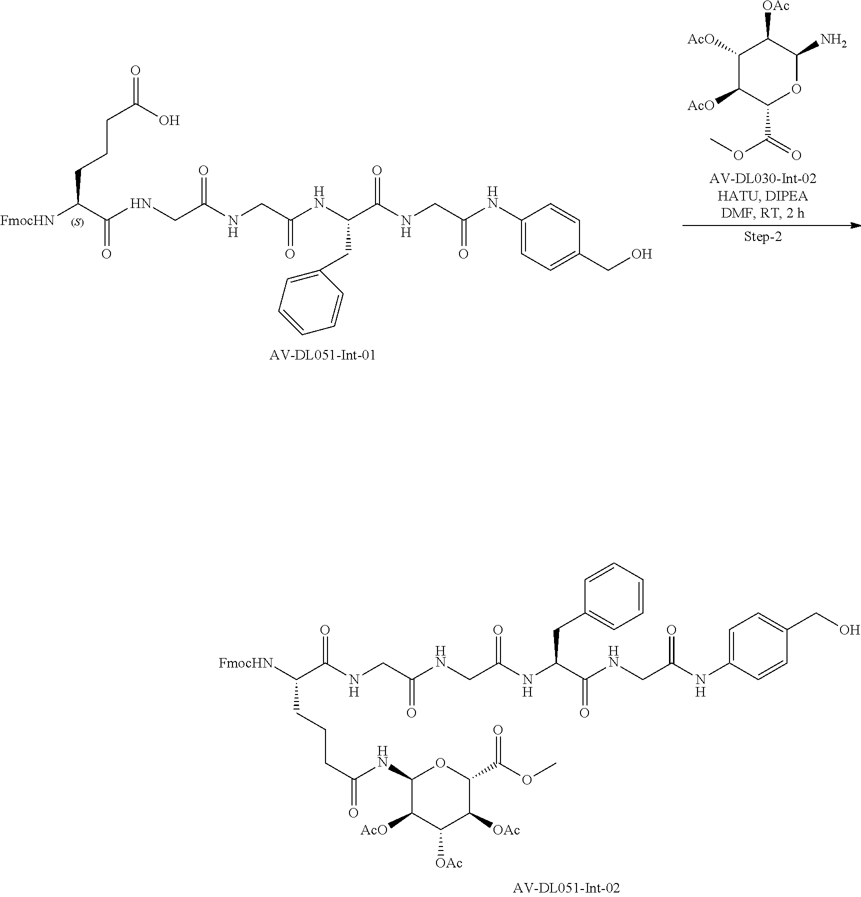

To a stirred solution of (5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-oic acid (AV-DL051-Int-01, 300 mg, 0.38 mmol), HATU (213 mg, 0.56 mmol) and DIPEA (0.2 mL, 1.12 mmol) in DMF (1 mL) was added (2S,3R,4S,5S,6S)-2-amino-6-(methoxycarbonyl)tetrahydro-2H-pyran-3,4,5-triyl triacetate (AV-DL030-Int-02, 186 mg, 0.56 mmol) at 0° C. and stirred at RT for 2 h. The reaction was monitored by LCMS. The resulting reaction mixture was concentrated under reduced pressure to get crude product. The crude product was purified with reverse phase Biotage (column 30 g, C18) eluted with 40% MeCN in water and fractions were lyophilized to get (2S,3R,4S,5S,6S)-2-((5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-02, 125 mg, 0.11 mmol, 30% yield) as an off white solid.

MS (ESI): 1122.52 [M+H]⁺.

### c. Synthesis of (2S,3R,4S,5S,6S)-2-((5S,14S)-14-((((9H-fluoren-9-yl)methoxy)carbonyl)amino)-5-benzyl-1-((4-((((4-nitrophenoxy)carbonyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-03)

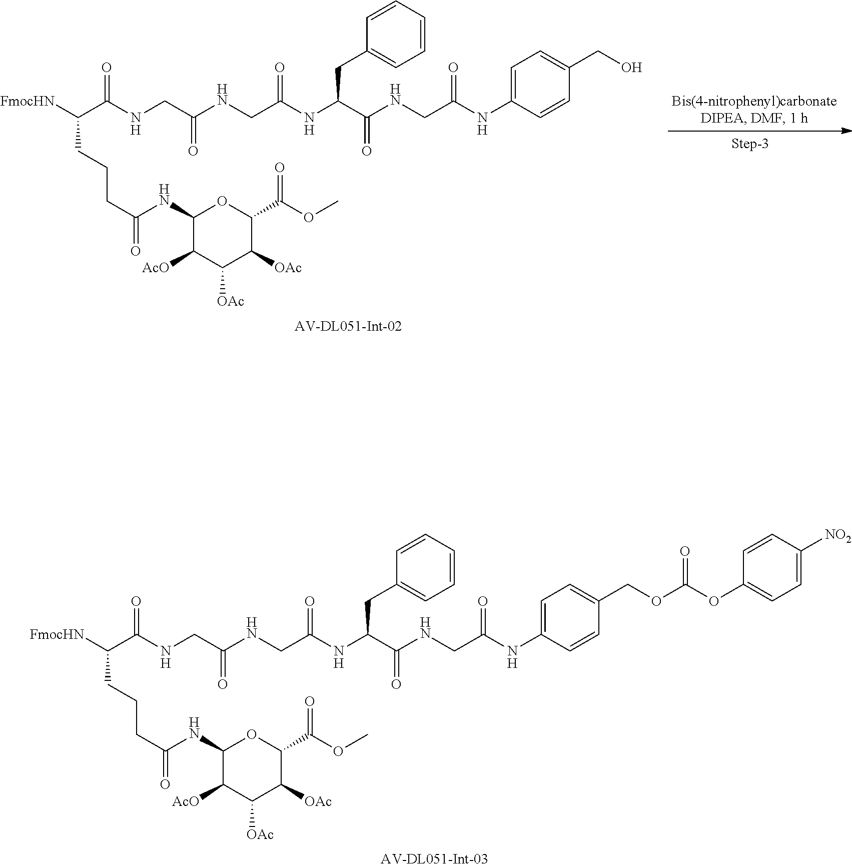

To a stirred solution of (2S,3R,4S,5S,6S)-2-((5S,14S)-14-((((9H-fluoren-9-yl)methoxy)carbonyl)amino)-5-benzyl-1-((4-(hydroxymethyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-02, 300 mg, 0.27 mmol) and bis(4-nitrophenyl)carbonate (245 mg, 0.81 mmol) in DMF (2 mL) was added DIPEA (0.14 mL, 0.81 mmol) at 0° C. and stirred at RT for 1 h. The reaction was monitored by TLC. The resulting reaction mixture was poured into ice cold water; solid was precipitated out which was washed with water (500 mL) and dried to get (2S,3R,4S,5S,6S)-2-((5S,14S)-14-((((9H-fluoren-9-yl)methoxy)carbonyl)amino)-5-benzyl-1-((4-((((4-nitrophenoxy)carbonyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-03, 300 mg, 0.233 mmol, 87% yield) as a pale yellow solid. This was used as such for the next step.

MS (ESI): 1288.42 [M+H]⁺.

### Synthesis of (2S,3R,4S,5S,6S)-2-((5S,14S)-14-((((9H-fluoren-9-yl)methoxy)carbonyl)amino)-5-benzyl-1-((4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl Triacetate (AV-DL051-Int-04)

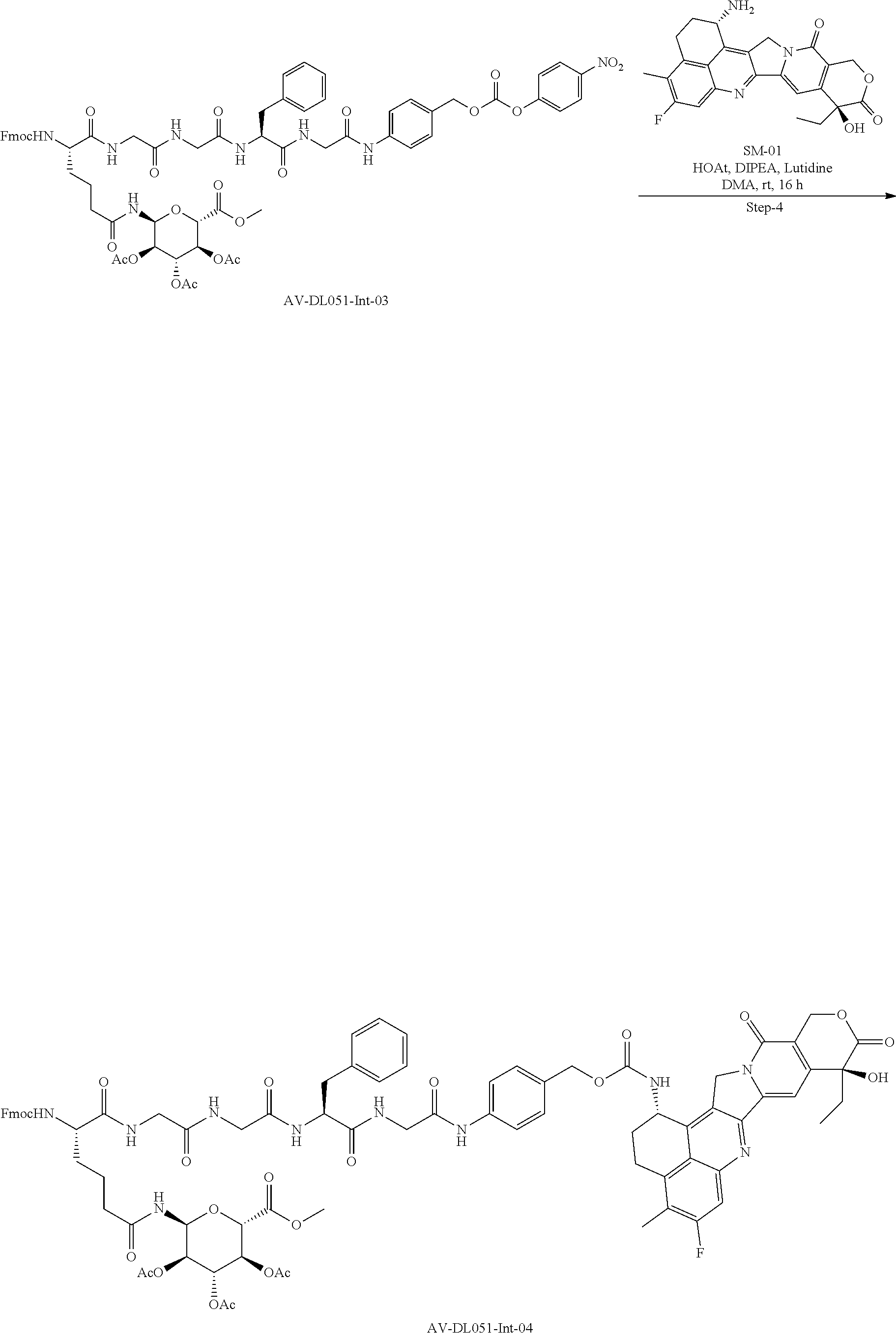

To a stirred solution of (2S,3R,4S,5S,6S)-2-((5S,14S)-14-((((9H-fluoren-9-yl)methoxy)carbonyl)amino)-5-benzyl-1-((4-((((4-nitrophenoxy)carbonyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-03, 300 mg, 0.24 mmol), (1S,9S)-1-amino-9-ethyl-5-fluoro-9-hydroxy-4-methyl-1,2,3,9,12,15-hexahydro-10H, 13H-benzo[de]pyrano[3′,4′:6,7]indolizino[1,2-b]quinoline-10,13-dione (SM-01, 94 mg, 0.18 mmol), and HOPO (130 mg, 1.17 mmol) in DMF (2 mL) was added DIPEA (0.21 mL, 1.17 mmol) at 0° C. and stirred at RT for 2 h. The reaction was monitored by TLC. The resulting reaction mixture was concentrated under reduced pressure to get crude product. The crude product was further purified by reverse phase Biotage (column 30 g, C18) eluted with 28% MeCN in water to get (2S,3R,4S,5S,6S)-2-((5S,14S)-14-((((9H-fluoren-9-yl)methoxy)carbonyl)amino)-5-benzyl-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-04, 110 mg, 0.069 mmol, 30% yield) as an off white solid.

MS (ESI): 1584.45 [M+H]⁺.

### d. Synthesis of (2S,3S,4S,5R,6S)-6-((5S,14S)-14-amino-5-benzyl-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic Acid (AV-DL051-Int-05)

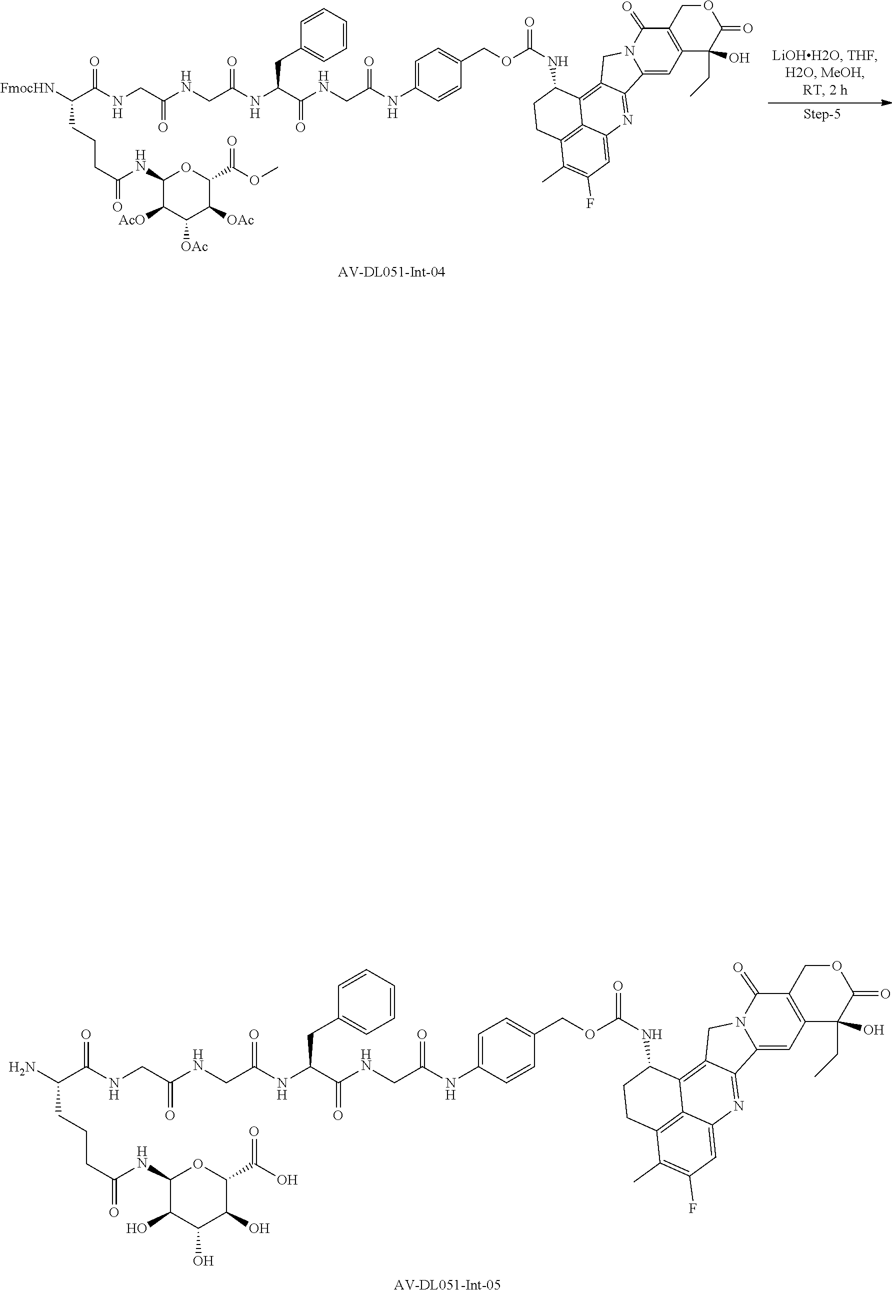

To a stirred solution of (2S,3R,4S,5S,6S)-2-((5S,14S)-14-((((9H-fluoren-9-yl)methoxy)carbonyl)amino)-5-benzyl-1-((4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL051-Int-04, 100 mg, 0.064 mmol) in THF:H₂O:MeOH (1:1:1)(2 mL) was added LiOH·H₂O (16 mg, 0.38 mmol) at 0° C. and stirred at RT for 2 h. The reaction was monitored by LCMS. The reaction mixture was concentrated under reduced pressure to get crude product. The crude product was further purified by reverse phase Biotage (column 12 g, C18) eluted with 18% MeCN in water to get (2S,3S,4S,5R,6S)-6-((5S,14S)-14-amino-5-benzyl-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL051-Int-05, 45 mg, 0.0368 mmol, quantitative yield) as an off white solid.

MS (ESI): 1221.49 [M+H]⁺.

### e. Synthesis of (2S,3S,4S,5R,6S)-6-((5S,14S)-5-benzyl-14-(2-(2,5-dioxo-2,5-dihydro-1H-pyrrol-1-yl)acetamido)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic Acid (AV-DL051)

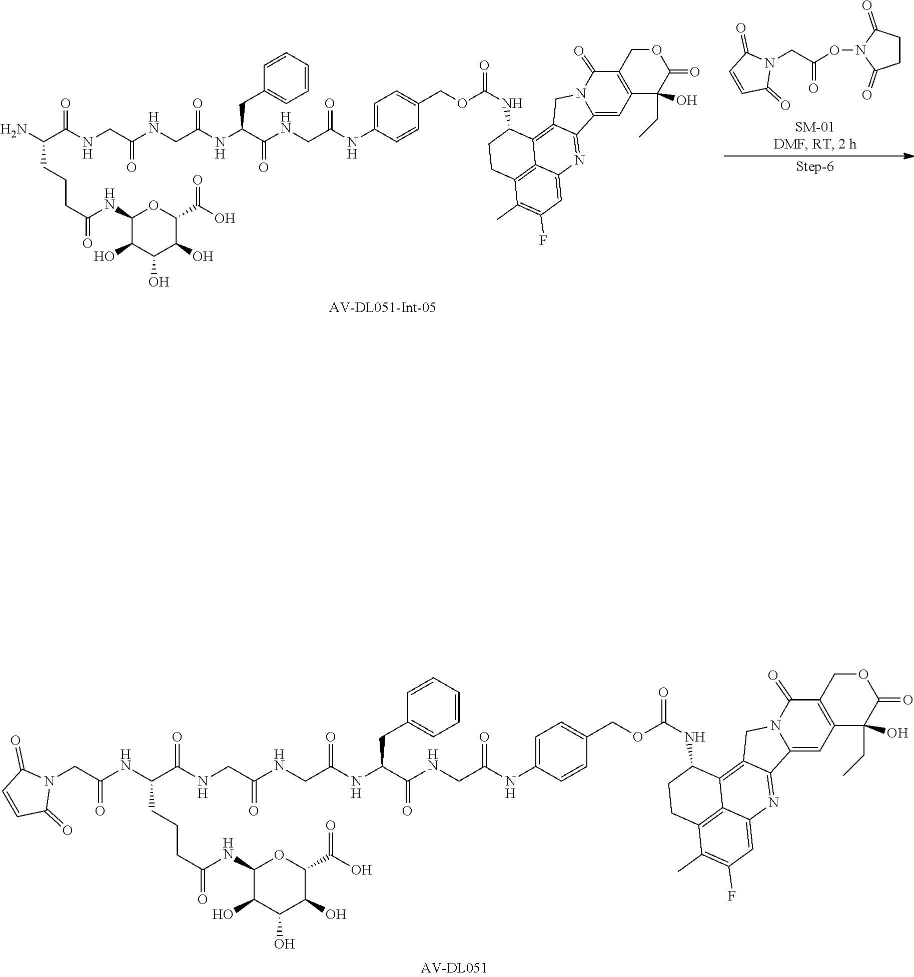

To a stirred solution of (2S,3S,4S,5R,6S)-6-((5S,14S)-14-amino-5-benzyl-1-((4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL051-Int-05, 40 mg, 0.033 mmol) in DMF (1 mL) was added 2,5-dioxopyrrolidin-1-yl 2-(2,5-dioxo-2,5-dihydro-1H-pyrrol-1-yl)acetate (SM-01, 16.5 mg, 0.066 mmol) at 0° C. and stirred at RT for 1 h. The reaction was monitored by LCMS. The resulting reaction mixture was concentrated under reduced pressure to get crude product. The crude product was further purified by RP Prep-HPLC to get (2S,3S,4S,5R,6S)-6-((5S,14S)-5-benzyl-14-(2-(2,5-dioxo-2,5-dihydro-1H-pyrrol-1-yl)acetamido)-1-((4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenyl)amino)-1,4,7,10,13-pentaoxo-3,6,9,12-tetraazaoctadecan-18-amido)-3,4,5-trihydroxytetrahydro-2H-pyran-2-carboxylic acid (AV-DL051, 2.7 mg, 0.002 mmol, 6% yield) as an off white solid.

MS (ESI): 1358.63 [M+H]⁺; ¹H NMR (400 MHz, DMSO-d₆): δ 9.90 (s, 1H), 8.44-8.38 (m, 2H), 8.37-8.19 (m, 2H), 8.10-7.99 (m, 1H), 7.78 (d, J=11.2 Hz, 1H), 7.61 (d, J=8.4 Hz, 1H), 7.37 (d, J=8.4 Hz, 1H), 7.31 (s, 1H), 7.29-7.16 (m, 2H), 7.05 (s, 2H), 6.52 (brs, 1H), 5.44 (s, 2H), 5.32-5.25 (m, 3H), 5.15-5.05 (m, 2H), 4.85-4.81 (m, 1H), 4.79-4.70 (m, 1H), 4.56-4.49 (m, 1H), 4.29-4.22 (m, 1H), 4.09 (d, J=4.8 Hz, 2H), 3.90-3.82 (m, 2H), 3.80-3.61 (m, 4H), 3.20-3.01 (m, 5H), 2.89-2.73 (m, 5H), 2.38-2.05 (m, 11H), 1.90-1.84 (m, 2H), 1.70-1.40 (m, 6H), 1.25-1.21 (m, 2H), 0.87 (t, J=7.2 Hz, 3H).

PREP-HPLC Conditions: X-Bridge,C-18 column, 150×19 mm×5 μm A: 0.1% FA in water B: Acetonitrile, 0/35, 12/60,22.1/98; diluent: MeCN, Water.

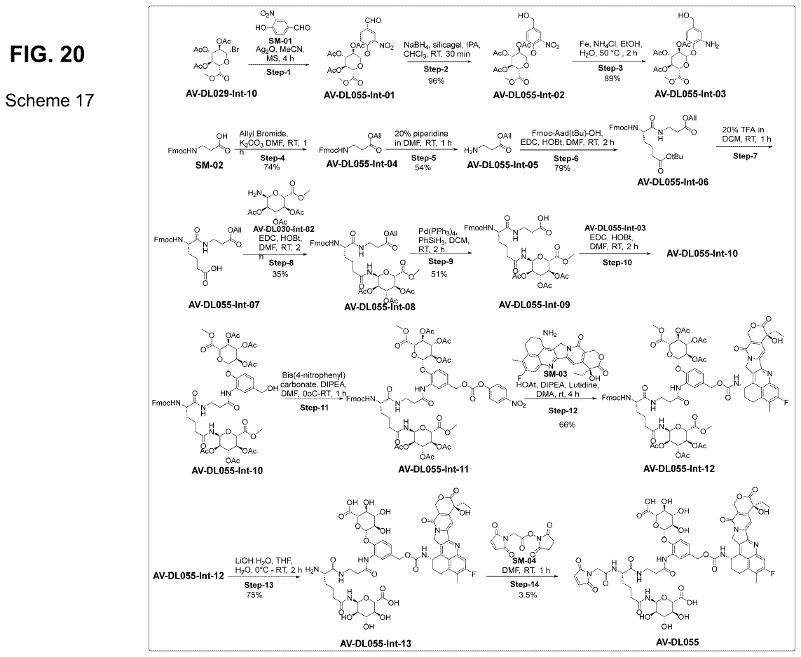

### XVII. Synthetic scheme for (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic Acid (AV-DL055)(Scheme 17, FIG. 20)

### a. Synthesis of (2S,3R,4S,5S,6S)-2-(4-formyl-2-nitrophenoxy)-6-(methoxycarbonyl)tetrahydro-2H-pyran-3,4,5-triyl Triacetate (AV-DL055-Int-02)

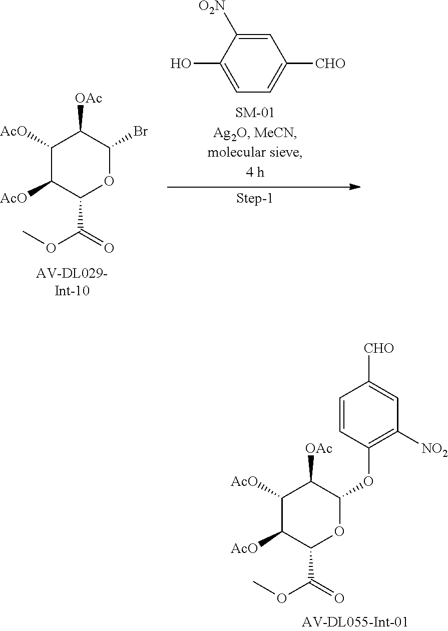 [SMILES: O=Cc1ccc(O)c([N+](=O)[O-])c1 ; COC(=O)[C@H]1O[C@@H](Br)[C@H](OC(C)=O)[C@@H](OC(C)=O)[C@@H]1OC(C)=O ; COC(=O)[C@H]1O[C@@H](Oc2ccc(C=O)cc2[N+](=O)[O-])[C@H](OC(C)=O)[C@@H](OC(C)=O)[C@@H]1OC(C)=O]

To a stirred solution (2S,3R,4S,5S,6S)-2-bromo-6-(methoxycarbonyl)tetrahydro-2H-pyran-3,4,5-triyl triacetate (AV-DL029-Int-10, 2.5 g, 6.32 mmol) and 4-hydroxy-3-nitrobenzaldehyde (SM-01, 1.2 g, 6.95 mmol) in CH₃CN at 0° C. was added molecular sieve (5 g) and Ag₂O (1.5 g, 6.38 mmol) at 0° C. The round bottom flask was covered with aluminium foil and stirred at RT for 4 h. The reaction was monitored by TLC. The reaction mixture was filter through celite pad and filtrate was concentrated under reduced pressure to get (2S,3R,4S,5S,6S)-2-(4-formyl-2-nitrophenoxy)-6-(methoxycarbonyl)tetrahydro-2H-pyran-3,4,5-triyl triacetate (AV-DL055-Int-02, 2.5 g, 5.18 mmol) as a pale yellow solid. This was used as such for the next step without further purification.

MS (ESI): 482.32 [M−H]⁺.

### b. Synthesis of (2S,3R,4S,5S,6S)-2-(4-(hydroxymethyl)-2-nitrophenoxy)-6-(methoxycarbonyl)tetrahydro-2H-pyran-3,4,5-triyl Triacetate (AV-DL055-Int-03)

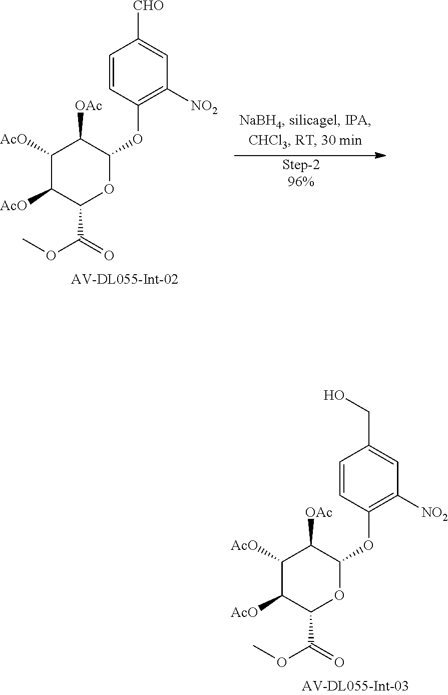 [SMILES: COC(=O)[C@H]1O[C@@H](Oc2ccc(C=O)cc2[N+](=O)[O-])[C@H](OC(C)=O)[C@@H](OC(C)=O)[C@@H]1OC(C)=O ; COC(=O)[C@H]1O[C@@H](Oc2ccc(CO)cc2[N+](=O)[O-])[C@H](OC(C)=O)[C@@H](OC(C)=O)[C@@H]1OC(C)=O]

To a stirred solution (2S,3R,4S,5S,6S)-2-(4-formyl-2-nitrophenoxy)-6-(methoxycarbonyl)tetrahydro-2H-pyran-3,4,5-triyl triacetate (AV-DL055-Int-02, 2.5 g, 5.18 mmol) and silica gel (60-120)(5 g) in CHCl₃:IPA (4:1)(62.5 mL) at 0° C. was added NaBH₄ (990 mg, 25.88 mmol) and stirred at RT for 30 min. The reaction mixture was filter through celite pad and filtrate was diluted with water (30 mL) and extracted with CHCl₃ (2×100 mL). The combined organic layer was washed with brine (100 mL), dried over anhydrous Na₂SO₄, filtered and concentrated under reduced pressure to obtain (2S,3R,4S,5S,6S)-2-(4-(hydroxymethyl)-2-nitrophenoxy)-6-(methoxycarbonyl)tetrahydro-2H-pyran-3,4,5-triyl triacetate (AV-DL055-Int-03, 2.4 g, 4.9 mmol, 96% yield) as a pale yellow solid.

MS (ESI): 503.19 [M+18]⁺.

### c. Synthesis of (2S,3R,4S,5S,6S)-2-(2-amino-4-(hydroxymethyl)phenoxy)-6-(methoxycarbonyl)tetrahydro-2H-pyran-3,4,5-triyl Triacetate (AV-DL055-Int-04)

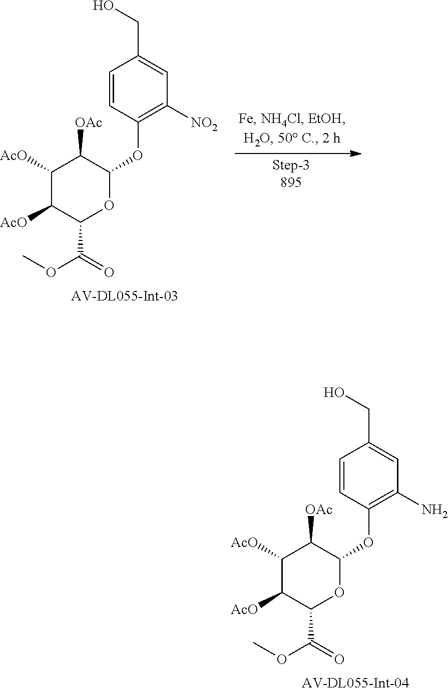 [SMILES: COC(=O)[C@H]1O[C@@H](Oc2ccc(CO)cc2[N+](=O)[O-])[C@H](OC(C)=O)[C@@H](OC(C)=O)[C@@H]1OC(C)=O ; COC(=O)[C@H]1O[C@@H](Oc2ccc(CO)cc2N)[C@H](OC(C)=O)[C@@H](OC(C)=O)[C@@H]1OC(C)=O]

To a stirred solution of (2S,3R,4S,5S,6S)-2-(4-(hydroxymethyl)-2-nitrophenoxy)-6-(methoxycarbonyl)tetrahydro-2H-pyran-3,4,5-triyl triacetate (AV-DL055-Int-03, 2.4 g, 4.96 mmol) in EtOH:H₂O (4:1)(10 mL) at 0° C. was added Fe powder (2.77 g, 12.4 mmol), NH₄Cl (2.62 g, 12.4 mmol) and stirred at 50° C. for 2 h. The reaction mixture was filter through celite pad, filtrate was diluted with water (20 mL) and extracted with CHCl₃ (2× 50 mL). The combined organic layer was washed with brine (20 mL), dried over anhydrous Na₂SO₄, filtered and concentrated under reduced pressure to get (2S,3R,4S,5S,6S)-2-(2-amino-4-(hydroxymethyl)phenoxy)-6-(methoxycarbonyl)tetrahydro-2H-pyran-3,4,5-triyl triacetate (AV-DL055-Int-03, 3 g, 4.4 mmol, 89% yield) as a brown gum.

MS (ESI): 456.30 [M+H]⁺.

### d. Synthesis of allyl 3-((((9H-fluoren-9-yl)methoxy)carbonyl)amino)propanoate (AV-DL055-Int-04)

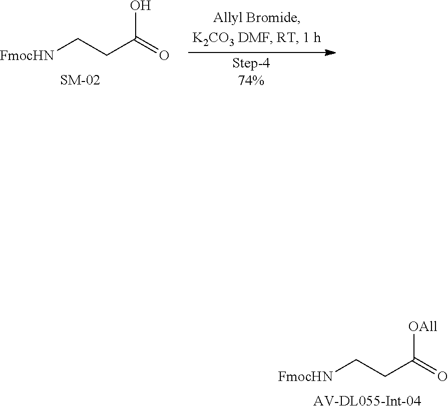

To a stirred solution of (((9H-fluoren-9-yl)methoxy)carbonyl)glycylglycyl-L-phenylalanylglycine (SM-02, 1.5 g, 2.68 mmol) and allyl bromide (0.34 mL, 4.03 mmol) in DMF (10 mL) was added K₂CO₃ (556 mg, 4.032 mmol) at 0° C. and stirred at RT for 16 h. The reaction was monitored by TLC. The resulting reaction mixture was concentrated under reduced pressure to get crude product. The crude product was purified with silica gel column chromatography eluted with 5% MeOH/DCM to get allyl 3-((((9H-fluoren-9-yl)methoxy)carbonyl)amino)propanoate (AV-DL055-Int-04, 2.5 g, 7.13 mmol, 74% yield) as an off-white solid.

MS (ESI): 352.30 [M+H]⁺.

### e. Synthesis of Allyl 3-aminopropanoate (AV-DL055-Int-05)

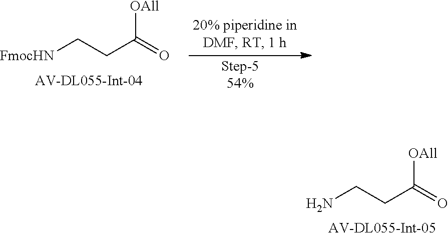

To a stirred solution of allyl 3-((((9H-fluoren-9-yl)methoxy)carbonyl)amino)propanoate (AV-DL055-Int-04, 2.5 g, 7.13 mmol) in DMF (20 mL) was added piperdine (3.4 mL, 32 mmol) at 0° C. and stirred at RT for 2 h. The reaction was monitored by TLC. The reaction mixture was concentrated under reduced pressure to get crude product. The crude product was triturated with diethyl ether (3×15 mL) and dried to get allyl 3-aminopropanoate (AV-DL055-Int-05, 500 mg, 3.85 mmol, 54% yield) as a brown liquid.

MS (ESI): 130.06 [M+H]⁺.

### f. Synthesis of tert-butyl(S)-5-((((9H-fluoren-9-yl)methoxy)carbonyl)amino)-6-((3-(allyloxy)-3-oxopropyl)amino)-6-oxohexanoate (AV-DL055-Int-06)

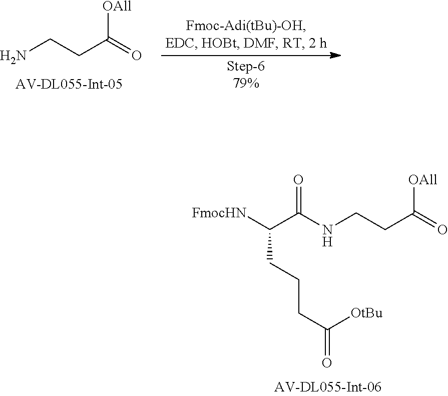

To a stirred solution of (E)-prop-1-en-1-yl 3-aminopropanoate (AV-DL055-Int-05, 500 mg, 3.88 mmol) and(S)-2-((((9H-fluoren-9-yl)methoxy)carbonyl)amino)-6-(tert-butoxy)-6-oxohexanoic acid (Fmoc-Adi(tBu)-OH, 500 mg, 1.14 mmol) in DMF (5 mL) was added EDC (744 mg, 3.88 mmol), HOBt (528 mg, 3.88 mmol) at 0° C. and stirred at RT for 2 h. The reaction was monitored by LCMS. The resulting reaction mixture was concentrated under reduced pressure to get crude product. The crude product was purified with reverse phase Biotage (column 60 g, C18) eluted with 60% MeCN in water and fractions were lyophilized to get tert-butyl(S)-5-((((9H-fluoren-9-yl)methoxy)carbonyl)amino)-6-((3-(allyloxy)-3-oxopropyl)amino)-6-oxohexanoate (AV-DL055-Int-06, 500 mg, 1.01 mmol, 79% yield) as a pale yellow liquid.

MS (ESI): 551.44 [M+H]⁺.

### g. Synthesis of(S)-5-((((9H-fluoren-9-yl)methoxy)carbonyl)amino)-6-((3-(allyloxy)-3-oxopropyl)amino)-6-oxohexanoic Acid (AV-DL055-Int-07)

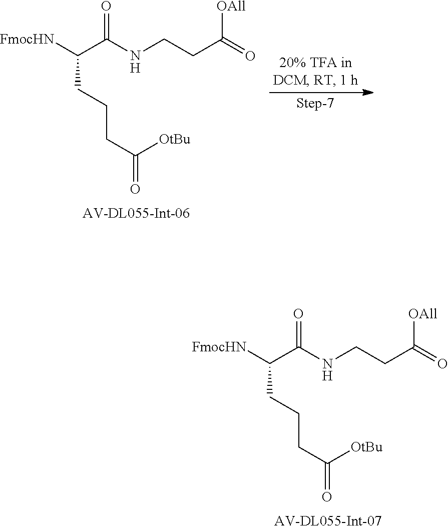

To tert-butyl (S,E)-5-((((9H-fluoren-9-yl)methoxy)carbonyl)amino)-6-oxo-6-((3-oxo-3-(prop-1-en-1-yloxy)propyl)amino)hexanoate (AV-DL055-Int-06, 300 mg, 0.47 mmol) was added 20% TFA in DCM (5 mL) and stirred at RT for 1 h. The reaction was monitored by LCMS. The resulting reaction mixture was concentrated under reduced pressure to get(S)-5-((((9H-fluoren-9-yl)methoxy)carbonyl)amino)-6-((3-(allyloxy)-3-oxopropyl)amino)-6-oxohexanoic acid (AV-DL055-Int-07, 300 mg, 0.61 mmol, quantitative yield) as an off white solid.

MS (ESI): 495.34 [M+H]⁺.

### h. Synthesis of (2S,3R,4S,5S,6S)-2-((S)-5-((((9H-fluoren-9-yl)methoxy)carbonyl)amino)-6-((3-(allyloxy)-3-oxopropyl)amino)-6-oxohexanamido)-6-(methoxycarbonyl)tetrahydro-2H-pyran-3,4,5-triyl triacetate (AV-DL055-Int-08)

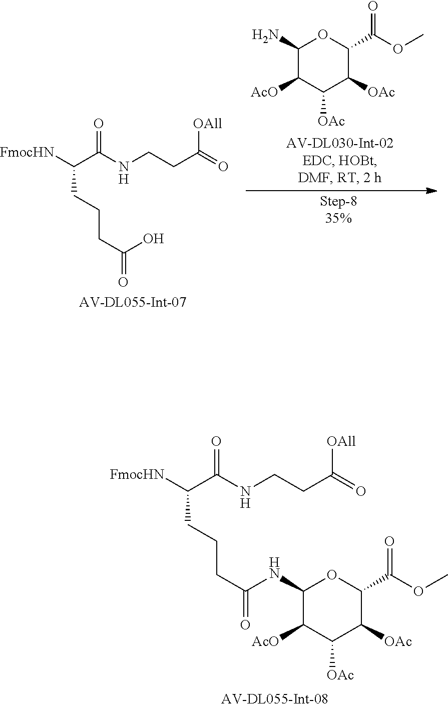

To a stirred solution of (S,E)-5-((((9H-fluoren-9-yl)methoxy)carbonyl)amino)-6-oxo-6-((3-oxo-3-(prop-1-en-1-yloxy)propyl)amino)hexanoic acid (AV-DL055-Int-07, 300 mg, 0.61 mmol), HATU (3488 mg, 0.92 mmol) and DIPEA (0.32 mL, 1.83 mmol) in DMF was added (2S,3R,4S,5S,6S)-2-amino-6-(methoxycarbonyl)tetrahydro-2H-pyran-3,4,5-triyl triacetate (AV-DL030-Int-02, 304 mg, 0.92 mmol) at 0° C. and stirred at RT for 2 h. The reaction was monitored by LCMS. The resulting reaction mixture was concentrated under reduced pressure to get crude product. The crude product was purified with reverse phase Biotage (column 30 g, C18) eluted with 41% MeCN in water and fractions were lyophilized to get (2S,3R,4S,5S,6S)-2-((S)-5-((((9H-fluoren-9-yl)methoxy)carbonyl)amino)-6-((3-(allyloxy)-3-oxopropyl)amino)-6-oxohexanamido)-6-(methoxycarbonyl)tetrahydro-2H-pyran-3,4,5-triyl triacetate (AV-DL055-Int-08, 175 mg, 0.22 mmol, 35% yield) as an off white solid.

MS (ESI): 810.16 [M+H]⁺.

### i. Synthesis of 3-((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanamido)propanoic Acid (AV-DL055-Int-09)

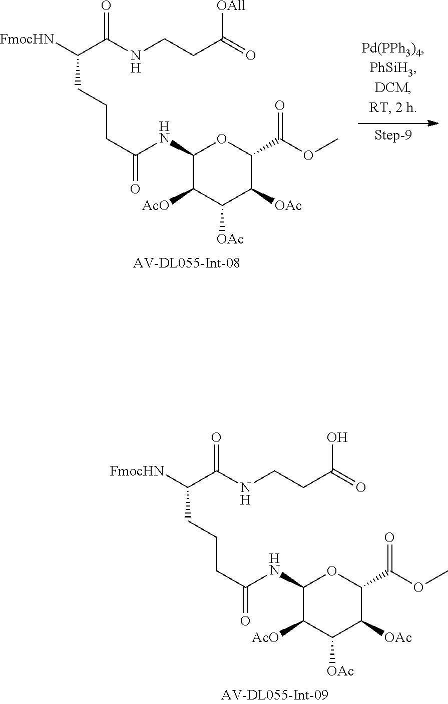

To a stirred solution of (2S,3R,4S,5S,6S)-2-((S)-5-((((9H-fluoren-9-yl)methoxy)carbonyl)amino)-6-((3-(allyloxy)-3-oxopropyl)amino)-6-oxohexanamido)-6-(methoxycarbonyl)tetrahydro-2H-pyran-3,4,5-triyl triacetate (AV-DL055-Int-08, 175 mg, 0.113 mmol) and phenyl silane (67 μL, 0.432 mmol) in DCM (5 mL) was added Pd(PPh₃)₄ (6.3 mg, 0.006 mmol) at 0° C. and stirred at RT for 1 h. The reaction was monitored by TLC. The resulting reaction mixture was concentrated under reduced pressure to get 3-((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanamido)propanoic acid (AV-DL055-Int-09, 175 mg, 0.227 mmol, quantitative yield) as a light brown semi solid. The crude was used as such for next step.

MS (ESI): 771.43 [M+H]⁺.

### j. Synthesis of (2S,3R,4S,5S,6S)-2-((S)-5-((((9H-fluoren-9-yl)methoxy)carbonyl)amino)-6-((3-((5-(hydroxymethyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl Triacetate (AV-DL055-Int-10)

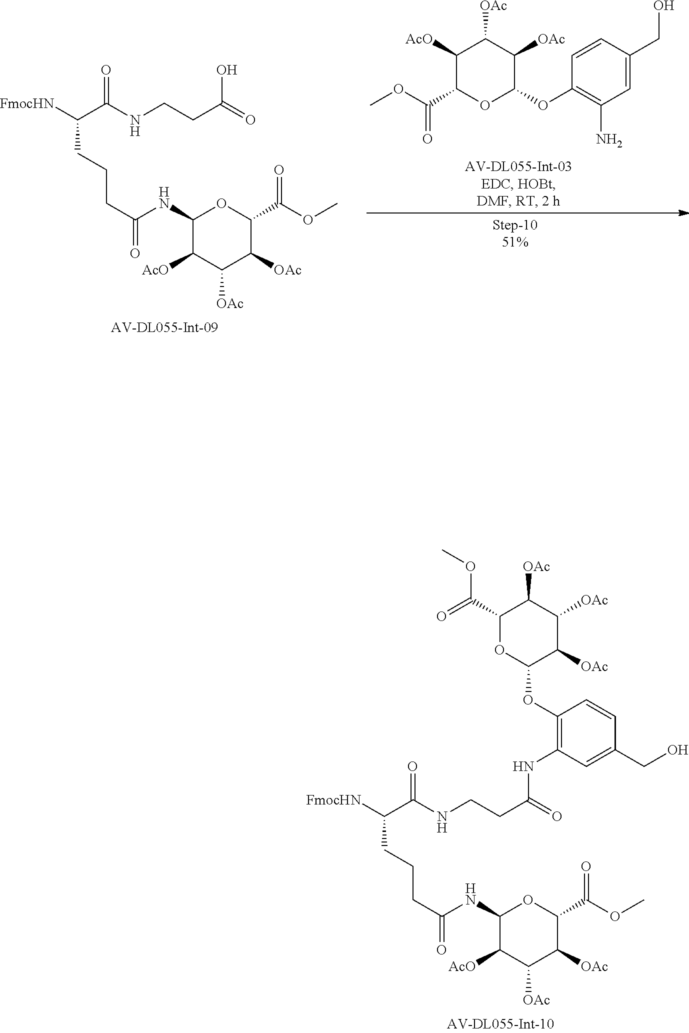

To a stirred solution of 3-((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanamido)propanoic acid (AV-DL055-Int-09, 175 mg, 0.228 mmol) and (2S,3R,4S,5S,6S)-2-(2-amino-4-(hydroxymethyl)phenoxy)-6-(methoxycarbonyl)tetrahydro-2H-pyran-3,4,5-triyl triacetate (AV-DL055-Int-03, 156 mg, 0.342 mmol) in DMF (3 mL) was added HATU (131 mg, 0.342 mmol) and DIPEA (0.12 mL, 0.684 mmol) at 0° C., and stirred at RT for 2 h. The reaction was monitored by LCMS. The resulting reaction mixture was concentrated under reduced pressure to get crude product. The crude product was purified with reverse phase Biotage (column 30 g, C18) eluted with 42% MeCN in water and fractions were lyophilized to get (2S,3R,4S,5S,6S)-2-((S)-5-((((9H-fluoren-9-yl)methoxy)carbonyl)amino)-6-((3-((5-(hydroxymethyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-10, 145 mg, 0.12 mmol, 51% yield) as an off white solid.

MS (ESI): 1207.33 [M+H]⁺.

### k. Synthesis of (2S,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-(methoxycarbonyl)tetrahydro-2H-pyran-3,4,5-triyl Triacetate (AV-DL055-Int-11)

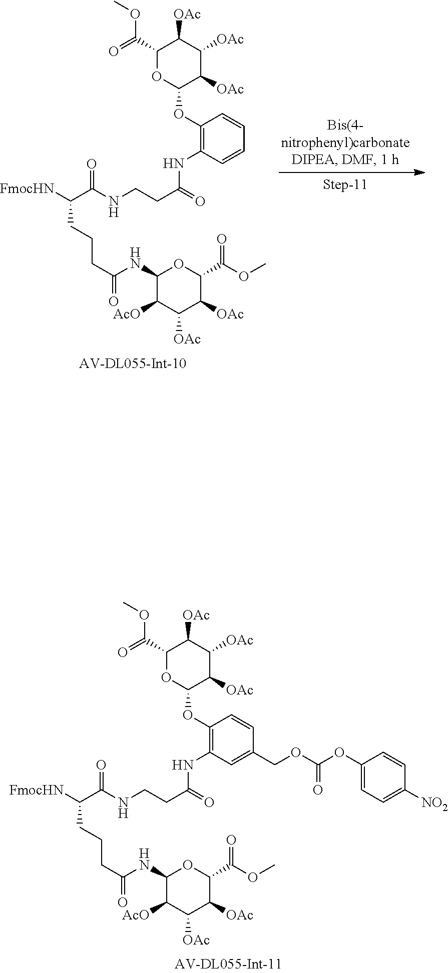

To a stirred solution of (2S,3R,4S,5S,6S)-2-((S)-5-((((9H-fluoren-9-yl)methoxy)carbonyl)amino)-6-((3-((5-(hydroxymethyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (250 mg, 0.21 mmol) and bis(4-nitrophenyl)carbonate (190 mg, 0.63 mmol) in DMF (5 mL) was added DIPEA (0.11 mL, 0.63 mmol) at 0° C. and stirred at RT for 1 h. The reaction was monitored by TLC. The resulting reaction mixture was poured into ice cold water (50 mL) and extracted with EtOAc (3×50 mL). The combined organic layer was dried over anhydrous Na₂SO₄, filtered and evaporated under reduced pressure to get (2S,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 250 mg, 0.19 mmol, quantitative yield), as a pale yellow solid. The crude product was used as such for the next step.

MS (ESI): 1372.89 [M+H]⁺.

### 1. Synthesis of (2S,3R,4S,5S,6S)-2-((S)-5-((((9H-fluoren-9-yl)methoxy)carbonyl)amino)-6-((3-((5-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-2-(((2S,3R,4S,5S,6S)-3,4,5-triacetoxy-6-(methoxycarbonyl)tetrahydro-2H-pyran-2-(methoxycarbonyl)tetrahydro-2H-pyran-3,4,5-triyl Triacetate (AV-DL055-Int-12)

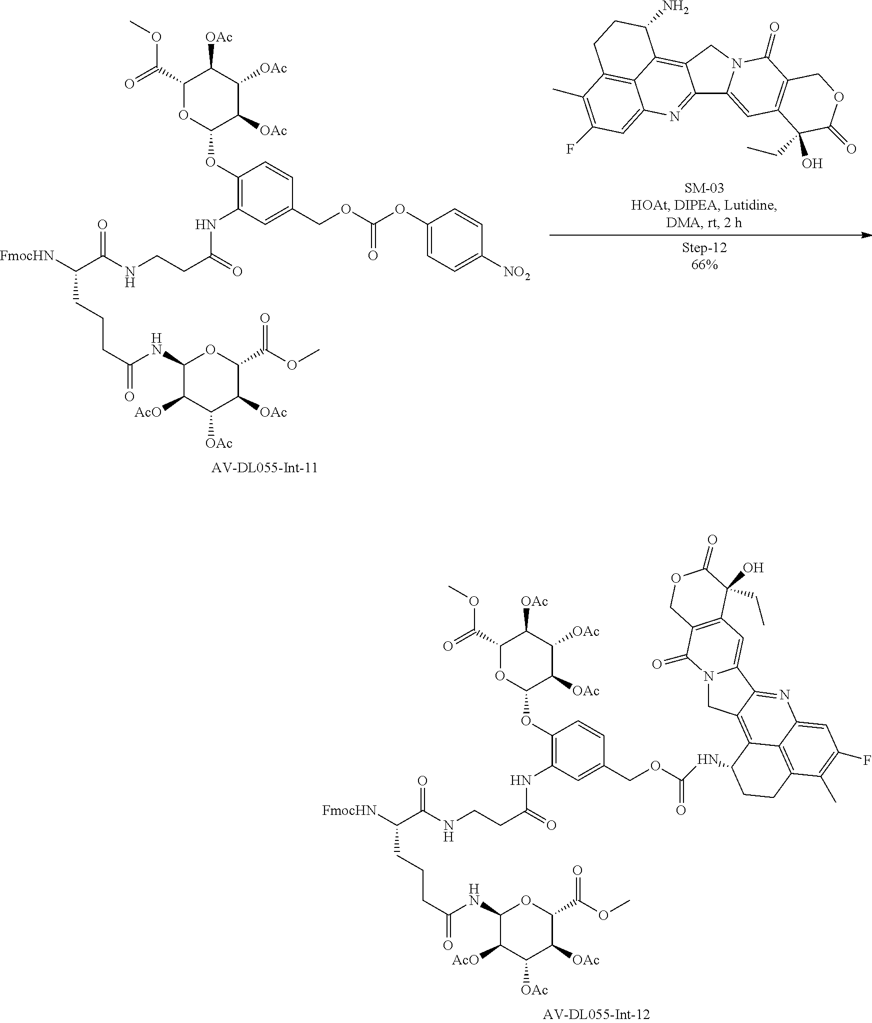

To a stirred solution of (2S,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 250 mg, 0.19 mmol), (1S,9S)-1-amino-9-ethyl-5-fluoro-9-hydroxy-4-methyl-1,2,3,9,12,15-hexahydro-10H, 13H-benzo[de]pyrano[3′,4′:6,7]indolizino[1,2-b]quinoline-10,13-dione (SM-03, 97 mg, 0.19 mmol), and 2,6-lutidine (0.09 mL g, 0.73 mmol) in DMA (5 mL) was added HOAt in DMA (0.28 mL, 0.28 mmol) and DIPEA (0.13 mL, 0.73 mmol) at 0° C., and stirred at RT for 2 h. The reaction was monitored by TLC. The resulting reaction mixture was concentrated under reduced pressure to get crude product. The crude product was further purified by reverse phase Biotage (column 30 g, C18) eluted with 30% MeCN in water to get (2S,3R,4S,5S,6S)-2-((S)-5-((((9H-fluoren-9-yl)methoxy)carbonyl)amino)-6-((3-((5-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-12, 200 mg, 0.12 mmol, 66% yield) as an off white solid.

MS (ESI): 1668.47 [M+H]⁺.

### m. Synthesis of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic Acid (AV-DL055-Int-13)

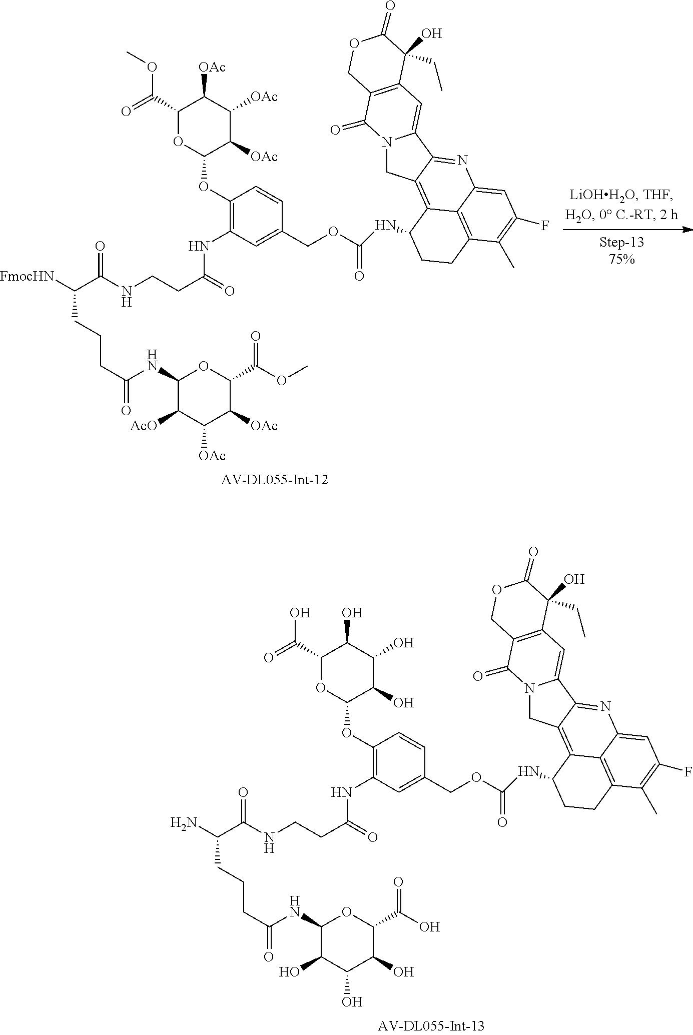

To a stirred solution of (2S,3R,4S,5S,6S)-2-((S)-5-((((9H-fluoren-9-yl)methoxy)carbonyl)amino)-6-((3-((5-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-12, 90 mg, 0.0539 mmol) in THF:H₂O:MeOH (1:1:1)(3 mL) was added LiOH·H₂O (22 mg, 0.539 mmol) at 0° C. and stirred at RT for 2 h. The reaction was monitored by LCMS. The reaction was concentrated under reduced pressure to get crude product. The crude product was further purified by reverse phase Biotage (column 10 g, C18) eluted with 10% MeCN in water to get (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid (AV-DL055-Int-13, 50 mg, 0.0429 mmol, 75% yield) as an off white solid.

MS (ESI): 1165.96 [M+H]⁺.

### n. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic Acid (AV-DL055)

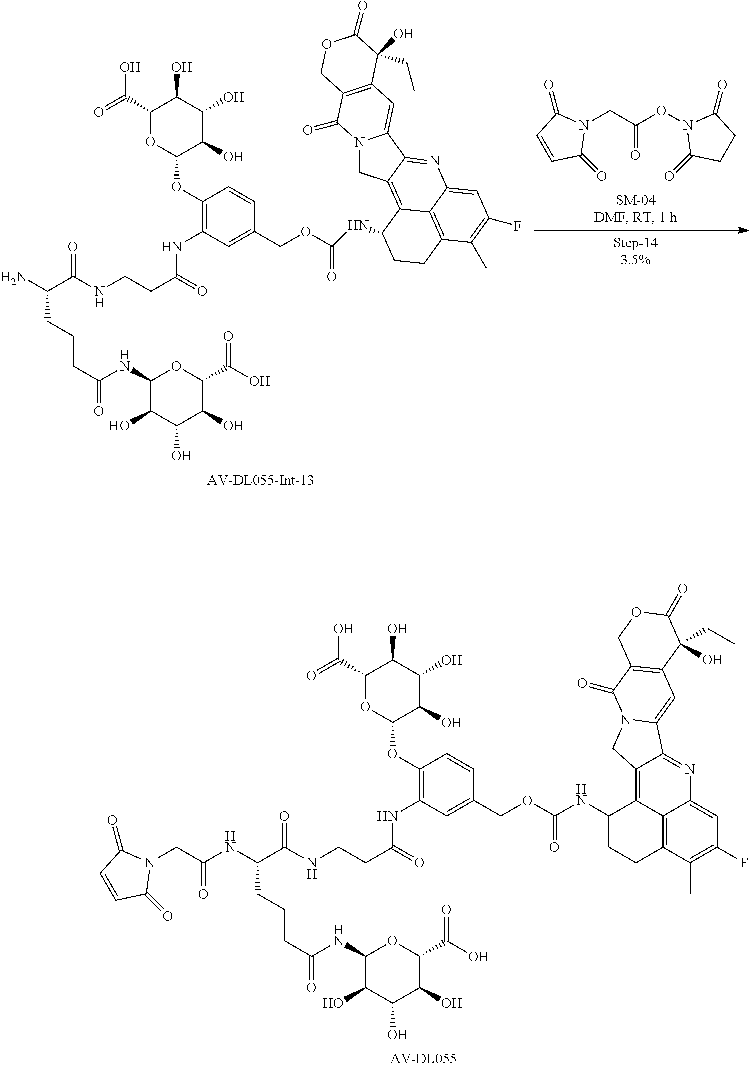 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@H]2O[C@H](C(=O)O)[C@@H](O)[C@H](O)[C@H]2O)c1)CC3 ; O=C(CN1C(=O)C=CC1=O)ON1C(=O)CCC1=O ; CC[C@@]1(O)C(=O)OCc2c1cc1n(c2=O)Cc2c-1nc1cc(F)c(C)c3c1c2C(NC(=O)OCc1ccc(O[C@@H]2O[C@H](C(=O)O)[C@@H](O)[C@H](O)[C@H]2O)c(NC(=O)CCNC(=O)[C@H](CCCC(=O)N[C@H]2O[C@H](C(=O)O)[C@@H](O)[C@H](O)[C@H]2O)NC(=O)CN2C(=O)C=CC2=O)c1)CC3]

To a stirred solution of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid (AV-DL055-Int-13, 50 mg, 0.0429 mmol) and 2,5-dioxopyrrolidin-1-yl 2-(2,5-dioxo-2,5-dihydro-1H-pyrrol-1-yl)acetate (10 mg, 0.0429 mmol) in DMF (2 mL) was added DIPEA (5 μL, 0.0429) and stirred at RT for 2 h. The reaction was monitored by LCMS. The resulting reaction mixture was concentrated under reduced pressure to get the crude. The crude product was further purified by RP Prep-HPLC to get (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid (AV-DL055, 2 mg, 0.0015 mmol, 3.5% yield) as an off-white solid.

MS (ESI): 1303.29 [M+H]⁺; ¹H NMR (400 MHz, DMSO-d₆): δ 12.59 (brs, 2H), 9.14 (brs, 1H), 8.50-8.46 (m, 1H), 8.36 (d, J=8.4 Hz, 1H), 8.18-8.17 (m, 1H), 8.08-8.02 (m, 2H), 8.78 (d, J=10.8 Hz, 1H), 7.31 (s, 1H), 7.10 (s, 2H), 7.07 (s, 2H), 6.53 (d, J=7.2 Hz, 2H), 5.90-5.80 (m, 1H), 5.50-5.25 (m, 6H), 5.13-5.02 (m, 3H), 4.88-4.70 (m, 2H), 4.21-4.05 (m, 3H), 3.80-3.65 (m, 1H), 3.28-3.01 (m, 13H), 2.33 (s, 3H), 2.22-2.05 (m, 5H), 1.90-1.79 (m, 3H), 1.5-1.44 (m, 4H), 0.89-0.83 (m, 3H).

PREP-HPLC Conditions: GEMINI C18 (250*21.2 mm) 5 uA: 0.1% FA in water; B: Acetonitrile: 0/25, 15/50,21/50,21.1/98; diluent: MeCN, Water.

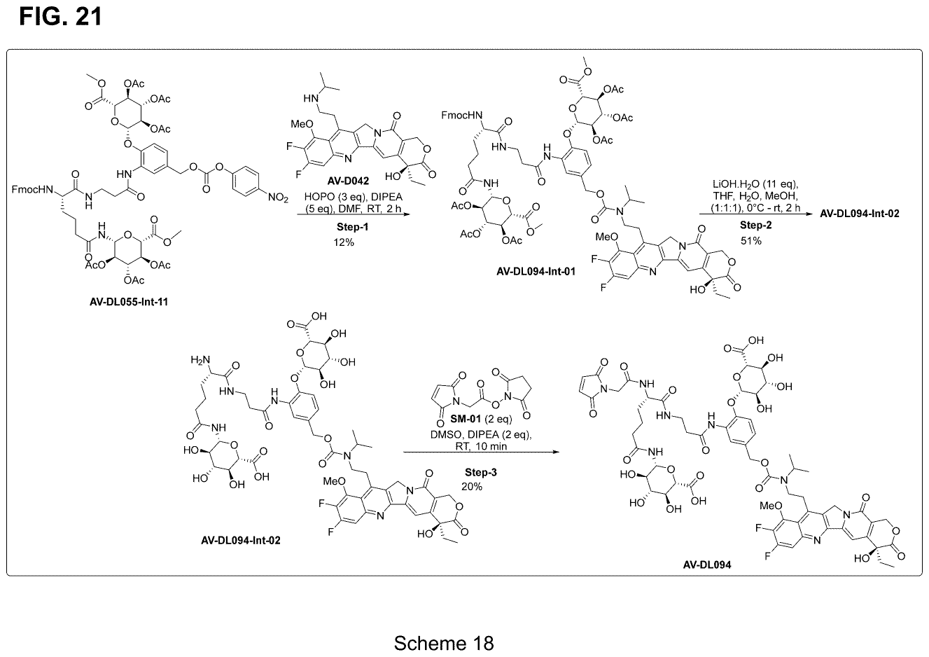

### XVIII. Synthetic scheme for (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL094)(Scheme 18, FIG. 21)

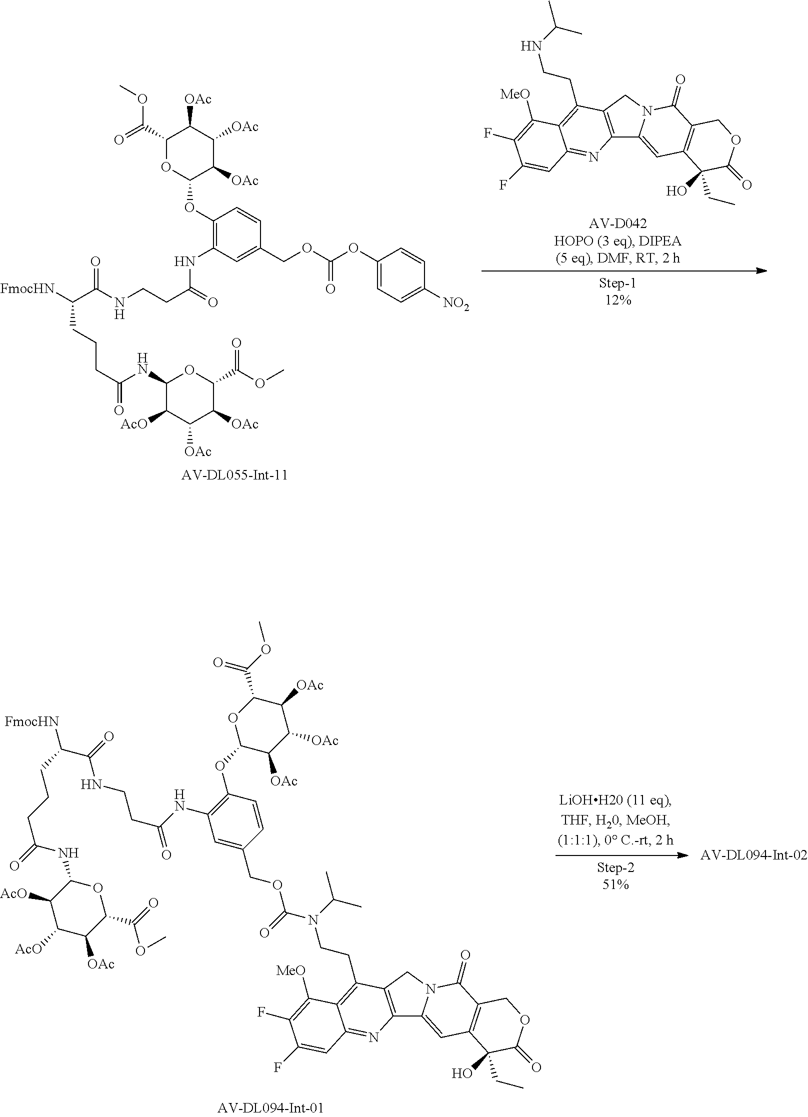

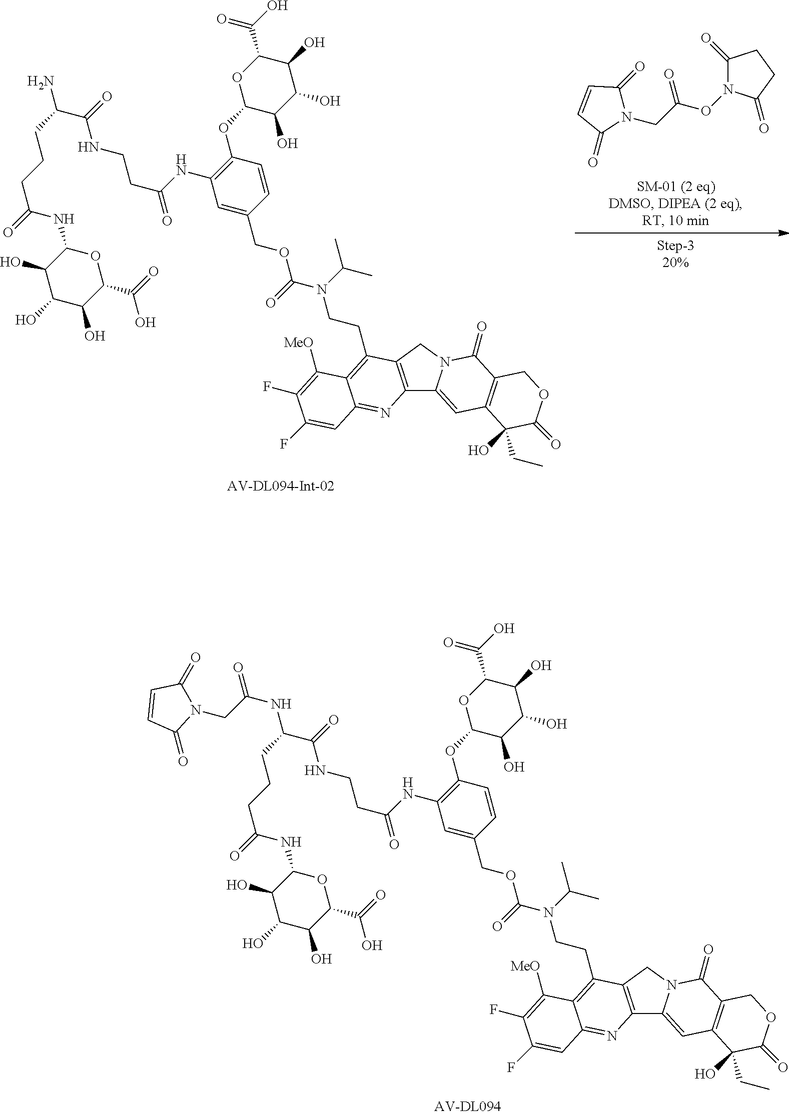 [SMILES: CC[C@@]1(O)C(=O)OCc2c1cc1n(c2=O)Cc2c-1nc1cc(F)c(F)c(OC)c1c2CCN(C(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@@H]2O[C@H](C(=O)O)[C@@H](O)[C@H](O)[C@H]2O)c1)C(C)C ; O=C(CN1C(=O)C=CC1=O)ON1C(=O)CCC1=O ; CC[C@@]1(O)C(=O)OCc2c1cc1n(c2=O)Cc2c-1nc1cc(F)c(F)c(OC)c1c2CCN(C(=O)OCc1ccc(O[C@@H]2O[C@H](C(=O)O)[C@@H](O)[C@H](O)[C@H]2O)c(NC(=O)CCNC(=O)[C@H](CCCC(=O)N[C@@H]2O[C@H](C(=O)O)[C@@H](O)[C@H](O)[C@H]2O)NC(=O)CN2C(=O)C=CC2=O)c1)C(C)C]

### a. Synthesis of (2R,3R,4S,5S,6S)-2-((S)-5-((((9H-fluoren-9-yl)methoxy)carbonyl)amino)-6-((3-((5-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl Triacetate (AV-DL094-Int-01)

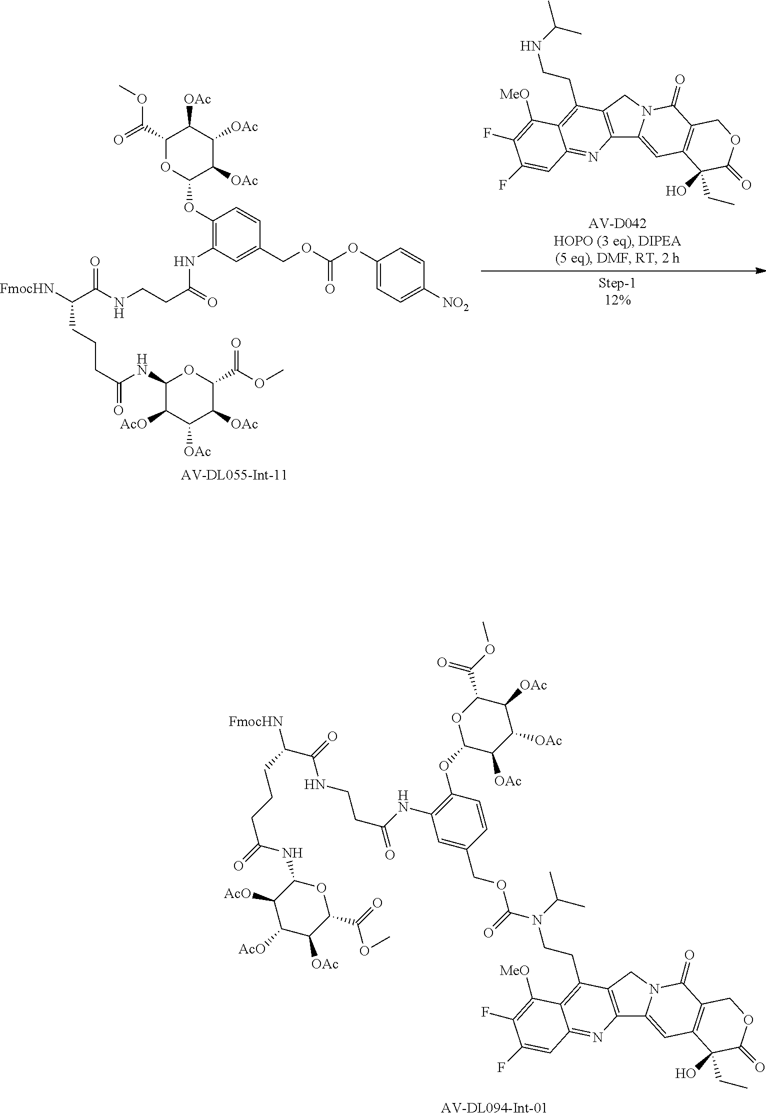

To a stirred solution of (2R,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 741 mg, 0.541 mmol), (S)-4-ethyl-8,9-difluoro-4-hydroxy-11-(2-(isopropylamino)ethyl)-10-methoxy-1,12-dihydro-14H-pyrano[3′,4′:6,7]indolizino[1,2-b]quinoline-3,14(4H)-dione (AV-D042, 270 mg, 0.541 mmol) in DMF (5 mL) were added DIPEA (0.5 mL, 2.70 mmol), HOPO (180 mg, 1.62 mmol) at 0° C. and stirred at RT for 2 h. The reaction was monitored by TLC. The resulting reaction mixture was concentrated under reduced pressure to get crude product. The crude product was further purified by reverse phase Biotage (column 12 g, C18) eluted with 80% MeCN in water to get (2R,3R,4S,5S,6S)-2-((S)-5-((((9H-fluoren-9-yl)methoxy)carbonyl)amino)-6-((3-((5-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL094-Int-01, 110 mg, 0.063 mmol, 12% yield) as a brown solid.

MS (ESI): 1732.87 [M+H]⁺.

### b. Synthesis of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL094-Int-02)

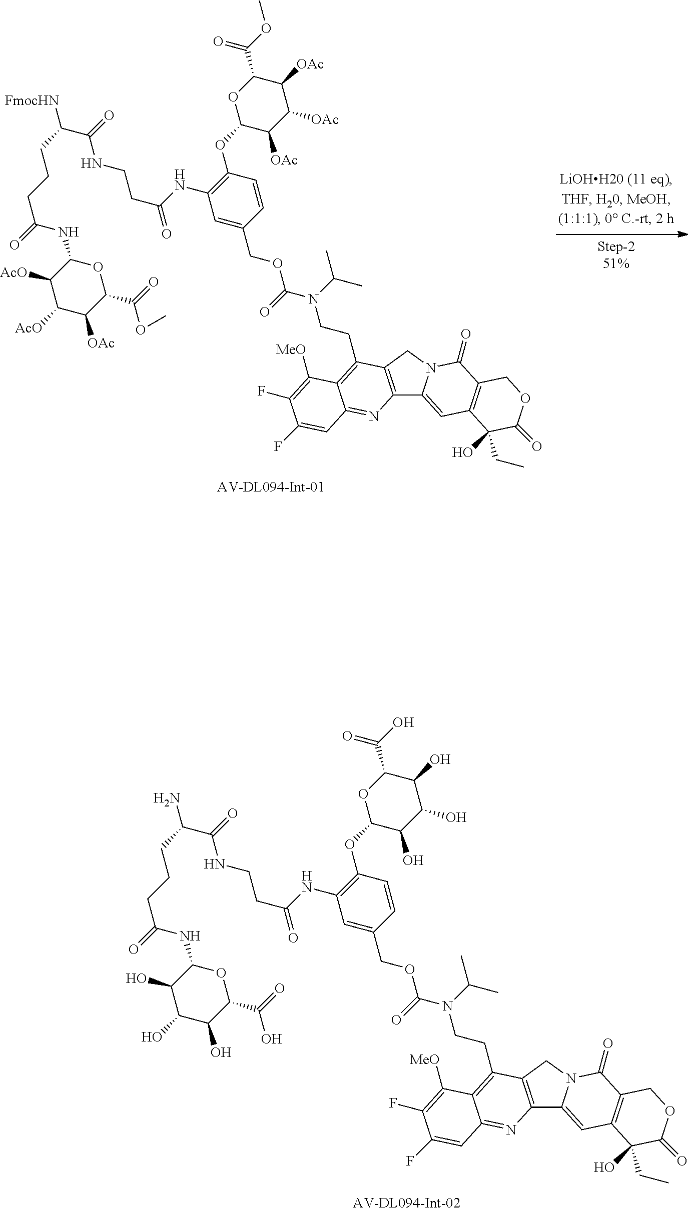

A stirred solution of (2R,3R,4S,5S,6S)-2-((S)-5-((((9H-fluoren-9-yl)methoxy)carbonyl)amino)-6-((3-((5-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL094-Int-01, 110 mg, 0.0635 mmol) in THF:H₂O:MeOH (1:1:1)(3 mL) was added LiOH·H₂O (26 mg, 0.635 mmol) at 0° C. and stirred at RT for 1 h. The reaction was monitored by LCMS. The reaction mixture was concentrated under reduced pressure to get crude product. The crude product was further purified by reverse phase Biotage (column 12 g, C18) eluted with 15% MeCN in water to get (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL094-Int-02, 40 mg, 0.0325 mmol, 51% yield) as an off white solid.

MS (ESI): 1230.64 [M+H]⁺.

### c. Synthesis of (2S,3R,4S,5S,6S)-2-(4-((((2-(((((S)-11-(2-((((4-((S)-2-((S)-2-((S)-6-(tert-butoxy)-2-(2-(2,5-dioxo-2,5-dihydro-1H-pyrrol-1-yl)acetamido)-6-oxohexanamido)-3-methylbutanamido)propanamido)benzyl)oxy)carbonyl)(isopropyl)amino)ethyl)-4-ethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl Triacetate (AV-DL094)

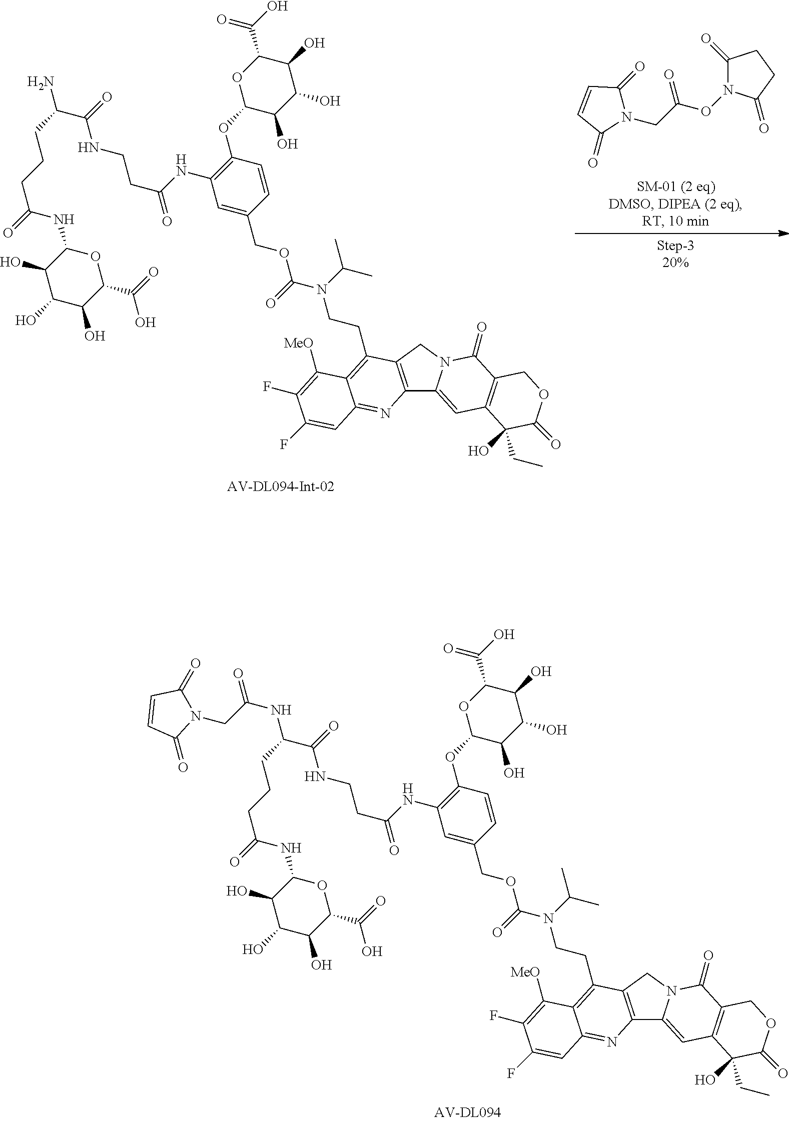 [SMILES: CC[C@@]1(O)C(=O)OCc2c1cc1n(c2=O)Cc2c-1nc1cc(F)c(F)c(OC)c1c2CCN(C(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@@H]2O[C@H](C(=O)O)[C@@H](O)[C@H](O)[C@H]2O)c1)C(C)C ; O=C(CN1C(=O)C=CC1=O)ON1C(=O)CCC1=O ; CC[C@@]1(O)C(=O)OCc2c1cc1n(c2=O)Cc2c-1nc1cc(F)c(F)c(OC)c1c2CCN(C(=O)OCc1ccc(O[C@@H]2O[C@H](C(=O)O)[C@@H](O)[C@H](O)[C@H]2O)c(NC(=O)CCNC(=O)[C@H](CCCC(=O)N[C@@H]2O[C@H](C(=O)O)[C@@H](O)[C@H](O)[C@H]2O)NC(=O)CN2C(=O)C=CC2=O)c1)C(C)C]

To a stirred solution (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-4-ethyl-8,9-difluoro-4-hydroxy-10-methoxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL094-Int-02, 40 mg, 0.0325 mmol) in DMSO (1 mL) was added 2,5-dioxopyrrolidin-1-yl 3-(2,5-dioxo-2,5-dihydro-1H-pyrrol-1-yl)propanoate (SM-01, 8.1 mg, 0.0325 mmol), DIPEA (11 μL, 0.0648 mmol) at 0° C. and stirred at RT for 10 min. The reaction was monitored by LCMS. The resulting reaction mixture was concentrated under reduced pressure to get crude. The crude was purified by prep HPLC and fractions were lyophilized to obtained (2S,3R,4S,5S,6S)-2-(4-((((2-(((((S)-11-(2-((((4-((S)-2-((S)-2-((S)-6-(tert-butoxy)-2-(2-(2,5-dioxo-2,5-dihydro-1H-pyrrol-1-yl)acetamido)-6-oxohexanamido)-3-methylbutanamido)propanamido)benzyl)oxy)carbonyl)(isopropyl)amino)ethyl)-4-ethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-6-(methoxycarbonyl)tetrahydro-2H-pyran-3,4,5-triyl triacetate (AV-DL094, 9.1 mg, 0.0067 mmol, 20% yield) as a pale yellow solid.

PREP-HPLC Conditions: GEMINI C18 (250*21.2*) 5μ (NEW), 250×19 mm×5 μm A: A: 0.1% TFA IN IN WATER; B: ACN:H₂O (80:20), 0/30, 13/56, 17/56, 17.1/98 @ 12 ML/MIN; diluent: MeCN, Water.

MS (ESI): 1367.64 [M+H]⁺: ¹H NMR (400 MHz, DMSO-d₆): δ 12.98 (brs, 2H), 9.15-9.01 (m, 1H), 8.42 (d, J=9.2 Hz, 1H), 8.36 (d, J=8.4 Hz, 1H), 8.20 (s, 1H), 8.08 (brs, 1H), 8.01-7.90 (m, 1H), 7.30 (s, 1H), 7.08 (s, 4H), 6.54 (s, 1H), 5.88 (brs, 1H), 5.50-5.30 (m, 6H), 5.09 (s, 3H), 4.90-4.70 (m, 2H), 4.22-4.09 (m, 8H), 3.91-3.85 (m, 1H), 3.60-3.40 m, 8H), 3.25-3.09 (m, 5H), 2.20-2.05 (m, 3H), 1.90-1.80 (m, 2H), 1.69-1.39 (m, 5H), 1.25-1.05 (m, 7H), 0.87 (t, J=7.2 Hz, 3H).

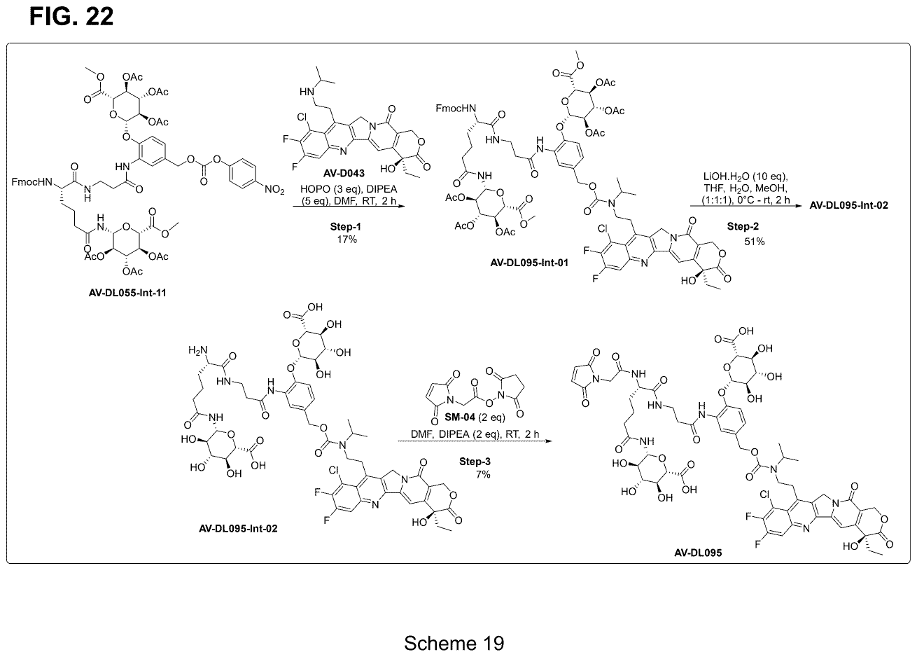

### XIX. Synthetic scheme for (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL095)(Scheme 19, FIG. 22)

### a. (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL095-Int-01)

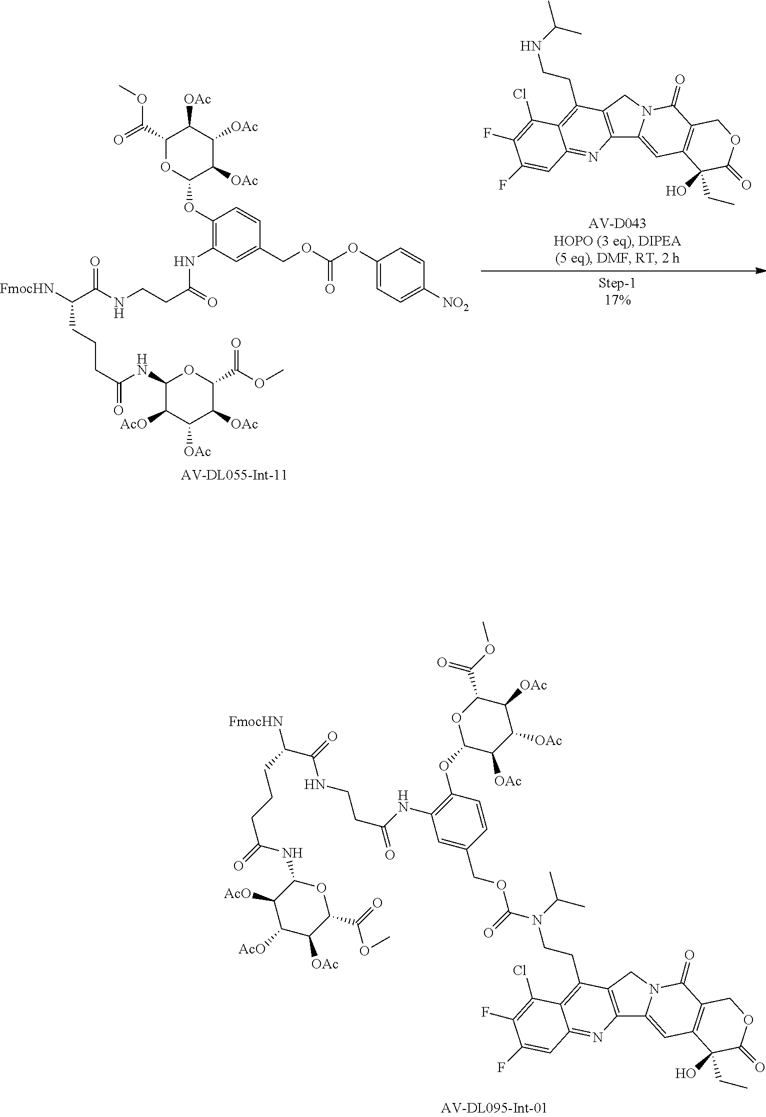

To a stirred solution of (2R,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 462 mg, 0.336 mmol), (S)-10-chloro-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D043, 170 mg, 0.336 mmol) in DMF (2 mL) were added DIPEA (0.4 mL, 1.68 mmol), HOPO (112 mg, 1.01 mmol) at 0° C. and stirred at RT for 2 h. The reaction was monitored by TLC. The resulting reaction mixture was concentrated under reduced pressure to get crude product. The crude product was further purified by reverse phase Biotage (column 12 g, C18) eluted with 85% MeCN in water to get (2S,3S,4S,5R,6S)-6-(2-(3-((S)-2-amino-6-(((2R,3R,4S,5S,6S)-6-carboxy-3,4,5 -trihydroxytetrahydro-2H-pyran-2-yl)amino)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL095-Int-01, 100 mg, 0.076 mmol, 17% yield) as a brown semi solid.

MS (ESI): 1737.84 [M+H]⁺.

### b. Synthesis of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL095-Int-02)

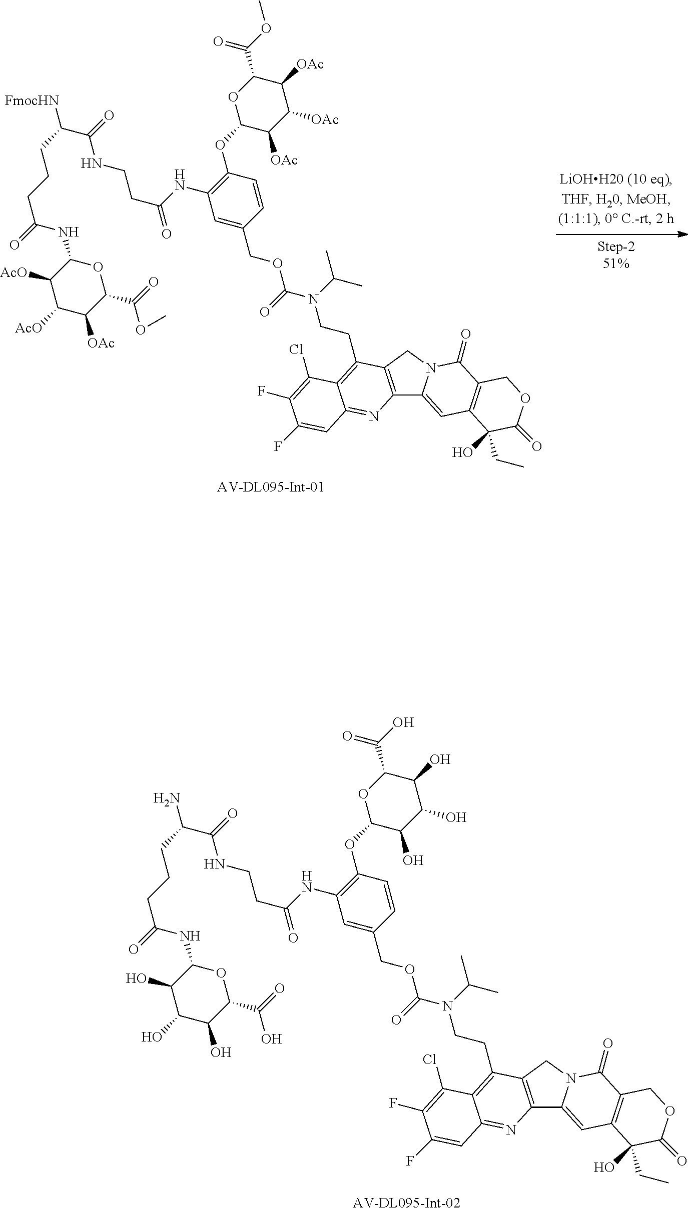

A stirred solution of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL095-Int-02, 110 mg, 0.0634 mmol) in THF:H₂O:MeOH (1:1:1)(3 mL) was added LiOH·H₂O (26 mg, 0.634 mmol) at 0° C. and stirred at RT for 1 h. The reaction was monitored by LCMS. The reaction mixture was concentrated under reduced pressure to get crude product. The crude product was further purified by reverse phase Biotage (column 12 g, C18) eluted with 25% MeCN in water to get (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11 -yl)ethyl)(isopropyl)carbamoyl)oxy)methyl)phenoxy)-3,4,5-trihydroxytetrahydro-2H-pyran-2-carboxylic acid (AV-DL095-Int-02, 40 mg, 0.0325 mmol, 51% yield) as an off white solid.

MS (ESI): 1235.0 [M+H]⁺.

### c. Synthesis of (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL095)

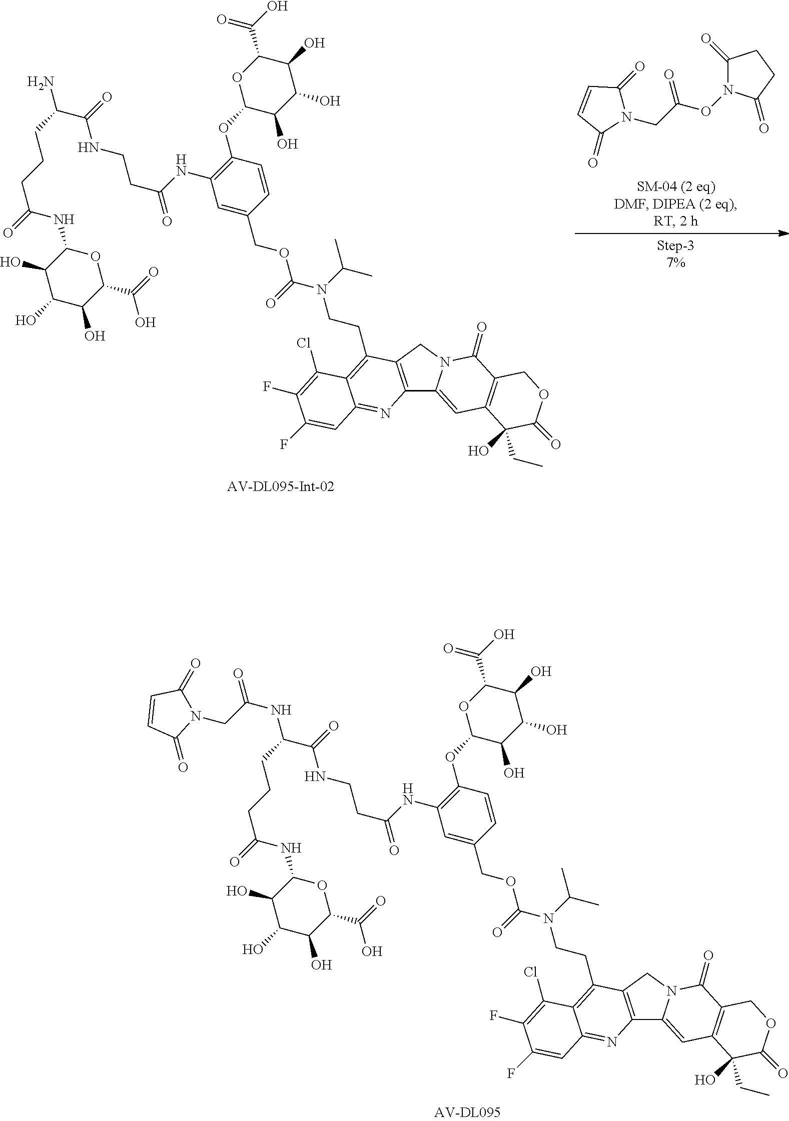 [SMILES: CC[C@@]1(O)C(=O)OCc2c1cc1n(c2=O)Cc2c-1nc1cc(F)c(F)c(Cl)c1c2CCN(C(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@@H]2O[C@H](C(=O)O)[C@@H](O)[C@H](O)[C@H]2O)c1)C(C)C ; O=C(CN1C(=O)C=CC1=O)ON1C(=O)CCC1=O ; CC[C@@]1(O)C(=O)OCc2c1cc1n(c2=O)Cc2c-1nc1cc(F)c(F)c(Cl)c1c2CCN(C(=O)OCc1ccc(O[C@@H]2O[C@H](C(=O)O)[C@@H](O)[C@H](O)[C@H]2O)c(NC(=O)CCNC(=O)[C@H](CCCC(=O)N[C@@H]2O[C@H](C(=O)O)[C@@H](O)[C@H](O)[C@H]2O)NC(=O)CN2C(=O)C=CC2=O)c1)C(C)C]

To a stirred solution of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL095-Int-02, 40 mg, 0.0324 mmol) in DMSO (1 mL) were added 2,5-dioxopyrrolidin-1-yl 3-(2,5-dioxo-2,5-dihydro-1H-pyrrol-1-yl)propanoate (SM-01, 8.0 mg, 0.0648 mmol), DIPEA (11 μL, 0.0648 mmol) at 0° C. and stirred at RT for 10 min. The reaction was monitored by LCMS. The resulting reaction mixture was concentrated under reduced pressure to get crude. The crude was purified by prep HPLC and fractions were lyophilized to obtained (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-10-chloro-4-ethyl-8,9-difluoro-4-hydroxy-3,14-dioxo-3,4,12,14-tetrahydro-1H-pyrano[3′,4′:6,7]indolizino[1,2-b]quinolin-11 -yl)ethyl)(isopropyl)carbamoyl)oxy)methyl)phenoxy)-3,4,5-trihydroxytetrahydro-2H-pyran-2-carboxylic acid (AV-DL095, 3 mg, 0.0022 mmol, 7% yield) as an off white solid.

PREP-HPLC Conditions: GEMINI C18 (250*21.2*) 5μ, 250×19 mm×5 μm A: 0.1% TFA IN IN WATER; B: ACN:H₂O (80:20), 0/30, 15/47,24/47,24.1/98; diluent: MeCN, Water.

MS (ESI): 1371.67 [M+H]⁺: ¹H NMR (400 MHz, DMSO-d₆): δ 13.01 (brs, 2H), 9.20-9.10 (m, 1H), 8.55-8.50 (m, 1H), 8.36 (d, J=8.4 Hz, 1H), 8.31-8.05 (m, 3H), 7.32 (s, 1H), 7.25-7.00 (m, 4H), 6.55 (s, 1H), 5.95 (brs, 1H), 5.51-5.41 (m, 4H), 5.15-5.04 (m, 3H), 4.85-4.67 (m, 2H), 4.22-4.10 (m, 2H), 4.09 (brs, 2H), 3.82-3.80 (m, 3H), 3.65-3.40 (m, 7H), 3.22-3.01 (m, 10H), 2.11-2.05 (m, 2H), 1.90-1.84 (m, 2H), 1.61-1.45 (m, 4H), 1.16-1.12 (m, 6H), 0.87 (t, J=7.2 Hz, 3H).

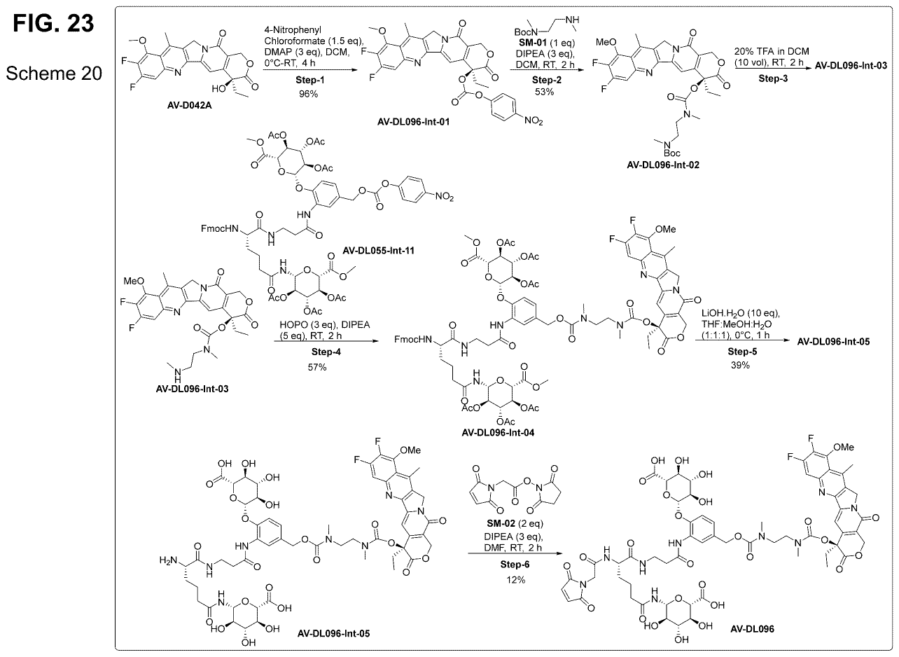

### XX. Synthetic scheme for (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-3,4,5-trihydroxytetrahydro-2H-pyran-2-carboxylic Acid (AV-DL096)(Scheme 20, FIG. 23)

### a. Synthesis of(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl (4-nitrophenyl) Carbonate (AV-DL096-Int-01)

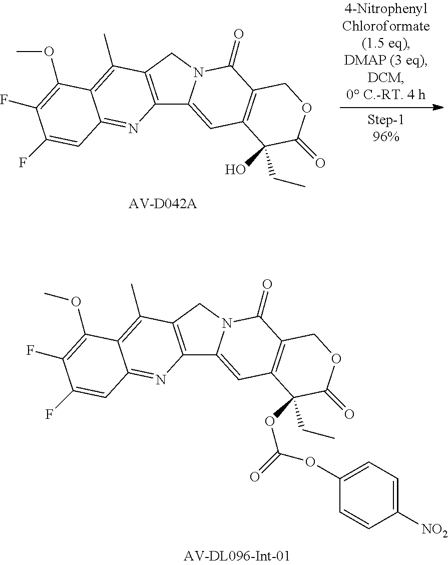

To a stirred solution of(S)-4-ethyl-8,9-difluoro-4-hydroxy-10-methoxy-11-methyl-1,12-dihydro-14H-pyrano[3′,4′:6,7]indolizino[1,2-b]quinoline-3,14(4H)-dione (AV-D042A, 300 mg, 0.701 mmol), 4-nitrophenyl Chloroformate (211 mg, 1.051 mmol) in DCM (12 mL) was added DMAP (256 mg, 2.10 mmol) at 0° C. and stirred at RT for 4 h. The reaction was monitored by TLC. The resulting reaction mixture was concentrated under reduced pressure and triturated with diethyl ether to get(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl (4-nitrophenyl)carbonate (AV-DL 096-Int-01, 400 mg, 0.076 mmol, 96% yield) as an off white solid. This was sued as such for the next step.

MS (ESI): 594.10 [M+H]⁺.

### b. Synthesis of(S)-tert-butyl (4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl) ethane-1,2-diylbis(methylcarbamate)(AV-DL096-Int-02)

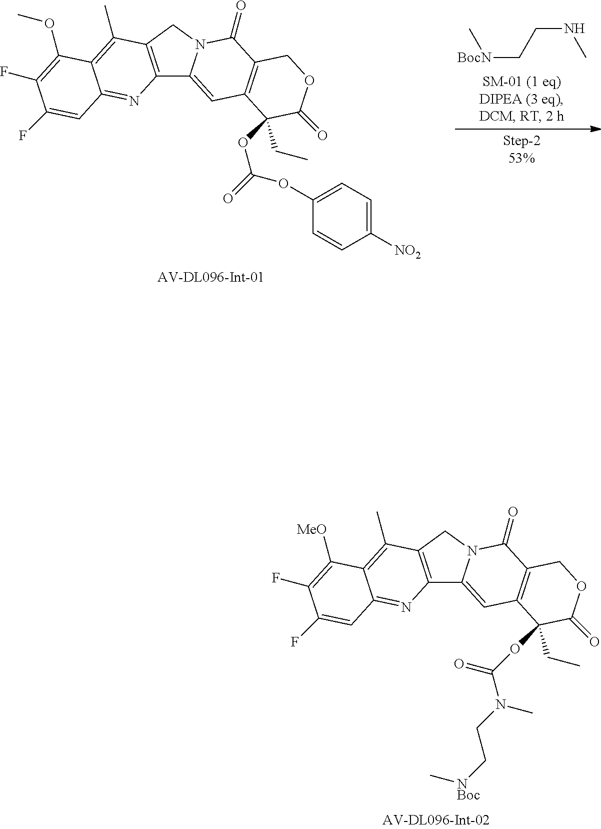

To a stirred solution of(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl (4-nitrophenyl) carbonate (AV-DL096-Int-01, 400 mg, 0.674 mmol) and tert-butyl methyl(2-(methylamino)ethyl)carbamate (SM-02, 126 mg, 0.674 mmol) in DCM (5 mL) was added DIPEA (0.3 mL, 2.024 mmol) at 0° C. and stirred at RT for 2 h. The reaction was monitored by TLC. The resulting reaction mixture was concentrated under reduced pressure to get crude product. The crude product was further purified by reverse phase Biotage (column 30 g, C18) eluted with 42% MeCN in water to get(S)-tert-butyl (4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl) ethane-1,2-diylbis(methylcarbamate)(AV-DL096-Int-02, 230 mg, 0.358 mmol, 53% yield) as an off white solid.

MS (ESI): 643.32 [M+H]⁺.

### c. Synthesis of(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL096-Int-03)

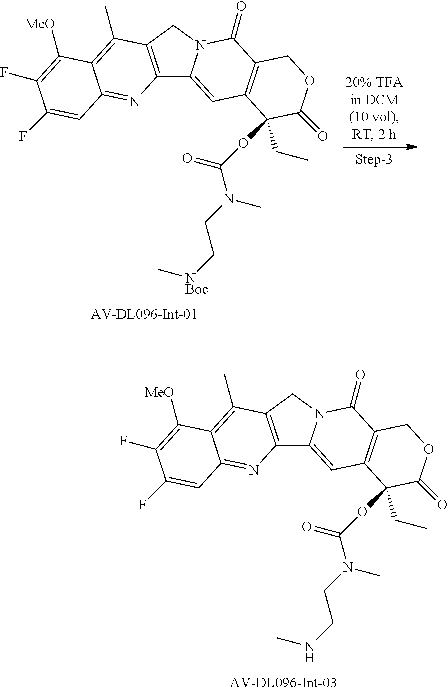 [SMILES: CC[C@@]1(OC(=O)N(C)CCN(C)C(=O)OC(C)(C)C)C(=O)OCc2c1cc1n(c2=O)Cc2c-1nc1cc(F)c(F)c(OC)c1c2C ; CC[C@@]1(OC(=O)N(C)CCNC)C(=O)OCc2c1cc1n(c2=O)Cc2c-1nc1cc(F)c(F)c(OC)c1c2C]

(S)-tert-Butyl (4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl) ethane-1,2-diylbis(methylcarbamate)(AV-DL096-Int-02, 230 mg, 0.358 mmol) was added to 20% TFA in DCM (0.23 mL) at 0° C. and stirred at RT for 2 h. The reaction was monitored by TLC. The resulting reaction mixture was concentrated under reduced pressure to get(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL096-Int-03, 194 mg, 0.382 mmol, quantitative yield) as a light brown solid.

MS (ESI): 543.29 [M+H]⁺.

### d. Synthesis of (2R,3R,4S,5S,6S)-2-((S)-5-((((9H-fluoren-9-yl)methoxy)carbonyl)amino)-6-((3-((5-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-(methoxycarbonyl)tetrahydro-2H-pyran-3,4,5-triyl triacetate (AV-DL096-Int-04)

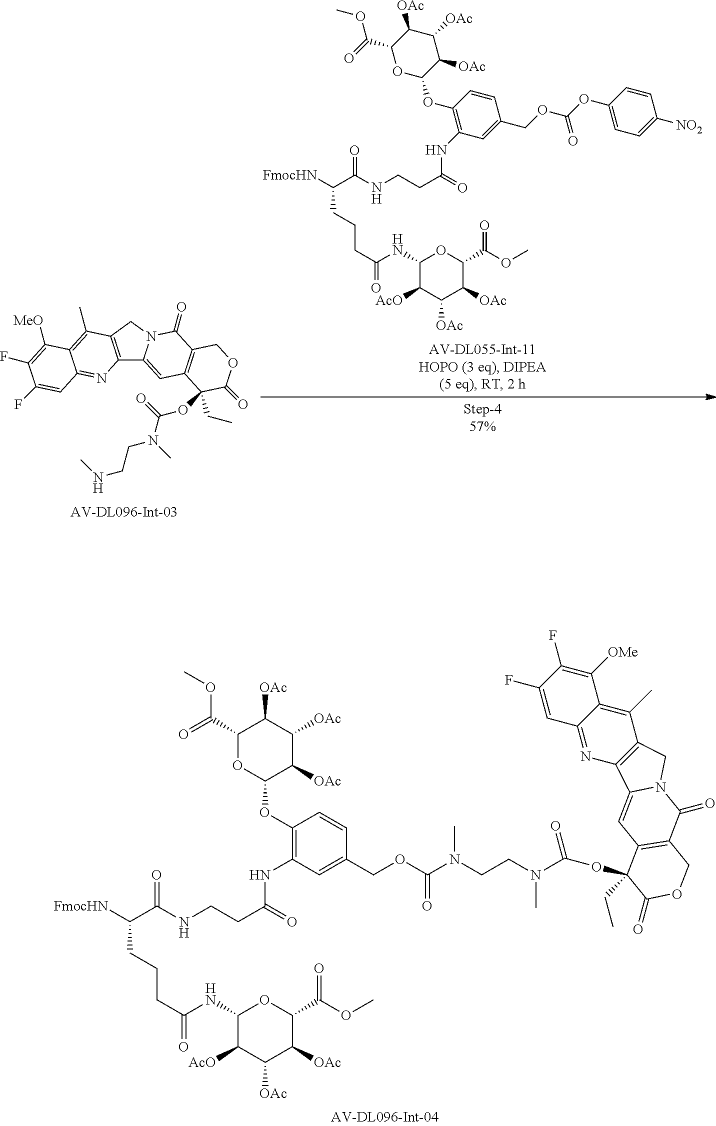

To a stirred solution of(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL096-Int-03, 160 mg, 0.295 mmol) and(S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL096-Int-03, 404 mg, 0.295 mmol) in DMF (5 mL) was added DIPEA (0.25 mL, 1.476 mmol), HOPO (98 mg, 0.886 mmol) at 0° C. and stirred at RT for 2 h. The reaction was monitored by TLC. The resulting reaction mixture was concentrated under reduced pressure to get the crude product. The crude product was further purified by reverse phase Biotage (column 30 g, C18) eluted with 80% MeCN in water to get (2R,3R,4S,5S,6S)-2-((S)-5-((((9H-fluoren-9-yl)methoxy)carbonyl)amino)-6-((3-((5-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL096-Int-04, 300 mg, 0.169 mmol, 57% yield) as an Off white solid.

MS (ESI): 1776.87 [M+H]⁺.

### e. Synthesis of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-3,4,5-trihydroxytetrahydro-2H-pyran-2-carboxylic Acid (AV-DL096-Int-05)

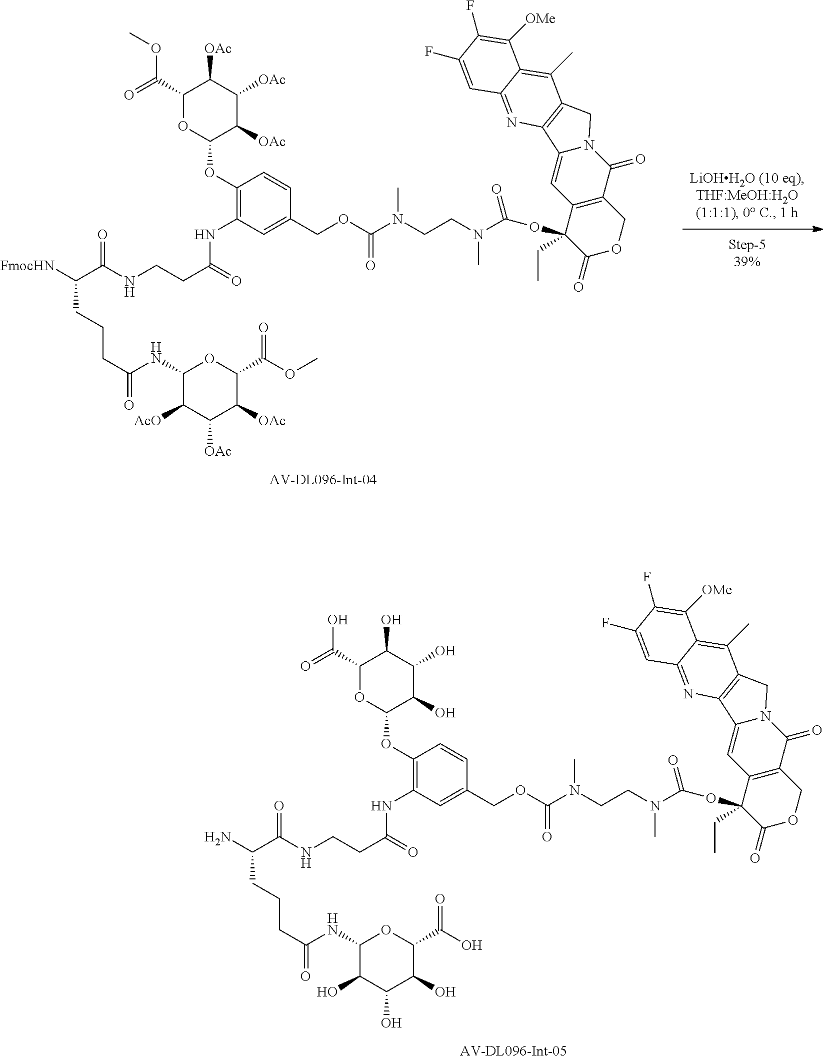

To a stirred solution of (2R,3R,4S,5S,6S)-2-((S)-5-((((9H-fluoren-9-yl)methoxy)carbonyl)amino)-6-((3-((5-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL096-Int-04, 300 mg, 0.169 mmol) in THF:H₂O:MeOH (1:1:1)(6 mL) was added LiOH·H₂O (70 mg, 1.69 mmol) at 0° C. and stirred at RT for 1 h. The reaction was monitored by LCMS. The reaction mixture was concentrated under reduced pressure to get crude product. The crude product was further purified by reverse phase Biotage (column 12 g, C18) eluted with 28% MeCN in water to get (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-3,4,5-trihydroxytetrahydro-2H-pyran-2-carboxylic acid (AV-DL096-Int-05, 85 mg, 0.0668 mmol, 39% yield) as an off white solid.

MS (ESI): 1273.68 [M+H]⁺.

### f. Synthesis of (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-3,4,5-trihydroxytetrahydro-2H-pyran-2-carboxylic Acid (AV-DL096)

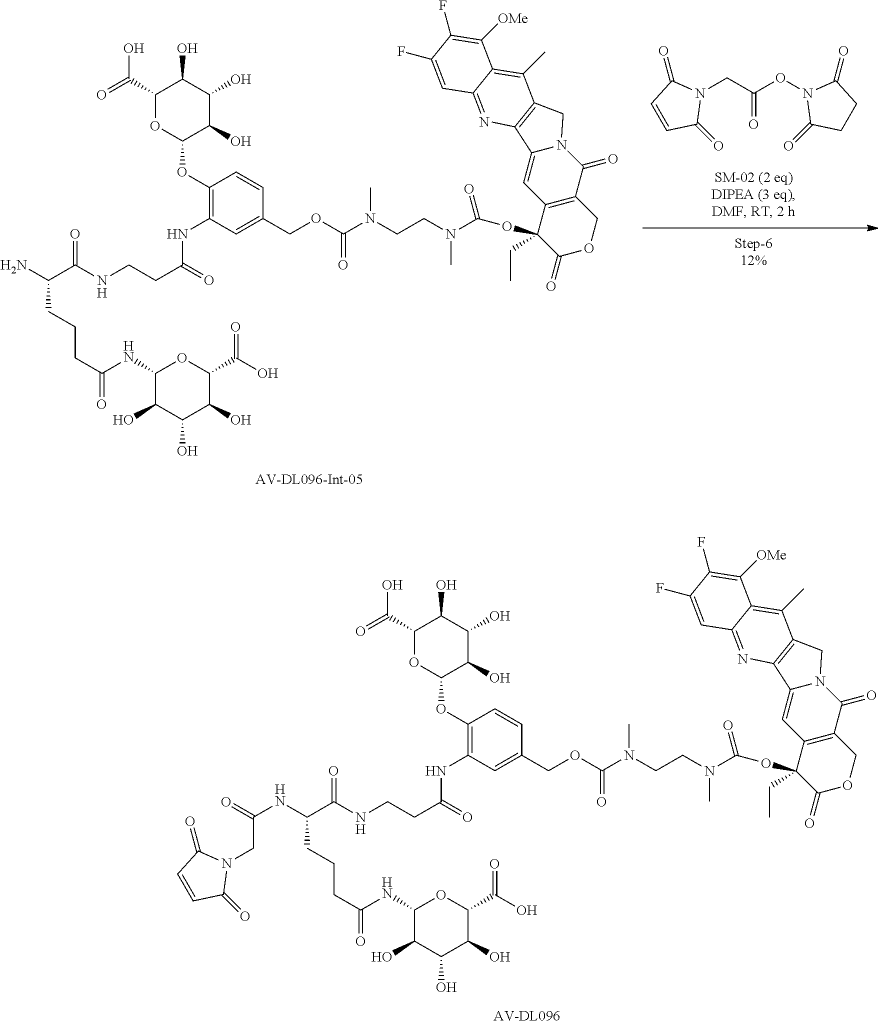 [SMILES: CC[C@@]1(OC(=O)N(C)CCN(C)C(=O)OCc2ccc(O[C@@H]3O[C@H](C(=O)O)[C@@H](O)[C@H](O)[C@H]3O)c(NC(=O)CCNC(=O)[C@@H](N)CCCC(=O)N[C@@H]3O[C@H](C(=O)O)[C@@H](O)[C@H](O)[C@H]3O)c2)C(=O)OCc2c1cc1n(c2=O)Cc2c-1nc1cc(F)c(F)c(OC)c1c2C ; O=C(CN1C(=O)C=CC1=O)ON1C(=O)CCC1=O ; CC[C@@]1(OC(=O)N(C)CCN(C)C(=O)OCc2ccc(O[C@@H]3O[C@H](C(=O)O)[C@@H](O)[C@H](O)[C@H]3O)c(NC(=O)CCNC(=O)[C@H](CCCC(=O)N[C@@H]3O[C@H](C(=O)O)[C@@H](O)[C@H](O)[C@H]3O)NC(=O)CN3C(=O)C=CC3=O)c2)C(=O)OCc2c1cc1n(c2=O)Cc2c-1nc1cc(F)c(F)c(OC)c1c2C]

To a stirred solution of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-3,4,5-trihydroxytetrahydro-2H-pyran-2-carboxylic acid (AV-DL096-Int-05, 100 mg, 0.0786 mmol) in DMSO (1 mL) were added 2,5-dioxopyrrolidin-1-yl 2-(2,5-dioxo-2,5-dihydro-1H-pyrrol-1-yl)acetate (SM-01, 20.0 mg, 0.0786 mmol), DIPEA (32 μL, 0.236 mmol) at 0° C. and stirred at RT for 10 min. The reaction was monitored by LCMS. The resulting reaction mixture was concentrated under reduced pressure to get crude product which was purified by RP prep HPLC and fractions were lyophilized to obtained (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-(((((S)-4-ethyl-8,9-difluoro-10-methoxy-11-methyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)phenoxy)-3,4,5-trihydroxytetrahydro-2H-pyran-2-carboxylic acid (AV-DL096, 12.5 mg, 0.0087 mmol, 12% yield) as an off white solid.

PREP-HPLC Conditions: X BRIDGE, C18, (250*19 mm) 5μ, A: 0.1% TFA IN IN WATER; B: ACN:H₂O (80:20), 0/25,15/50, 18/50, 18.1/95; diluent: MeCN, Water.

MS (ESI): 1410.77 [M+H]⁺: ¹H NMR (400 MHz, DMSO-d₆): δ 12.80 (brs, 2H), 9.01-8.93 (m, 1H), 8.43-8.36 (m, 2H), 8.36 (d, J=8.4 Hz, 1H), 8.15-8.03 (m, 2H), 7.71-7.93 (m, 1H), 7.08 (s, 2H), 6.95-6.87 (m, 3H), 5.89-5.70 (m, 1H), 5.50-5.15 (m, 8H), 5.15-4.40 (m, 7H), 4.25-4.01 (m, 7H), 3.90-3.50 (m, 6H), 3.30-3.05 (m, 6H), 2.91 (s, 3H), 2.78 (s, 3H), 2.81-2.60 (m, 6H), 1.70-1.38 (m, 5H), 0.95-0.75 (m, 3H).

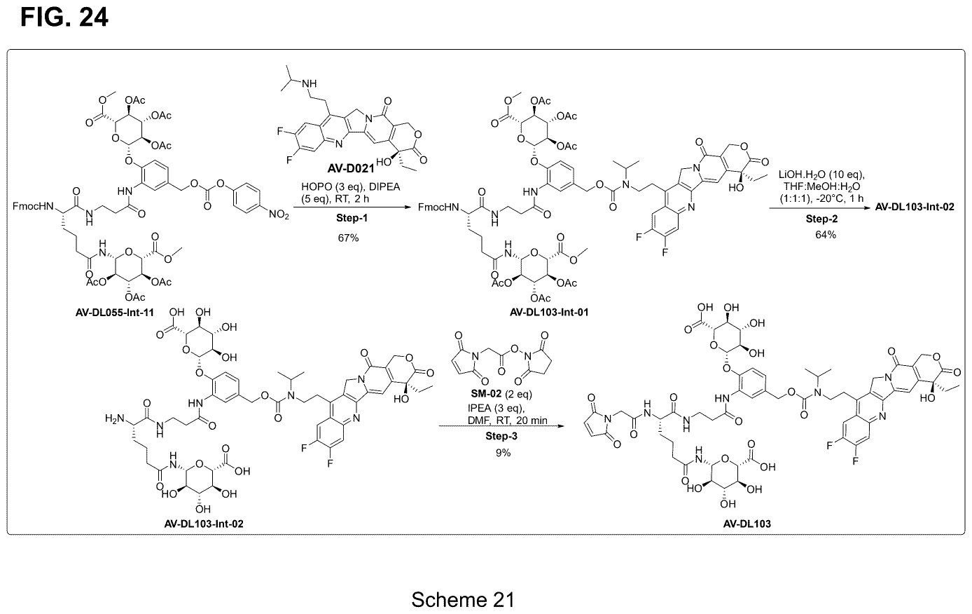

### XXI. Synthetic scheme for (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL103)(Scheme 21, FIG. 24)

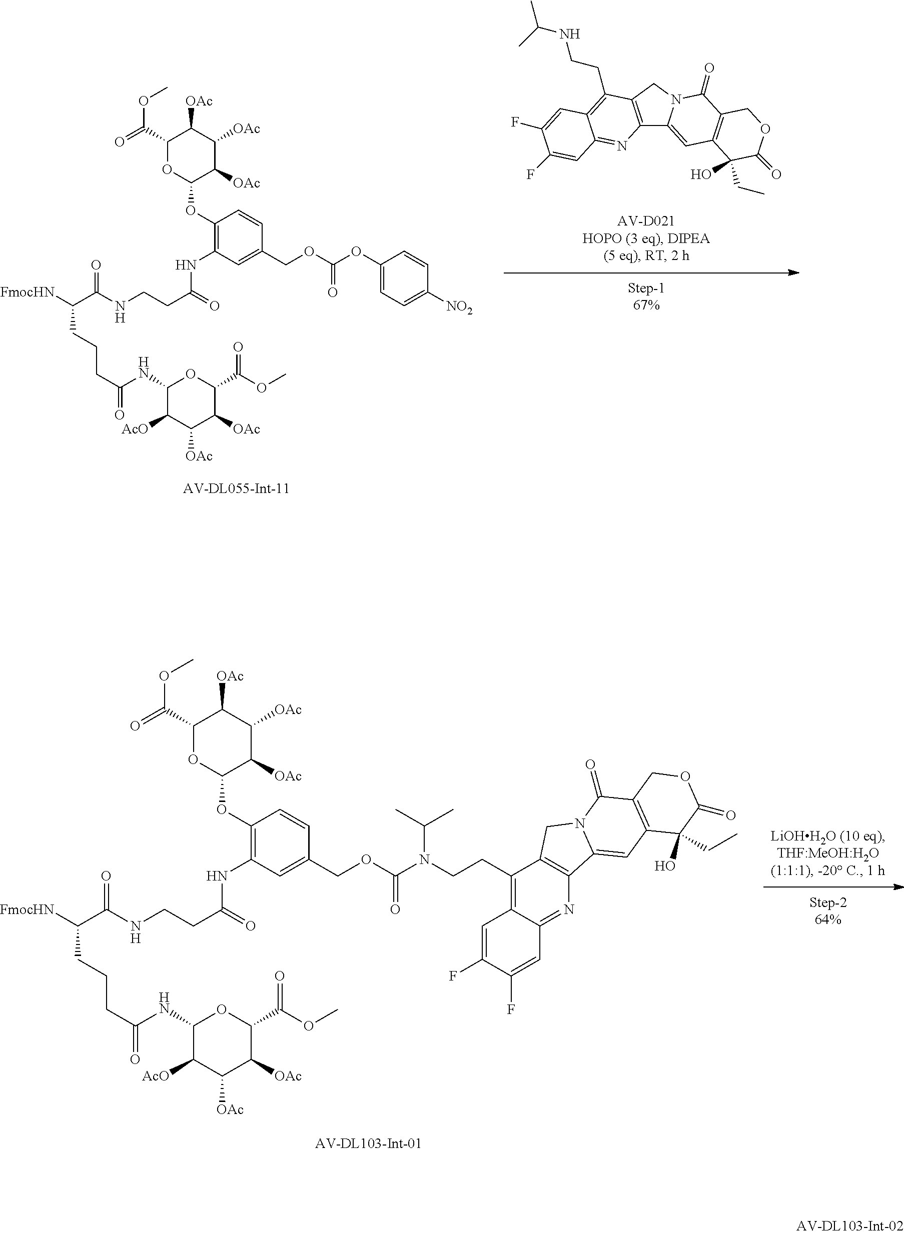

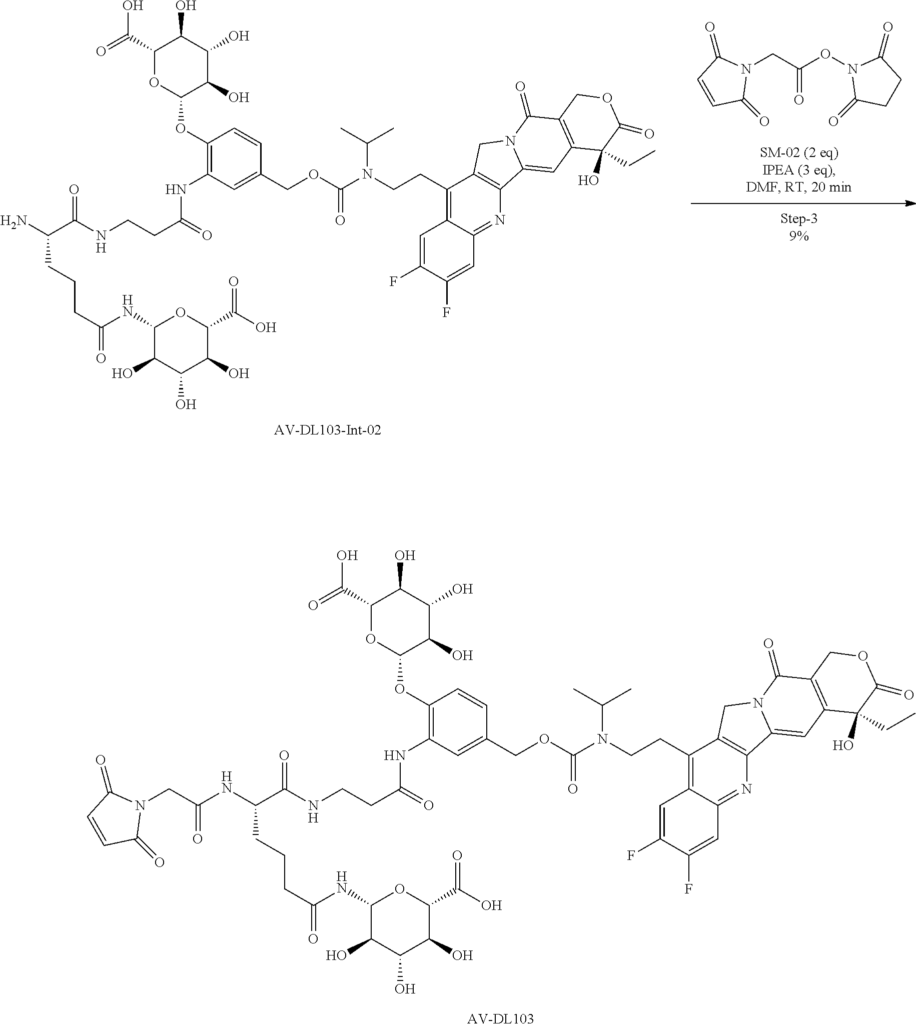 [SMILES: CC[C@@]1(O)C(=O)OCc2c1cc1n(c2=O)Cc2c-1nc1cc(F)c(F)cc1c2CCN(C(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@@H]2O[C@H](C(=O)O)[C@@H](O)[C@H](O)[C@H]2O)c1)C(C)C ; O=C(CN1C(=O)C=CC1=O)ON1C(=O)CCC1=O ; CC[C@@]1(O)C(=O)OCc2c1cc1n(c2=O)Cc2c-1nc1cc(F)c(F)cc1c2CCN(C(=O)OCc1ccc(O[C@@H]2O[C@H](C(=O)O)[C@@H](O)[C@H](O)[C@H]2O)c(NC(=O)CCNC(=O)[C@H](CCCC(=O)N[C@@H]2O[C@H](C(=O)O)[C@@H](O)[C@H](O)[C@H]2O)NC(=O)CN2C(=O)C=CC2=O)c1)C(C)C]

### a. Synthesis of (2R,3R,4S,5S,6S)-2-((S)-5-((((9H-fluoren-9-yl)methoxy)carbonyl)amino)-6-((3-((5-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl Triacetate (AV-DL103-Int-01)

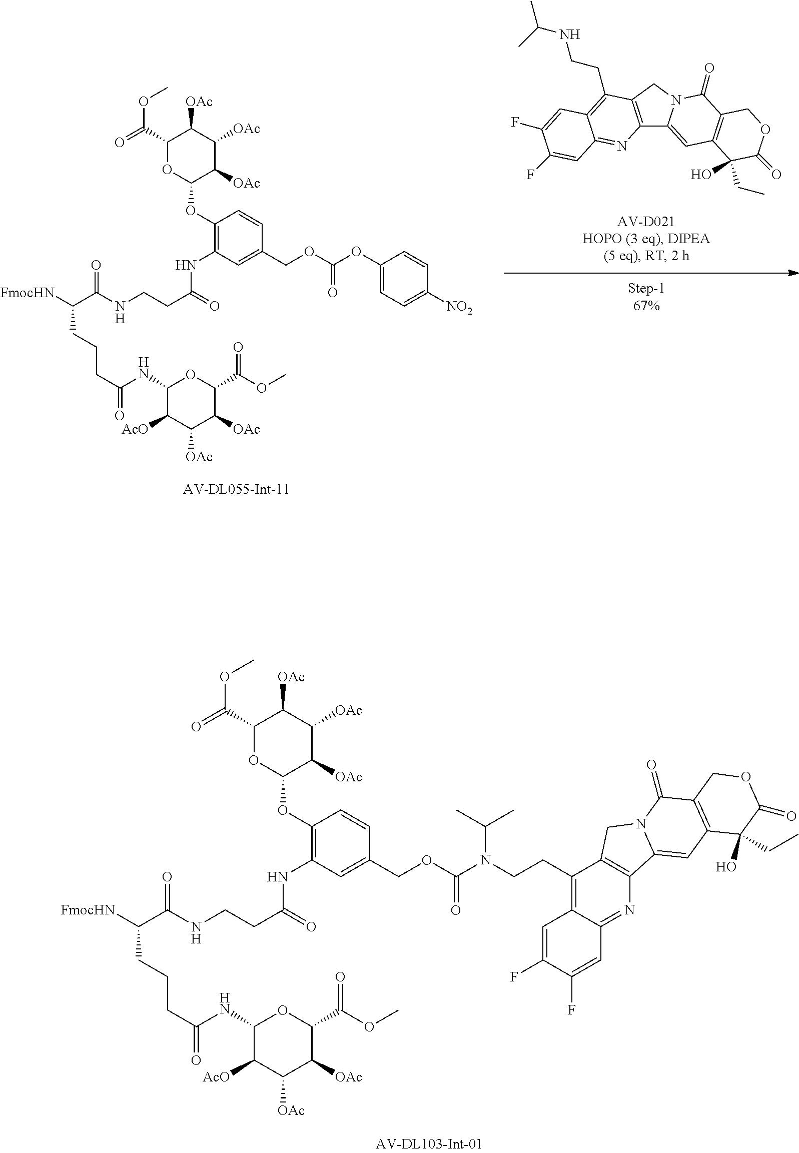

To a stirred solution of (2R,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 143 mg, 0.104 mmol), (S)-4-ethyl-8,9-difluoro-4-hydroxy-11-(2-(isopropylamino)ethyl)-1,12-dihydro-14H-pyrano[3′,4′:6,7]indolizino[1,2-b]quinoline-3,14(4H)-dione (AV-D021, 100 mg, 0.208 mmol) in DMF (2 mL) were added DIPEA (0.18 mL, 1.04 mmol), HOPO (70 mg, 0.624 mmol) at 0° C. and stirred at RT for 2 h. The reaction was monitored by TLC. The resulting reaction mixture was concentrated under reduced pressure to get the crude product. The crude product was further purified by reverse phase Biotage (column 30 g, C18) eluted with 50% MeCN in water to get (2R,3R,4S,5S,6S)-2-((S)-5-((((9H-fluoren-9-yl)methoxy)carbonyl)amino)-6-((3-((5-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL103-Int-01, 120 mg, 0.0705 mmol, 67% yield) as a brown solid.

MS (ESI): 1703.01 [M+H]⁺.

### b. Synthesis of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL103-Int-02)

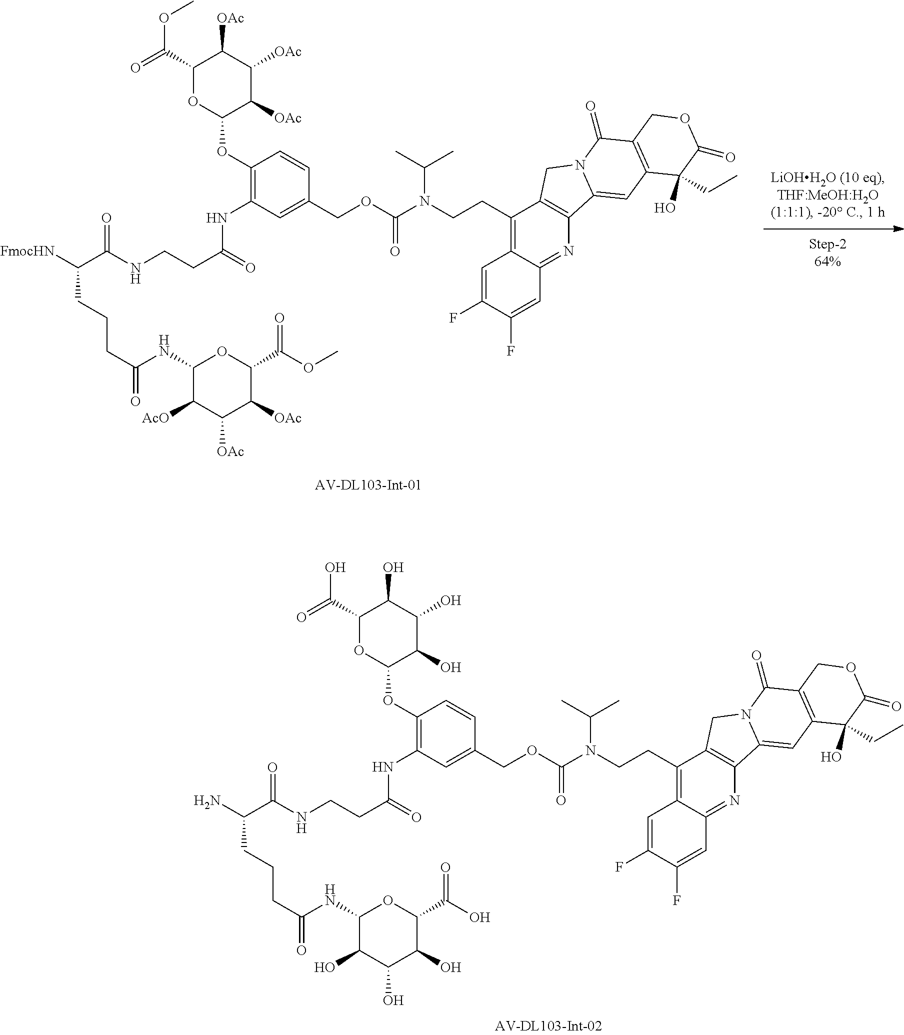

A stirred solution of (2R,3R,4S,5S,6S)-2-((S)-5-((((9H-fluoren-9-yl)methoxy)carbonyl)amino)-6-((3-((5-((((2-((S)-4-ethyl-8,9-difluoro-4-hydroxy-3,14-dioxo- 3,4,12,14-tetrahydro-1H-pyrano[3′,4′:6,7]indolizino[1,2-b]quinolin-11-yl)ethyl)(isopropyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL103-Int-01, 110 mg, 0.064 mmol) in THF:H₂O:MeOH (2:1:0.5, 2 mL) was added LiOH·H₂O (27 mg, 0.64 mmol) at 0° C. and stirred at RT for 1 h. The reaction was monitored by LCMS. The reaction mixture was concentrated under reduced pressure to get crude product. The crude product was further purified by reverse phase Biotage (column 12 g, C18) eluted with 22% MeCN in water to get (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL103-Int-02, 50 mg, 0.0417 mmol, 64% yield) as an off white solid.

MS (ESI): 1200.67 [M+H]⁺.

### c. Synthesis of (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic Acid (AV-DL103)

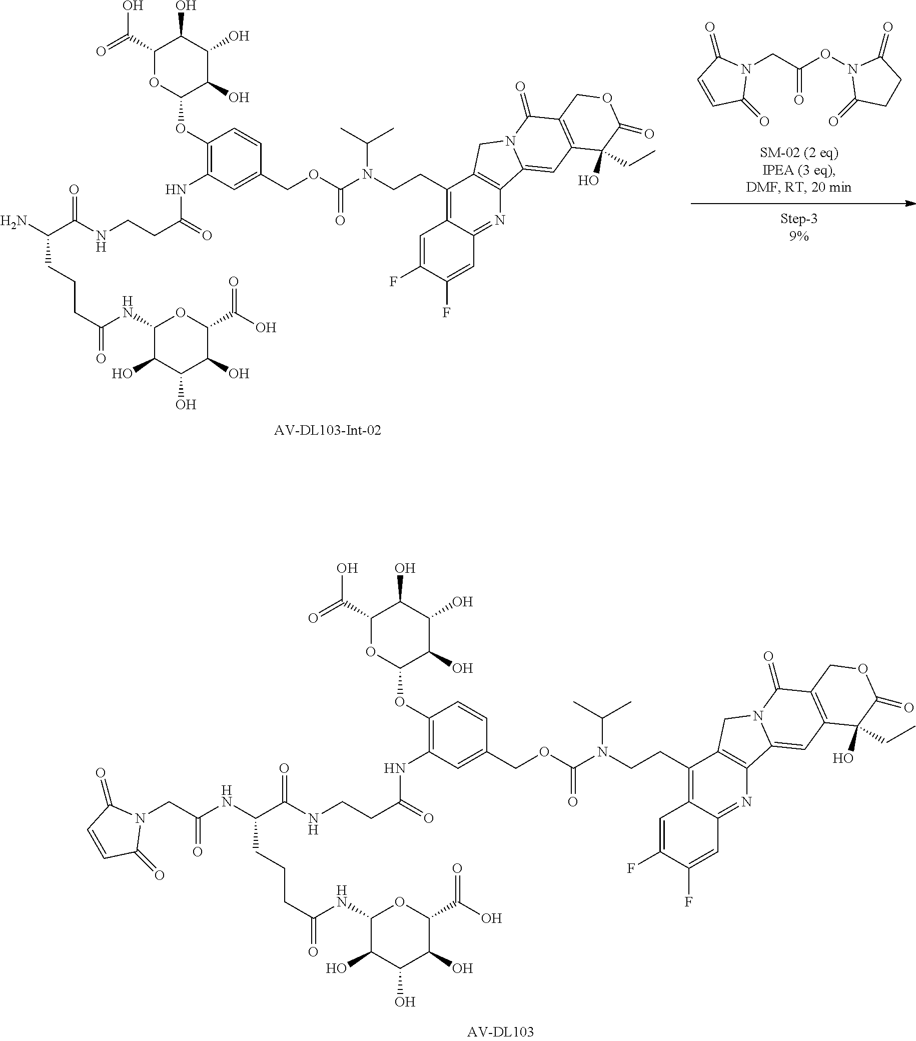 [SMILES: CC[C@@]1(O)C(=O)OCc2c1cc1n(c2=O)Cc2c-1nc1cc(F)c(F)cc1c2CCN(C(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@@H]2O[C@H](C(=O)O)[C@@H](O)[C@H](O)[C@H]2O)c1)C(C)C ; O=C(CN1C(=O)C=CC1=O)ON1C(=O)CCC1=O ; CC[C@@]1(O)C(=O)OCc2c1cc1n(c2=O)Cc2c-1nc1cc(F)c(F)cc1c2CCN(C(=O)OCc1ccc(O[C@@H]2O[C@H](C(=O)O)[C@@H](O)[C@H](O)[C@H]2O)c(NC(=O)CCNC(=O)[C@H](CCCC(=O)N[C@@H]2O[C@H](C(=O)O)[C@@H](O)[C@H](O)[C@H]2O)NC(=O)CN2C(=O)C=CC2=O)c1)C(C)C]

To a stirred solution of (2S,3S,4S,5R,6S)-6-(2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)-4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL103-Int-02, 50 mg, 0.0417 mmol) in DMF (1 mL) was added 2,5 -dioxopyrrolidin-1-yl 2-(2,5-dioxo-2,5-dihydro-1H-pyrrol-1-yl)acetate (SM-02, 10.5 mg, 0.0834 mmol), DIPEA (12 μL, 0.125 mmol) at 0° C. and stirred at RT for 20 minutes. The reaction was monitored by LCMS. The resulting reaction mixture was concentrated under reduced pressure to get crude product. The crude was purified by RP prep HPLC and fractions were lyophilized to get (2S,3S,4S,5R,6S)-6-(2-(3-((S)-6-(((2R,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((2-((S)-4-ethyl-8,9-difluoro-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (AV-DL103, 5.2 mg, 0.00389 mmol, 9% yield) as an off white solid.

PREP-HPLC Conditions: GEMINI,C18, (250*21.2 mm) 5μ, A: 0.1% TFA IN IN WATER; B: A CN:H₂O (60:40), 0/40,18/70,18.1/98@12 ML/MIN; diluent: MeCN, Water.

MS (ESI): 1338.69 [M+H]⁺: ¹H NMR (400 MHz, DMSO-d₆): δ 12.74 (brs, 2H), 9.13 (brs, 1H), 8.60-8.00 (m, 6H), 7.31 (s, 1H), 7.28-6.90 (m, 5H), 6.53 (s, 1H), 5.85 (s, 1H), 5.50-5.22 (m, 5H), 5.20-4.70 (m, 7H), 4.30-4.02 (m, 4H), 3.90-3.80 (m, 1H), 3.59 (d, J=9.2 Hz, 1H), 3.50-3.05 (m, 9H), 2.65-2.50 (m, 2H), 2.20-2.00 (m, 2H), 1.90-1.80 (m, 3H), 1.65-1.35 (m, 5H), 1.25-1.09 (m, 7H), 0.88 (t, J=7.2 Hz, 3H).

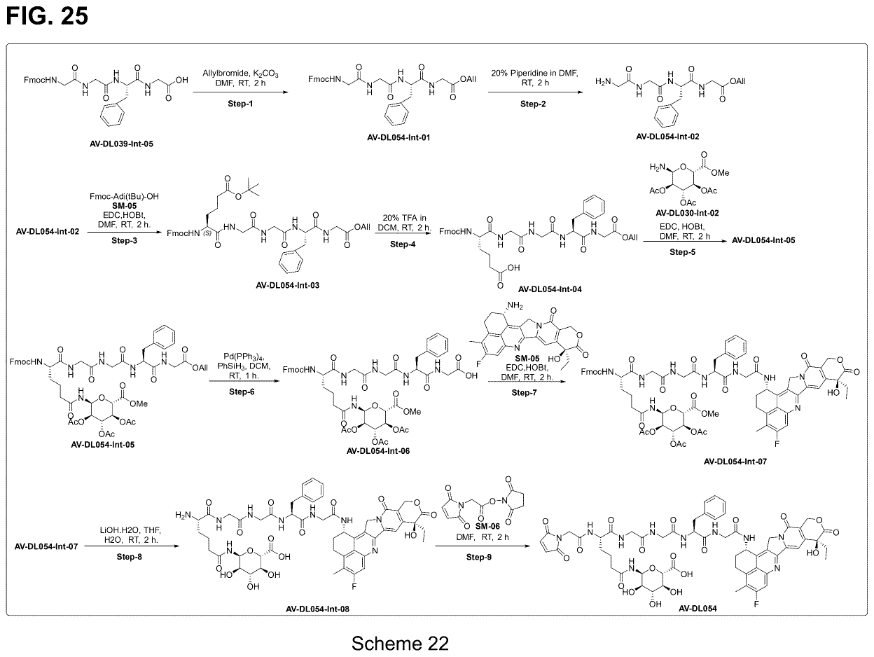

### XXII. Synthetic scheme for (2S,3S,4S,5R,6S)-6-((5S,14S)-5-benzyl-14-(2-(2,5-dioxo-2,5-dihydro-1H-pyrrol-1-yl)acetamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-3,4,5-trihydroxytetrahydro-2H-pyran-2-carboxylic acid (AV-DL054)(Scheme 22, FIG. 25)

### a. Synthesis of allyl (((9H-fluoren-9-yl)methoxy)carbonyl)glycylglycyl-L-phenylalanylglycinate (AV-DL054-Int-01)

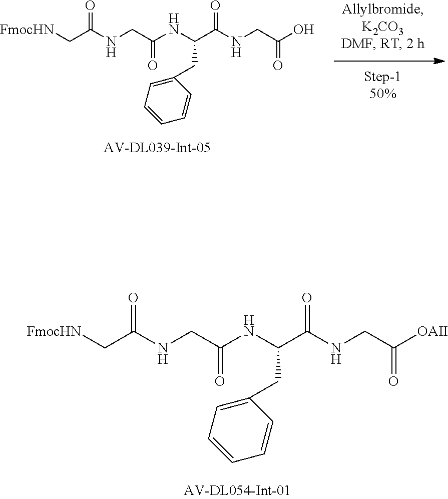

To a stirred solution of (((9H-fluoren-9-yl)methoxy)carbonyl)glycylglycyl-L-phenylalanylglycine (AV-DI039-Int-05, 1.5 g, 2.68 mmol) and allyl bromide (0.34 mL, 4.03 mmol) in DMF (10 mL) was added K₂CO₃ (556 mg, 4.032 mmol) at 0° C. and stirred at RT for 16 h. The reaction was monitored by TLC. The resulting reaction mixture was concentrated under reduced pressure to get crude product. The crude product was purified with silica gel column chromatography eluted with 5% MeOH/DCM and dried to get allyl (((9H-fluoren-9-yl)methoxy)carbonyl)glycylglycyl-L-phenylalanylglycinate (AV-DL054-Int-01, 800 mg, 1.34 mmol, 50% yield) as an off-white solid.

MS (ESI): 599.48 [M+H]⁺.

### Synthesis of Allyl glycylglycyl-L-phenylalanylglycinate (AV-DL054-Int-02)

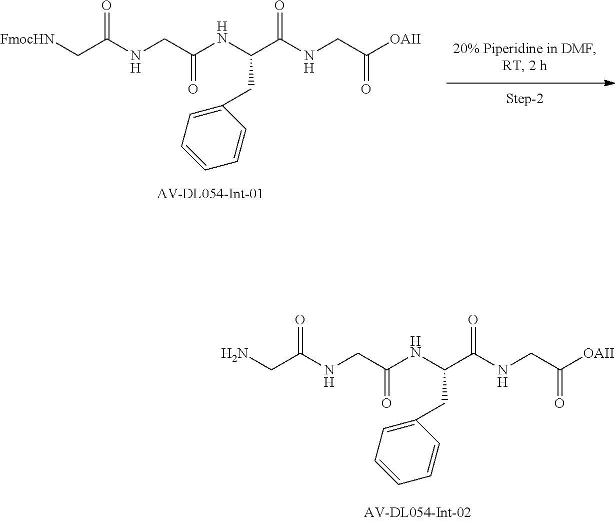

To a stirred solution of allyl (((9H-fluoren-9-yl)methoxy)carbonyl)glycylglycyl-L-phenylalanylglycinate (AV-DL054-Int-01, 800 μm, 1.34 mmol) in DMF (8 mL) was added piperdine (0.6 mL, 6.02 mmol) at 0° C. and stirred at RT for 2 h. The reaction was monitored by TLC. The reaction mixture was concentrated under reduced pressure to get crude product. The crude product was triturated with diethyl ether (3×100 mL) and dried to get allyl glycylglycyl-L-phenylalanylglycinate (AV-DL054-Int-02, 500 mg, 1.32 mmol, quantitative yield) as a pale yellow semi solid. This was used as such for the next step.

MS (ESI): 377.15 [M+H]⁺.

### b. Synthesis of 1-allyl 18-(tert-butyl)(5S,14S)-14-((((9H-fluoren-9-yl)methoxy)carbonyl)amino)-5-benzyl-4,7,10,13-tetraoxo-3,6,9,12-tetraazaoctadecanedioate (AV-DL054-Int-03)

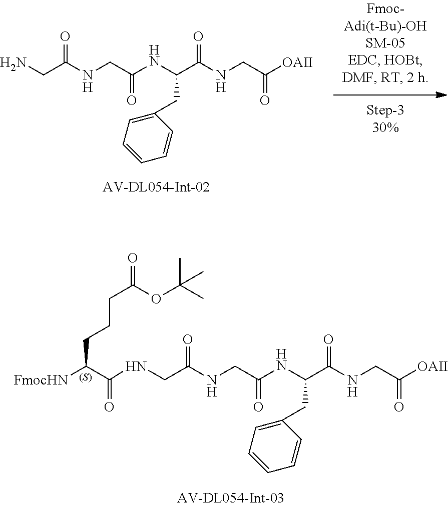

To a stirred solution of allyl glycylglycyl-L-phenylalanylglycinate (AV-DL054-Int-02, 500 mg, 1.329 mmol) and(S)-2-((((9H-fluoren-9-yl)methoxy)carbonyl)amino)-6-(tert-butoxy)-6-oxohexanoic acid (Fmoc-Adi(tBu)-OH, 580 mg, 1.329 mmol) in DMF (5 mL) was added EDC·HCl (507 mg, 507 mmol), HOBt (356 mg, 2.64 mmol) at 0° C. and stirred at RT for 2 h. The reaction was monitored by LCMS. The resulting reaction mixture was concentrated under reduced pressure to get crude product. The crude product was purified by reverse phase Biotage (column 30 g, C18) eluted with 45% MeCN in water and fractions were lyophilized to get 1-allyl 18-(tert-butyl)(5S,14S)-14-((((9H-fluoren-9-yl)methoxy)carbonyl)amino)-5-benzyl-4,7,10,13-tetraoxo-3,6,9,12-tetraazaoctadecanedioate (AV-DL054-Int-03, 300 mg, 0.376 mmol, 30% yield) as an off white solid.

MS (ESI): 798.39 [M+H]⁺.

### c. Synthesis of (9S,18S)-18-((((9H-fluoren-9-yl)methoxy)carbonyl)amino)-9-benzyl-5,8,11,14,17-pentaoxo-4-oxa-7,10,13,16-tetraazadocos-1-en-22-oic Acid (AV-DL054-Int-04)

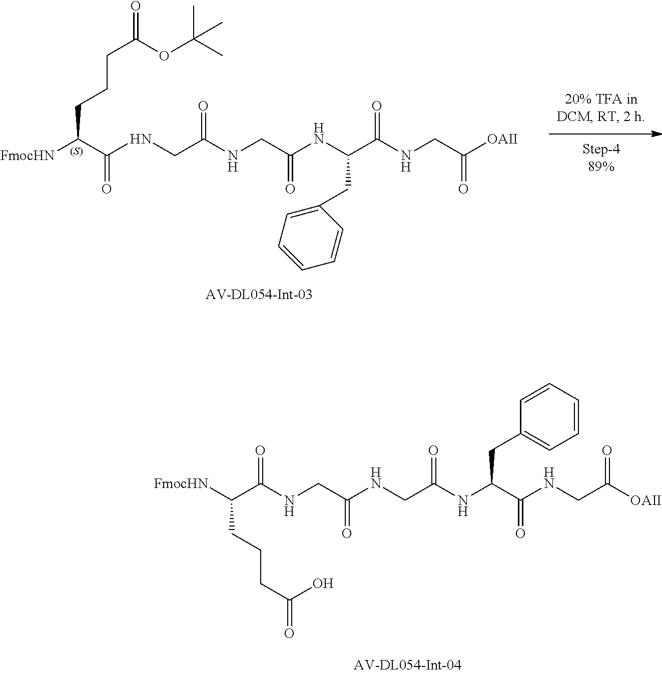

To a stirred solution of 1-allyl 18-(tert-butyl)(5S,14S)-14-((((9H-fluoren-9-yl)methoxy)carbonyl)amino)-5-benzyl-4,7,10,13-tetraoxo-3,6,9,12-tetraazaoctadecanedioate (AV-DL054-Int-03, 300 mg, 0.376 mmol) in 20% TFA in DCM (5 mL) was stirred at RT for 2 h. The reaction was monitored by LCMS. The resulting reaction mixture was concentrated under reduced pressure to get (9S,18S)-18-((((9H-fluoren-9-yl)methoxy)carbonyl)amino)-9-benzyl-5,8,11,14,17-pentaoxo-4-oxa-7,10,13,16-tetraazadocos-1-en-22-oic acid (AV-DL054-Int-04, 250 mg, 0.337 mmol, 89% yield) as an off white solid.

MS (ESI): 742.39 [M+H]⁺.

### d. Synthesis of (2S,3R,4S,5S,6S)-2-((9S,18S,E)-18-((((9H-fluoren-9-yl)methoxy)carbonyl)amino)-9-benzyl-5,8,11,14,17-pentaoxo-4-oxa-7,10,13,16-tetraazadocos-2-en-22-amido)-6-(methoxycarbonyl)tetrahydro-2H-pyran-3,4,5-triyl Triacetate (AV-DL054-Int-05)

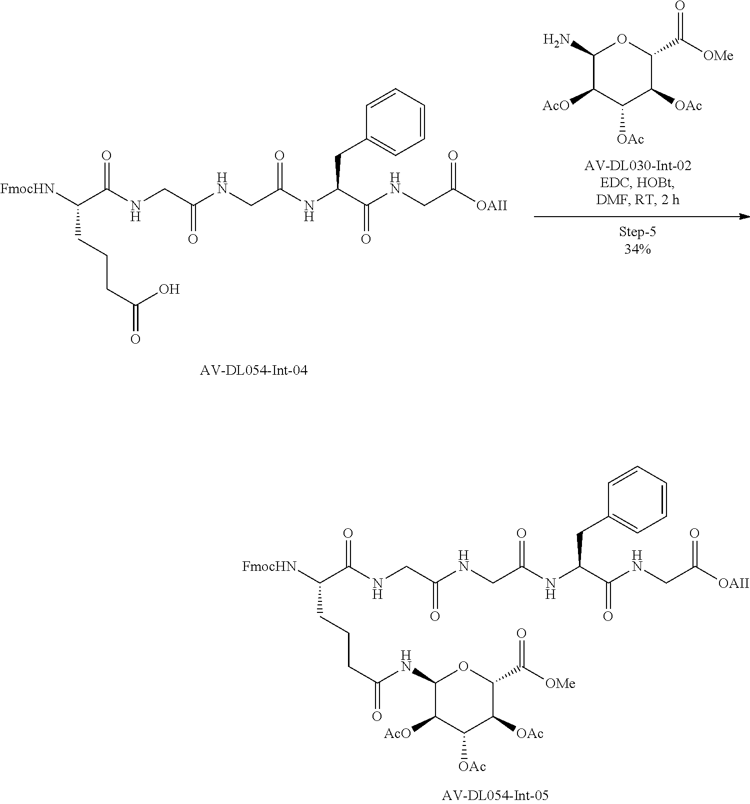

To a stirred solution of (9S,18S)-18-((((9H-fluoren-9-yl)methoxy)carbonyl)amino)-9-benzyl-5,8,11,14,17-pentaoxo-4-oxa-7,10,13,16-tetraazadocos-1-en-22-oic acid (AV-DL054-Int-04, 250 mg, 0.337 mmol), EDC·HCl (128 mg, 0.674 mmol) and HOBt (93 mg, 0.674 mmol) in DMF was added (2S,3R,4S,5S,6S)-2-amino-6-(methoxycarbonyl)tetrahydro-2H-pyran-3,4,5-triyl triacetate (AV-DL030-Int-02, 250 mg, 1.01 mmol) at 0° C. and stirred at RT for 2 h. The reaction was monitored by LCMS. The resulting reaction mixture was concentrated under reduced pressure to get crude product. The crude product was purified with reverse phase Biotage (column 30 g, C18) eluted with 34% MeCN in water and fractions were lyophilized to get (2S,3R,4S,5S,6S)-2-((9S,18S,E)-18-((((9H-fluoren-9-yl)methoxy)carbonyl)amino)-9-benzyl-5,8,11,14,17-pentaoxo-4-oxa-7,10,13,16-tetraazadocos-2-en-22-amido)-6-(methoxycarbonyl)tetrahydro-2H-pyran-3,4,5-triyl triacetate (AV-DL054-Int-05, 120 mg, 0.113 mmol, 34% yield) as an off white solid.

MS (ESI): 1058.2 [M+H]⁺.

### e. Synthesis of ((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanoyl)glycylglycyl-L-phenylalanylglycine (AV-DL054-Int-06)

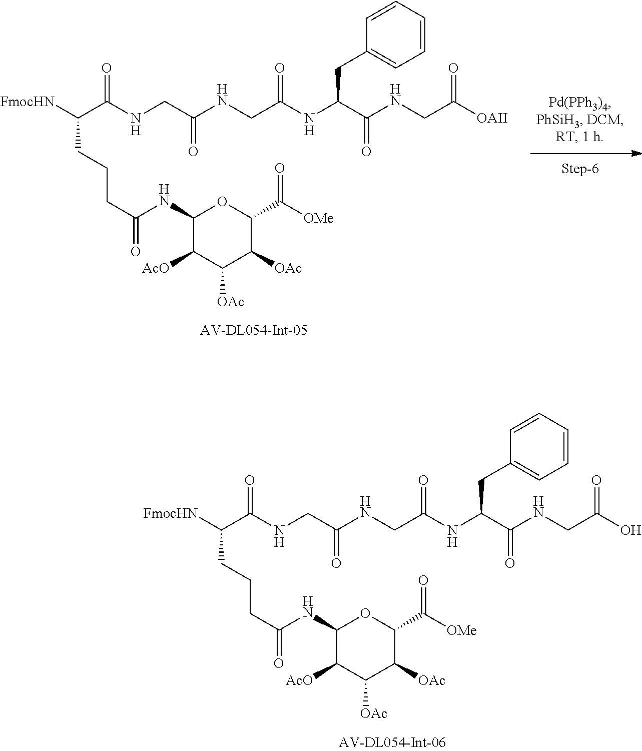

To a stirred solution of (2S,3R,4S,5S,6S)-2-((9S,18S,E)-18-((((9H-fluoren-9-yl)methoxy)carbonyl)amino)-9-benzyl-5,8,11,14,17-pentaoxo-4-oxa-7,10,13,16-tetraazadocos-2-en-22-amido)-6-(methoxycarbonyl)tetrahydro-2H-pyran-3,4,5-triyl triacetate (AV-DL054-Int-05, 120 mg, 0.113 mmol) and phenyl silane (28 μL, 0.226 mmol) in DCM (2 mL) was added Pd(PPh₃)₄ (3.2 mg, 0.0028 mmol) at 0° C. and stirred at RT for 1 h. The reaction was monitored by TLC. The resulting reaction mixture was concentrated under reduced pressure to get ((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanoyl)glycylglycyl-L-phenylalanylglycine (AV-DL054-Int-06, 115 mg, 0.133 mmol, quantitative yield) as a light brown semi solid. The crude was as such used for the next step.

MS (ESI): 1017.76 [M+H]⁺.

### f. Synthesis of (2S,3R,4S,5S,6S)-2-((5S,14S)-14-((((9H-fluoren-9-yl)methoxy)carbonyl)amino)-5-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-6-(methoxycarbonyl)tetrahydro-2H-pyran-3,4,5-triyl Triacetate (AV-DL054-Int-07)

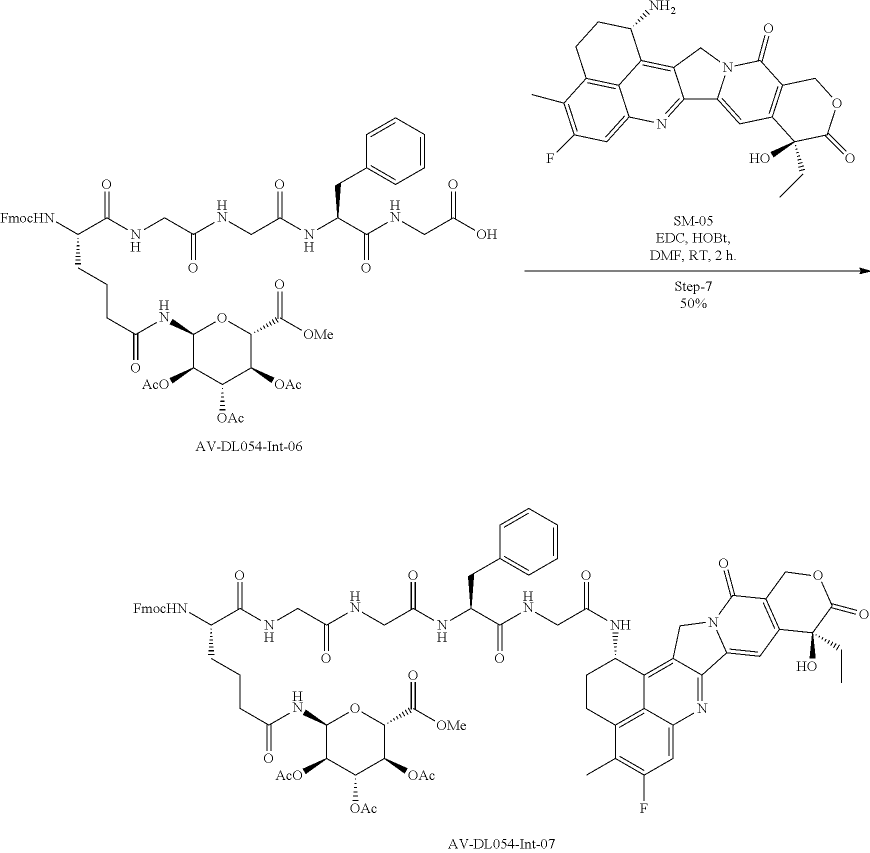

To a stirred solution of ((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanoyl)glycylglycyl-L-phenylalanylglycine (AV-DL054-Int-06, 115 mg, 0.113 mmol) and (1S,9S)-1-amino-9-ethyl-5-fluoro-9-hydroxy-4-methyl-1,2,3,9,12,15-hexahydro-10H, 13H-benzo[de]pyrano[3′,4′:6,7]indolizino[1,2-b]quinoline-10,13-dione (SM-05, 49 mg, 0.113 mmol) in DMF (3 mL) was added EDC·HCl (42 mg, 0.226 mmol), HOBt (31 mg, 0.226 mmol) at 0° C. and stirred at RT for 2 h. The reaction was monitored by LCMS. The resulting reaction mixture was concentrated under reduced pressure to get crude product. The crude product was purified with reverse phase Biotage (column 12 g, C18) eluted with 36% MeCN in water and fractions were lyophilized to get (2S,3R,4S,5S,6S)-2-((5S,14S)-14-((((9H-fluoren-9 -yl)methoxy)carbonyl)amino)-5-benzyl-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL054-Int-07, 80 mg, 0.56 mmol, 50% yield) as an off white solid.

MS (ESI): 1434.18 [M+H]⁺.

### g. Synthesis of (2S,3S,4S,5R,6S)-6-((5S,14S)-14-amino-5-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-3,4,5-trihydroxytetrahydro-2H-pyran-2-carboxylic Acid (AV-DL054-Int-08)

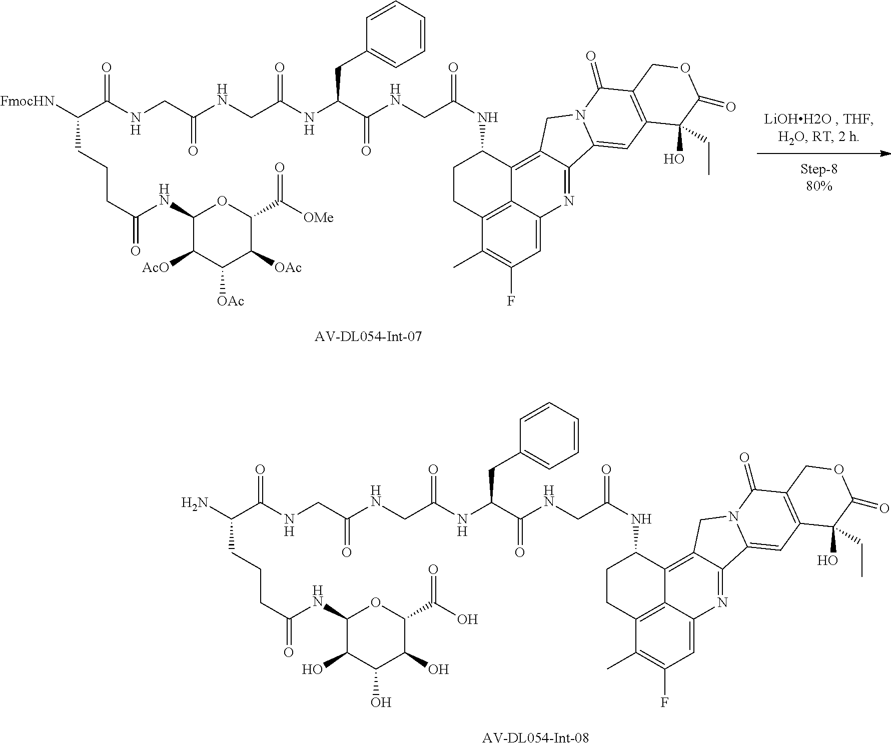

To a stirred solution of (2S,3R,4S,5S,6S)-2-((5S,14S)-14-((((9H-fluoren-9-yl)methoxy)carbonyl)amino)-5-benzyl-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-6-(methoxycarbonyl)tetrahydro-2H-pyran-3,4,5-triyl triacetate (AV-DL054-Int-07, 100 mg, 0.068 mmol) in THF:H₂O:MeOH (1:1:1)(3 mL) was added LiOH·H₂O (17.4 mg, 0.42 mmol) at 0° C. and stirred at RT for 2 h. The reaction was monitored by LCMS. The reaction was concentrated under reduced pressure to get crude product. The crude product was further purified by reverse phase Biotage (column 12 g, C18) eluted with 20% MeCN in water to get (2S,3S,4S,5R,6S)-6-((5S,14S)-14-amino-5-benzyl-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-3,4,5-trihydroxytetrahydro-2H-pyran-2-carboxylic acid (AV-DL054-Int-08, 60 mg, 0.056 mmol, 80% yield) as an off white solid.

MS (ESI): 1071.82 [M+H]⁺.

### h. Synthesis of (2S,3S,4S,5R,6S)-6-((5S,14S)-5-benzyl-14-(2-(2,5-dioxo-2,5-dihydro-1H-pyrrol-1-yl)acetamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-3,4,5-trihydroxytetrahydro-2H-pyran-2-carboxylic Acid (AV-DL054)

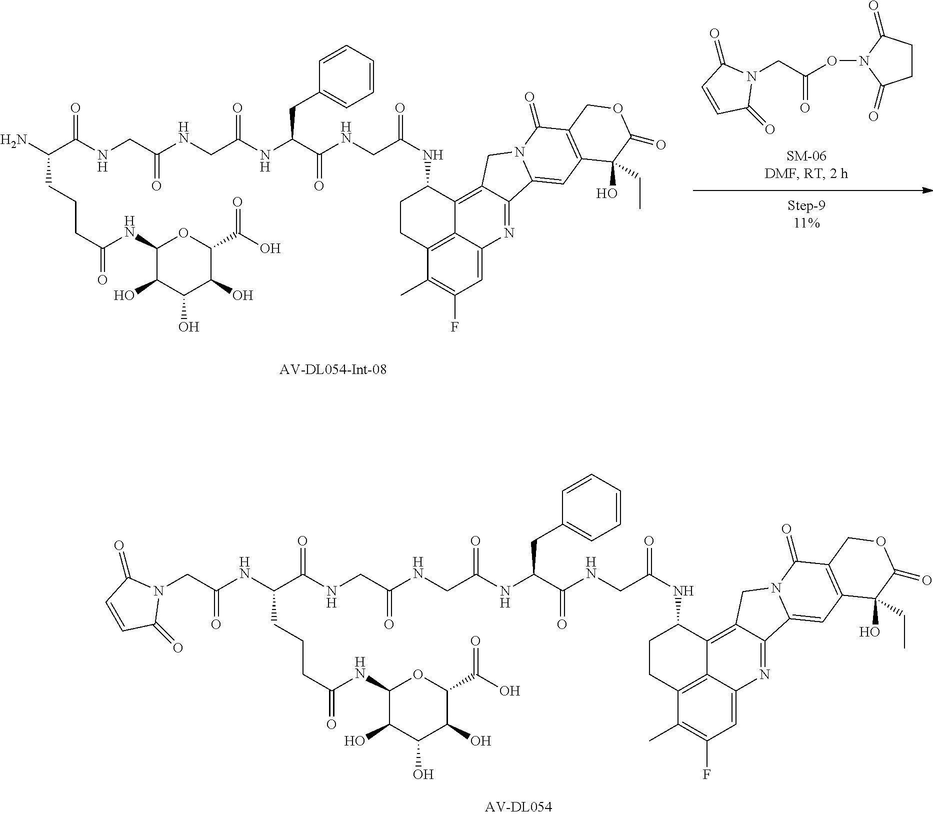 [SMILES: CC[C@@]1(O)C(=O)OCc2c1cc1n(c2=O)Cc2c-1nc1cc(F)c(C)c3c1c2[C@@H](NC(=O)CNC(=O)[C@H](Cc1ccccc1)NC(=O)CNC(=O)CNC(=O)[C@@H](N)CCCC(=O)N[C@H]1O[C@H](C(=O)O)[C@@H](O)[C@H](O)[C@H]1O)CC3 ; O=C(CN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](NC(=O)CNC(=O)[C@H](Cc1ccccc1)NC(=O)CNC(=O)CNC(=O)[C@H](CCCC(=O)N[C@H]1O[C@H](C(=O)O)[C@@H](O)[C@H](O)[C@H]1O)NC(=O)CN1C(=O)C=CC1=O)CC3]

To a stirred solution of (2S,3S,4S,5R,6S)-6-((5S,14S)-14-amino-5-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-3,4,5-trihydroxytetrahydro-2H-pyran-2-carboxylic acid (AV-DL054-Int-08, 60 mg, 0.056 mmol) and 2,5-dioxopyrrolidin-1-yl 2-(2,5-dioxo-2,5-dihydro-1H-pyrrol-1-yl)acetate (14 mg, 0.056 mmol) in DMF (3 mL) was added DIPEA (29 μL, 0.168) and stirred at RT for 2 h. The reaction was monitored by LCMS. The resulting reaction mixture was concentrated under reduced pressure to get the crude product which was further purified by RP Prep-HPLC to get (2S,3S,4S,5R,6S)-6-((5S,14S)-5-benzyl-14-(2-(2,5-dioxo-2,5-dihydro-1H-pyrrol-1-yl)acetamido)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,4,7,10,13-pentaoxo-3,6,9,12-tetraazaoctadecan-18-amido)-3,4,5-trihydroxytetrahydro-2H-pyran-2-carboxylic acid (AV-DL054, 7.6 mg, 0.063 mmol, 11% yield) as an off white solid.

MS (ESI): 1209.20 [M+H]⁺; ¹H NMR (400 MHz, DMSO-d₆): δ 8.44-8.39 (m, 3H), 8.34 (t, J=5.2 Hz, 1H), 8.28 (t, J=5.2 Hz, 1H), 8.10 (d, J=7.6 Hz, 1H), 8.00 (t, J=5.6 Hz, 1H), 7.80 (d, J=11.2 Hz, 1H), 7.32 (s, 1H), 7.25-7.10 (m, 5H), 7.05 (s, 2H), 6.52 (brs, 1H), 5.61-5.55 (m, 1H), 5.41 (d, J=6.4 Hz, 2H), 5.24 (d, J=4.8 Hz, 2H), 4.74 (t, J=8.8 Hz, 1H), 4.47-4.40 (m, 1H), 4.30-4.22 (m, 1H), 4.10 (d, J=6.0 Hz, 2H), 3.80-3.50 (m, 8H), 3.33-2.95 (m, 6H), 2.80-2.70 (m, 1H), 2.41 (s, 3H), 2.25-2.08 (m, 4H), 1.92-1.79 (m, 3H), 1.70-1.40 (m, 5H), 0.90-0.80 (m, 3H); —COOH peak was not observed.

PREP-HPLC Conditions: Shim-pack C18 (250*20.0 mm) 5μ: 0.1% FA in water; B: Acetonitrile: 0/20,2/20,12/45, 18/45, 18.1/98; diluent: MeCN, Water.

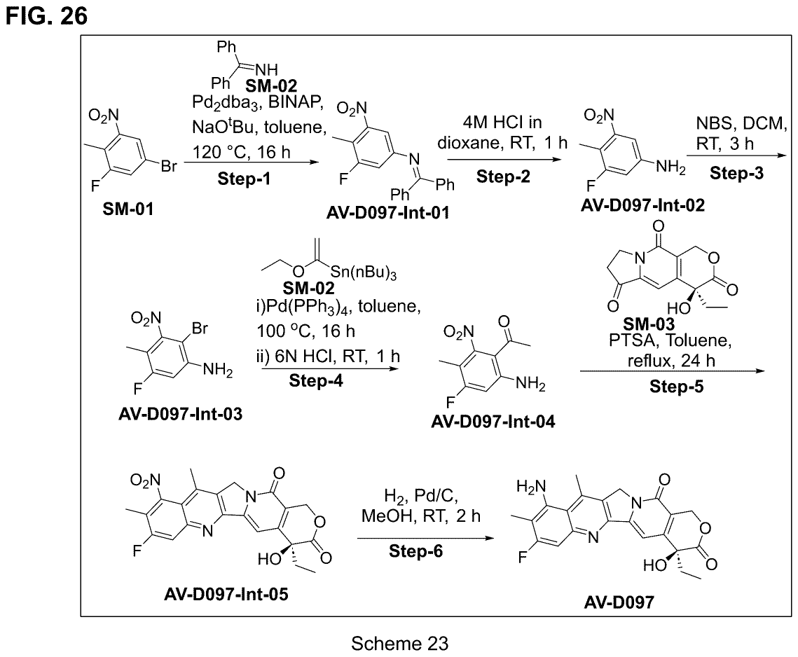

### XXIII. Synthetic scheme for(S)-10-amino-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D097, Scheme 23, FIG. 26)

### a. Synthesis of N-(3-fluoro-4-methyl-5-nitrophenyl)-1,1-diphenylmethanimine (AV-D097-Int-01)

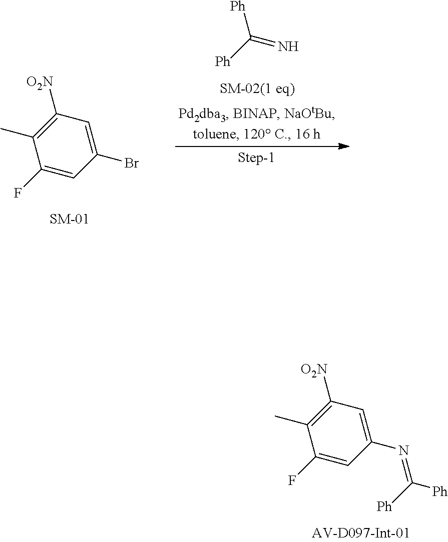 [SMILES: N=C(c1ccccc1)c1ccccc1 ; Cc1c(F)cc(Br)cc1[N+](=O)[O-] ; Cc1c(F)cc(N=C(c2ccccc2)c2ccccc2)cc1[N+](=O)[O-]]

To a stirred solution of 5-bromo-1-fluoro-2-methyl-3-nitrobenzene (SM-01, 2 g, 8.45 mmol) and 4-diphenylmethanimine (2.32 g, 12.82 mmol) in toluene (20 mL) was sequentially added Pd (dba) 3 (782 mg, 0.854 mmol), BINAP (1.061 g, 1.708 mmol), and NaO^{t}Bu (2.4 g, 25.62 mmol) at 0° C. and stirred at 120° C. for 16 h. The reaction was monitored by TLC. The resulting reaction mixture was concentrated under reduced pressure and triturated with diethyl ether to get N-(3-fluoro-4-methyl-5-nitrophenyl)-1,1-diphenylmethanimine (AV-D097-Int-01, 4 g crude) as a brown gum. This was used as such used for the next step.

MS (ESI): 335.02 [M+H]⁺.

### b. Synthesis of 3-fluoro-4-methyl-5-nitroaniline (AV-D097-Int-02)

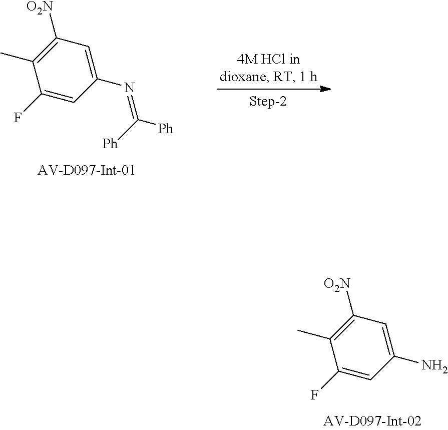 [SMILES: Cc1c(F)cc(N=C(c2ccccc2)c2ccccc2)cc1[N+](=O)[O-] ; Cc1c(F)cc(N)cc1[N+](=O)[O-]]

To N-(3-fluoro-4-methyl-5-nitrophenyl)-1,1-diphenylmethanimine (AV-D097-Int-01, 4 g, 14.97 mmol) was added 4 M HCl in dioxane (40 mL) and stirred at room temperature for 1 h. The reaction was monitored by TLC. The resulting reaction mixture was concentrated under reduced pressure to get crude product. The crude product was further purified by silica gel column chromatography eluted with 7% ethyl acetate in hexanes to obtain 3-fluoro-4-methyl-5-nitroaniline (AV-D097-Int-02, 600 mg, 3.529 mmol, 41% yield for two steps) as a yellow solid.

MS (ESI): 170.88 [M+H]⁺: ¹H NMR (400 MHz, DMSO-d₆): δ 6.98 (dd, J=1.2, 2.0 Hz, 1H), 6.66 (dd, J=2.4, 12.0 Hz, 1H), 5.86 (brs, 2H), 2.16 (d, J=2.0 Hz, 3H).

### c. Synthesis of 2-bromo-5-fluoro-4-methyl-3-nitroaniline (AV-D097-Int-03)

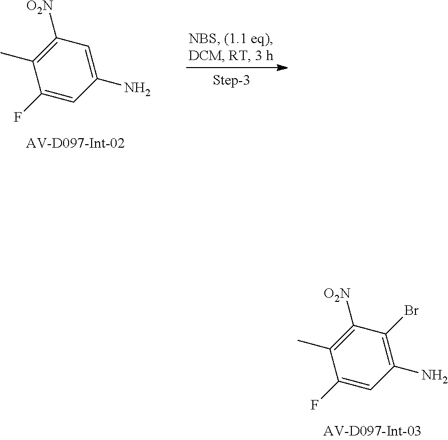

To a stirred solution of 3-fluoro-4-methyl-5-nitroaniline (AV-D097-Int-02, 1.4 g, 8.23 mmol) in DCM (14 mL) was added NBS (1.45 g, 8.235 mmol) at RT. The reaction was stirred at room temperature for 3 h. The reaction mixture was passed through celite bed and washed with DCM (30 mL). The filtrate was evaporated under reduced pressure and residue was purified by silica gel column chromatography eluted with 3% ethyl acetate in hexanes to obtain 2-bromo-5-fluoro-4-methyl-3-nitroaniline (AV-D097-Int-03, 900 mg, 3.63 mmol, 45% yield) as a brown semi solid.

¹H NMR (400 MHz, DMSO-d₆): δ 6.76 (d, J=12.0 Hz, 1H), 6.09 (brs, 2H), 2.00 (d, J=1.2 Hz, 3H).

### d. Synthesis of 1-(6-amino-4-fluoro-3-methyl-2-nitrophenyl) ethan-1-one (AV-D097-Int-04)

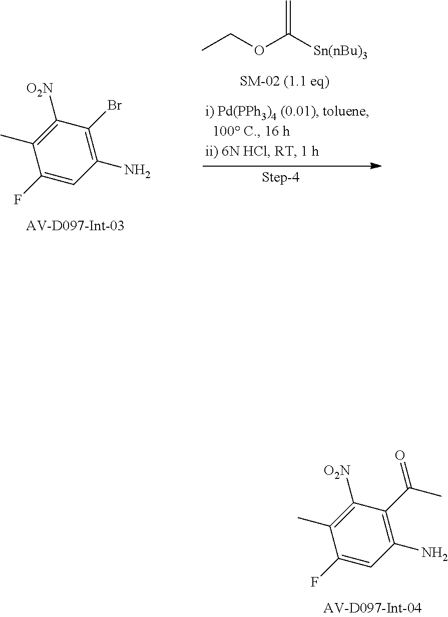

To a stirred solution of 2-bromo-5-fluoro-4-methyl-3-nitroaniline (AV-D097-Int-03, 900 mg, 3.64 mmol) in toluene (10 mL) was added tributyl(1-ethoxyvinyl) tin (1.3 mL, 3.643 mmol) and Pd(PPh₃)₄ (42 mg, 0.036 mmol) under nitrogen. The reaction was stirred at 100° C. for 16 h. The reaction mixture was quenched with 2N HCl. The reaction was passed through celite bed and washed with 10% methanol in DCM (50 mL). The filtrate was evaporated under reduced pressure and residue was purified by silica gel column chromatography eluted with 40% hexanes in ethyl acetate to obtain 1-(6-amino-4-fluoro-3-methyl-2-nitrophenyl) ethan-1-one (AV-D097-Int-04, 400 mg, 1.89 mmol, 51% yield) as a brown semi solid.

MS (ESI): 212.95 [M+H]⁺: ¹H NMR (400 MHz, DMSO-d₆): δ 6.6.79 (d, J=12.0 Hz, 1H), 6.32 (brs, 2H), 2.40 (s, 3H), 2.00 (d, J=1.2 Hz, 3H).

### e. Synthesis of(S)-4-ethyl-8-fluoro-4-hydroxy-9,11-dimethyl-10-nitro-1,12-dihydro-14H-pyrano[3′,4′:6,7]indolizino[1,2-b]quinoline-3,14(4H)-dione (AV-D097-Int-05)

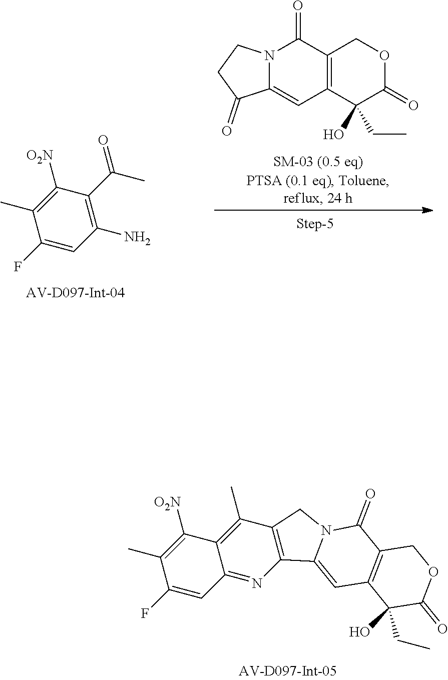 [SMILES: CC[C@@]1(O)C(=O)OCc2c1cc1n(c2=O)CCC1=O ; CC(=O)c1c(N)cc(F)c(C)c1[N+](=O)[O-] ; CC[C@@]1(O)C(=O)OCc2c1cc1n(c2=O)Cc2c-1nc1cc(F)c(C)c([N+](=O)[O-])c1c2C]

To a stirred solution of 1-(6-amino-4-fluoro-3-methyl-2-nitrophenyl) ethan-1-one (AV-D092-Int-04, 500 mg, 2.35 mmol) and(S)-4-ethyl-4-hydroxy-7,8-dihydro-1H-pyrano[3,4-f]indolizine-3,6,10 (4H)-trione (SM-03, 310 mg, 1.18 mmol) in toluene (10 mL) was added p-toluenesulfonic acid (90 mg, 0.47 mmol) at room temperature and stirred at 110° C. for 16 h. The reaction was monitored by TLC. The resulting reaction mixture was concentrated under reduced pressure to get the crude product. The crude product was further purified by reverse phase Biotage (column 30 g, C18) eluted with 90% MeCN in water to get(S)-4-ethyl-8-fluoro-4-hydroxy-9,11-dimethyl-10-nitro-1,12-dihydro-14H-pyrano[3′,4′:6,7]indolizino[1,2-b]quinoline-3,14(4H)-dione (AV-D097-Int-05, 400 mg, 0.91 mmol, 77% yield) as a brown solid.

MS (ESI): 440.12 [M+H]⁺: ¹H NMR (400 MHz, DMSO-d₆): δ 8.21 (d, J=10.0 Hz, 1H), 7.32 (s, 1H), 6.57 (brs, 1H), 5.44 (brs, 2H), 5.30 (brs, 2H), 2.53 (s, 3H), 2.36 (s, 3H), 1.99-1.81 (m, 2H), 0.87 (t, J=7.6 Hz, 3H).

### f. Synthesis of(S)-10-amino-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-DL097)

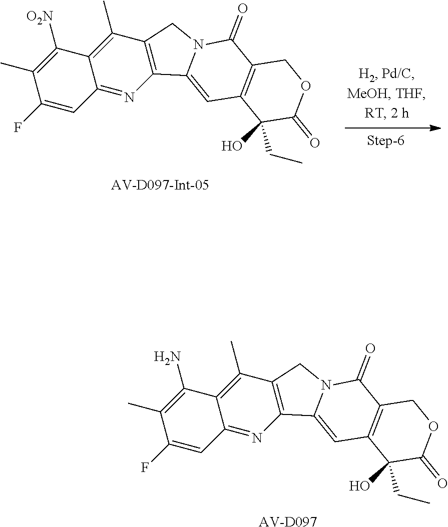

A stirred solution of(S)-4-ethyl-8-fluoro-4-hydroxy-9,11-dimethyl-10-nitro-1,12-dihydro-14H-pyrano[3′,4′:6,7]indolizino[1,2-b]quinoline-3,14(4H)-dione (AV-D097-Int-07, 120 mg, 0.273 mmol) in THF:MeOH (1:1)(2 mL) was treated with 10% Pd/C (40 mg, 50% wet) and kept under H₂ balloon atmosphere for 16 h. The reaction was monitored by TLC. Reaction mixture was filtered through celite bed and washed with MeOH. The filtrate was evaporated under reduced pressure and further purified by RP prep HPLC to get(S)-10-amino-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D097, 8 mg, 0.019 mmol, 7% yield) as a yellow solid.

PREP-HPLC Conditions: GEMINI, C18, (250*21.2 mm) 5μ, A: 0.1% FA IN IN WATER; B: A CN:H₂O (80:20) 0/45, 12/65,12.05/98@12 ML/MIN; diluent: A CN+THF+DMSO

MS (ESI): 410.16 [M+H]⁺: ¹H NMR (400 MHz, DMSO-d₆): δ 7.23 (s, 1H), 7.12 (d, J=11.2 Hz, 1H), 6.48 (brs, 1H), 5.64 (brs, 2H), 5.42 (brs, 2H), 5.21 (brs, 2H), 3.01 (s, 3H), 2.19 (s, 3H), 1.89-1.82 (m, 2H), 0.87 (t, J=7.2 Hz, 3H).

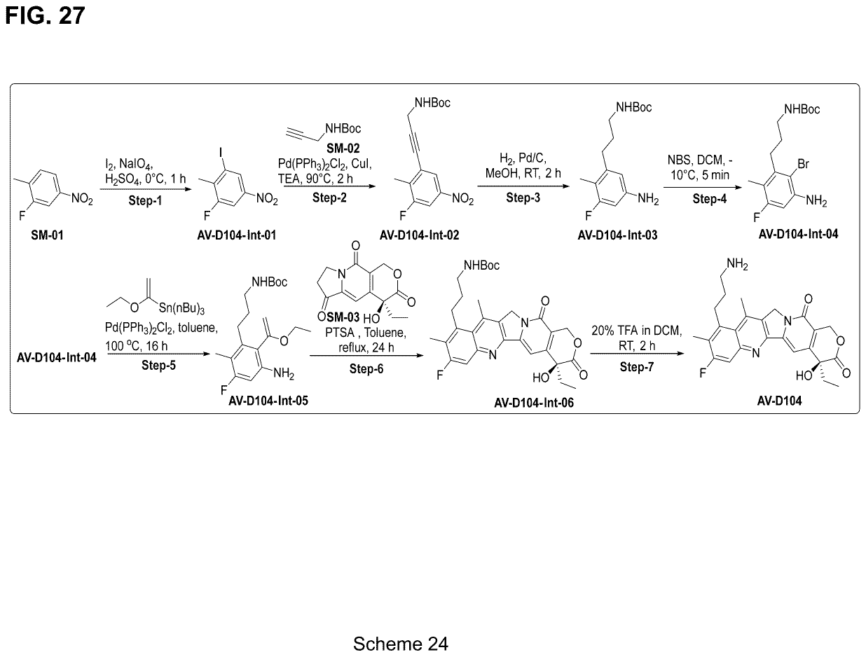

### XXIV. Synthetic Scheme for(S)-10-(3-aminopropyl)-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D104, Scheme 24, FIG. 27)

### a. Synthesis of 1-fluoro-3-iodo-2-methyl-5-nitrobenzene (AV-D104-Int-01)

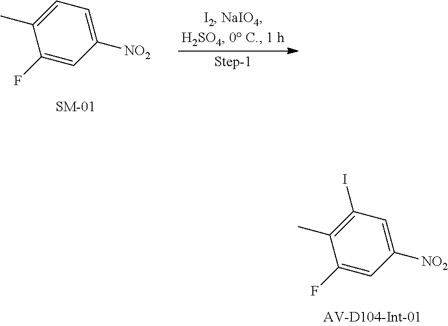

Sodium periodate (2.74 g, 12.89 mmol) was added in 3 equal portions to a mixture of concentrate sulfuric acid (100 mL) and iodine powder (8.15 g, 32.23 mmol) at 0° C. Then, the reaction mixture was allowed to warm up to room temperature over 1 h. This oxidiser solution was slowly added to a mixture of 2-fluoro-4-nitrotoluene (10 g, 64.46 mol) in concentrate sulfuric acid (10 mL) at 0° C. and stirred the mixture for 1 h at 25~30° C. before pouring the solution into ice water (500 mL) containing sodium thiosulphate, solid was precipitated and filtered to obtain crude. The crude product was further purified by reverse phase Biotage (C18 column, 12 g) eluted with 42% MeCN in water to get 1-fluoro-3-iodo-2-methyl-5-nitrobenzene (AV-D104-Int-01, 6 g, 0.067 mmol, 48% yield) as a pale yellow solid.

¹H NMR (400 MHz, CDCl₃): δ 8.49 (brs, 1H), 7.90 (dd, J=2.4, 8.8 Hz, 1H), 2.46 (d, J=2.8 Hz, 3H).

### b. Synthesis of tert-butyl (3-(3-fluoro-2-methyl-5-nitrophenyl)prop-2-yn-1-yl)carbamate (AV-D104-Int-02)

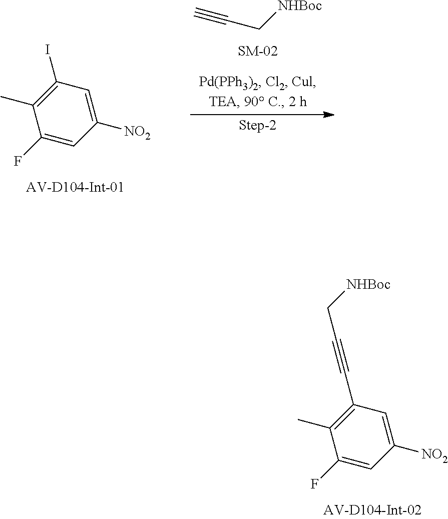 [SMILES: C#CCNC(=O)OC(C)(C)C ; Cc1c(F)cc([N+](=O)[O-])cc1I ; Cc1c(F)cc([N+](=O)[O-])cc1C#CCNC(=O)OC(C)(C)C]

To a stirred solution of 1-fluoro-3-iodo-2-methyl-5-nitrobenzene (AV-D104-Int-01, 1 g, 3.55 mmol) and tert-butyl prop-2-yn-1-ylcarbamate (1.4 g, 8.89 mmol) in TEA (10 mL) was added CuI (68 mg, 0.356 mmol) at room temperature (RT) and degassed with nitrogen for 10 min. Then, was added Pd(PPh₃)₂Cl₂ (62 mg, 0.025 mmol), de-gassed for another 5 min and reaction was stirred at 90° C. for 2 h. The reaction was cooled to RT, diluted with water (10 mL) and extracted with EtOAc (2×30 mL). The combined organic layer was washed with brine (50 mL), dried over anhydrous Na₂SO₄, filtered and concentrated under reduced pressure to obtain crude compound. The crude compound was purified by silica gel column chromatography (SiO₂, 100-200 mesh, 20% EtOAc/hexane) to afford tert-butyl (3-(3-fluoro-2-methyl-5-nitrophenyl)prop-2-yn-1-yl)carbamate (AV-D104-Int-02, 1.1 g, 3.57 mmol, 90% yield) as a brown solid.

¹H NMR (400 MHz, CDCl₃): δ 8.07 (brs, 1H), 7.84 (dd, J=2.0, 8.8 Hz, 1H), 4.82 (brs, 1H), 4.21 (d, J=5.2 Hz, 2H), 2.42 (d, J=2.8 Hz, 3H), 1.48 (s, 9H).

### c. Synthesis of tert-butyl (3-(5-amino-3-fluoro-2-methylphenyl)propyl)carbamate (AV-D104-Int-03)

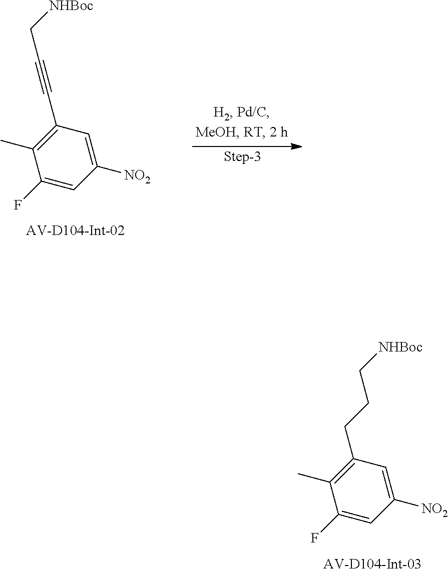 [SMILES: Cc1c(F)cc([N+](=O)[O-])cc1C#CCNC(=O)OC(C)(C)C ; Cc1c(F)cc([N+](=O)[O-])cc1CCCNC(=O)OC(C)(C)C]

To a stirred solution of tert-butyl (3-(3-fluoro-2-methyl-5-nitrophenyl)prop-2-yn-1-yl)carbamate (AV-D104-Int-02, 1.6 g, 5.194 mmol) in MeOH (16 mL) was added 10% Pd/C (500 mg, 30% by weight, 50% wet). The reaction was kept under H₂ balloon atmosphere for 4 h while being monitored by TLC. The reaction was filtered through celite bed and the celite bed was washed with MeOH (3×50 mL). The filtrate was concentrated under reduced pressure to get tert-butyl (3-(5-amino-3-fluoro-2-methylphenyl)propyl)carbamate (AV-D104-Int-03, 1.5 g, 4.177 mmol, quantitative yield) as a brown gummy. The crude as such used for next step.

MS (ESI): 283.14 [M+H]⁺; ¹H NMR (400 MHz, DMSO-d₆): δ 6.87 (t, J=5.2 Hz, 1H), 6.19 (d, J=2.0 Hz, 1H), 6.12 (dd, J=2.0, 12.0 Hz, 1H), 5.04 (brs, 2H), 2.93 (q, J=6.8 Hz, 2H), 2.39 (t, J=7.6 Hz, 2H), 1.94 (d, J=2.0 Hz, 3H), 1.55 (quint, 2H), 1.37 (s, 9H).

### d. Synthesis of tert-butyl (3-(3-amino-2-bromo-5-fluoro-6-methylphenyl)propyl)carbamate (AV-D104-Int-04)

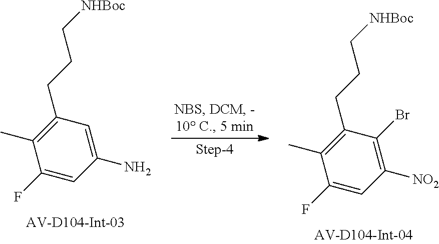 [SMILES: Cc1c(F)cc(N)cc1CCCNC(=O)OC(C)(C)C ; Cc1c(F)cc([N+](=O)[O-])c(Br)c1CCCNC(=O)OC(C)(C)C]

To a stirred solution of tert-butyl (3-(3-fluoro-2-methyl-5-aminophenyl)prop-2-yn-1-yl)carbamate (AV-D104-Int-03, 2 g, 7.092 mmol), in DCM (700 mL) was added NBS (1.262 g, 7.092 mmol) at −10° C. The reaction mixture was stirred at same temperature for 5 min. The reaction was monitored by TLC. The reaction was diluted with water (20 mL) and extracted with DCM (2×100 mL). The combined organic layer was washed with brine (50 mL), dried over anhydrous Na₂SO₄, filtered and concentrated under reduced pressure to obtain crude compound. The crude compound was purified by silica gel column chromatography (SiO₂, 100-200 mesh, 10% EtOAc/hexane) to afford tert-butyl (3-(3-amino-2-bromo-5-fluoro-6-methylphenyl)propyl)carbamate (AV-D104-Int-04, 1.9 g, 5.277 mmol, 74% yield) as a brown solid.

MS (ESI): 360.96 [M+H]⁺; ¹H NMR (400 MHz, DMSO-d₆): δ 6.92. (t, J=5.6 Hz, 1H), 6.46 (d, J=12.0 Hz, 1H), 5.34 (brs, 2H), 2.99 (q, J=6.4 Hz, 2H), 2.70-2.65 (m, 2H), 2.06 (d, J=1.6 Hz, 3H), 1.57-1.50 (m, 2H), 1.38 (s, 9H).

### e. Synthesis of tert-butyl (3-(3-amino-2-(1-ethoxyvinyl)-5-fluoro-6-methylphenyl)propyl)carbamate (AV-D104-Int-05)

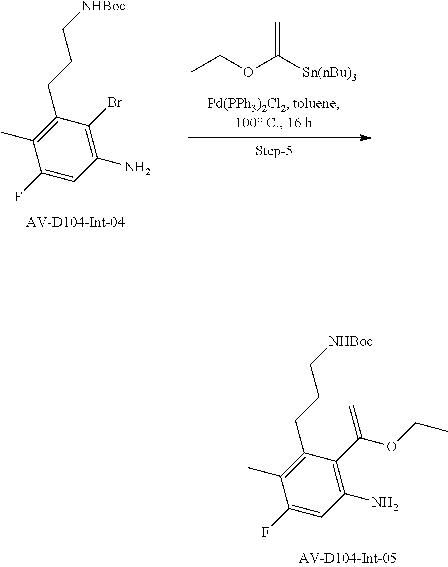

To a stirred solution of tert-butyl (3-(3-amino-2-bromo-5-fluoro-6-methylphenyl)propyl)carbamate (AV-D104-Int-04, 2*1 g, 2.77 mmol) and tributyl(1-ethoxyvinyl) stannane (SM-02, 2*1.5 g, 5.5 mmol) in toluene (2*10 mL) was added Pd(PPh₃)₄ (2*96 mg, 0.037 mmol) at room temperature. The reaction mixture was stirred at 110° C. for 16 h. The reaction was monitored by TLC. The reaction was cooled to 25° C. and the reaction was diluted with water (20 mL) and extracted with EtOAc (2×30 mL). The combined organic layer was washed with brine (30 mL), dried over anhydrous Na₂SO₄, filtered and concentrated under reduced pressure to obtain crude compound. The crude compound was purified by silica gel column chromatography (SiO₂, 100-200 mesh, 15% EtOAc/hexane) to afford tert-butyl (3-(3-amino-2-(1-ethoxyvinyl)-5-fluoro-6-methylphenyl)propyl)carbamate (AV-D104-Int-05, 1.1 g, 3.125 mmol, 56% yield) as a brown semi solid.

MS (ESI): 353.2 [M+H]⁺;

### f. Synthesis of tert-butyl(S)-(3-(4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamate (AV-D104-Int-06)

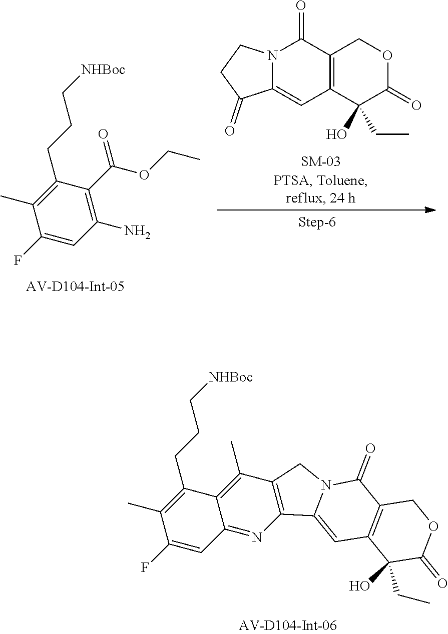

To a stirred solution of tert-butyl (3-(3-amino-2-(1-ethoxyvinyl)-5-fluoro-6-methylphenyl)propyl)carbamate (AV-D104-Int-05, 100 mg, 0.308 mmol), and(S)-4-ethyl-4 -hydroxy-7,8-dihydro-1H-pyrano[3,4-f]indolizine-3,6,10 (4H)-trione (SM-03, 41 mg, 0.154 mmol) in toluene (4 mL) was added p-toluenesulfonic acid (4 mg, 0.0308 mmol) at room temperature and the reaction mixture was heated at 110° C. for 16 h. The reaction was monitored by TLC. The resulting reaction mixture was concentrated under reduced pressure to get tert-butyl (S)-(3-(4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamate (AV-D104-Int-06, 110 mg, 0.199 mmol), as a brown gummy. The crude as such used for next step.

MS (ESI): 552.39 [M+H]⁺;

### g. Synthesis of(S)-10-(3-aminopropyl)-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D104)

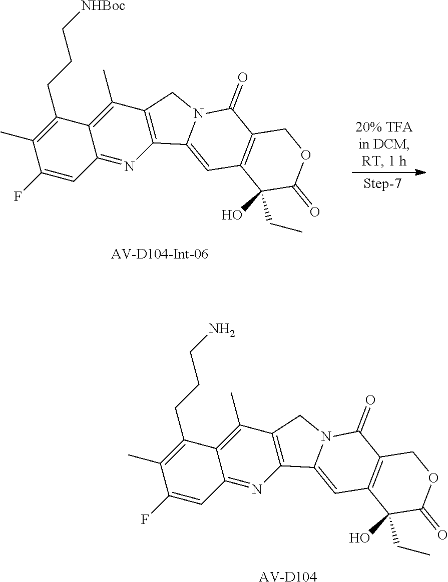 [SMILES: CC[C@@]1(O)C(=O)OCc2c1cc1n(c2=O)Cc2c-1nc1cc(F)c(C)c(CCCNC(=O)OC(C)(C)C)c1c2C ; CC[C@@]1(O)C(=O)OCc2c1cc1n(c2=O)Cc2c-1nc1cc(F)c(C)c(CCCN)c1c2C ; "AV-D104"]

Tert-butyl(S)-(3-(4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamate (AV-D104-Int-06, 110 mg, 0.199 mmol) in 20% TFA in DCM (2 mL) was stirred at RT for 1 h. The reaction was monitored by LCMS. The resulting reaction mixture was concentrated to get crude product. The crude product was further purified by prep HPLC to get(S)-10-(3-aminopropyl)-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D104, 9 mg, 0.0199 mmol, 11% yield) as a light brown solid.

PREP-HPLC Conditions: GEMINI,C18, (250*21.5 mm) 5μ: Buffer-A: 0.2% FA IN WATER; B: Acetonitrile, H₂O (80:20), 0/20, 12/35.5, 12.1/98@12 ML/MIN; diluent: ACN+H₂O

MS (ESI): 450.24 [M−H]⁻; ¹H NMR (400 MHz, DMSO-d₆): δ 7.78. (d, J=10.4 Hz, 1H), 7.28 (s 1H), 6.54 (brs, 1H), 5.44 (s, 2H), 5.29 (s, 2H), 3.25-3.15 (m, 2H), 2.97 (s, 3H), 2.96-2.93 (m, 2H), 2.45 (s, 3H), 1.90-1.81 (m, 4H), 0.87 (t, J=7.2 Hz, 3H).

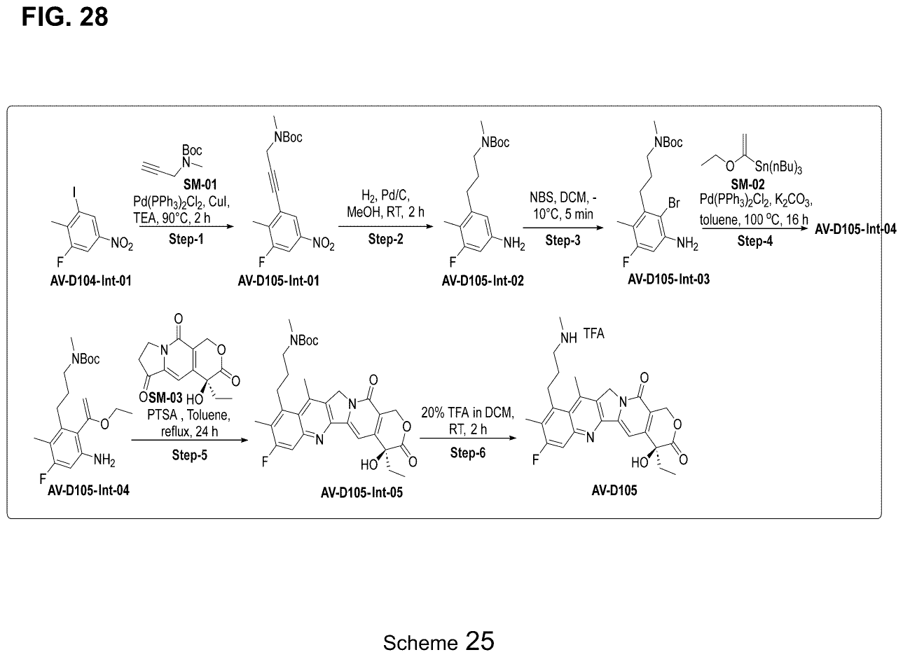

### XXV. Synthetic scheme for(S)-4-ethyl-8-fluoro-4-hydroxy-9,11-dimethyl-10-(3-(methylamino)propyl)-1,12-dihydro-14H-pyrano[3′,4′:6,7]indolizino[1,2-b]quinoline-3,14(4H)-dione (AV-D105, Scheme 25, FIG. 28)

### a. Synthesis of tert-butyl (3-(3-fluoro-2-methyl-5-nitrophenyl)prop-2-yn-1-yl)(methyl)carbamate (AV-D105-Int-01)

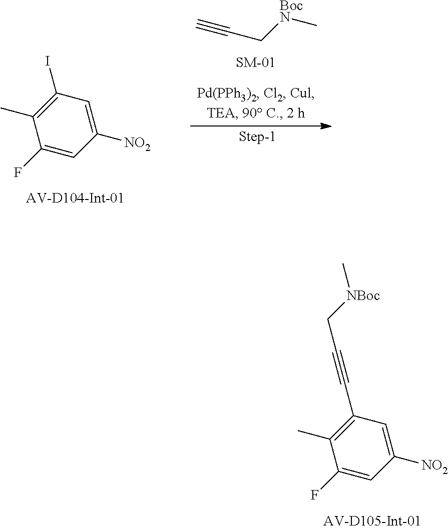 [SMILES: C#CCN(C)C(=O)OC(C)(C)C ; Cc1c(F)cc([N+](=O)[O-])cc1I ; Cc1c(F)cc([N+](=O)[O-])cc1C#CCN(C)C(=O)OC(C)(C)C]

To a stirred solution of 1-fluoro-3-iodo-2-methyl-5-nitrobenzene (AV-D104-Int-01, 2.3 g, 8.18 mmol) and tert-butyl methyl(prop-2-yn-1-yl)carbamate (SM-01, 3.4 g, 20.46 mmol) in TEA (20 mL), was added CuI (155 mg, 0.818 mmol) and degassed with nitrogen for 5 min. then, Pd(PPh₃)₂Cl₂ (143 mg, 0.205 mmol) was at room temperature and degassed with nitrogen for 10 min, and the reaction mixture was stirred at 90° C. for 2 h. The reaction was diluted with water (10 mL) and extracted with EtOAc (2×30 mL). The combined organic layer was washed with brine (100 mL), dried over anhydrous Na₂SO₄, filtered and concentrated under reduced pressure to obtain crude compound. The crude compound was purified by silica gel column chromatography (SiO₂, 100-200 mesh, 5% EtOAc/hexane) to afford tert-butyl (3-(3-fluoro-2-methyl-5-nitrophenyl)prop-2-yn-1-yl)(methyl)carbamate (AV-D105-Int-01, 2.0 g, 6.211 mmol, 76% yield) as a brown solid.

¹H NMR (400 MHz, CDCl₃): δ 8.07 (d, J=0.8 Hz, 1H), 7.84 (dd, J=2.0, 8.8 Hz, 1H), 4.32 (s, 2H), 2.99 (s, 3H), 2.43 (d, J=2.4 Hz, 3H), 1.49 (s, 9H).

### b. Synthesis of tert-butyl (3-(5-amino-3-fluoro-2-methylphenyl)propyl)(methyl)carbamate (AV-D105-Int-02)

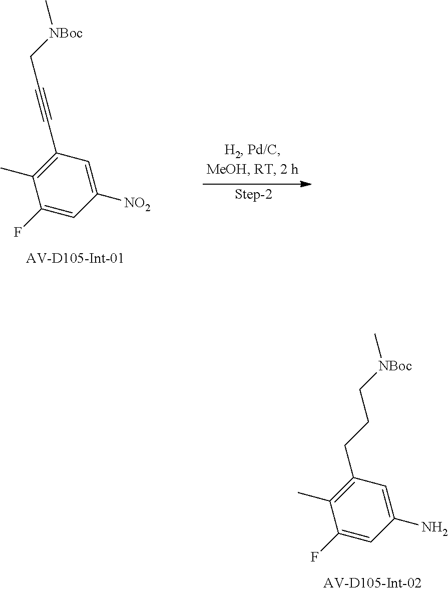 [SMILES: Cc1c(F)cc([N+](=O)[O-])cc1C#CCN(C)C(=O)OC(C)(C)C ; Cc1c(F)cc(N)cc1CCCN(C)C(=O)OC(C)(C)C]

To a stirred solution of tert-butyl (3-(3-fluoro-2-methyl-5-nitrophenyl)prop-2-yn-1-yl)(methyl)carbamate (AV-D105-Int-01, 500 mg, 1.552 mmol) in MeOH (5 mL) was added 10% Pd/C (150 mg, 30% by weight, 50% wet). The reaction was stirred under H₂ balloon atmosphere for 4 h while being monitored by TLC. The reaction was filtered through celite bed and washed with MeOH (30 mL). The filtrate was concentrated under reduced pressure to get tert-butyl (3-(5-amino-3-fluoro-2-methylphenyl)propyl)(methyl)carbamate (AV-D105-Int-02, 500 mg, 1.337 mmol, quantitative yield) as a brown gummy. The crude as such used for next step. MS (ESI): 297.19 [M+H]⁺.

### c. Synthesis of tert-butyl (3-(3-amino-2-bromo-5-fluoro-6-methylphenyl)propyl)(methyl)carbamate (AV-D105-Int-03)

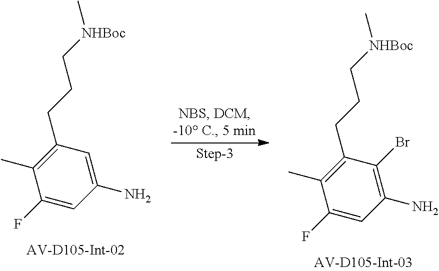

To a stirred solution of tert-butyl (3-(5-amino-3-fluoro-2-methyl phenyl)propyl)(methyl)carbamate (AV-D105-Int-02, 850 mg, 2.87 mmol) in DCM (255 mL), was added NBS (508 mg, 2.87 mmol) at −10° C. The reaction mixture was stirred at same temperature for 5 min. The reaction was diluted with water (20 mL) and extracted with DCM (2×100 mL). The combined organic layer was washed with brine (50 mL), dried over anhydrous Na₂SO₄, filtered and concentrated under reduced pressure to obtain crude compound. The crude compound was purified by silica gel column chromatography (SiO₂, 100-200 mesh, 10% EtOAc/hexane) to afford tert-butyl (3-(3-amino-2-bromo-5-fluoro-6-methylphenyl)propyl)(methyl)carbamate (AV-D105-Int-03, 500 mg, 1.34 mmol, 50% yield) as a brown solid.

MS (ESI): 377.12 [M+2H]⁺;

### d. Synthesis of tert-butyl (3-(3-amino-2-(1-ethoxyvinyl)-5-fluoro-6-methylphenyl)propyl)(methyl)carbamate (AV-D105-Int-04)

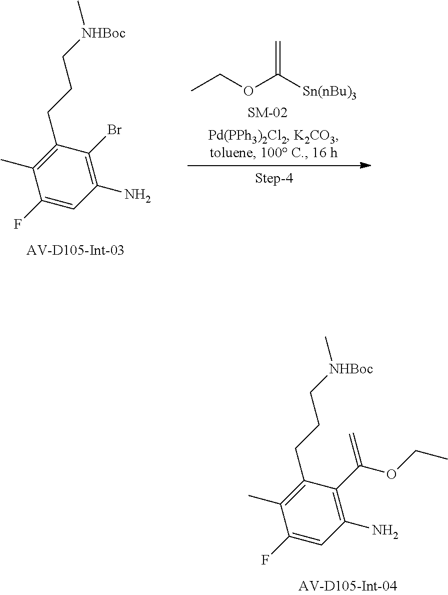

To a stirred solution of tert-butyl (3-(3-amino-2-bromo-5-fluoro-6-methylphenyl)propyl)(methyl)carbamate (AV-D105-Int-03, 100 mg, 0.267 mmol) and tributyl(1-ethoxyvinyl) stannane (SM-02, 144 mg, 0.401 mmol) in 1,4-dioxane (2 mL), was added K₂CO₃ (110 mg, 0.802 mmol) and de-gassed the mixture with nitrogen or 10 min. Then, Pd(PPh₃)₂Cl₂ (9.3 mg, 0.033 mmol) was added at room temperature and de-gassed the mixture for 10 min. The reaction mixture was stirred at 110° C. for 16 h. The reaction was monitored by TLC. The reaction mixture was cooled to 25° C., was diluted with water (2 mL) and extracted with EtOAc (2×30 mL). The combined organic layer was washed with brine (10 mL), dried over anhydrous Na₂SO₄, filtered and concentrated under reduced pressure to obtain crude compound. The crude compound was purified by silica gel column chromatography (SiO₂, 100-200 mesh, 10% EtOAc/hexane) to afford tert-butyl (3-(3-amino-2-(1-ethoxyvinyl)-5-fluoro-6-methylphenyl)propyl)(methyl)carbamate (AV-D105-Int-04, 50 mg, 0.137 mmol, 55% yield) as a brown semi solid.

MS (ESI): 367.26 [M+H]⁺;

### c. Synthesis of tert-butyl(S)-(3-(4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-D105-Int-05)

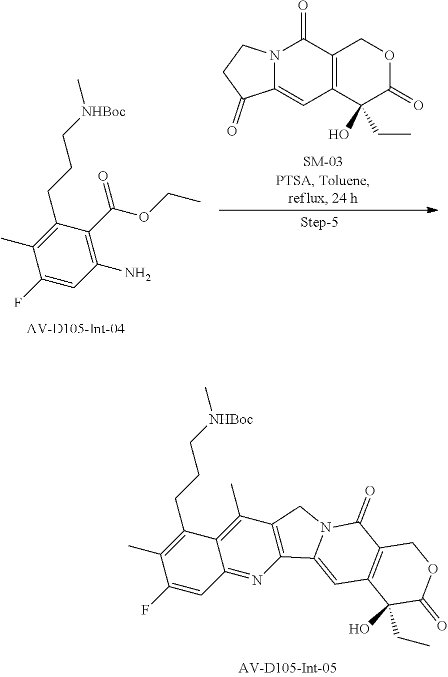

To a stirred tert-butyl (3-(3-amino-2-(1-ethoxyvinyl)-5-fluoro-6-methylphenyl)propyl)(methyl)carbamate (AV-D105-Int-04, 50 mg, 0.136 mmol), and(S)-4-ethyl-4-hydroxy-7,8-dihydro-1H-pyrano[3,4-f]indolizine-3,6,10 (4H)-trione (SM-03, 17 mg, 0.154 mmol) in toluene (5 mL) was added p-toluenesulfonic acid (2.5 mg, 0.0136 mmol) at room temperature and the reaction mixture was heated at 110° C. for 24 h. The reaction was monitored by TLC. The resulting reaction mixture was concentrated under reduced pressure to get tert-butyl (S)-(3-(4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-D105-Int-05, 30 mg, 0.0531 mmol), as a brown solid. The crude as such used for next step.

MS (ESI): 566.44 [M+H]⁺;

### f. Synthesis of(S)-4-ethyl-8-fluoro-4-hydroxy-9,11-dimethyl-10-(3-(methylamino)propyl)-1,12-dihydro-14H-pyrano[3′,4′:6,7]indolizino[1,2-b]quinoline-3,14(4H)-dione (AV-D105)

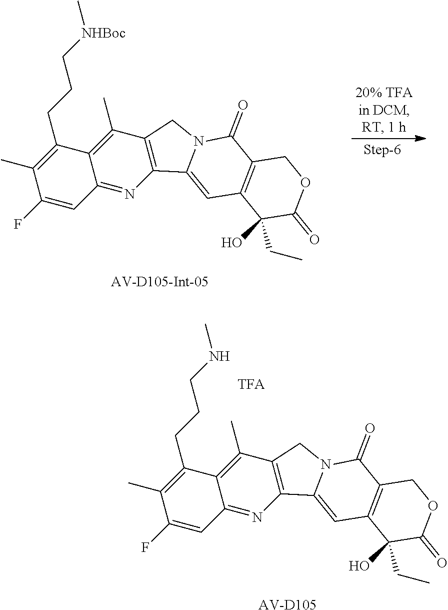

A solution of tert-butyl(S)-(3-(4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-D105-Int-05, 100 mg, 0.177 mmol) in 20% TFA in DCM (1 mL) was stirred at RT for 1 h. The reaction was monitored by LCMS. The resulting reaction mixture was concentrated to get crude product. The crude product was further purified by prep HPLC to get(S)-4-ethyl-8-fluoro-4-hydroxy-9,11-dimethyl-10-(3-(methylamino)propyl)-1,12-dihydro-14H-pyrano[3′,4′:6,7]indolizino[1,2-b]quinoline-3,14(4H)-dione (AV-D105, 3 mg, 0.0064 mmol, 3.5% yield) as a light brown solid (TFA salt).

PREP-HPLC Conditions: GEMINI, C18, (250*21.5 mm) 5μ: buffer-A: 0.1% TFA IN WATER; B: Acetonitrile, H₂O (80:20), 0/5,20/55,22/55,22.1/98@12 ML/MIN; diluent: ACN+H₂O+THF

MS (ESI): 466.24 [M+H]⁺; ¹H NMR (400 MHz, DMSO-d₆): δ 8.34 (brs, 2H), 7.82 (d, J=10.4 Hz, 1H), 7.29 (s, 1H), 6.53 (s, 1H), 5.44 (s, 2H), 5.31 (s, 2H), 3.32-3.24 (m, 2H), 3.11 (t, J=7.6 Hz, 2H), 2.98 (s, 3H), 2.67 (s, 3H), 2.49 (s, 3H), 1.89-1.83 (m, 4H), 0.87 (t, J=7.2 Hz, 3H).

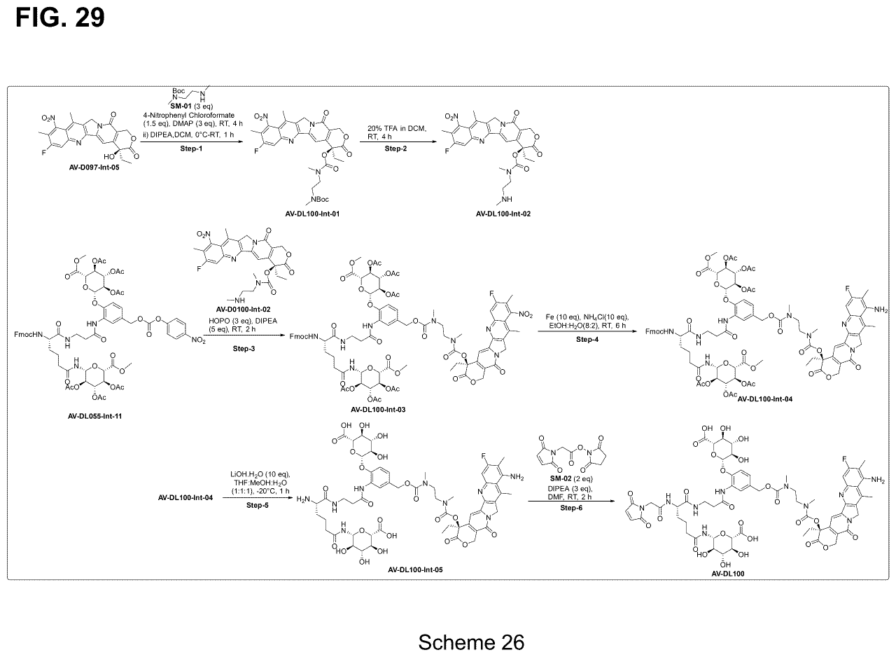

### XXVI. Synthetic scheme for (2S,3S,4S,5R,6R)-6-((S)-6-((3-((5-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-6-carboxy-3,4,5-trihydroxytetrahydro-2H-pyran-2-yl)oxy)phenyl)amino)-3-oxopropyl)amino)-5-(2-(2,5-dioxo-2,5-dihydro-1H-pyrrol-1-yl)acetamido)-6-oxohexanamido)-3,4,5-trihydroxytetrahydro-2H-pyran-2-carboxylic Acid (AV-DL100, Scheme 26, FIG. 29)

### a. Synthesis of(S)-tert-butyl (4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl) ethane-1,2-diylbis(methylcarbamate)(AV-DL100-Int-01)

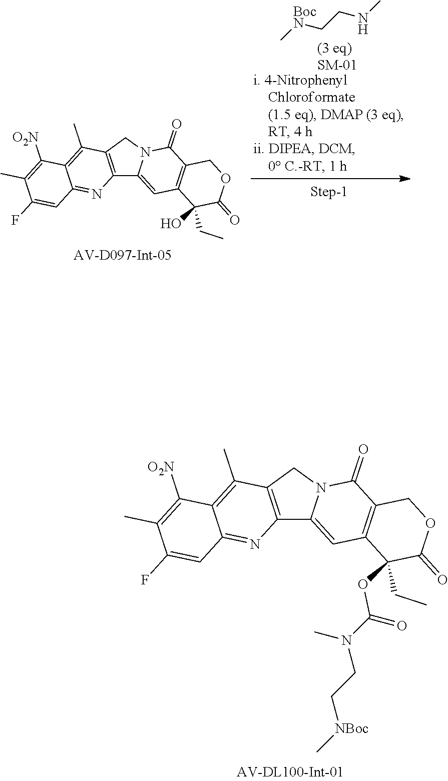

To a stirred solution of(S)-4-ethyl-8-fluoro-4-hydroxy-9,11-dimethyl-10-nitro-1,12-dihydro-14H-pyrano[3′,4′:6,7]indolizino[1,2-b]quinoline-3,14(4H)-dione (AV-D097-Int-05, 250 mg, 0.569 mmol), 4-nitrophenyl Chloroformate (228 mg, 1.138 mmol) in DCM (12 mL) was added DMAP (347 mg, 2.847 mmol) at 0° C. and stirred at RT for 1 h. The reaction was monitored by TLC. The reaction mixture was further treated with tert-butyl methyl(2-(methylamino)ethyl)carbamate (319 mg, 1.688 mmol) and DIPEA (0.2 mL, 1.688 mmol) in DCM and stirred at RT for 1 h. The resulting reaction mixture was concentrated under reduced pressure and triturated with diethyl ether to get crude product. The crude product was further purified by reverse phase Biotage (column 30 g, C18) eluted with 62% MeCN in water to get (S)-tert-butyl (4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl) ethane-1,2-diylbis(methylcarbamate)(AV-DL100-Int-01, 340 mg, 0.520 mmol, 91% yield) as an off white solid, The crude was used as such for the next step.

MS (ESI): 654.28 [M+H]⁺.

### b. Synthesis of(S)-4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL100-Int-02)

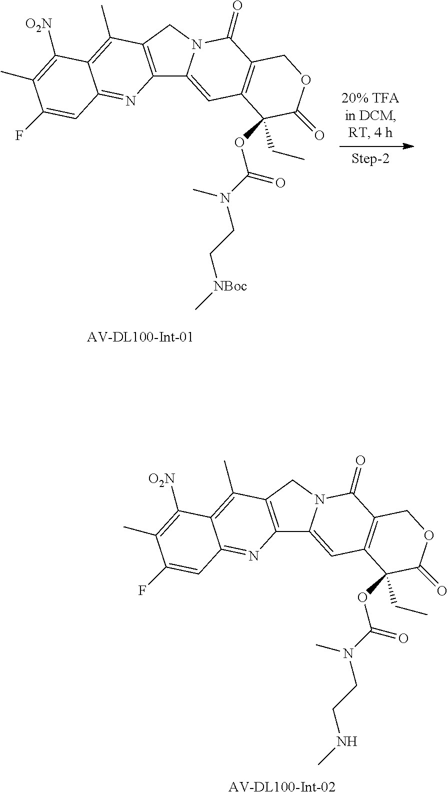 [SMILES: CC[C@@]1(OC(=O)N(C)CCN(C)C(=O)OC(C)(C)C)C(=O)OCc2c1cc1n(c2=O)Cc2c-1nc1cc(F)c(C)c([N+](=O)[O-])c1c2C ; CC[C@@]1(OC(=O)N(C)CCNC)C(=O)OCc2c1cc1n(c2=O)Cc2c-1nc1cc(F)c(C)c([N+](=O)[O-])c1c2C]

To (S)-tert-butyl (4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl) ethane-1,2-diylbis(methylcarbamate)(AV-DL100-Int-01, 250 mg, 0.383 mmol) was added 20% TFA in DCM (2.5 mL) at 0° C. and stirred at RT for 4 h. The reaction was monitored by TLC. The resulting reaction mixture was concentrated under reduced pressure to get(S)-4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL100-Int-02, 200 mg, 0.362 mmol, quantitative yield) as a light brown solid.

MS (ESI): 554.28 [M+H]⁺.

### c. Synthesis of (2R,3R,4S,5S,6S)-2-((S)-5-((((9H-fluoren-9-yl)methoxy)carbonyl)amino)-6-((3-((5-((((2-(((((S)-4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-(methoxycarbonyl)tetrahydro-2H-pyran-3,4,5-triyl Triacetate (AV-DL100-Int-03)

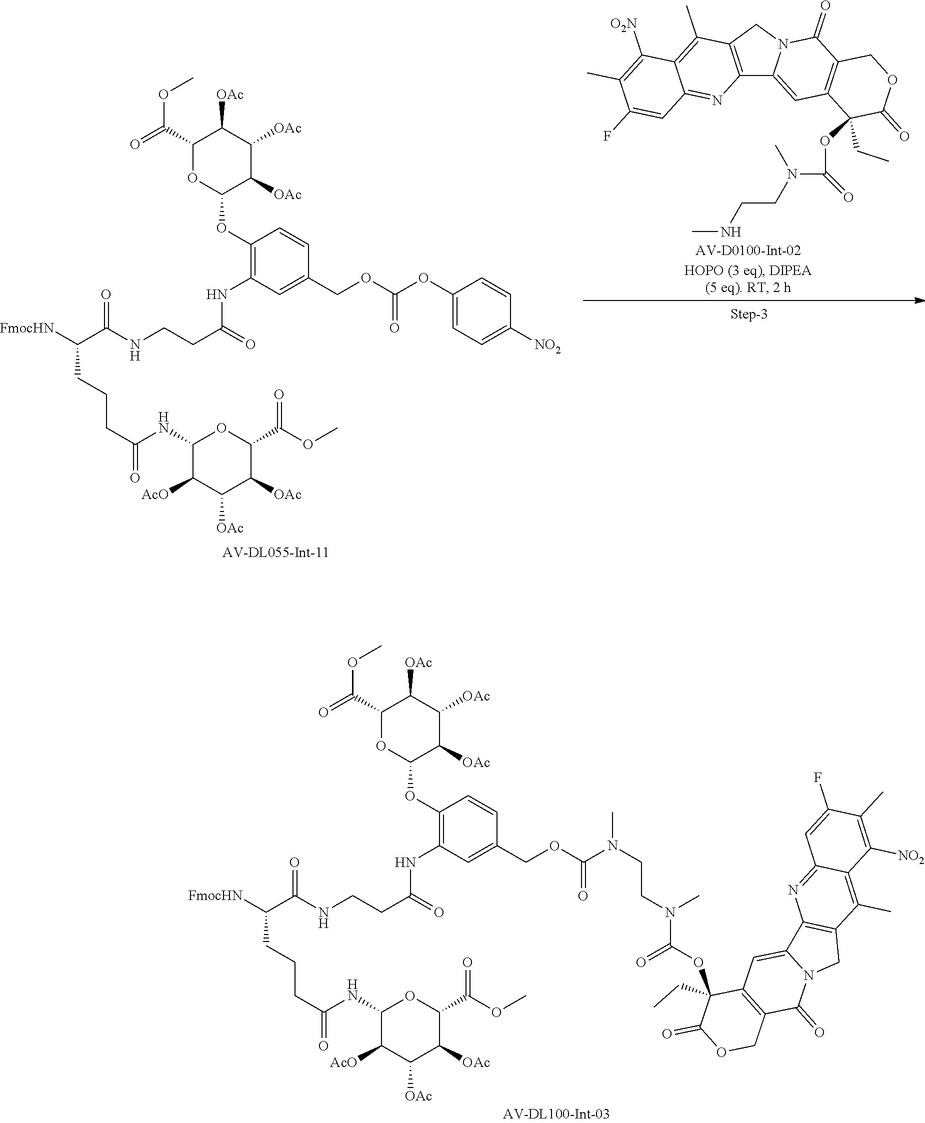

To a stirred solution of (2R,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, XVII.k, 250 mg, 0.36 mmol), (S)-4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl methyl(2-(methylamino)ethyl)carbamate (AV-DL100-Int-02, 200 mg, 0.36 mmol) in DMF (5 mL) were added DIPEA (0.30 mL, 1.80 mmol), HOPO (120 mg, 1.08 mmol) at 0° C. and stirred at RT for 2 h. The reaction was monitored by TLC. The resulting reaction mixture was concentrated under reduced pressure to get the crude product. The crude product was further purified by reverse phase Biotage (column 30 g, C18) eluted with 40% MeCN in water to get (2R,3R,4S,5S,6S)-2-((S)-5-((((9H-fluoren-9-yl)methoxy)carbonyl)amino)-6-((3-((5-((((2-(((((S)-4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL100-Int-03, 350 mg, 0.196 mmol, 54% yield) as a light brown solid.

MS (ESI): 1787.69 [M+H]⁺.

### d. Synthesis of (2R,3R,4S,5S,6S)-2-((S)-5-((((9H-fluoren-9-yl)methoxy)carbonyl)amino)-6-((3-((5-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-(methoxycarbonyl)tetrahydro-2H-pyran-3,4,5-triyl Triacetate (AV-DL100-Int-04)

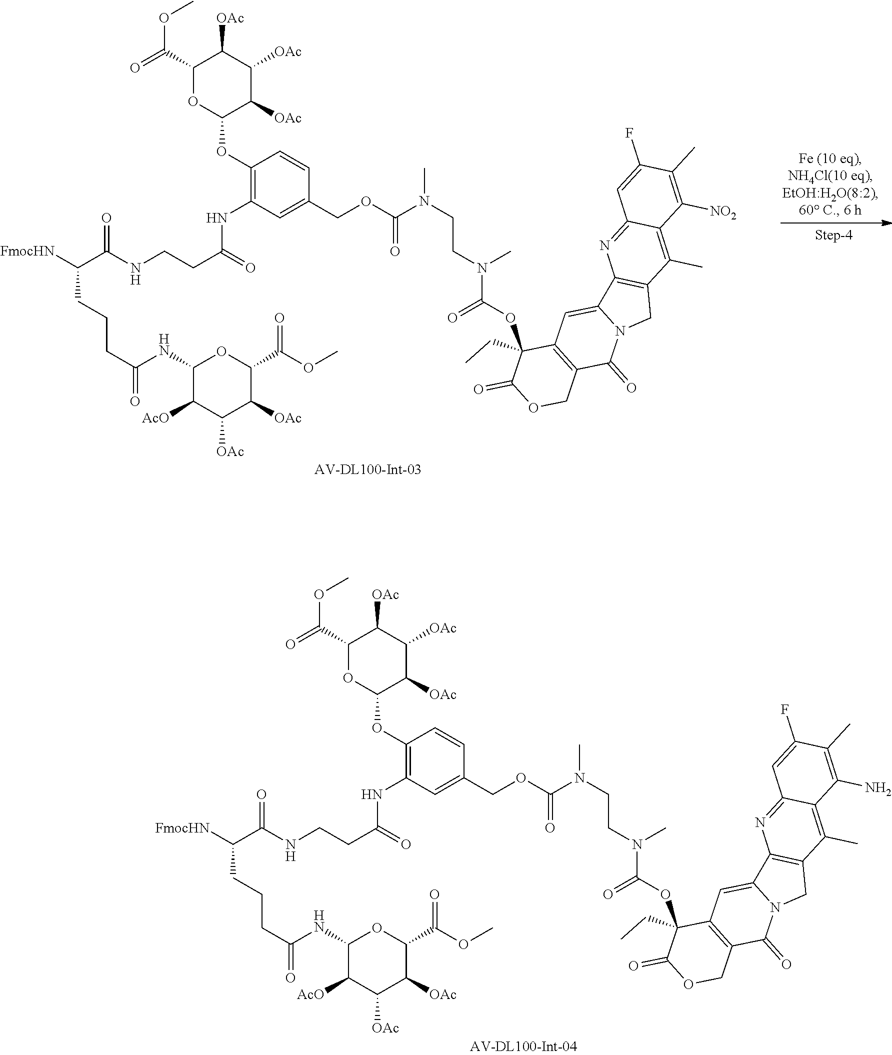

To a stirred solution of (2R,3R,4S,5S,6S)-2-((S)-5-((((9H-fluoren-9-yl)methoxy)carbonyl)amino)-6-((3-((5-((((2-(((((S)-4-ethyl-8-fluoro-9,11-dimethyl-10-nitro-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL100-Int-03, 250 mg, 0.14 mmol) in EtOH:H₂O (1:1)(4 mL) was added Fe powder (38 mg, 0.14 mmol) and NH₄Cl (38 mg, 0.14 mmol) at RT, and stirred at 60° C. for 6 h. The reaction was monitored by TLC. The reaction mixture was filtered through celite bed and washed with EtOH and concentrated under reduced pressure to get (2R,3R,4S,5S,6S)-2-((S)-5-((((9H-fluoren-9-yl)methoxy)carbonyl)amino)-6-((3-((5-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL100-Int-04, 240 mg, 0.138 mmol) as a yellow gum.

MS (ESI): 1756.78 [M+H]⁺.

### e. Synthesis of (2S,3S,4S,5R,6S)-6-(4-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)phenoxy)-3,4,5-trihydroxytetrahydro-2H-pyran-2-carboxylic Acid (AV-DL100-Int-05)

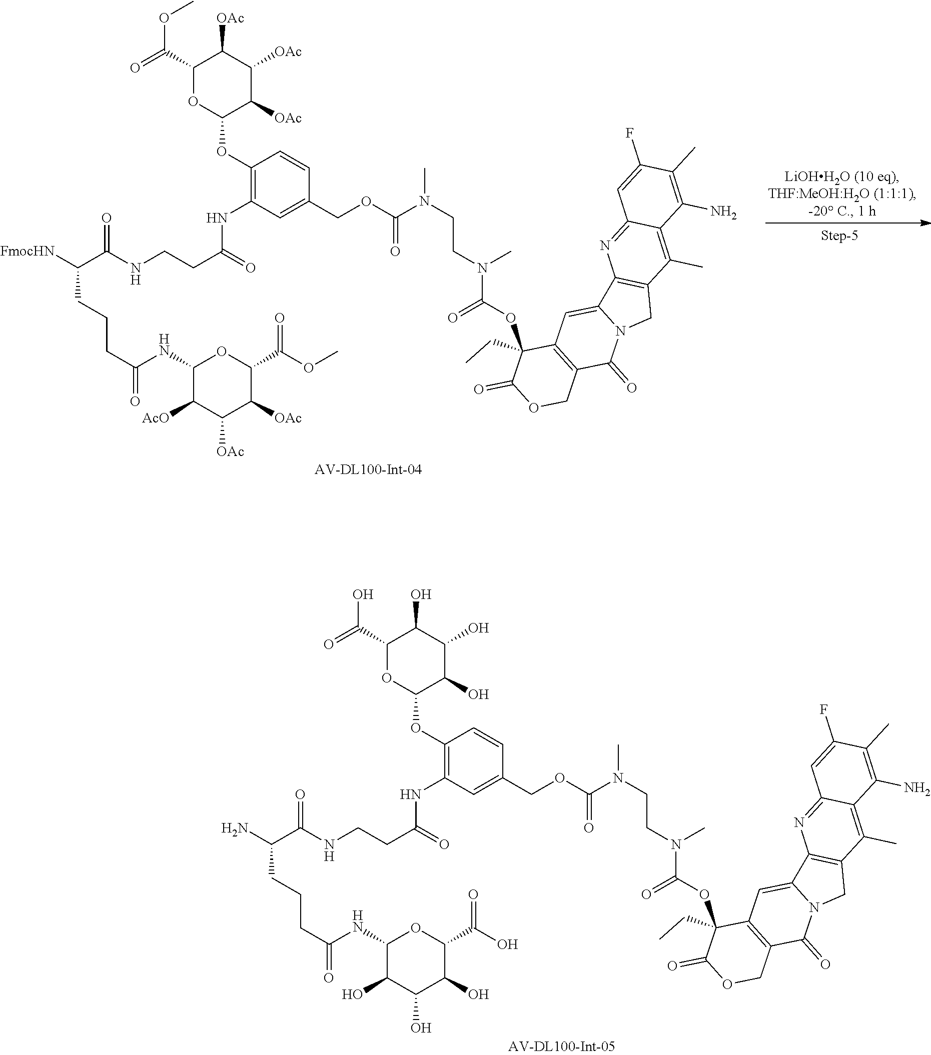

To a stirred solution of (2R,3R,4S,5S,6S)-2-((S)-5-((((9H-fluoren-9-yl)methoxy)carbonyl)amino)-6-((3-((5-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL100-Int-04, 240 mg, 0.169 mmol) in THF:H₂O:MeOH (1:1:1)(5 mL) was added LiOH·H₂O (70 mg, 1.641 mmol) at 0° C. and stirred at RT for 1 h. The reaction was monitored by LCMS. The reaction mixture was concentrated under reduced pressure to get crude product. The crude product was further purified by reverse phase Biotage (column 12 g, C18) eluted with 28% MeCN in water to get (2S,3S,4S,5R,6S)-6-(4-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)phenoxy)-3,4,5-trihydroxytetrahydro-2H-pyran-2-carboxylic acid (AV-DL100-Int-05, 100 mg, 0.0798 mmol, 28% yield) as a pale yellow solid.

MS (ESI): 1255.61 [M+H]⁺.

### f. Synthesis of (2S,3S,4S,5R,6R)-6-((S)-6-((3-((5-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-6-carboxy-3,4,5-trihydroxytetrahydro-2H-pyran-2-yl)oxy)phenyl)amino)-3-oxopropyl)amino)-5-(2-(2,5-dioxo-2,5-dihydro-1H-pyrrol-1-yl)acetamido)-6-oxohexanamido)-3,4,5-trihydroxytetrahydro-2H-pyran-2-carboxylic Acid (AV-DL100)

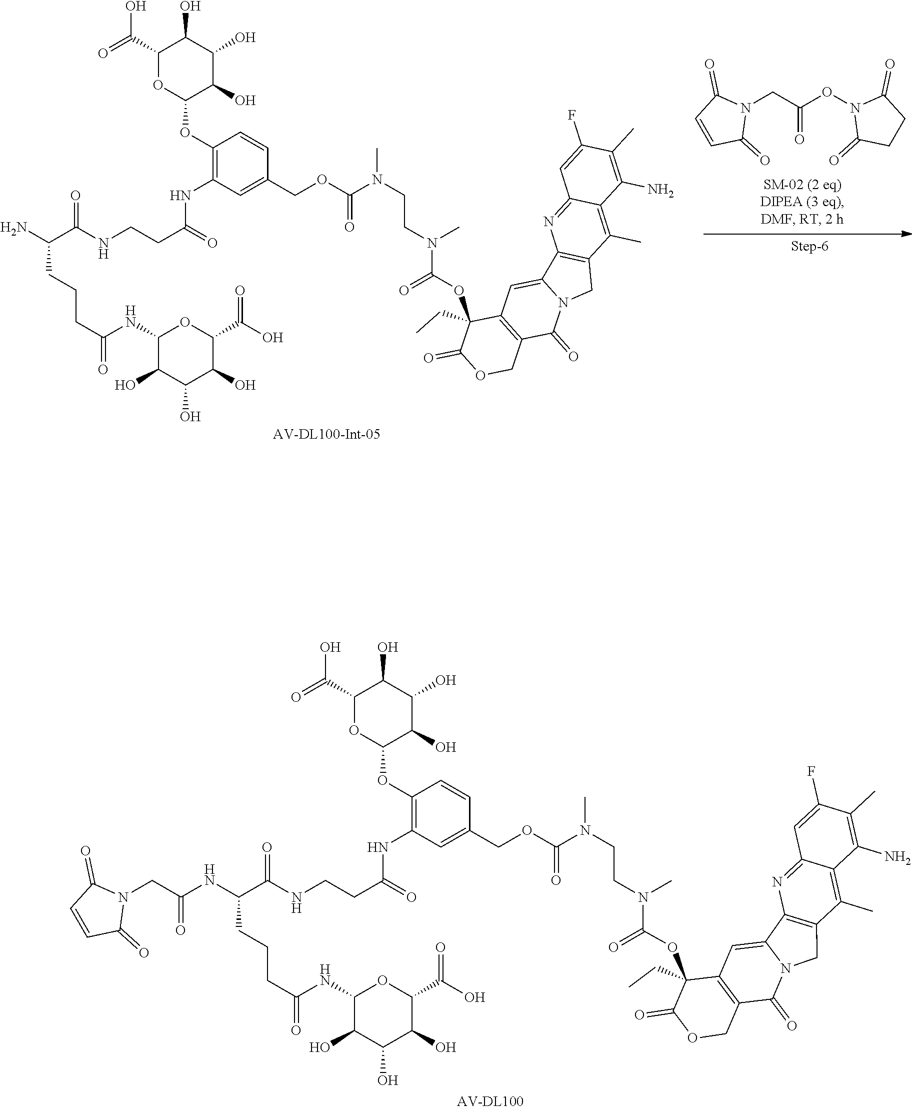 [SMILES: CC[C@@]1(OC(=O)N(C)CCN(C)C(=O)OCc2ccc(O[C@@H]3O[C@H](C(=O)O)[C@@H](O)[C@H](O)[C@H]3O)c(NC(=O)CCNC(=O)[C@@H](N)CCCC(=O)N[C@@H]3O[C@H](C(=O)O)[C@@H](O)[C@H](O)[C@H]3O)c2)C(=O)OCc2c1cc1n(c2=O)Cc2c-1nc1cc(F)c(C)c(N)c1c2C ; O=C(CN1C(=O)C=CC1=O)ON1C(=O)CCC1=O ; CC[C@@]1(OC(=O)N(C)CCN(C)C(=O)OCc2ccc(O[C@@H]3O[C@H](C(=O)O)[C@@H](O)[C@H](O)[C@H]3O)c(NC(=O)CCNC(=O)[C@H](CCCC(=O)N[C@@H]3O[C@H](C(=O)O)[C@@H](O)[C@H](O)[C@H]3O)NC(=O)CN3C(=O)C=CC3=O)c2)C(=O)OCc2c1cc1n(c2=O)Cc2c-1nc1cc(F)c(C)c(N)c1c2C]

To a stirred solution of (2S,3S,4S,5R,6S)-6-(4-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(3-((S)-2-amino-6-(((2R,3R,4S,5S,6S)-6-carboxy-3,4,5-trihydroxytetrahydro-2H-pyran-2-yl)amino)-6-oxohexanamido)propanamido)phenoxy)-3,4,5-trihydroxytetrahydro-2H-pyran-2-carboxylic acid (AV-DL100-Int-05, 100 mg, 0.079 mmol) in DMF (2.5 mL) was added 2,5-dioxopyrrolidin-1-yl 2-(2,5-dioxo-2,5-dihydro-1H-pyrrol-1-yl)acetate (SM-02, 19 mg, 0.079 mmol), DIPEA (4 μL, 0.237 mmol) at 0° C. and stirred at RT for 1 h. The reaction was monitored by LCMS. The resulting reaction mixture was concentrated under reduced pressure to get crude product which was purified by RP prep HPLC and fractions were lyophilized to get (2S,3S,4S,5R,6R)-6-((S)-6-((3-((5-((((2-(((((S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl)oxy)carbonyl)(methyl)amino)ethyl)(methyl)carbamoyl)oxy)methyl)-2-(((2S,3R,4S,5S,6S)-6-carboxy-3,4,5-trihydroxytetrahydro-2H-pyran-2-yl)oxy)phenyl)amino)-3-oxopropyl)amino)-5-(2-(2,5-dioxo-2,5-dihydro-1H-pyrrol-1-yl)acetamido)-6-oxohexanamido)-3,4,5-trihydroxytetrahydro-2H-pyran-2-carboxylic acid (AV-DL100, 4.5 mg, 0.0032 mmol, 4% yield) as a light brown solid

PREP-HPLC Conditions: X BRIDGE, C18, (250*19 mm) 5μ, A: 0.1% TFA IN IN WATER; B: A CN:H₂O (80:20), 0/20,15/45,17/45, 17.1/98; diluent: MeCN, Water, THF.

MS (ESI): 1393.76 [M+H]⁺: ¹H NMR (400 MHz, DMSO-d₆): δ 12.74 (brs, 2H), 9.05 (brs, 1H), 8.42-8.32 (m, 2H), 8.25-7.95 (m, 2H), 7.20-6.95 (m, 7H), 5.90-5.55 (m, 3H), 5.49-5.30 (m, 4H), 5.29-4.90 (m, 6H), 4.83-4.70 (m, 2H), 4.30-4.05 (m, 3H), 3.91-3.70 (m, 1H), 3.69-3.40 (m, 5H), 3.30-2.90 (m, 11H), 2.79-2.65 (m, 5H), 2.22-1.95 (m, 10H), 1.65-1.40 (m, 4H), 0.95-0.80 (m, 3H).

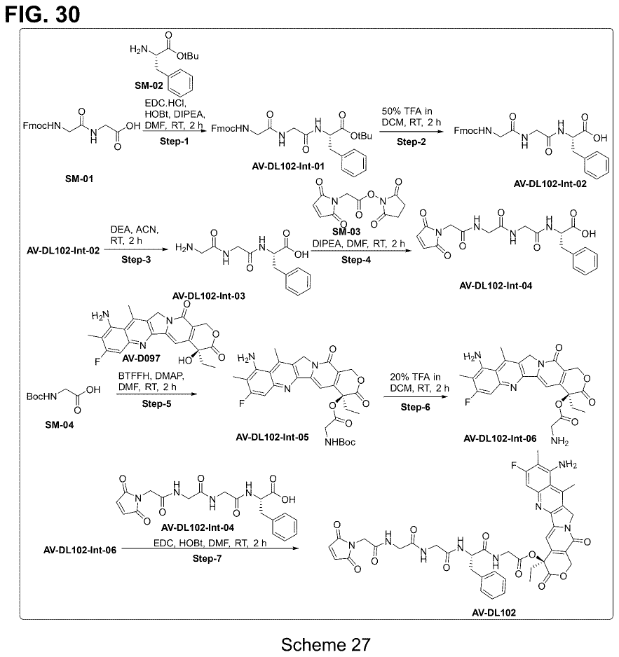

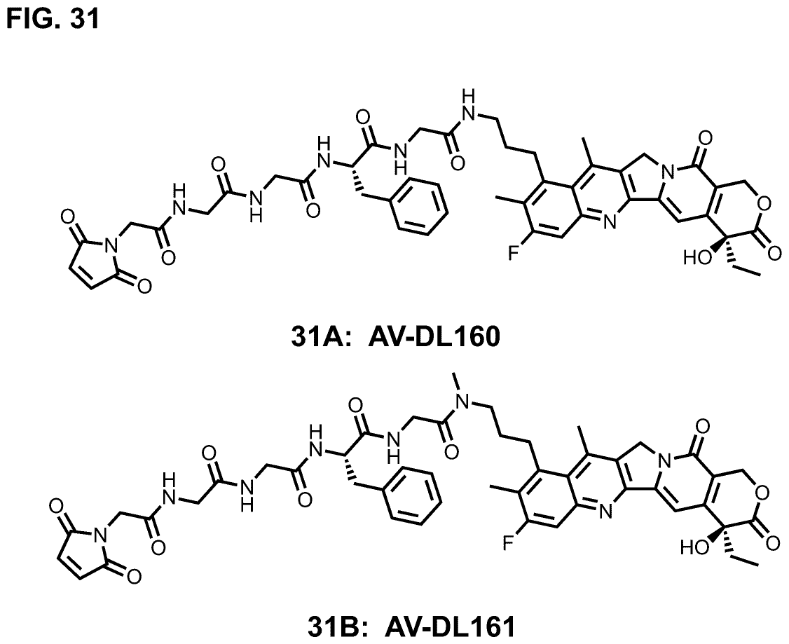

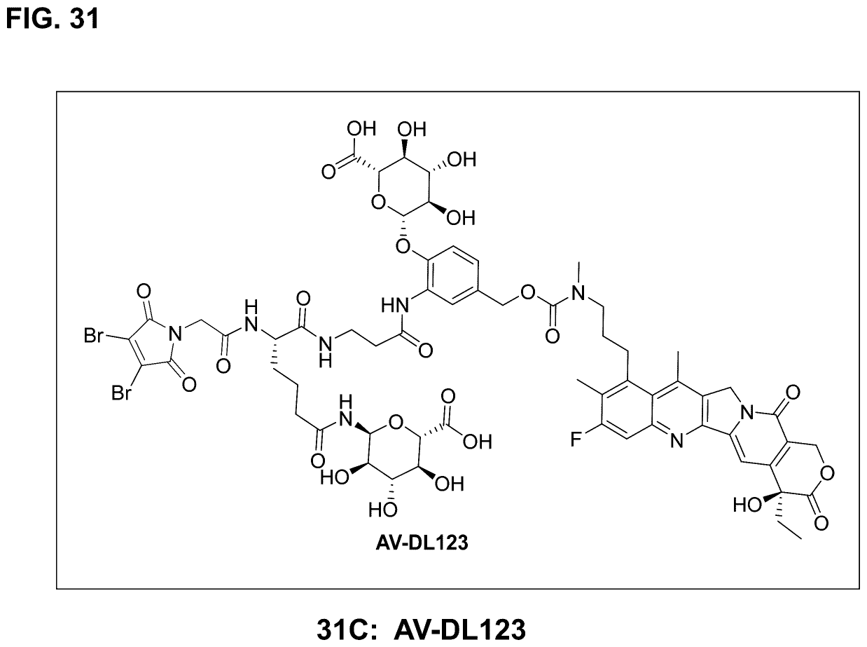

### XXVII. Synthetic scheme for(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl(2-(2,5-dioxo-2,5-dihydro-1H-pyrrol-1-yl) acetyl)glycylglycyl-L-phenylalanylglycinate (AV-DL102, Scheme 27, FIG. 30)

### a. Synthesis of tert-butyl (((9H-fluoren-9-yl)methoxy)carbonyl)glycylglycyl-L-phenylalaninate (AV-DL102-Int-01)

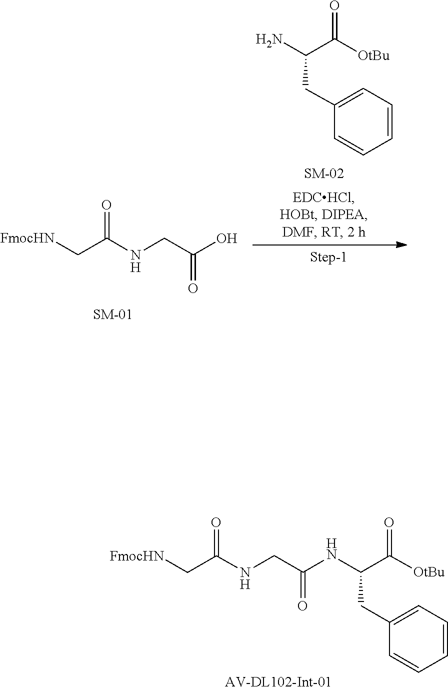

To a stirred solution of (((9H-fluoren-9-yl)methoxy)carbonyl)glycylglycine (SM-01, 5 g, 14.12 mmol), tert-butyl L-phenylalaninate (3.6 g, 14.12 mmol) and DIPEA (3.7 mL, 21.19) in DMF (50 mL) was added EDC·HCl (4 g, 21.18 mmol), HOBt (2.86 g, 21.86 mmol) at 0° C. and stirred at RT for 2 h. The reaction was monitored by TLC. The resulting reaction mixture was concentrated under reduced pressure to get crude product. The crude product was further purified by RP Biotage and fractions were lyophilized to get tert-butyl (((9H-fluoren-9-yl)methoxy)carbonyl)glycylglycyl-L-phenylalaninate (AV-DL102-Int-01, 6.5 g, 11.67 mmol, 82%) as an off-white solid.

MS (ESI): 558.40 [M+H]⁺.

### g. Synthesis of (((9H-fluoren-9-yl)methoxy)carbonyl)glycylglycyl-L-phenylalanine (AV-DL102-Int-02)

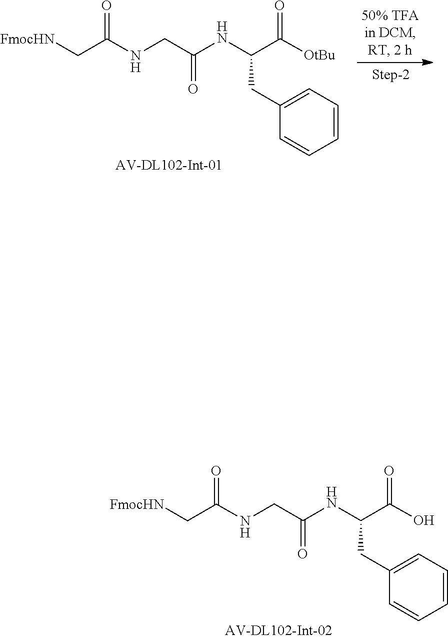

A solution of tert-butyl (((9H-fluoren-9-yl)methoxy)carbonyl)glycylglycyl-L-phenylalaninate (AV-DL102-Int-01, 4 g, 8.98 mmol) in 50% TFA in DCM (40 mL) was stirred at RT for 2 h. The reaction was monitored by LCMS. The resulting reaction mixture was concentrated under reduced pressure to get (((9H-fluoren-9-yl)methoxy)carbonyl)glycylglycyl-L-phenylalanine (AV-DL102-Int-02, 3.6 g, 7.18 mmol, 80% yield) as an off white solid.

MS (ESI): 502.34 [M+H]⁺.

### h. Synthesis of Glycylglycyl-L-phenylalanine (AV-DL102-Int-03)

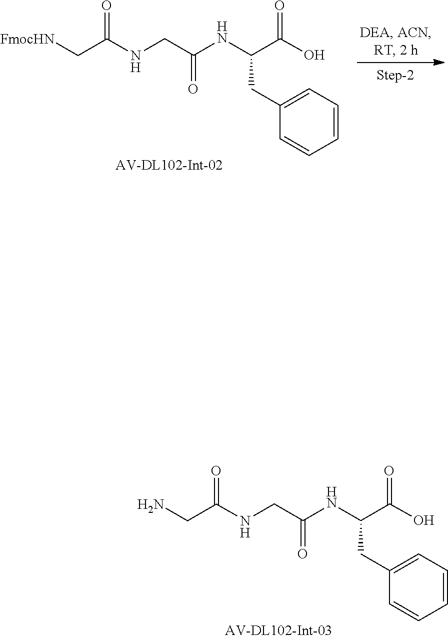

(((9H-Fluoren-9-yl)methoxy)carbonyl)glycylglycyl-L-phenylalanine (AV-DL102-Int-02, 3.6 g, 7.18 mmol) in 25% diethylamine in CH₃CN(36 mL) at 0° C. was stirred at RT for 2 h. The reaction was monitored by TLC. The reaction was concentrated under reduced pressure to get crude product. The crude product was triturated with diethyl ether (3×72 mL) and dried to get glycylglycyl-L-phenylalanine (AV-DL102-Int-03, 1.7 g, 6.09 mmol, quantitative yield) as an off white solid.

MS (ESI): 280.22 [M+H]⁺.

### i. Synthesis of (2-(2,5-dioxo-2,5-dihydro-1H-pyrrol-1-yl) acetyl)glycylglycyl-L-phenylalanine (AV-DL102-Int-04)

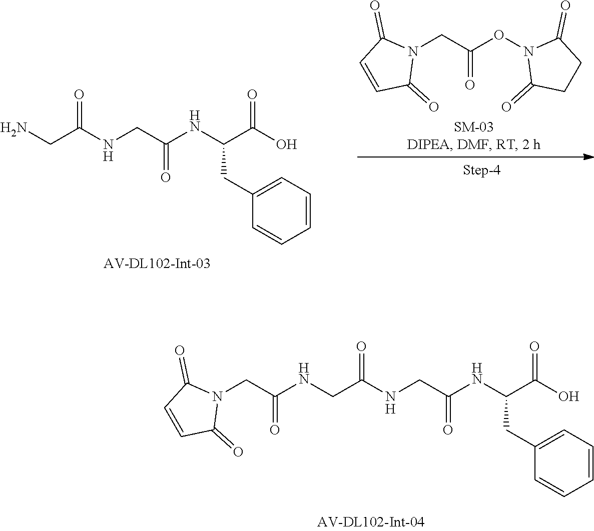 [SMILES: NCC(=O)NCC(=O)N[C@@H](Cc1ccccc1)C(=O)O ; O=C(CN1C(=O)C=CC1=O)ON1C(=O)CCC1=O ; O=C(CNC(=O)CN1C(=O)C=CC1=O)NCC(=O)N[C@@H](Cc1ccccc1)C(=O)O]

To a stirred solution of glycylglycyl-L-phenylalanine (AV-DL102-Int-03, 250 mg, 0.89 mmol), and DIPEA (0.46 mL, 2.68 mmol) in DMF (5 mL) was added 2,5-dioxopyrrolidin-1-yl 2-(2,5-dioxo-2,5-dihydro-1H-pyrrol-1-yl)acetate (SM-03, 225 mg, 0.89 mmol) at 0° C. and stirred at RT for 2 h. The reaction was monitored by LCMS. The resulting reaction mixture was concentrated under reduced pressure to get crude product. The crude product was further purified by RP Biotage and fractions were lyophilized to get (2-(2,5-dioxo-2,5-dihydro-1H-pyrrol-1-yl) acetyl)glycylglycyl-L-phenylalanine (AV-DL102-Int-04, 150 mg, 0.36 mmol, 40% yield) as an off white solid.

MS (ESI): 417.31 [M+H]⁺.

### j. Synthesis of(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl (tert-butoxycarbonyl)glycinate (AV-DL102-Int-05)

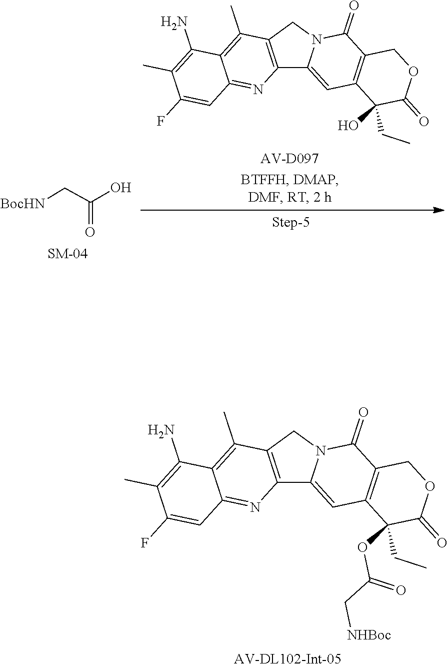

To a stirred solution of (tert-butoxycarbonyl)glycine (SM-04, 156 mg, 0.88 mmol), (S)-10-amino-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D097, 360 mg, 0.88 mmol) in DMF (6 mL) were added DIPEA (0.6 mL, 2.64 mmol), DMAP (12 mg, 0.088 mmol), bis(tetramethylene) formamidinium hexafluorophosphate (BIFFH, 834 mg, 2.64 mmol) sequentially at 0° C. and stirred at RT for 2 h. The reaction was monitored by TLC. The resulting reaction mixture was concentrated under reduced pressure to get crude product which was purified by RP Biotage and fractions were lyophilized to get(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl (tert-butoxycarbonyl)glycinate (AV-DL102-Int-05, 100 mg, 0.177 mmol, 20% yield) as a pale yellow solid.

MS (ESI): 410.16 [M+H]⁺.

### k. Synthesis of(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl glycinate (AV-DL035-Int-06)

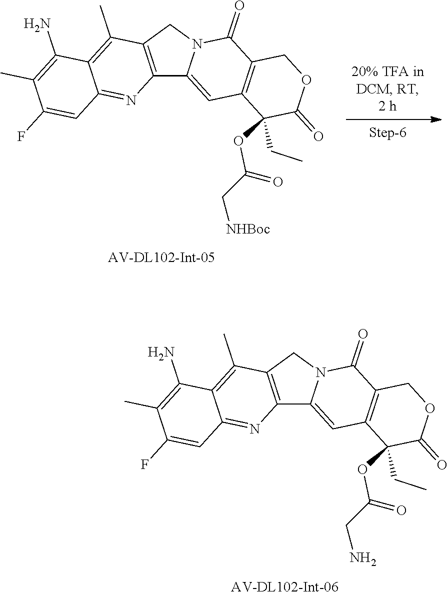 [SMILES: CC[C@@]1(OC(=O)CNC(=O)OC(C)(C)C)C(=O)OCc2c1cc1n(c2=O)Cc2c-1nc1cc(F)c(C)c(N)c1c2C ; CC[C@@]1(OC(=O)CN)C(=O)OCc2c1cc1n(c2=O)Cc2c-1nc1cc(F)c(C)c(N)c1c2C]

A solution of(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl (tert-butoxycarbonyl)glycinate (AV-DL102-Int-05, 40 mg, 0.070 mmol) in 20% TFA in DCM (1 mL) was stirred at RT for 2 h. The reaction was monitored by LCMS. The resulting reaction mixture was concentrated under reduced pressure to get(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl glycinate (AV-DL035-Int-06, 35 mg, 0.075 mmol, quantitative yield) as a brown solid.

MS (ESI): 467.31 [M+H]⁺.

### l. Synthesis of(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl(2-(2,5-dioxo-2,5-dihydro-1H-pyrrol-1-yl) acetyl)glycylglycyl-L-phenylalanylglycinate (AV-DL102)

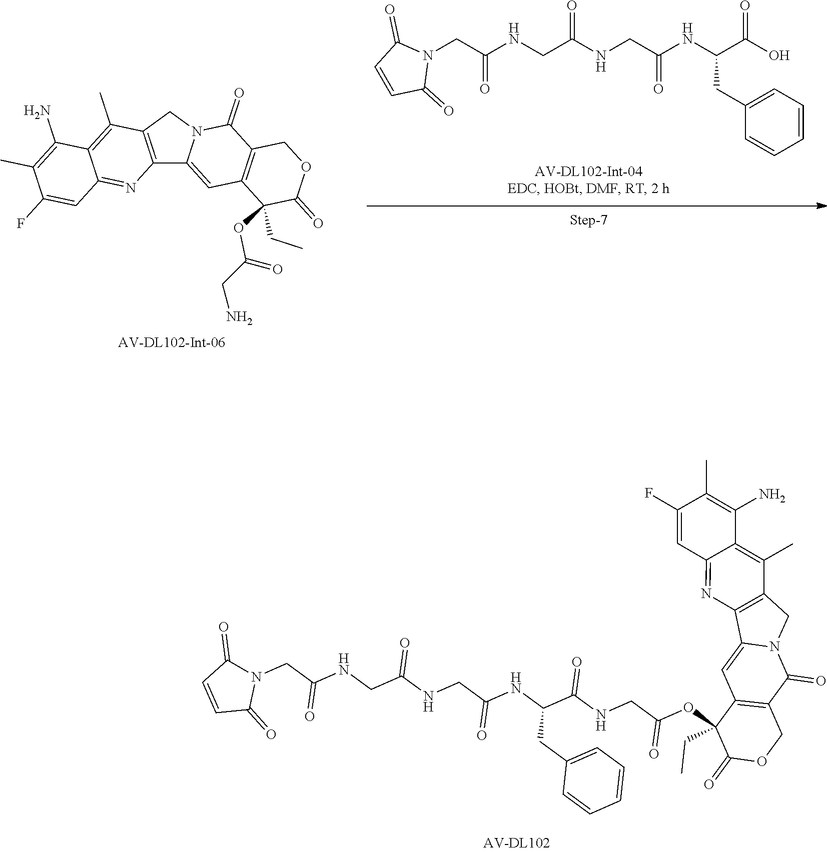 [SMILES: CC[C@@]1(OC(=O)CN)C(=O)OCc2c1cc1n(c2=O)Cc2c-1nc1cc(F)c(C)c(N)c1c2C ; O=C(CNC(=O)CN1C(=O)C=CC1=O)NCC(=O)N[C@@H](Cc1ccccc1)C(=O)O ; CC[C@@]1(OC(=O)CNC(=O)[C@H](Cc2ccccc2)NC(=O)CNC(=O)CNC(=O)CN2C(=O)C=CC2=O)C(=O)OCc2c1cc1n(c2=O)Cc2c-1nc1cc(F)c(C)c(N)c1c2C]

To a stirred solution (2-(2,5-dioxo-2,5-dihydro-1H-pyrrol-1-yl) acetyl)glycylglycyl-L-phenylalanine (AV-DL102-Int-04, 54 mg, 0.128 mmol) and(S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl glycinate (AV-DL102-Int-06, 60 mg, 0.128 mmol) in DMF (0.5 mL) were added EDC·HCl (38 mg, 0.192 mmol), HOBt (26 mg, 0.192) at 0° C. and stirred at RT for 2 h. The reaction was monitored by LCMS. The resulting reaction mixture was concentrated under reduced pressure to get crude product. The crude was purified by RP prep HPLC and fractions were lyophilized to get (S)-10-amino-4-ethyl-8-fluoro-9,11-dimethyl-3,14-dioxo-3,4,12,14-tetrahydro-1H-pyrano[3′,4′:6,7]indolizino[1,2-b]quinolin-4-yl(2-(2,5-dioxo-2,5-dihydro-1H-pyrrol-1-yl) acetyl)glycylglycyl-L-phenylalanylglycinate (AV-DL102, 5.7 mg, 0.0066 mmol, 5%) as a pale yellow solid.

PREP-HPLC Conditions: X-Bridge, C-18 column, 250×19 mm×5 μm A: 0.2% FA in water B: Acetonitrile: H₂O, 0/30, 15/80, 15.05/98; diluent: MeCN, Water, THF.

MS (ESI): 865.4 [M+H]⁺. ¹H NMR (400 MHz, DMSO-d₆): δ 8.60 (t, J=5.6 Hz, 1H), 8.42 (t, J=5.6 Hz, 1H), 8.17 (d, J=8.4 Hz, 1H), 8.05 (t, J=6.0 Hz, 1H), 7.30-7.17 (m, 4H), 7.18-7.16 (m, 1H), 7.11 (s, 1H), 7.08 (s, 2H), 7.05 (s, 1H), 5.67 (s, 2H), 5.49 (s, 2H), 5.22 (s, 2H), 4.57-4.46 (m, 1H), 4.18 (dd, J=5.6, 18.0 Hz, 1H), 4.10-4.04 (m, 3H), 3.74-3.67 (m, 3H), 3.55 (dd, J=5.6, 16.8 Hz, 1H), 3.10-2.98 (m, 4H), 2.79-2.70 (m, 1H), 2.19 (s, 3H), 2.15-2.10 (m, 2H), 0.89 (t, J=7.2 Hz, 3H).

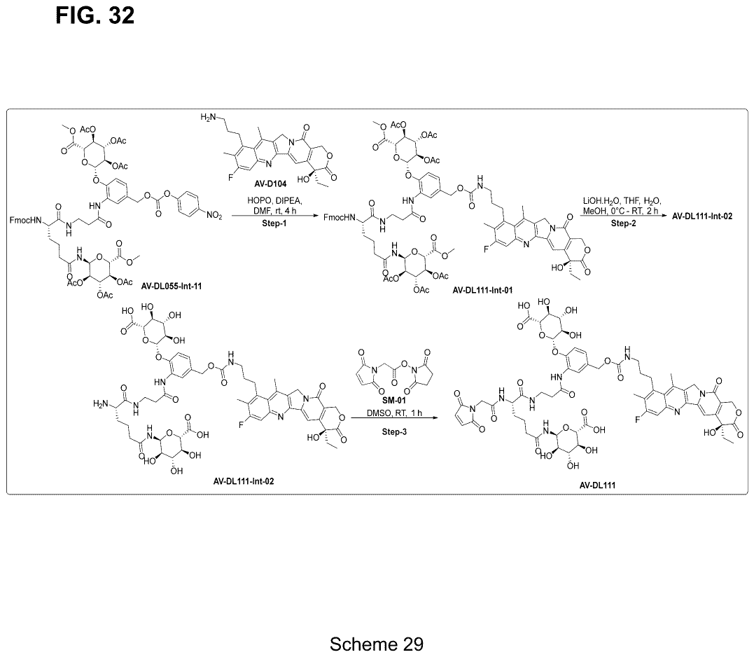

### XXVIII. Synthetic scheme for (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic Acid (AV-DL111, Scheme 29, FIG. 32)

### a. Synthesis of (2S,3R,4S,5S,6S)-2-((S)-5-((((9H-fluoren-9-yl)methoxy)carbonyl)amino)-6-((3-((5-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl Triacetate (AV-DL111-Int-01)

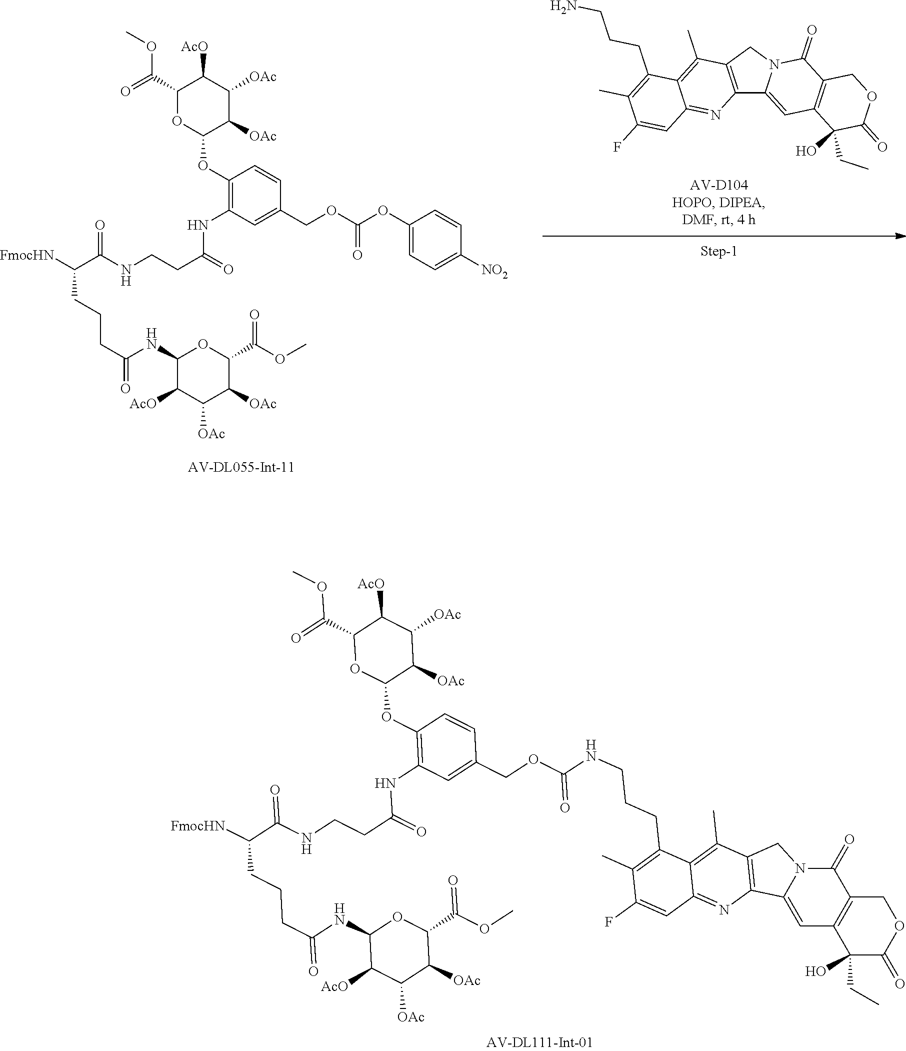

To a stirred solution of (2S,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, Example XVII.k, 300 mg, 0.2186 mmol), (S)-10-(3-aminopropyl)-4-ethyl-8-fluoro-4-hydroxy-9,11-dimethyl-1,12-dihydro-14H-pyrano[3′,4′:6,7]indolizino[1,2-b]quinoline-3,14(4H)-dione (AV-D104, Example XXIV, 78 mg, 0.1749 mmol) in ACN:THF (1:1)(5 mL) were added HOPO (72 mg, 0.6558 mmol) and DIPEA (0.12 mL, 0.6558 mmol) at 0° C., and stirred at RT for 2 h. The reaction was monitored by LCMS. The resulting reaction mixture was concentrated under reduced pressure to get crude product. The crude product was further purified by reverse phase Biotage (column 20 g, C18) eluted with 40% MeCN in water to get (2S,3R,4S,5S,6S)-2-((S)-5-((((9H-fluoren-9-yl)methoxy)carbonyl)amino)-6-((3-((5-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL111-Int-01), 120 mg, 0.071 mmol, 66% yield) as a light brown solid.

MS (ESI): 1684.56 [M+H]⁺.

### b. Synthesis of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic Acid (AV-DL111-Int-02)

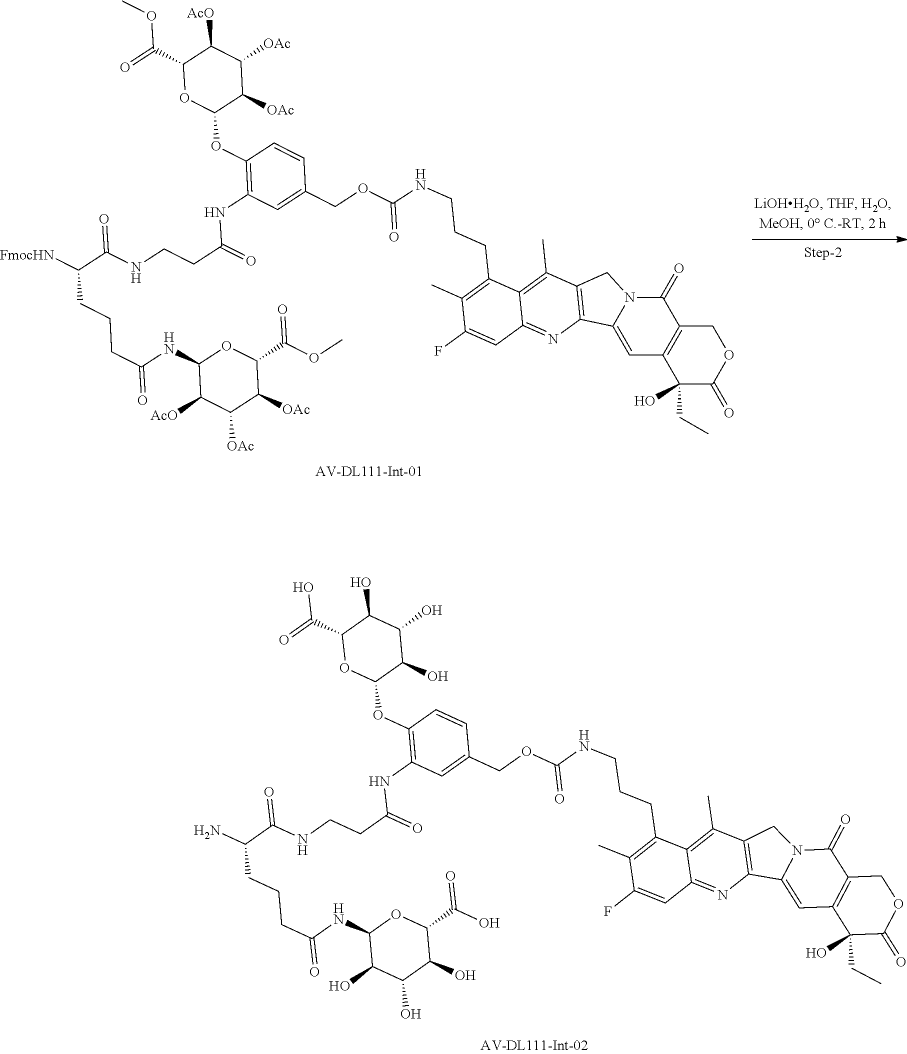

To a stirred solution of (2S,3R,4S,5S,6S)-2-((S)-5-((((9H-fluoren-9-yl)methoxy)carbonyl)amino)-6-((3-((5-((((3-((S)-4-ethyl-8-fluoro-4-hydroxy-9,11-dimethyl- 3,14-dioxo-3,4,12,14-tetrahydro-1H-pyrano[3′,4′:6,7]indolizino[1,2-b]quinolin-10-yl)propyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL111-Int-01, 120 mg, 0.07130 mmol) in THF:H₂O:MeOH (1:1:1)(3 mL) was added LiOH·H₂O (29 mg, 0.7130 mmol) at 0° C. and stirred at same the temperature for 2 h. The reaction was monitored by LCMS. The reaction was concentrated under reduced pressure to get crude product. The crude product was further purified by reverse phase Biotage (column 20 g, C18) eluted with 40% MeCN in water to get (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic acid (AV-DL111-Int-02, 40 mg, 0.0338 mmol, 95% yield) as an off white solid.

MS (ESI): 1182.55 [M+H]⁺.

### c. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic Acid (AV-DL111)

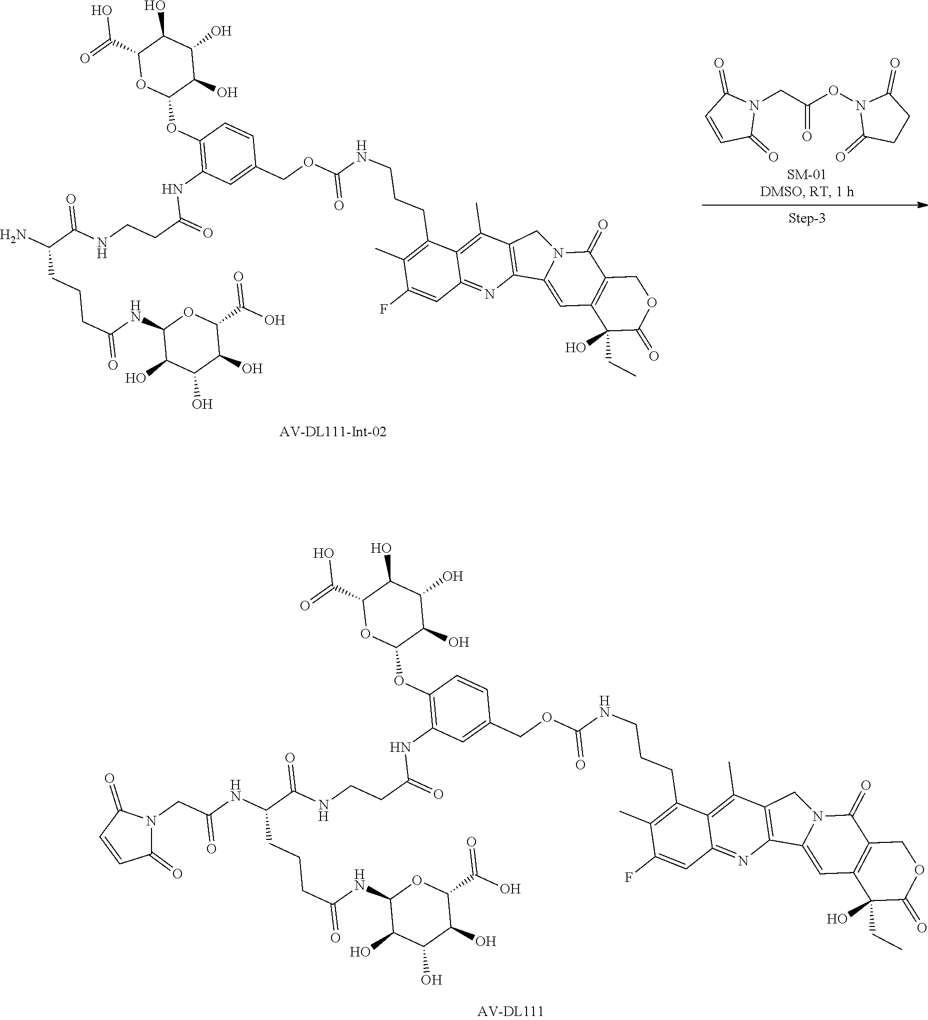 [SMILES: CC[C@@]1(O)C(=O)OCc2c1cc1n(c2=O)Cc2c-1nc1cc(F)c(C)c(CCCNC(=O)OCc3ccc(O[C@@H]4O[C@H](C(=O)O)[C@@H](O)[C@H](O)[C@H]4O)c(NC(=O)CCNC(=O)[C@@H](N)CCCC(=O)N[C@H]4O[C@H](C(=O)O)[C@@H](O)[C@H](O)[C@H]4O)c3)c1c2C ; O=C(CN1C(=O)C=CC1=O)ON1C(=O)CCC1=O ; CC[C@@]1(O)C(=O)OCc2c1cc1n(c2=O)Cc2c-1nc1cc(F)c(C)c(CCCNC(=O)OCc3ccc(O[C@@H]4O[C@H](C(=O)O)[C@@H](O)[C@H](O)[C@H]4O)c(NC(=O)CCNC(=O)[C@H](CCCC(=O)N[C@H]4O[C@H](C(=O)O)[C@@H](O)[C@H](O)[C@H]4O)NC(=O)CN4C(=O)C=CC4=O)c3)c1c2C]

To a stirred solution of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic acid (AV-DL111-Int-02, 40 mg, 0.03384 mmol) in DMSO (1.0 mL) was added 2,5-dioxopyrrolidin-1-yl 2-(2,5-dioxo-2,5-dihydro-1H-pyrrol-1 -yl)acetate (17 mg, 0.06768 mmol) and stirred at 40° C. for 1 h. The reaction was monitored by LCMS. The resulting reaction mixture was diluted with water (0.5 mL) and purified by RP Prep-HPLC to get (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic acid (AV-DL111, 12.5 mg, 0.0095 mmol, 28% yield) as a pale yellow solid.

PREP-HPLC Conditions: GEMINI C18 (250*21.2 mm) 5μ buffer-A: 0.2% FA IN WATER, B: 0.2% FA IN ACN:H₂O (80:20); 0/10,23/60,23.1/98@10 ML/MIN, Water

MS (ESI): 1319.51 [M+H]⁺: ¹H NMR (400 MHz, DMSO-d₆): δ 12.51 (brs, 2H), 9.15 (brs, 1H), 8.45 (d, J=8.8 Hz, 1H), 8.37 (d, J=8.0 Hz, 1H), 8.24 (s, 1H), 8.08 (brt, 1H), 7.78 (d, J=10.4 Hz, 1H), 7.47 (brt, 1H), 7.29 (s, 1H), 7.12-7.04 (m, 4H), 6.52 (s, 1H), 5.88 (brs, 1H), 5.46 (s, 2H), 5.30 (brm, 3H), 5.14 (brs, 1H), 5.13-4.98 (m, 3H), 4.85 (brm, 1H), 4.75 (brm, 1H), 4.25 (brm, 1H), 4.11 (s, 2H), 3.86 (d, J=9.2 Hz, 1H), 3.59 (d, J=9.6 Hz, 1H), 3.48 (m, 2H), 3.28-3.11 (m, 10H), 2.95 (s, 3H), 2.56 (brm, 2H), 2.44 (s, 3H), 2.35 (brs, 2H), 1.90-1.87 (m, 2H), 1.75 (brs, 2H), 1.65 (brs, 1H), 1.53-1.48 (m, 4H), 0.89 (t, J=7.2 Hz, 3H).

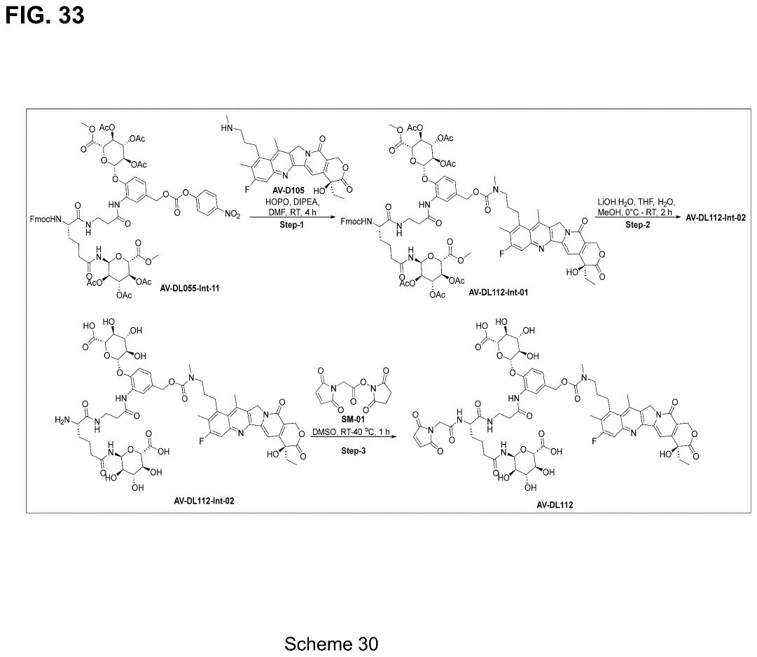

### XXIX. Synthetic scheme for (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)phenoxy)-3,4,5-trihydroxytetrahydro-2H-pyran-2-carboxylic acid (AV-DL112, Scheme 30, FIG. 33)

### a. Synthesis of (2S,3R,4S,5S,6S)-2-((S)-5-((((9H-fluoren-9-yl)methoxy)carbonyl)amino)-6-((3-((5-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL112-Int-01)

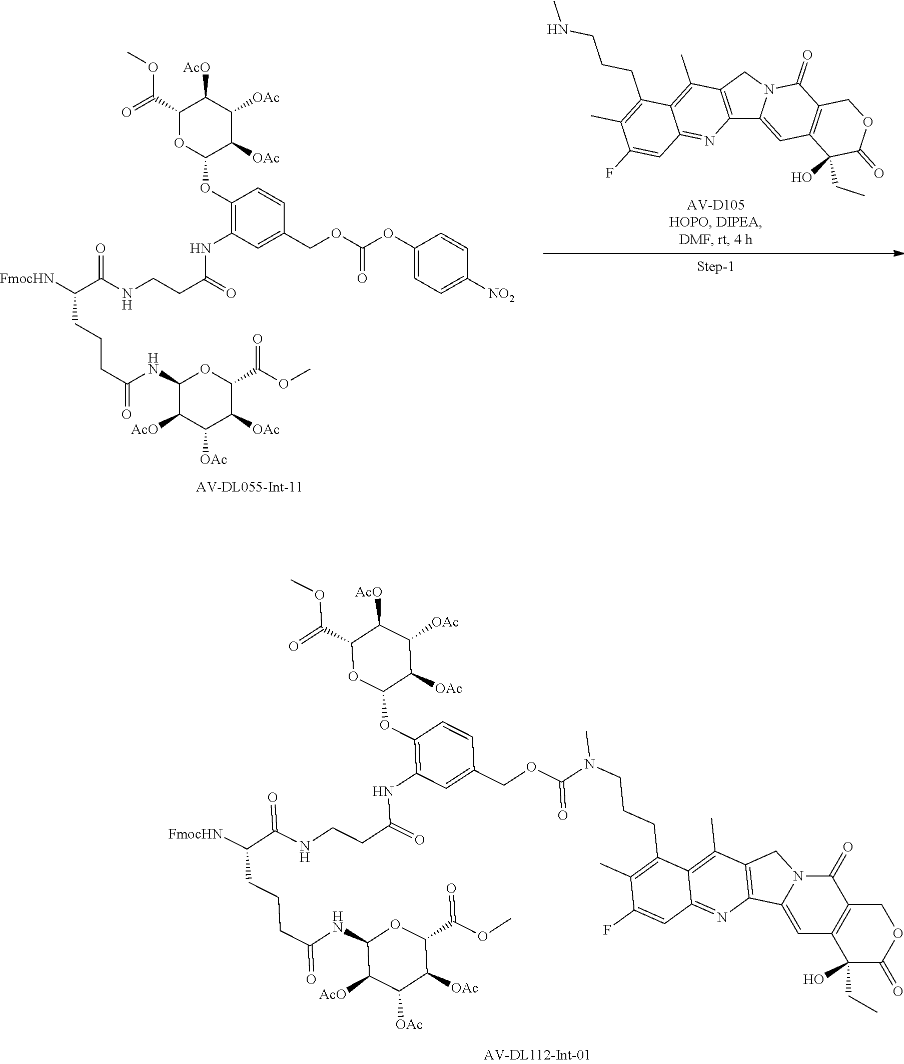

To a stirred solution of (2S,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 200 mg, 0.14 mmol), (S)-4-ethyl-8-fluoro-4-hydroxy-9,11-dimethyl-10-(3-(methylamino)propyl)-1,12-dihydro-14H-pyrano[3′,4′:6,7]indolizino[1,2-b]quinoline-3,14(4H)-dione (AV-D105, 67 mg, 0.14 mmol) in ACN:THF (1:1)(6 mL) were added HOPO (48 mg, 0.43 mmol) and DIPEA (0.12 mL, 0.72 mmol) at 0° C. and the mixture was stirred at RT for 2 h by monitoring the reaction by LCMS. Then, the reaction mixture was concentrated under reduced pressure. The crude product was purified by reverse phase Biotage (column 20 g, C18) eluted with 50% MeCN in water to afford (2S,3R,4S,5S,6S)-2-((S)-5-((((9H-fluoren-9-yl)methoxy)carbonyl)amino)-6-((3-((5-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL112-Int-01, 100 mg, 0.06 mmol, 34% yield) as an off white solid.

MS (ESI): 1699.14 [M+H]⁺.

### b. Synthesis of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-pyran-2-carboxylic Acid (AV-DL112-Int-02)

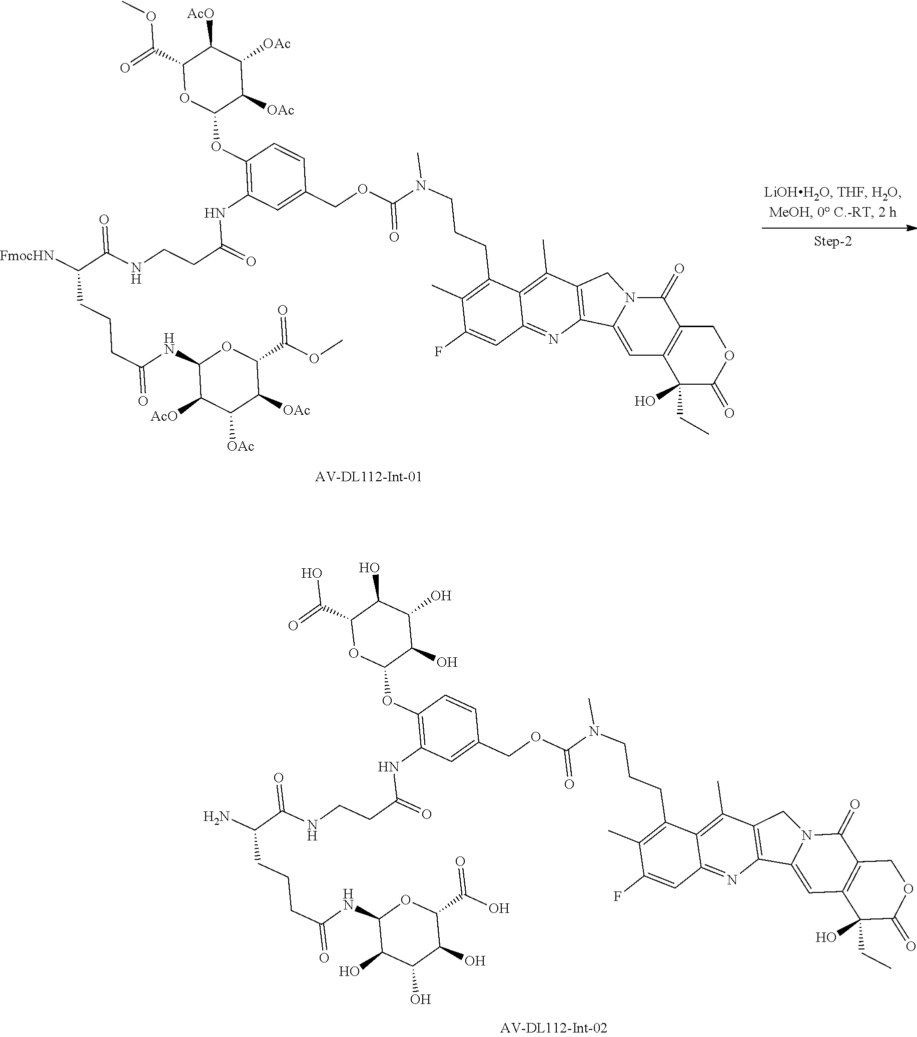

To a stirred solution of (2S,3R,4S,5S,6S)-2-((S)-5-((((9H-fluoren-9-yl)methoxy)carbonyl)amino)-6-((3-((5-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL112-Int-01, 90 mg, 0.05 mmol) in THF:H₂O:MeOH (1:1:1, 3 mL) was added LiOH·H₂O (22 mg, 0.53 mmol) at 0° C. and stirred at room temperature for 2 h. The reaction was monitored by LCMS. The mixture was concentrated under reduced pressure to get crude product. The crude product was purified by reverse phase Biotage (column 20 g, C18) eluted with 50% MeCN in water to get (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)phenoxy)-3,4,5-trihydroxytetrahydro-2H-pyran-2-carboxylic acid (AV-DL112-Int-02, 45 mg, 0.04 mmol, 71% yield) as an off white solid.

MS (ESI): 1196.74 [M+H]⁺.

### c. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-pyran-2-carboxylic Acid (AV-DL112)

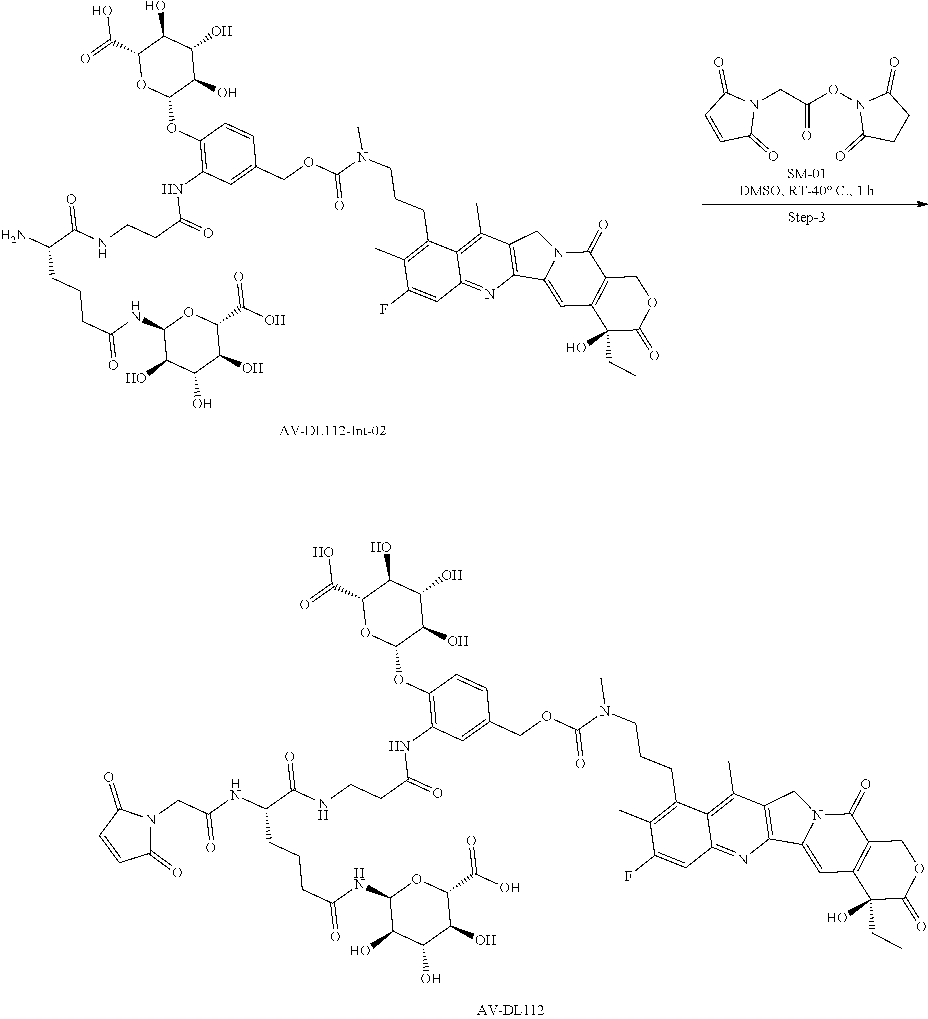 [SMILES: CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@@H](N)CCCC(=O)N[C@H]4O[C@H](C(=O)O)[C@@H](O)[C@H](O)[C@H]4O)c3)c1c2C ; O=C(CN1C(=O)C=CC1=O)ON1C(=O)CCC1=O ; CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@H](CCCC(=O)N[C@H]4O[C@H](C(=O)O)[C@@H](O)[C@H](O)[C@H]4O)NC(=O)CN4C(=O)C=CC4=O)c3)c1c2C]

To a stirred solution of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)phenoxy)-3,4,5-trihydroxytetrahydro-2H-pyran-2-carboxylic acid (AV-DL112-Int-02, 45 mg, 0.04 mmol) in DMSO (1.0 mL) at room temperature was added 2,5-dioxopyrrolidin-1-yl 2-(2,5-dioxo-2,5-dihydro-1H-pyrrol-1-yl)acetate (SM-01, 19 mg, 0.07 mmol) and stirred at 40° C. for 1 h. The reaction was monitored by LCMS. The resulting reaction mixture was diluted with water (0.5 mL) and purified by RP Prep-HPLC to afford (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)phenoxy)-3,4,5-trihydroxytetrahydro-2H-pyran-2-carboxylic acid (AV-DL112, 6.6 mg, 0.005 mmol, 12% yield) as an off white solid.

PREP-HPLC Conditions: Column: Gemini,C18, (250*21.5 mm) 5μ; Mobile phase-A::0.2% formic acid in water; Mobile phase-B::0.2% formic acid in ACN:H₂O (80:20); Wavelength: PDA; Gradient (T/% B): 0/20,15/57,21/57,21.1/98; Diluent: ACN:H₂O, Flowrate: 12 mL/min.

MS (ESI): 1333.73 [M+H]⁺; ¹H NMR (400 MHz, DMSO-d₆): δ 12.5 (brs, 1H), 9.14-9.10 (m, 1H), 8.44 (d, J=8.8 Hz, 1H), 8.36 (d, J=7.6 Hz, 1H), 8.20 (brs, 1H), 8.07 (brs, 1H), 7.77 (d, J=10.0 Hz, 1H), 7.28 (s, 1H), 7.09-7.03 (m, 4H), 6.51 (s, 1H), 5.82 (brs, 1H), 5.44 (s, 2H), 5.29-5.22 (m, 3H), 5.14 (brs, 1H), 5.01 (s, 3H), 4.81 (brs, 1H), 4.73 (t, J=8.8 Hz, 1H), 4.19-4.11 (m, 1H), 4.09-4.06 (m, 2H), 3.82-3.79 (m, 1H), 3.57 (d, J=9.6 Hz, 1H), 3.43- 3.33 (m, 5H), 3.28-3.09 (m, 10H), 2.94-2.76 (m, 7H), 2.49-2.32 (m, 4H), 2.10-2.04 (m, 2H), 1.90-1.72 (m, 4H), 1.62-1.54 (m, 1H), 1.52-1.40 (m, 3H), 0.87 (t, J=7.2 Hz, 3H).

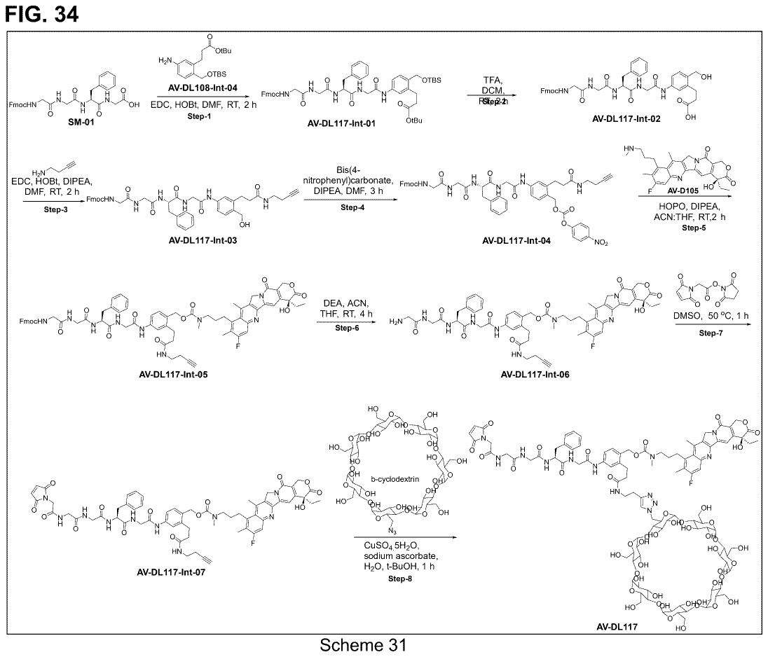

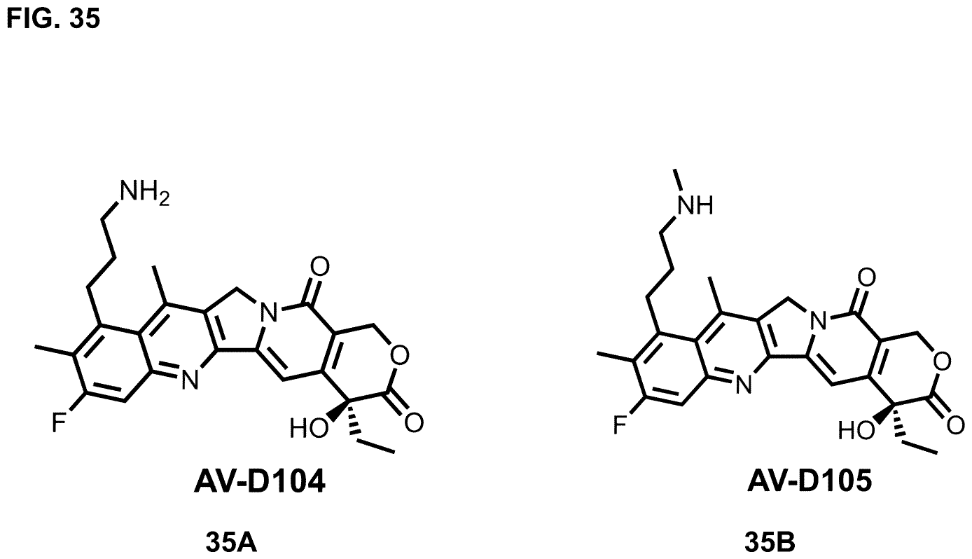

### XXX. Synthetic Scheme for AV-DL117 (Scheme 31, FIG. 34)

### a. Synthesis of tert-butyl tert-butyl(S)-3-(5-(11-benzyl-1-(9H-fluoren-9-yl)-3,6,9,12-tetraoxo-2-oxa-4,7,10,13-tetraazapentadecan-15-amido)-2-(((tert-butyldimethylsilyl)oxy)methyl)phenyl)propanoate (AV-DL117-Int-01)

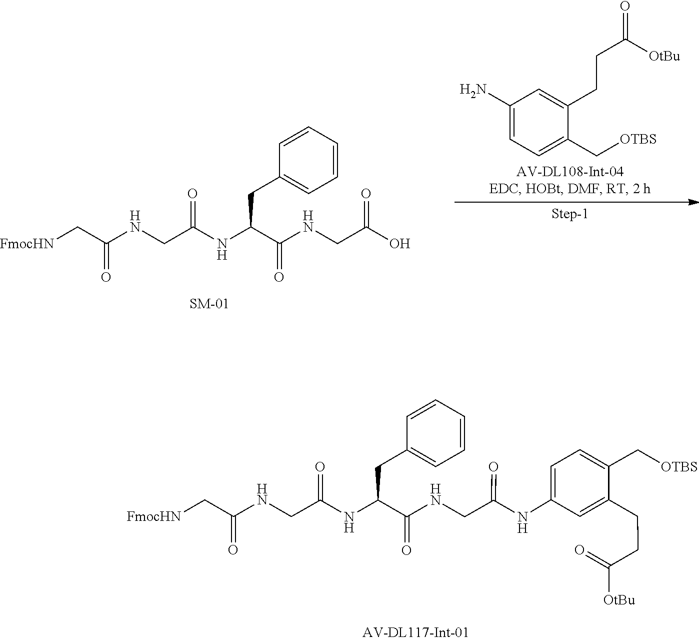

To a stirred solution of (((9H-fluoren-9-yl)methoxy)carbonyl)glycylglycyl-L-phenylalanylglycine (SM-01, 850 mg, 1.52 mmol) and tert-butyl 3-(5-amino-2-(((tert-butyldimethylsilyl)oxy)methyl)phenyl)propanoate (AV-DL108-Int-04, 667 mg, 1.83 mmol) in DMF (10 mL) were added EDC (436 mg, 2.28 mmol), HOBT (514 mg, 3.81 mmol) at 0° C. and stirred at RT for 2 h. The reaction was monitored by TLC. The resulting reaction mixture was concentrated under reduced pressure. The crude compound was purified by reverse phase Biotage (column 30 g, C18) eluted with 80% MeCN in water and pure fractions were lyophilized to afford tert-butyl(S)-3-(5-(11-benzyl-1-(9H-fluoren-9-yl)-3,6,9,12-tetraoxo-2-oxa-4,7,10,13-tetraazapentadecan-15-amido)-2-(((tert-butyldimethylsilyl)oxy)methyl)phenyl)propanoate (AV-DL117-Int-01, 600 mg, 0.66 mmol, 43% yield) as an off white solid.

MS (ESI): 928.77 [M+23]⁺.

### b. Synthesis of(S)-3-(5-(11-benzyl-1-(9H-fluoren-9-yl)-3,6,9,12-tetraoxo-2-oxa-4,7,10,13-tetraazapentadecan-15-amido)-2-(hydroxymethyl)phenyl)propanoic Acid (AV-DL117-Int-02)

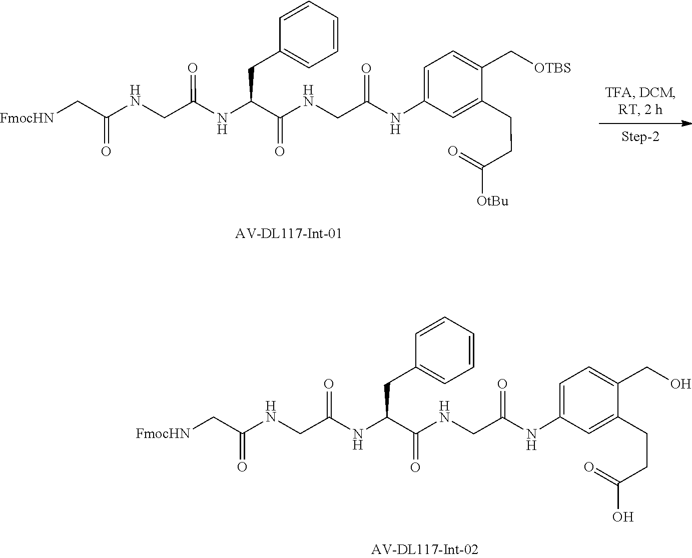

To tert-butyl(S)-3-(5-(11-benzyl-1-(9H-fluoren-9-yl)-3,6,9,12-tetraoxo-2-oxa-4,7,10,13-tetraazapentadecan-15-amido)-2-(((tert-butyldimethylsilyl)oxy)methyl)phenyl)propanoate (AV-DL117-Int-01, 600 mg, 0.66 mmol) was added 20% TFA in DCM (10 mL) at room temperature and stirred for 2 h. The reaction was monitored by TLC. The resulting reaction mixture was concentrated under reduced pressure. The crude compound was triturated with diethyl ether to afford(S)-3-(5-(11-benzyl-1-(9H-fluoren-9-yl)-3,6,9,12-tetraoxo-2-oxa-4,7,10,13-tetraazapentadecan-15-amido)-2-(hydroxymethyl)phenyl)propanoic acid (AV-DL117-Int-02, 450 mg, 0.61 mmol) as an off white solid.

MS (ESI): 736.54 [M+H]⁺.

### c. Synthesis of (9H-fluoren-9-yl)methyl(S)-(2-((2-((1-((2-((3-(3-(but-3-yn-1-ylamino)-3-oxopropyl)-4-(hydroxymethyl)phenyl)amino)-2-oxoethyl)amino)-1-oxo-3-phenylpropan-2-yl)amino)-2-oxoethyl)amino)-2-oxoethyl)carbamate (AV-DL117-Int-03)

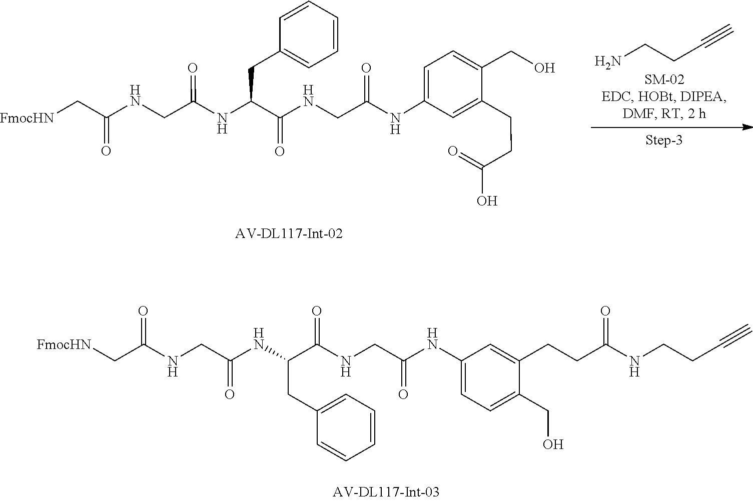

To a stirred solution(S)-3-(5-(11-benzyl-1-(9H-fluoren-9-yl)-3,6,9,12-tetraoxo-2-oxa-4,7,10,13-tetraazapentadecan-15-amido)-2-(hydroxymethyl)phenyl)propanoic acid (AV-DL117-Int-02, 300 mg, 0.41 mmol) and but-3-yn-1-amine (SM-02, 42 mg, 0.41 mmol) in DMF (5 mL) were added EDC (116 mg, 0.61 mmol), HOBT (137 mg, 1.02 mmol) and DIPEA (0.2 mL, 1.22 mmol) at 0° C. and stirred at RT for 2 h. The reaction was monitored by TLC. The resulting reaction mixture was concentrated under reduced pressure. The crude compound was purified by reverse phase Biotage (column 30 g, C18) eluted with 60% MeCN in water and fractions were lyophilized to afford (9H-fluoren-9-yl)methyl(S)-(2-((2-((1-((2-((3-(3-(but-3-yn-1-ylamino)-3-oxopropyl)-4-(hydroxymethyl)phenyl)amino)-2-oxoethyl)amino)-1-oxo-3-phenylpropan-2-yl)amino)-2-oxoethyl)amino)-2-oxoethyl)carbamate (AV-DL117-Int-03, 240 mg, 0.30 mmol, 74% yield) as an off white solid.

MS (ESI): 787.58 [M+H]⁺.

### d. Synthesis of (9H-fluoren-9-yl)methyl(S)-(2-((2-((1-((2-((3-(3-(but-3-yn-1-ylamino)-3-oxopropyl)-4-((((4-nitrophenoxy)carbonyl)oxy)methyl)phenyl)amino)-2-oxoethyl)amino)-1-oxo-3-phenylpropan-2-yl)amino)-2-oxoethyl)amino)-2-oxoethyl)carbamate (AV-DL117-Int-04)

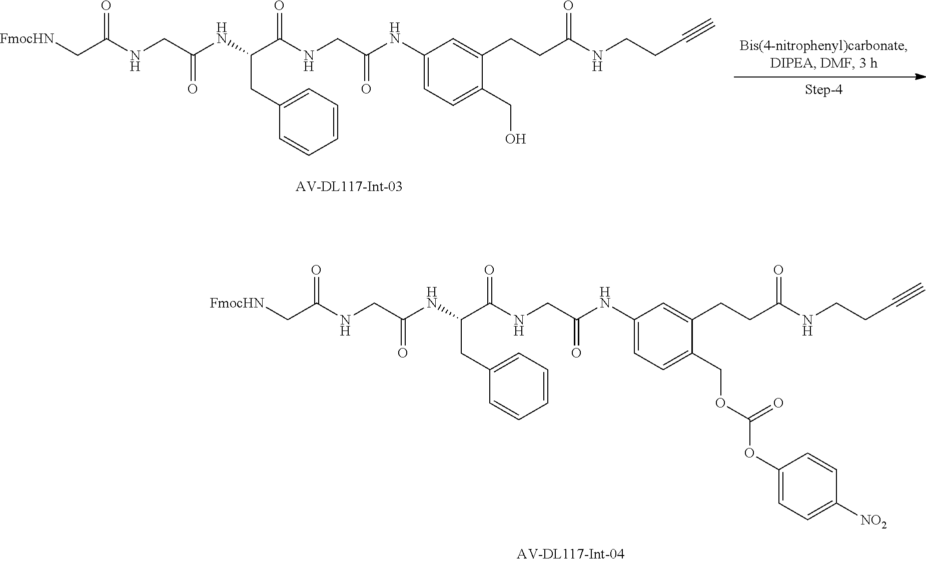

To a stirred solution of (9H-fluoren-9-yl)methyl(S)-(2-((2-((1-((2-((3-(3-(but-3-yn-1-ylamino)-3-oxopropyl)-4-(hydroxymethyl)phenyl)amino)-2-oxoethyl)amino)-1-oxo-3-phenylpropan-2-yl)amino)-2-oxoethyl)amino)-2-oxoethyl)carbamate (AV-DL117-Int-03, 240 mg, 0.30 mmol) and bis(4-nitrophenyl)carbonate (278 mg, 0.91 mmol) in DMF (5 mL) was added DIPEA (0.16 mL, 0.91 mmol) at 0° C. and stirred at RT for 2 h. The reaction was monitored by TLC. The resulting reaction mixture was concentrated under reduced pressure. The crude compound was triturated with diethyl ether (3×20 mL) and dried to afford (9H-fluoren-9-yl)methyl(S)-(2-((2-((1-((2-((3-(3-(but-3-yn-1-ylamino)-3-oxopropyl)-4-((((4-nitrophenoxy)carbonyl)oxy)methyl)phenyl)amino)-2-oxoethyl)amino)-1-oxo-3-phenylpropan-2-yl)amino)-2-oxoethyl)amino)-2-oxoethyl)carbamate (AV-DL117-Int-04, 220 mg, 0.23 mmol, 77% yield) as a pale yellow solid.

MS (ESI): 952.59 [M+H]⁺.

### e. Synthesis of 4-((S)-11-benzyl-1-(9H-fluoren-9-yl)-3,6,9,12-tetraoxo-2-oxa-4,7,10,13-tetraazapentadecan-15-amido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-05)

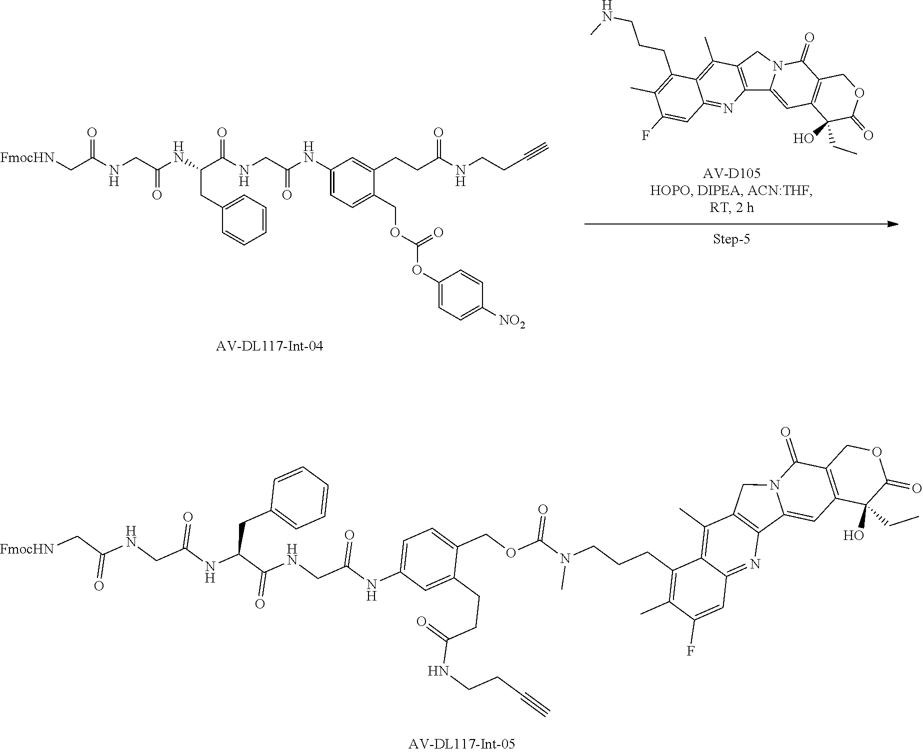

To a stirred solution of (9H-fluoren-9-yl)methyl(S)-(2-((2-((1-((2-((3-(3-(but-3-yn-1-ylamino)-3-oxopropyl)-4-((((4-nitrophenoxy)carbonyl)oxy)methyl)phenyl)amino)-2-oxoethyl)amino)-1-oxo-3-phenylpropan-2-yl)amino)-2-oxoethyl)amino)-2-oxoethyl)carbamate (AV-DL117-Int-04, 100 mg, 0.10 mmol) and(S)-4-ethyl-8-fluoro-4-hydroxy-9,11-dimethyl-10-(3-(methylamino)propyl)-1,12-dihydro-14H-pyrano[3′,4′:6,7]indolizino[1,2-b]quinoline- 3,14(4H)-dione (AV-D105, 49 mg, 0.10 mmol) in ACN:THF (5 mL, 50 vol.) was added HOPO (35 mg, 0.31 mmol) and DIPEA (0.1 mL, 0.52 mmol) at 0° C. and stirred at RT for 2 h. The reaction was monitored by TLC. The resulting reaction mixture was concentrated under reduced pressure. The crude compound was purified by reverse phase Biotage (column 12 g, C18) eluted with 60% MeCN in water to afford 4-((S)-11-benzyl-1-(9H-fluoren-9-yl)-3,6,9,12-tetraoxo-2-oxa-4,7,10,13-tetraazapentadecan-15-amido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-05, 40 mg, 0.03 mmol, 29% yield) as an off white solid.

MS (ESI): 1279.88 [M+H]⁺.

### f. Synthesis of 4-(2-((S)-2-(2-(2-aminoacetamido)acetamido)-3-phenylpropanamido)acetamido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-06)

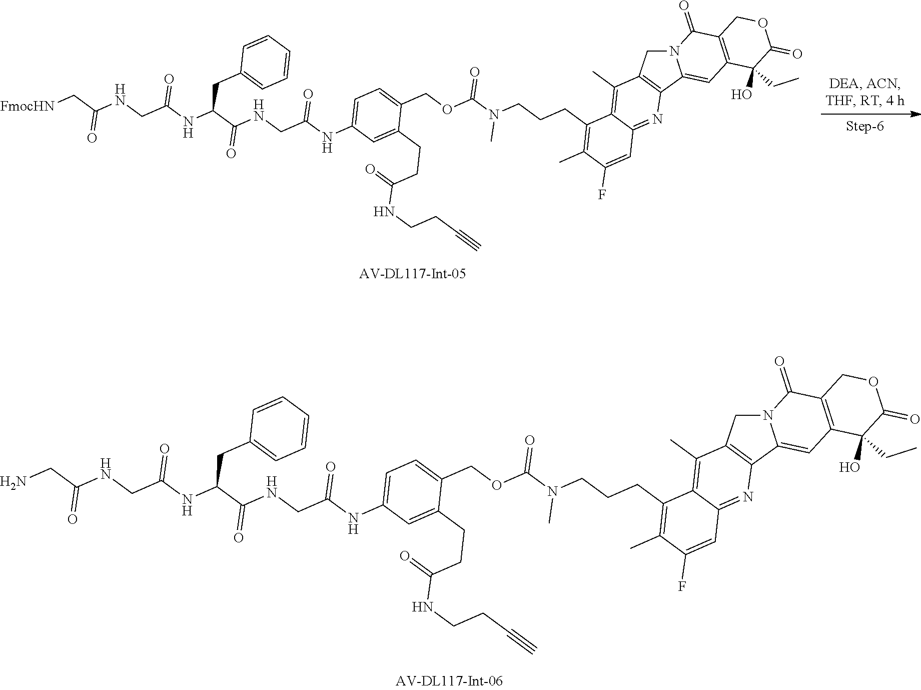

To a stirred solution of 4-((S)-11-benzyl-1-(9H-fluoren-9-yl)-3,6,9,12-tetraoxo-2-oxa-4,7,10,13-tetraazapentadecan-15-amido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-05, 90 mg, 0.07 mmol) in ACN:THF (1:1, 4 mL) was added diethyl amine (0.02 mL, 0.21 mmol) at 0° C. and stirred at RT for 4 h. The reaction was monitored by TLC. The reaction mixture was concentrated under reduced pressure to afford 4-(2-((S)-2-(2-(2-aminoacetamido)acetamido)-3-phenylpropanamido)acetamido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-06, 50 mg, 0.05 mmol, 67% yield) as a light brown solid.

MS (ESI): 1056.84 [M+H]⁺.

### g. Synthesis of 4-((S)-5-benzyl-14-(2,5-dioxo-2,5-dihydro-1H-pyrrol-1-yl)-4,7,10,13-tetraoxo-3,6,9,12-tetraazatetradecanamido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-07)

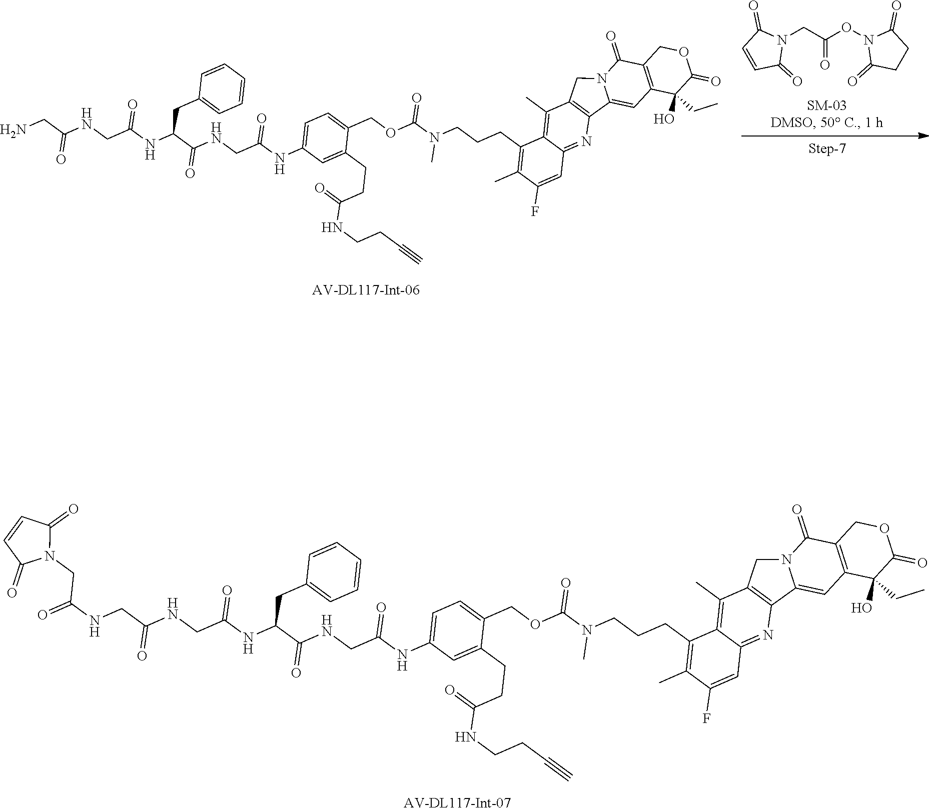 [SMILES: C#CCCNC(=O)CCc1cc(NC(=O)CNC(=O)[C@H](Cc2ccccc2)NC(=O)CNC(=O)CN)ccc1COC(=O)N(C)CCCc1c(C)c(F)cc2nc3c(c(C)c12)Cn1c-3cc2c(c1=O)COC(=O)[C@]2(O)CC ; O=C(CN1C(=O)C=CC1=O)ON1C(=O)CCC1=O ; C#CCCNC(=O)CCc1cc(NC(=O)CNC(=O)[C@H](Cc2ccccc2)NC(=O)CNC(=O)CNC(=O)CN2C(=O)C=CC2=O)ccc1COC(=O)N(C)CCCc1c(C)c(F)cc2nc3c(c(C)c12)Cn1c-3cc2c(c1=O)COC(=O)[C@]2(O)CC]

To a stirred solution of 4-(2-((S)-2-(2-(2-aminoacetamido)acetamido)-3-phenylpropanamido)acetamido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-06, 50 mg, 0.03 mmol) in DMSO (1 mL) was added 2,5-dioxopyrrolidin-1-yl 2-(2,5-dioxo-2,5-dihydro-1H-pyrrol-1-yl)acetate (SM-03, 25 mg, 0.09 mmol) and stirred at 50° C. for 1 h. The reaction was monitored by LCMS. The resulting reaction mixture was diluted with water (0.5 mL) and purified by reverse phase Biotage (column 12 g, C18) eluted with 40% MeCN in water to afford 4-((S)-5-benzyl-14-(2,5-dioxo-2,5-dihydro-1H-pyrrol-1-yl)-4,7,10,13-tetraoxo-3,6,9,12-tetraazatetradecanamido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamate (AV-DL117-Int-07, 20 mg, 0.016 mmol, 50% yield) as an off white solid.

MS (ESI): 1194.04 [M+H]⁺.

### Synthesis of AV-DL117

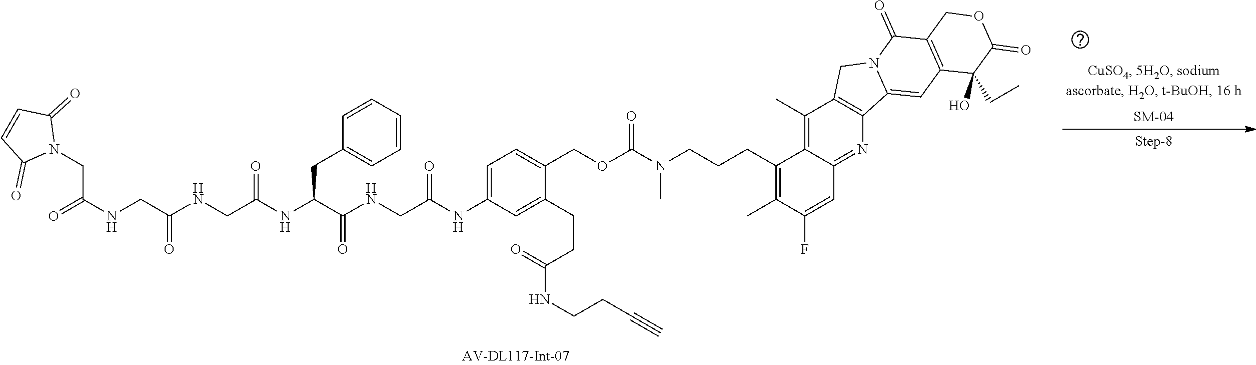 [SMILES: C#CCCNC(=O)CCc1cc(NC(=O)CNC(=O)[C@H](Cc2ccccc2)NC(=O)CNC(=O)CNC(=O)CN2C(=O)C=CC2=O)ccc1COC(=O)N(C)CCCc1c(C)c(F)cc2nc3c(c(C)c12)Cn1c-3cc2c(c1=O)COC(=O)[C@]2(O)CC]

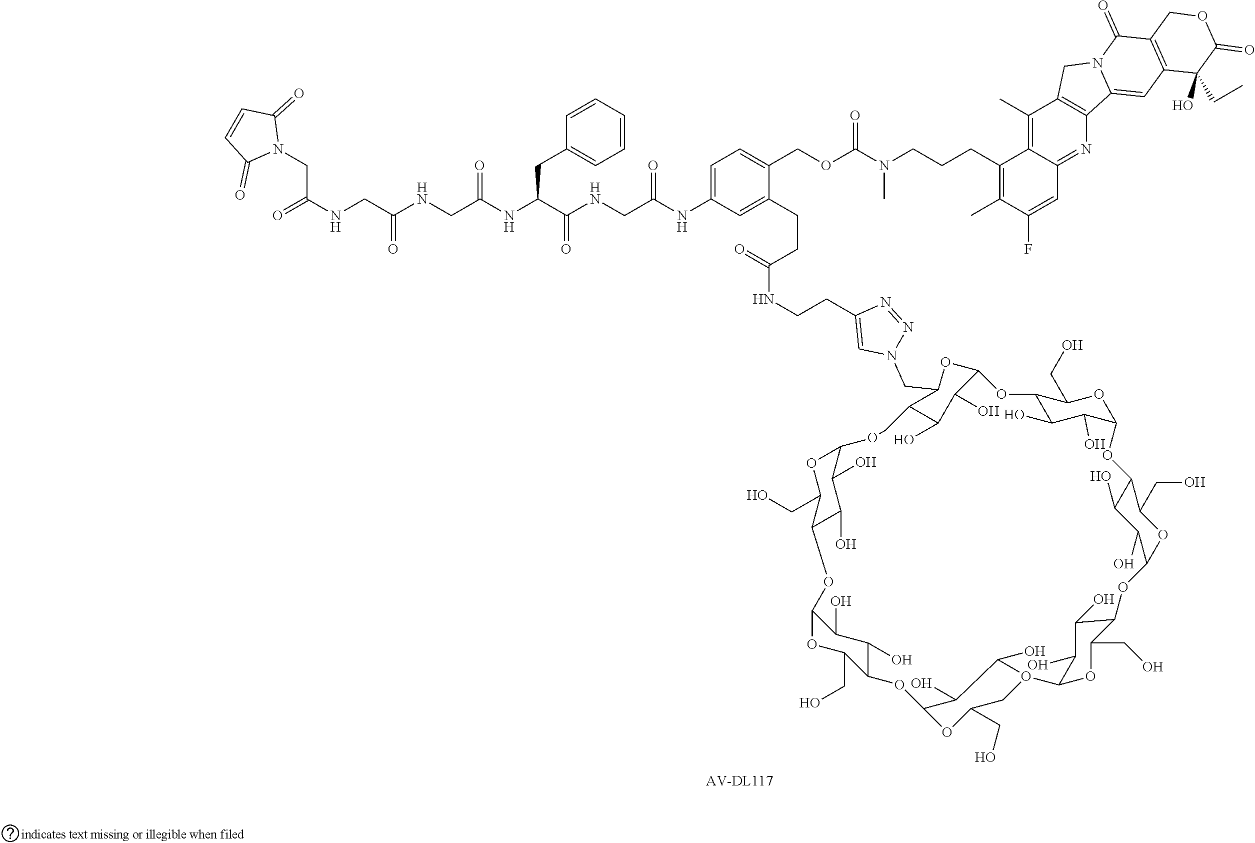

To a stirred solution of 4-((S)-5-benzyl-14-(2,5-dioxo-2,5-dihydro-1H-pyrrol-1-yl)-4,7,10,13-tetraoxo-3,6,9,12-tetraazatetradecanamido)-2-(3-(but-3-yn-1-ylamino)-3-oxopropyl)benzyl (3-((S)-4-ethyl-8-fluoro-4-hydroxy-9,11-dimethyl-3,14-dioxo-3,4,12,14 -tetrahydro-1H-pyrano[3′,4′:6,7]indolizino[1,2-b]quinolin-10-yl)propyl)(methyl)carbamate (AV-DL117-Int-07, 20 mg, 0.016 mmol) and mono-(6-azido-6-deoxy)-beta-cyclodextrin (SM-04, 38 mg, 0.033 mmol) in t-butanol and water (1:1, 0.5 mL) were added Sodium ascorbate (0.6 mg, 0.033 mmol) followed by CuSO₄·5H₂O (0.53 mg, 0.033 mmol) in water (0.5 mL) at the room temperature and the mixture was stirred for 16 h. The reaction was monitored by LCMS. The reaction mixture was lyophilized and the crude compound was purified by RP prep HPLC to afford AV-DL117 (7 mg, 0.0029 mmol, 18% yield) as an off white solid.

PREP-HPLC Conditions: GEMINI C18 (250*21.2 mm) 5μ, A: 0.2% formic acid in water, B: 0.2% formic acid in ACN:H₂O (80:20); Gradient (T/% B): 0/25,20/55,22/55,22.1/98; Diluent: MeCN, Water, THF; Flowrate: 10 mL/min.

MS (ESI): 1178.04 [(M+2H)/2]⁺²; ¹H NMR (400 MHz, DMSO-d₆): δ 9.80 (s, 1H), 8.47-8.42 (m, 1H), 8.40-8.36 (m, 1H), 8.19-8.11 (m, 2H), 8.04-7.97 (m, 1H), 7.80-7.74 (m, 2H), 7.49-7.40 (m, 2H), 7.28-7.4 (m, 8H), 7.07 (s, 2H), 6.52 (s, 1H), 5.80-5.78 (m, 1H), 5.77-5.65 (m, 15H), 5.44 (s, 2H), 5.30-5.16 (m, 2H), 5.10-5.02 (m, 4H), 4.84-4.72 (m, 8H), 4.58-4.46 (m, 9H), 4.30-4.28 (m, 1H), 4.09 (s, 2H), 4.01-3.82 (m, 4H), 3.76-3.52 (m, 30H), 3.43-3.36 (m, 5H), 3.22-3.20 (m, 2H), 3.09-3.01 (m, 2H), 2.93 (s, 3H), 2.90-2.61 (m, 10H), 2.49-2.30 (m, 6H), 1.89-1.72 (m, 4H), 0.87 (t, J=7.2 Hz, 3H).

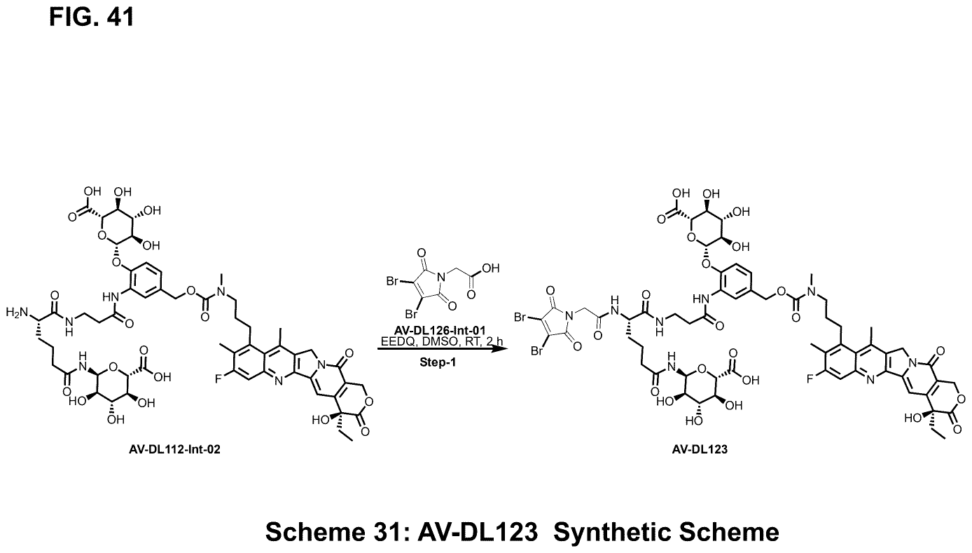

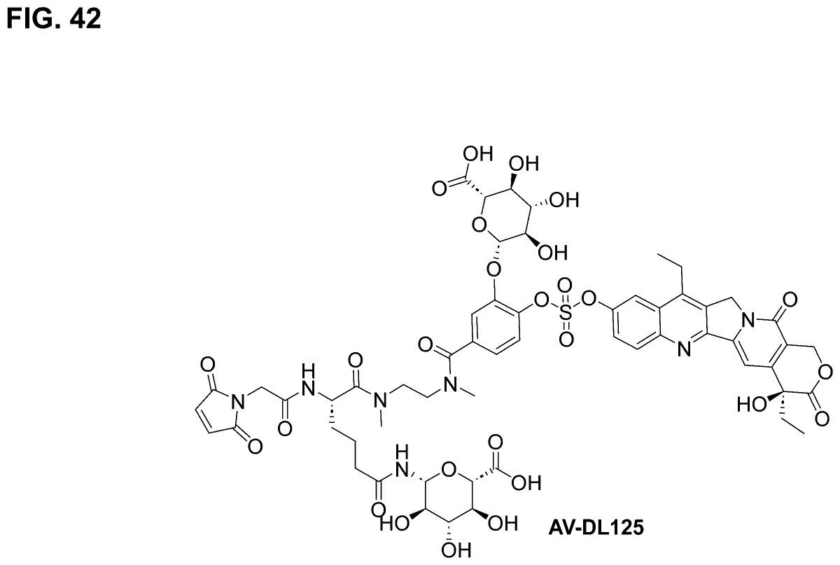

### XXXI. Synthetic Scheme for AV-DL123 (Scheme 31, FIG. 41)

### a. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-pyran-2-carboxylic Acid (AV-DL123)

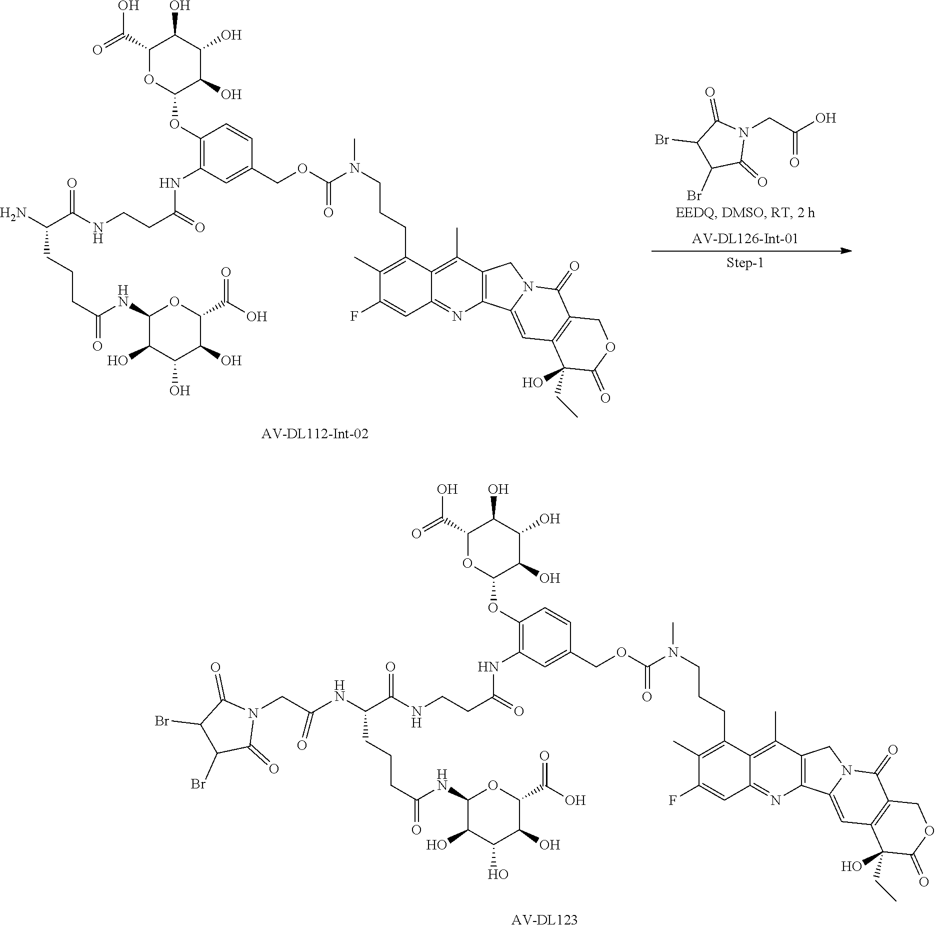 [SMILES: CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@@H](N)CCCC(=O)N[C@H]4O[C@H](C(=O)O)[C@@H](O)[C@H](O)[C@H]4O)c3)c1c2C ; O=C(O)CN1C(=O)C(Br)C(Br)C1=O ; CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@H](CCCC(=O)N[C@H]4O[C@H](C(=O)O)[C@@H](O)[C@H](O)[C@H]4O)NC(=O)CN4C(=O)C(Br)C(Br)C4=O)c3)c1c2C]

To a stirred solution of 2-(3,4-dibromo-2,5-dioxo-2,5-dihydro-1H-pyrrol-1-yl) acetic acid (AV-DL126-Int-01, 7.8 mg, 0.025 mmol) in DMSO (0.5 mL) was added EEDQ (7.4 mg, 0.03 mmol) at room temperature and stirred for 5 min, was added ((2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)carbamoyl)oxy)methyl)phenoxy)-3,4,5-trihydroxytetrahydro-2H-pyran-2-carboxylic acid (AV-DL112-Int-02, 30 mg, 0.025 mmol) in DMSO (0.5 mL) at room temperature and stirred for 2 h (monitored by LCMS). The reaction mixture was diluted with water (0.2 mL) and purified on Biotage flash system by reverse phase C18 column (12g), eluted with 60% MeCN in water to afford 15 mg of semi pure compound which was re-purified by RP prep HPLC and fractions were lyophilized to afford (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid (AV-DL123, 3 mg, 0.002 mmol, 8% yield) as a pale yellow solid.

LCMS (ESI, m/z): 1490.31 [M+H]⁺: ¹H NMR (400 MHz, DMSO-d₆): δ 12.78 (brs, 2H), 9.11-9.06 (m, 1H), 8.40 (d, J=8.8 Hz, 2H), 8.22 (brs, 1H), 8.08 (brs, 1H), 7.77 (d, J=10.4 Hz, 1H), 7.28 (s, 1H), 7.08-7.03 (m, 2H), 6.50 (brs, 1H), 5.82 (brs, 1H), 5.44 (s, 3H), 5.40-5.12 (m, 6H), 5.01 (s, 2H), 4.85 (brs, 1H), 4.75 (t, J=8.8 Hz, 1H), 4.21-4.18 (m, 3H), 3.88 (d, J=9.2 Hz, 1H), 3.59 (d, J=10.2 Hz, 1H), 3.46-3.40 (m, 4H), 3.25-3.16 (m, 3H), 3.10 (t, J= 8.8 Hz, 1H), 2.96-2.90 (m, 6H), 2.82 (brs, 2H), 2.60-2.53 (m, 2H), 2.44-2.37 (m, 3H), 2.20-2.07 (m, 3H), 1.89-1.75 (m, 4H), 1.64-1.57 (m, 1H), 1.52-1.40 (m, 4H), 0.88 (t, J=7.2 Hz, 3H).

Prep-HPLC conditions: Column: GEMINI C18 (250*21.2 mm) 5μ; Buffer-A: 0.1% TFA in water, ACN:H₂O (80:20); Gradient (T/% B): 0/35,12/70,20.01/98; Flow Rate: 10 mL/min.; Solubility: ACN+H₂O.

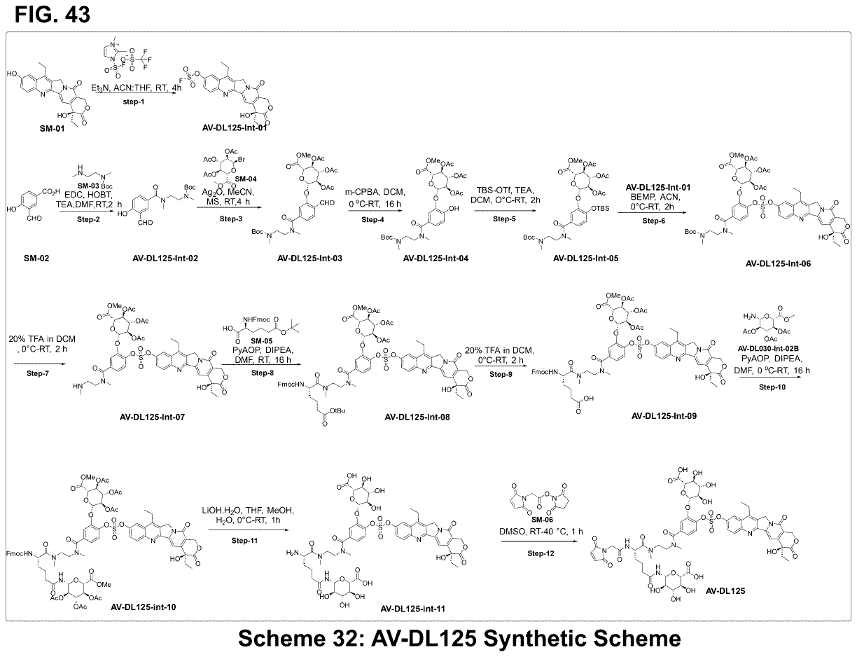

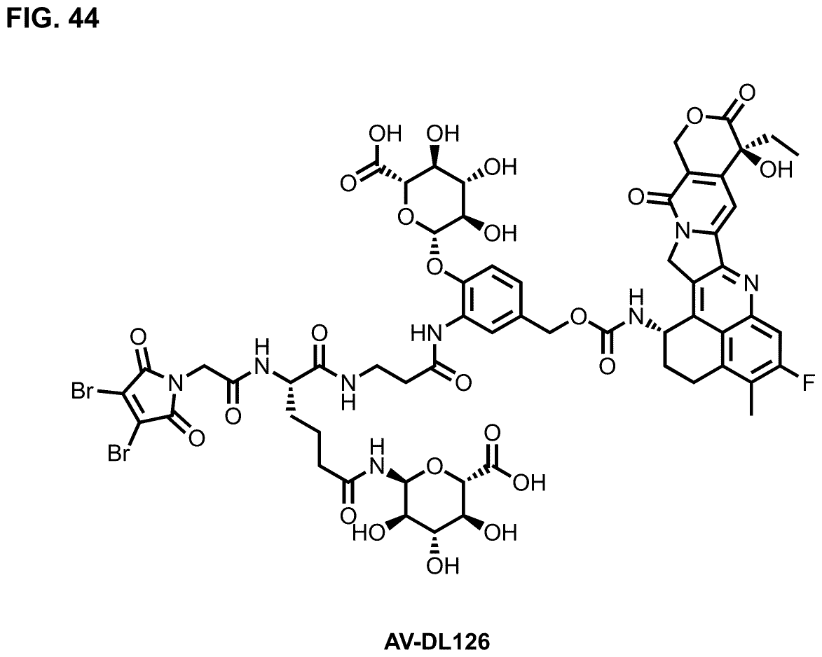

### XXXII. Synthetic Scheme for AV-DL125 (Scheme 32, FIG. 43)

### a. Synthesis of (2S,3S,4S,5R,6S)-6-(4-((2-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-N-methyl-6-oxohexanamido)ethyl)(methyl)carbamoyl)-2-(((((S)-4,11-diethyl-4-hydroxy-3,14-dioxo-3,4,12,14-tetrahydro-1H-pyrano[3′,4′:6,7]indolizino[1,2-b]quinolin-9-yl)oxy) sulfonyl)oxy)phenoxy)-3,4,5-trihydroxytetrahydro-2H-pyran-2-carboxylic Acid (AV-DL125)

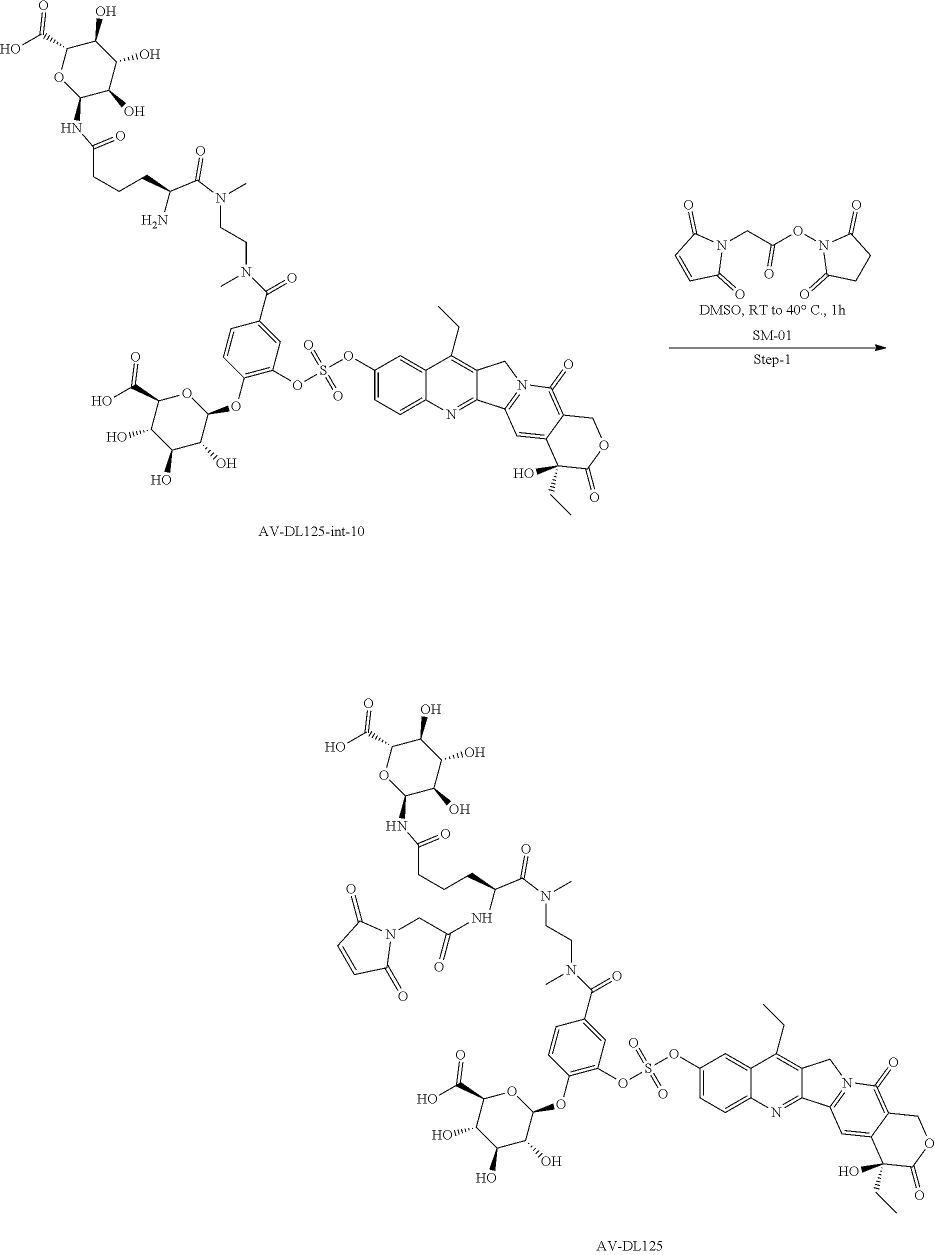

To a stirred solution of (2S,3S,4S,5R,6S)-6-(4-((2-((S)-2-amino-6-(((2S,3R,4S,5S,6S)-6-carboxy-3,4,5-trihydroxytetrahydro-2H-pyran-2-yl)amino)-N-methyl-6-oxohexanamido)ethyl)(methyl)carbamoyl)-2-(((((S)-4,11-diethyl-4-hydroxy-3,14-dioxo-3,4,12,14-tetrahydro-1H-pyrano[3′,4′:6,7]indolizino[1,2-b]quinolin-9-yl)oxy) sulfonyl)oxy)phenoxy)-3,4,5-trihydroxytetrahydro-2H-pyran-2-carboxylic acid (AV-DL125-Int-10, 60 mg, 51.1 μmol) in DMSO (0.6 mL) at room temperature was added 2,5-dioxopyrrolidin-1-yl 2-(2,5-dioxo-2,5-dihydro-1H-pyrrol-1-yl)acetate (SM-01, 19.3 mg, 76.7 μmol) and stirred at 40° C. for 1 h. The reaction was monitored by LCMS. The resulting reaction mixture was diluted with water (0.5 mL) and purified by RP Prep-HPLC to afford (2S,3S,4S,5R,6S)-6-(4-((2-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-N-methyl-6 -oxohexanamido)ethyl)(methyl)carbamoyl)-2-(((((S)-4,11-diethyl-4-hydroxy-3,14-dioxo-3,4,12,14-tetrahydro-1H-pyrano[3′,4′:6,7]indolizino[1,2-b]quinolin-9-yl)oxy) sulfonyl)oxy)phenoxy)-3,4,5-trihydroxytetrahydro-2H-pyran-2-carboxylic acid (AV-DL125, 5.6 mg, 4.27 μmol, 8% yield) as an off white solid.

LCMS (ESI): 1310.3 [M+H]⁺;

PREP-HPLC Conditions: Column: Gemini C18 (250*21.5 mm) 5μ; Mobile phase-A: 0.1% formic acid in water; Mobile phase-B: 0.1% formic acid in ACN:H₂O (80:20); Gradient (T/% B): 0/20,15/57,21/57,21.1/98; Diluent: ACN:H₂O, Flowrate: 12 mL/min.

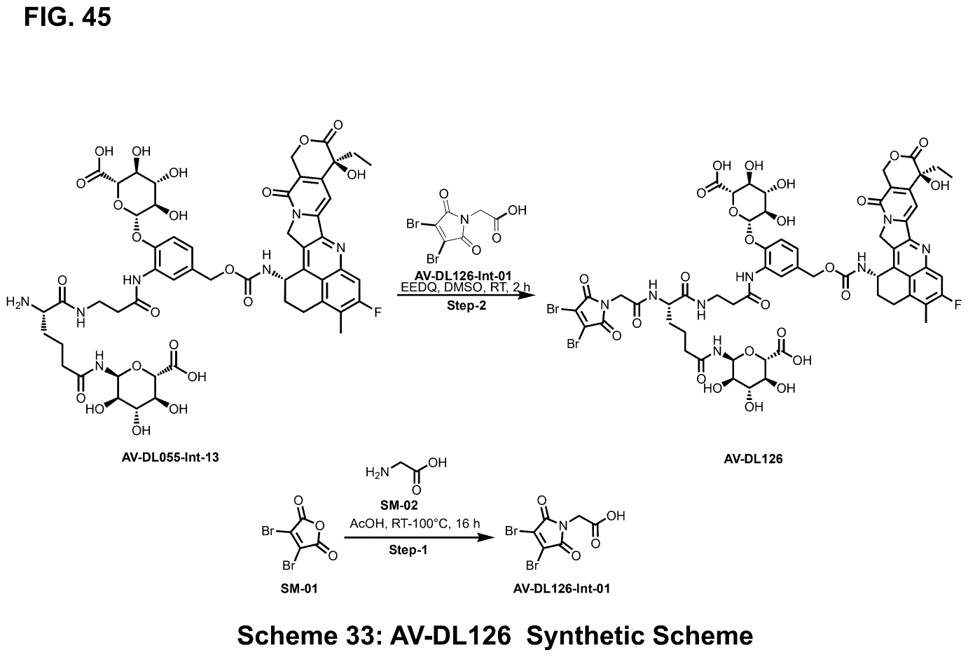

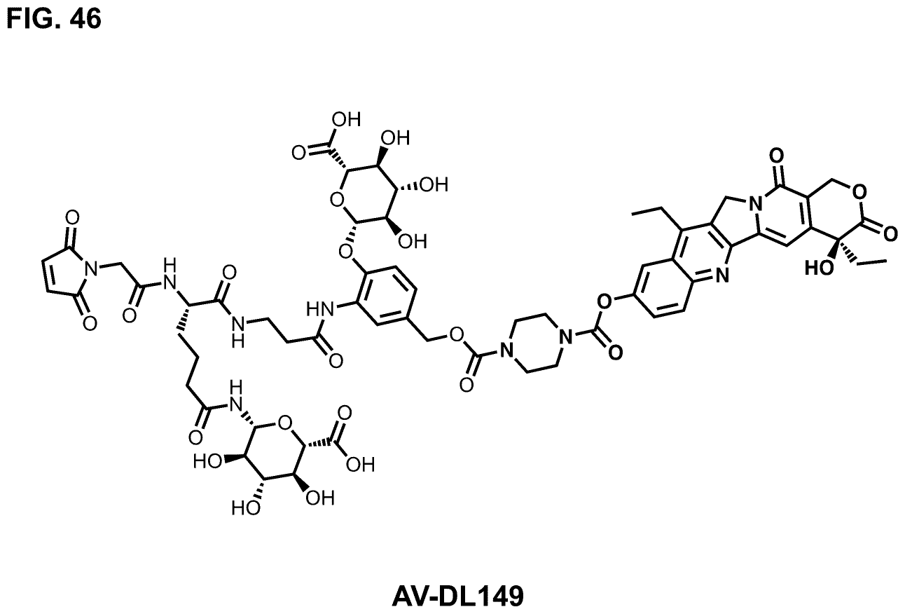

### XXXII. Synthetic Scheme for AV-DL126 (Scheme 33, FIG. 45)

### a. Synthetic scheme of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic Acid (AV-DL126)

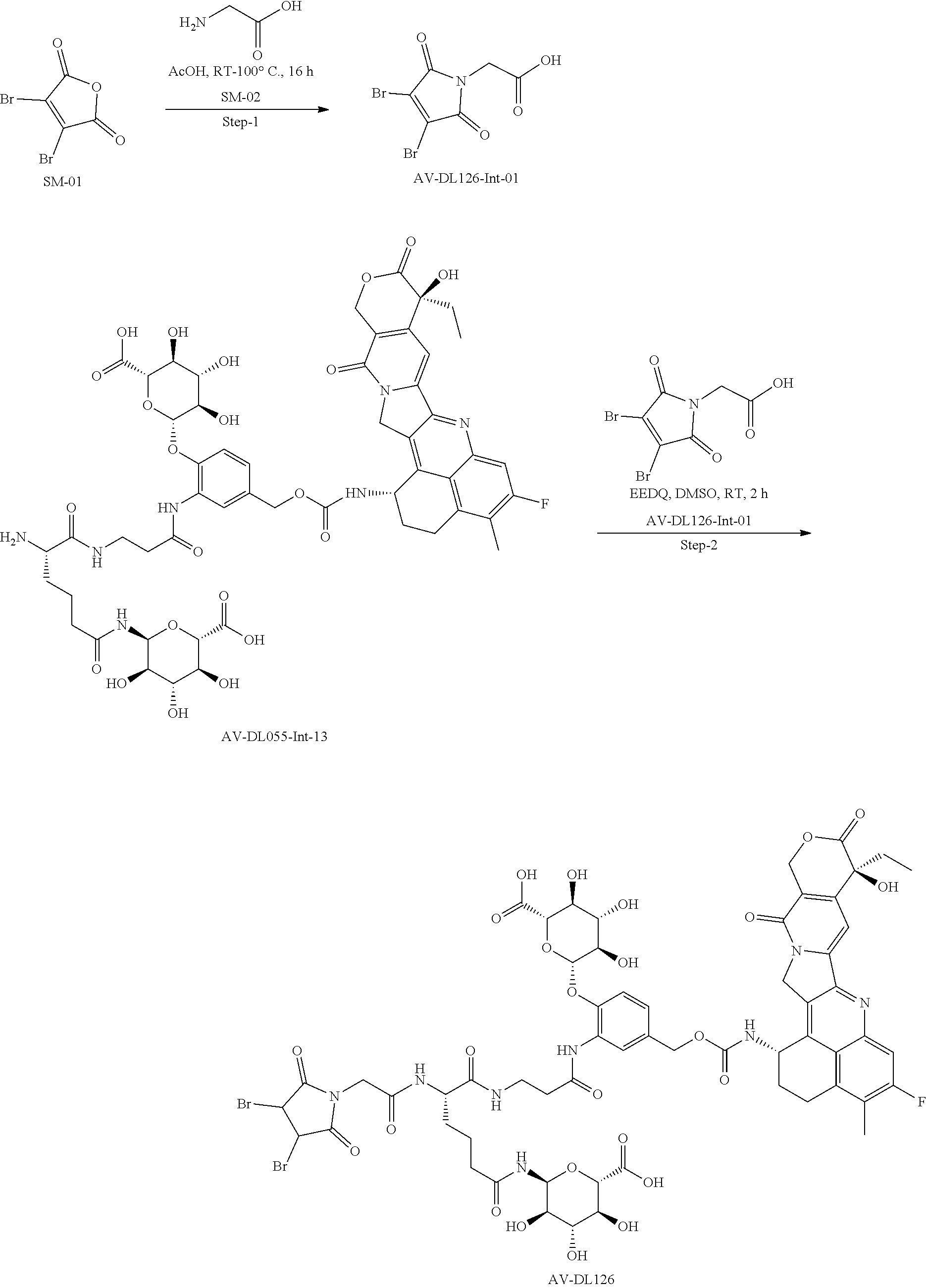

### Synthesis of 2-(3,4-dibromo-2,5-dioxo-2,5-dihydro-1H-pyrrol-1-yl) acetic Acid (AV-DL126-Int-01)

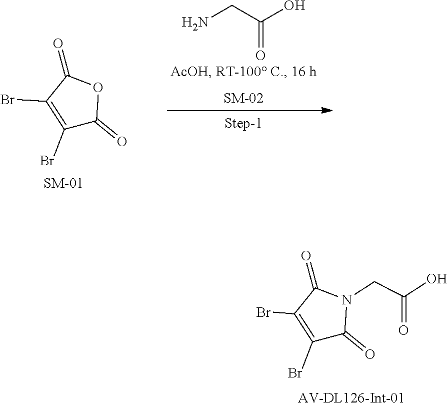 [SMILES: O=C1OC(=O)C(Br)=C1Br ; NCC(=O)O ; O=C(O)CN1C(=O)C(Br)=C(Br)C1=O]

To a stirred solution of 3,4-dibromofuran-2,5-dione (SM-01, 1.0 g, 3.92 mmol) in acetic acid (20 mL) at room temperature was added glycine (SM-02, 294 mg, 3.92 mmol) and the reaction mixture was stirred at 100° C. for 16 h. Then, the reaction mixture was cooled to room temperature and concentrated under reduced pressure to get crude product. The crude product was purified by silica gel column chromatography (SiO₂, 100-200 mesh, 5% MeOH/DCM) and dried to afford 2-(3,4-dibromo-2,5-dioxo-2,5-dihydro-1H-pyrrol-1-yl) acetic acid (AV-DL126-Int-01, 1.1 g, 3.52 mmol, 90% yield) as a light brown solid.

MS (ESI): 312.07 [M+H]⁺.

### b. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic Acid (AV-DL126)

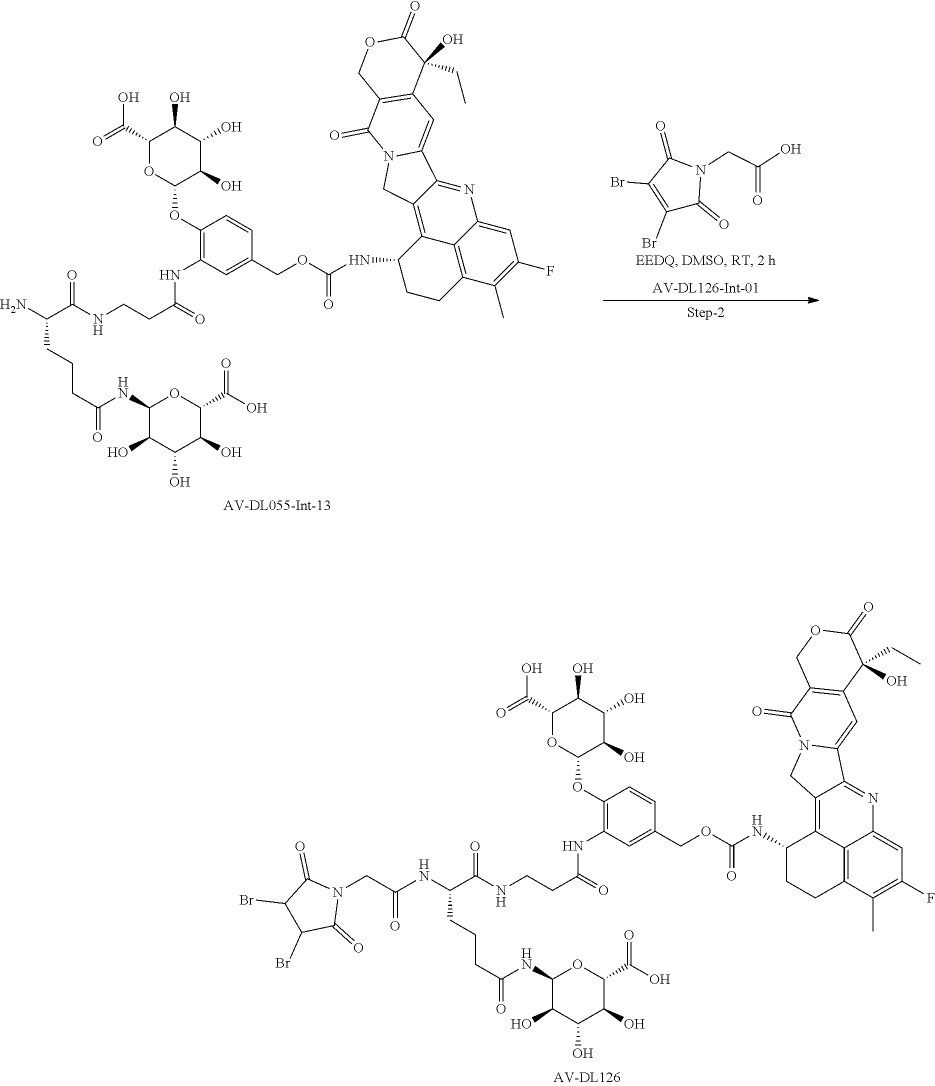 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCNC(=O)[C@@H](N)CCCC(=O)N[C@H]2O[C@H](C(=O)O)[C@@H](O)[C@H](O)[C@H]2O)c1)CC3 ; O=C(O)CN1C(=O)C(Br)=C(Br)C1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCNC(=O)[C@H](CCCC(=O)N[C@H]2O[C@H](C(=O)O)[C@@H](O)[C@H](O)[C@H]2O)NC(=O)CN2C(=O)C(Br)C(Br)C2=O)c1)CC3]

To a stirred solution of 2-(3,4-dibromo-2,5-dioxo-2,5-dihydro-1H-pyrrol-1-yl) acetic acid (AV-DL126-Int-01, 2*5.3 mg, 0.017 mmol) in DMSO (0.5 mL) was added EEDQ (2*5 mg, 0.02 mmol) at room temperature and stirred for 5 mints, was added (2S,3S,4S,5R,6S)-6-(2-(3-((S)- 6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid (AV-DL055-Int-13, 2*20 mg, 0.017 mmol) in DMSO (0.5 mL) at room temperature and stirred for 2 h (The reaction was monitored by LCMS). The reaction mixture was diluted with water (0.2 mL) and purified by reverse phase Biotage (column 12g, C18) eluted with 50% MeCN in water to afford 30 mg of semi pure compound which was re-purified by RP prep HPLC and fractions were lyophilized to afford (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid (AV-DL126, 13 mg, 0.009 mmol, 26% yield) as a pale yellow solid.

LCMS (ESI, m/z): 1459.75 [M+H]⁺: ¹H NMR (400 MHz, DMSO-d₆): δ 12.78 (brs, 2H), 9.09 (brs, 1H), 8.41 (d, J=8.8 Hz, 2H), 8.18 (s, 1H), 8.10-8.04 (m, 2H), 7.78 (d, J=10.8 Hz, 1H), 7.31 (s, 1H), 7.10 (s, 2H), 6.51 (brs, 1H), 5.82 (brs, 1H), 5.44 (s, 2H), 5.40-5.21 (m, 6H), 5.10-5.06 (m, 3H), 4.86 (d, J=7.2 Hz, 1H), 4.74 (t, J=8.8 Hz, 1H), 4.21-4.16 (m, 3H), 3.89 (d, J=9.6 Hz, 1H), 3.59 (d, J=9.2 Hz, 1H), 3.45-3.27 (m, 5H), 3.25-3.17 (m, 4H), 3.14-3.08 (m, 2H), 2.56-2.51 (m, 2H), 2.33 (s, 3H), 2.21-2.12 (m, 2H), 2.10-2.04 (m, 2H), 1.90-1.82 (m, 2H), 1.61-1.52 (m, 1H), 1.51-1.38 (m, 3H), 0.88 (t, J=7.2 Hz, 3H).

Prep-HPLC conditions: Column: GEMINI C18 (250*21.2 mm) 5μ; Buffer A: 0.1% TFA in water, ACN:H₂O (70:30); Gradient (T/% B): 0/35, 12/60, 18/960,18.1/98; Flow Rate: 10 mL/min.; Solubility: ACN+H₂O.

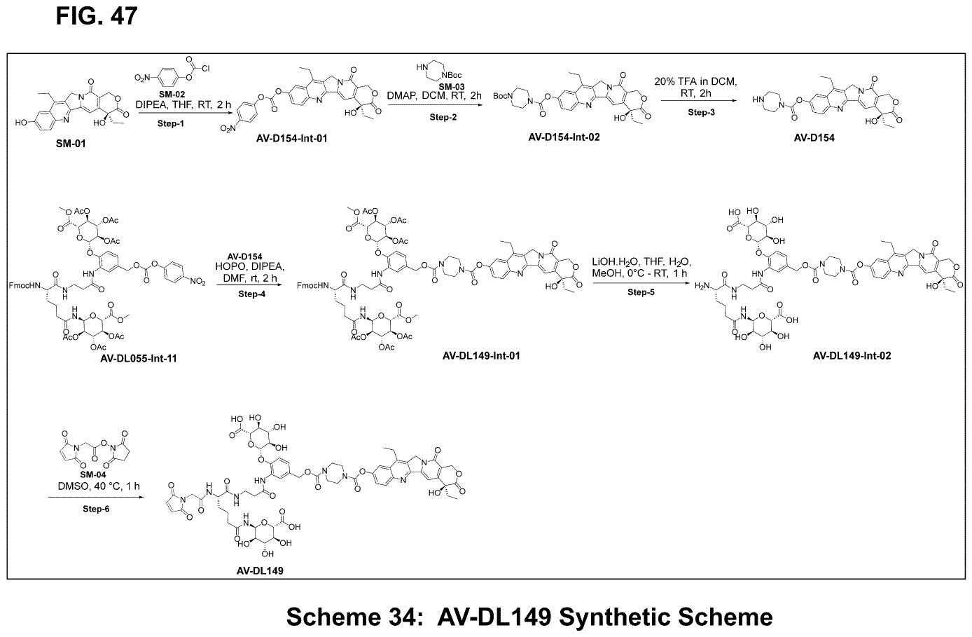

### XXXIV. Synthetic Scheme for AV-DL149 (Scheme 34, FIG. 47)

### a. Synthetic scheme for (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-(((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)carbonyl) piperazine-1-carbonyl)oxy)methyl)phenoxy)-3,4,5-trihydroxytetrahydro-2H-pyran-2-carboxylic Acid (AV-DL149)

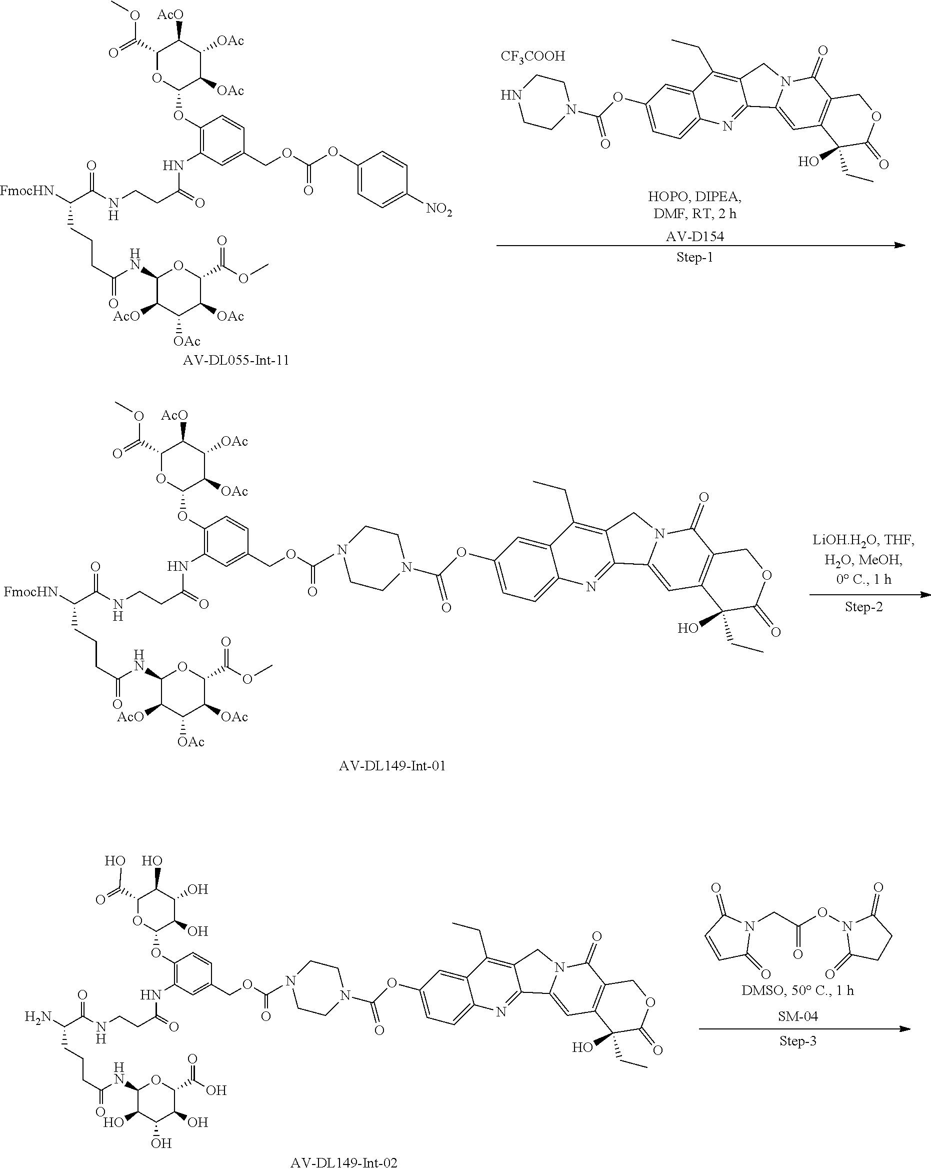

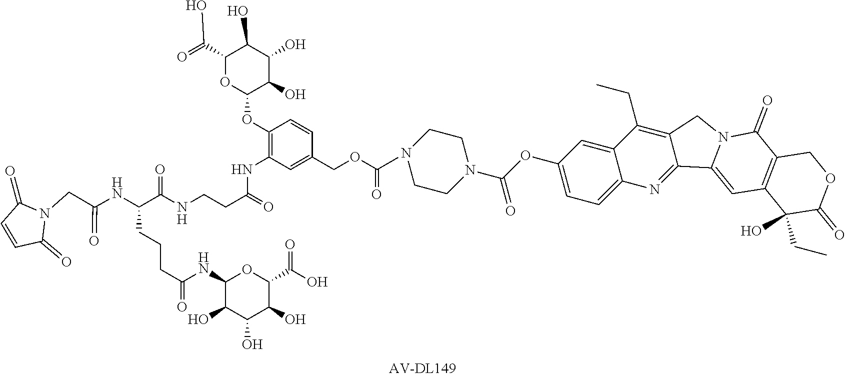 [SMILES: CCc1c2c(nc3ccc(OC(=O)N4CCN(C(=O)OCc5ccc(O[C@@H]6O[C@H](C(=O)O)[C@@H](O)[C@H](O)[C@H]6O)c(NC(=O)CCNC(=O)[C@H](CCCC(=O)N[C@H]6O[C@H](C(=O)O)[C@@H](O)[C@H](O)[C@H]6O)NC(=O)CN6C(=O)C=CC6=O)c5)CC4)cc13)-c1cc3c(c(=O)n1C2)COC(=O)[C@]3(O)CC]

### b. Synthesis of 1-(3-(3-((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanamido)propanamido)-4-(((2S,3R,4S,5S,6S)-3,4,5-triacetoxy-6-(methoxycarbonyl)tetrahydro-2H-pyran-2-yl)oxy)benzyl) 4-((S)-4,11-diethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (AV-DL149-Int-01)

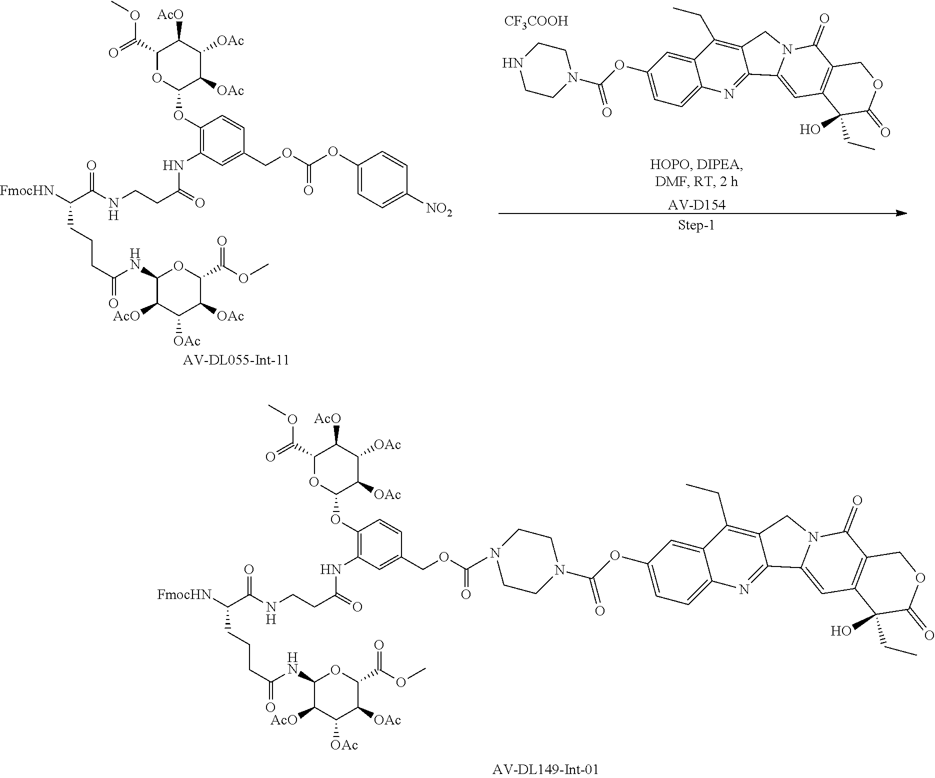

To a stirred solution of (2S,3R,4S,5S,6S)-2-((S)-5-((((9H-fluoren-9-yl)methoxy)carbonyl)amino)-6-((3-((5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-3-oxopropyl)amino)-6-oxohexanamido)-6-(methoxycarbonyl)tetrahydro-2H-pyran-3,4,5-triyl triacetate (AV-DL055-Int-11, 200 mg, 0.145 mmol), (S)-4,11-diethyl-4-hydroxy-3,14-dioxo-3,4,12,14-tetrahydro-1H-pyrano[3′,4′:6,7]indolizino[1,2-b]quinolin-9-yl piperazine-1-carboxylate trifluoroacetate (AV-D154, 73 mg, 0.118 mmol) in DMF (10 mL) were added HOPO (48 mg, 0.435 mmol) and DIPEA (0.12 mL, 0.725 mmol) at RT, and the mixture was stirred at same temperature for 2 h (monitored by LCMS). The resulting reaction mixture was concentrated under reduced pressure to get crude product, which was purified on Biotage flash system by reverse C18 column (40 g), eluted with 50% MeCN in water to get 1-(3-(3-((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanamido)propanamido)-4-(((2S,3R,4S,5S,6S)-3,4,5-triacetoxy-6-(methoxycarbonyl)tetrahydro-2H-pyran-2-yl)oxy)benzyl) 4-((S)-4,11-diethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (AV-DL149-Int-01, 140 mg, 0.080 mmol, 68% yield) as an off-white solid.

MS (ESI): 1738.22 [M+H]⁺.

### c. Synthesis of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-(((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)carbonyl) piperazine-1-carbonyl)oxy)methyl)phenoxy)-3,4,5-trihydroxytetrahydro-2H-pyran-2-carboxylic Acid (AV-DL149-Int-02)

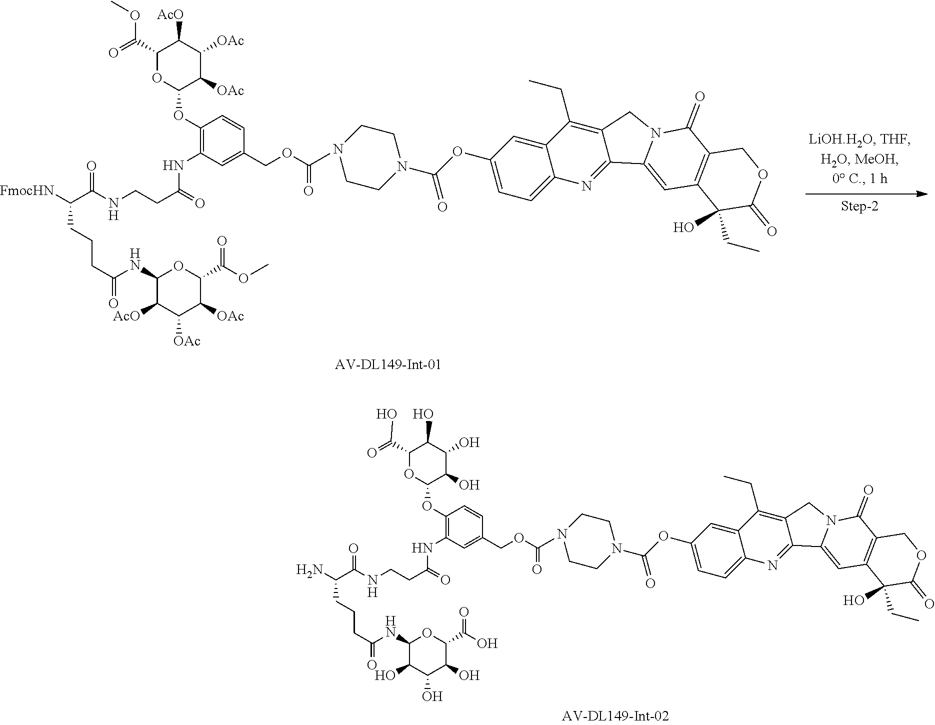

To a stirred solution of 1-(3-(3-((S)-2-((((9H-fluoren-9-yl)methoxy)carbonyl)amino)-6-oxo-6-(((2S,3R,4S,5S,6S)-3,4,5-triacetoxy-6-(methoxycarbonyl)tetrahydro-2H-pyran-2-yl)amino)hexanamido)propanamido)-4-(((2S,3R,4S,5S,6S)-3,4,5-triacetoxy-6-(methoxycarbonyl)tetrahydro-2H-pyran-2-yl)oxy)benzyl) 4-((S)-4,11-diethyl-4-hydroxy-3,14-dioxo-3,4,12,14-tetrahydro-1H-pyrano[3′,4′:6,7]indolizino[1,2-b]quinolin-9-yl) piperazine-1,4-dicarboxylate (AV-DL149-Int-01, 100 mg, 0.057 mmol) in THF:H₂O:MeOH (1:1:1, 6 mL) was added LiOH·H₂O (24 mg, 0.575 mmol) at 0° C. and stirred at same temperature for 1 h (monitored by LCMS). The mixture was concentrated under reduced pressure to get crude product, which was purified on Biotage flash system by reverse C18 column (20 g), eluted with 40% MeCN in water to get (2S,3S,4S,5R,6S)-6-(2-(3-((S)-2-amino-6-(((2S,3R,4S,5S,6S)-6 -carboxy-3,4,5-trihydroxytetrahydro-2H-pyran-2-yl)amino)-6-oxohexanamido)propanamido)-4-(((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)carbonyl) piperazine-1-carbonyl)oxy)methyl)phenoxy)-3,4,5-trihydroxytetrahydro-2H-pyran-2-carboxylic acid (AV-DL149-Int-02, 40 mg, 0.032 mmol, 56% yield) as an off-white solid.

MS (ESI): 1235.72 [M+H]⁺.

### d. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-(((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)carbonyl) piperazine-1-carbonyl)oxy)methyl)phenoxy)-3,4,5-trihydroxytetrahydro-2H-pyran-2-carboxylic Acid (AV-DL149)

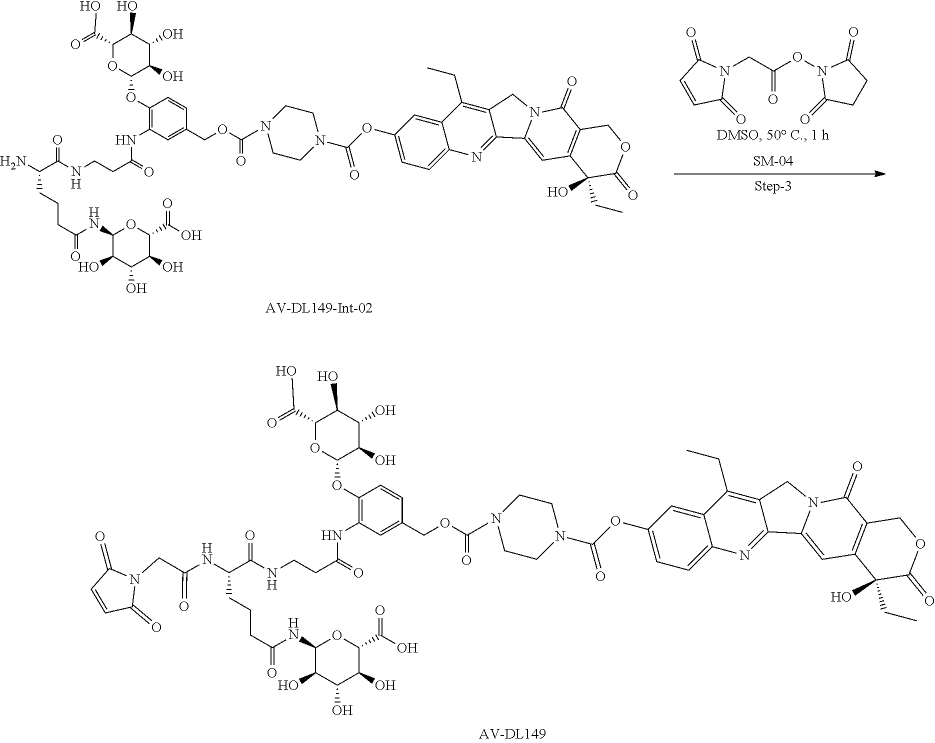 [SMILES: CCc1c2c(nc3ccc(OC(=O)N4CCN(C(=O)OCc5ccc(O[C@@H]6O[C@H](C(=O)O)[C@@H](O)[C@H](O)[C@H]6O)c(NC(=O)CCNC(=O)[C@@H](N)CCCC(=O)N[C@H]6O[C@H](C(=O)O)[C@@H](O)[C@H](O)[C@H]6O)c5)CC4)cc13)-c1cc3c(c(=O)n1C2)COC(=O)[C@]3(O)CC ; O=C(CN1C(=O)C=CC1=O)ON1C(=O)CCC1=O ; CCc1c2c(nc3ccc(OC(=O)N4CCN(C(=O)OCc5ccc(O[C@@H]6O[C@H](C(=O)O)[C@@H](O)[C@H](O)[C@H]6O)c(NC(=O)CCNC(=O)[C@H](CCCC(=O)N[C@H]6O[C@H](C(=O)O)[C@@H](O)[C@H](O)[C@H]6O)NC(=O)CN6C(=O)C=CC6=O)c5)CC4)cc13)-c1cc3c(c(=O)n1C2)COC(=O)[C@]3(O)CC]

To a stirred solution of (2S,3S,4S,5R,6S)-6-(2-(3-((S)-2-amino-6-(((2S,3R,4S,5S,6S)-6-carboxy-3,4,5-trihydroxytetrahydro-2H-pyran-2-yl)amino)-6-oxohexanamido)propanamido)-4-(((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)carbonyl) piperazine-1-carbonyl)oxy)methyl)phenoxy)-3,4,5-trihydroxytetrahydro-2H-pyran-2-carboxylic acid (AV-DL149-Int-02, 40 mg, 0.032 mmol) in DMSO (1.0 mL) at room temperature was added 2,5-dioxopyrrolidin-1-yl 2-(2,5-dioxo-2,5-dihydro-1H-pyrrol-1-yl)acetate (SM-04, 24 mg, 0.097 mmol) and stirred at 50° C. for 1 h. The reaction was monitored by LCMS. The resulting reaction mixture was diluted with water (0.2 mL) and purified by RP Prep-HPLC to afford (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(2,5-dioxo-2,5-dihydro-1H-pyrrol-1-yl)acetamido)-6-oxohexanamido)propanamido)-4-(((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)carbonyl) piperazine-1-carbonyl)oxy)methyl)phenoxy)-3,4,5-trihydroxytetrahydro-2H-pyran-2-carboxylic acid (AV-DL149, 5.7 mg, 0.004 mmol, 13% yield) as a yellow solid.

MS (ESI): 1372.82 [M+H]⁺; ¹H NMR (400 MHz, DMSO-d₆+D20 and VT @90° C.): δ 8.19 (d, J=8.8 Hz, 1H), 8.15 (d, J=1.6 Hz, 1H), 7.99 (d, J=2.4 Hz, 1H), 7.68 (dd, J=2.4, 9.2 Hz, 1H), 7.40 (s, 1H), 8.07 (brs, 1H), 7.14 (d, J=8.4 Hz, 1H), 7.08 (dd, J=2.0, 9.2 Hz, 1H), 6.95 (s, 2H), 5.50-5.34 (m, 2H), 5.32 (s, 2H), 5.08 (s, 2H), 5.01 (s, 3H), 4.84 (d, J=7.6 Hz, 1H), 4.79 (d, J=8.8 Hz, 1H), 4.23-4.18 (m, 1H), 4.12 (s, 2H), 3.89 (d, J=10.4 Hz, 1H), 4.68 (d, J=10.4 Hz, 1H), 3.66-3.62 (m, 4H), 3.60-3.55 (m, 3H), 3.54-3.48 (m, 2H), 3.46-3.38 (m, 5H), 3.32-3.28 (m, 1H), 3.23-3.18 (m, 3H), 2.59 (t, J=7.6 Hz, 2H), 2.22-2.12 (m, 2H), 1.96-1.85 (m, 2H), 1.75-1.67 (m, 1H), 1.62-1.47 (m, 3H), 1.33 (t, J=7.6 Hz, 3H), 0.91 (t, J=7.6 Hz, 3H).

Prep-HPLC conditions: Column: GEMINI C18 (250*21.2 mm) 5μ; Buffer A: 0.1% TFA in water, ACN:H₂O (80:20); Gradient (T/% B): 0/30,16/60, 16.05/98; Flow Rate: 10 mL/min.; Solubility: ACN+H₂O.

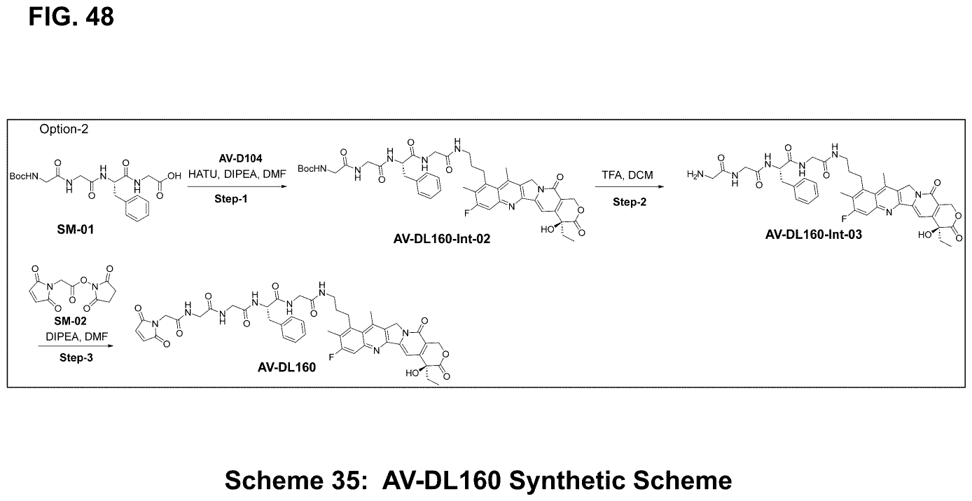

### XXXI. Synthetic Scheme for AV-DL160 (Scheme 35, FIG. 48)

### a. Synthetic scheme of(S)-2-(2-(2-(2-(2,5-dioxo-2,5-dihydro-1H-pyrrol-1-yl)acetamido)acetamido)acetamido)-N-(2-((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)amino)-2-oxoethyl)-3-phenylpropanamide (AV-DL160)

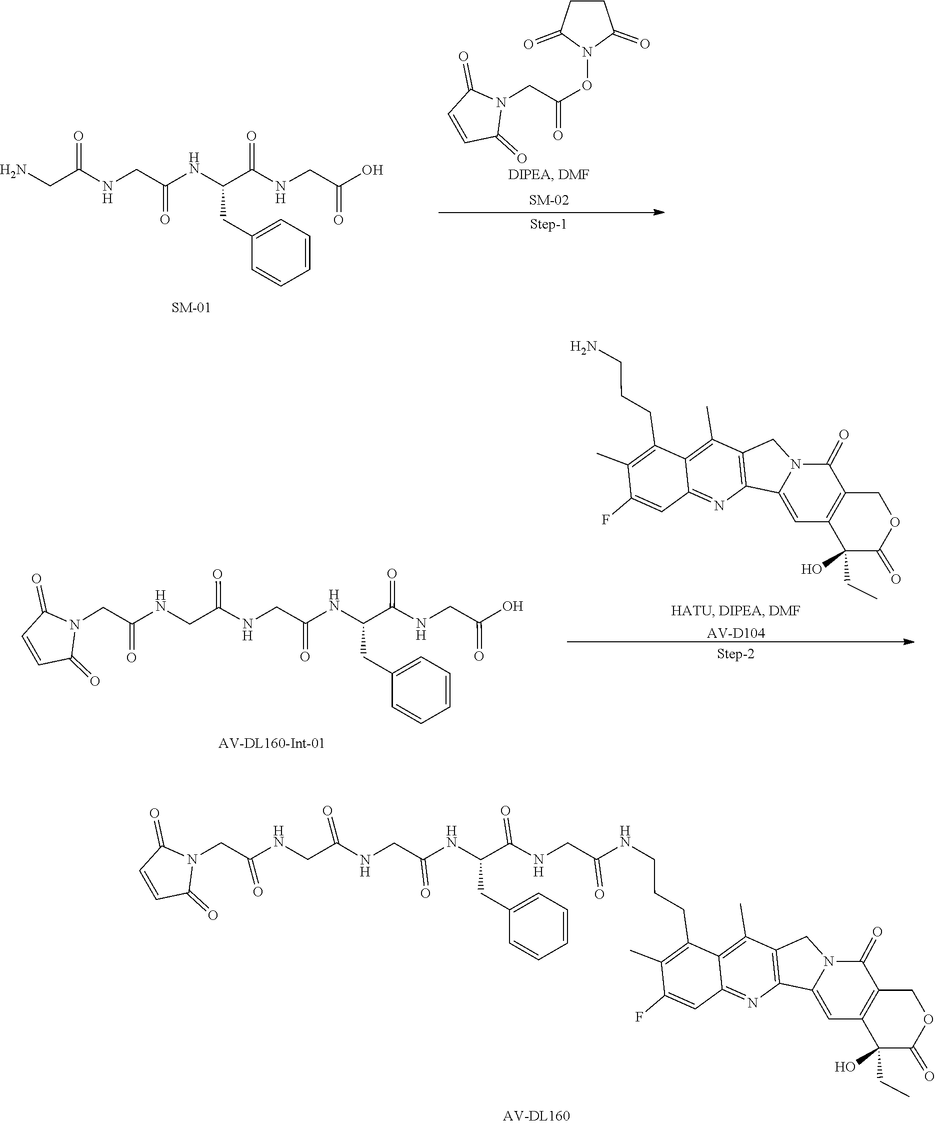 [SMILES: NCC(=O)NCC(=O)N[C@@H](Cc1ccccc1)C(=O)NCC(=O)O ; O=C(CN1C(=O)C=CC1=O)ON1C(=O)CCC1=O ; O=C(O)CNC(=O)[C@H](Cc1ccccc1)NC(=O)CNC(=O)CNC(=O)CN1C(=O)C=CC1=O ; CC[C@@]1(O)C(=O)OCc2c1cc1n(c2=O)Cc2c-1nc1cc(F)c(C)c(CCCN)c1c2C ; "AV-D104" ; CC[C@@]1(O)C(=O)OCc2c1cc1n(c2=O)Cc2c-1nc1cc(F)c(C)c(CCCNC(=O)CNC(=O)[C@H](Cc3ccccc3)NC(=O)CNC(=O)CNC(=O)CN3C(=O)C=CC3=O)c1c2C]

### b. Synthesis of (2-(2,5-dioxo-2,5-dihydro-1H-pyrrol-1-yl) acetyl)glycylglycyl-L-phenylalanylglycine (AV-DL160-Int-01)

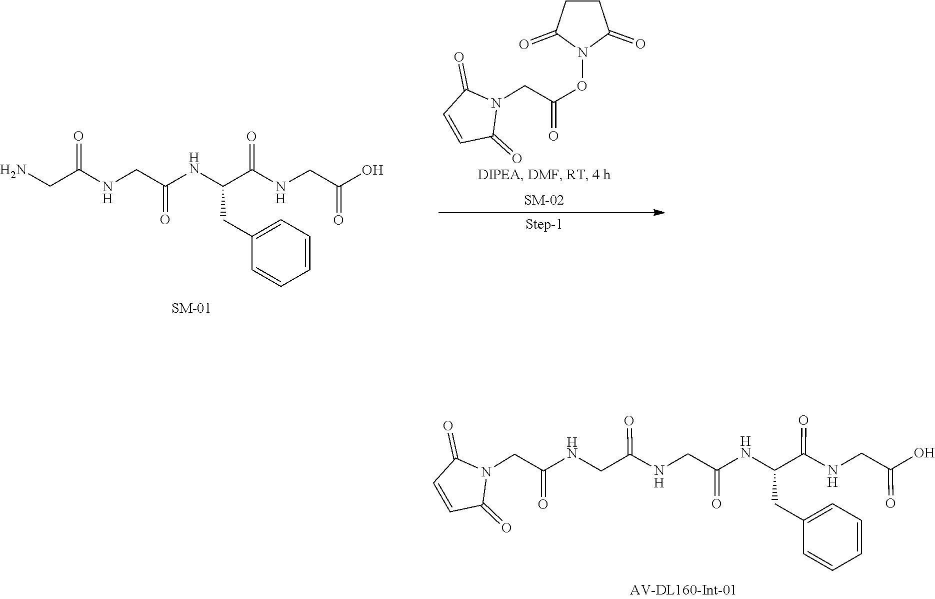 [SMILES: NCC(=O)NCC(=O)N[C@@H](Cc1ccccc1)C(=O)NCC(=O)O ; O=C(CN1C(=O)C=CC1=O)ON1C(=O)CCC1=O ; O=C(O)CNC(=O)[C@H](Cc1ccccc1)NC(=O)CNC(=O)CNC(=O)CN1C(=O)C=CC1=O]

To a stirred solution of glycylglycyl-L-phenylalanylglycine (SM-01, 1.0 g, 2.976 mmol, 1 eq), DIPEA (1.03 mL, 5.952 mmol, 2 eq) in DMF (10 mL) was added 2,5-dioxopyrrolidin-1-yl 2-(2,5-dioxo-2,5-dihydro-1H-pyrrol-1-yl)acetate (SM-02, 0.9 g, 3.571 mmol, 1.2 eq) at room temperature and stirred at same temperature for 4 h (monitored by LCMS). Then, the reaction mixture was concentrated under reduced pressure to get crude product. The crude product was purified on Biotage flash system by reverse phase C-18 column (60 g), eluted with 20-25% MeCN in water and fractions were lyophilized to afford (2-(2,5-dioxo-2,5-dihydro-1H-pyrrol-1-yl) acetyl)glycylglycyl-L-phenylalanylglycine (AV-DL160-Int-01)(0.82 g, 1.733 mmol, 58% yield) as a colorless gummy solid.

MS (ESI): 474.12 [M+H]⁺.

### c. Synthesis of(S)-2-(2-(2-(2-(2,5-dioxo-2,5-dihydro-1H-pyrrol-1-yl)acetamido)acetamido)acetamido)-N-(2-((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)amino)-2-oxoethyl)-3-phenylpropanamide (AV-DL160)

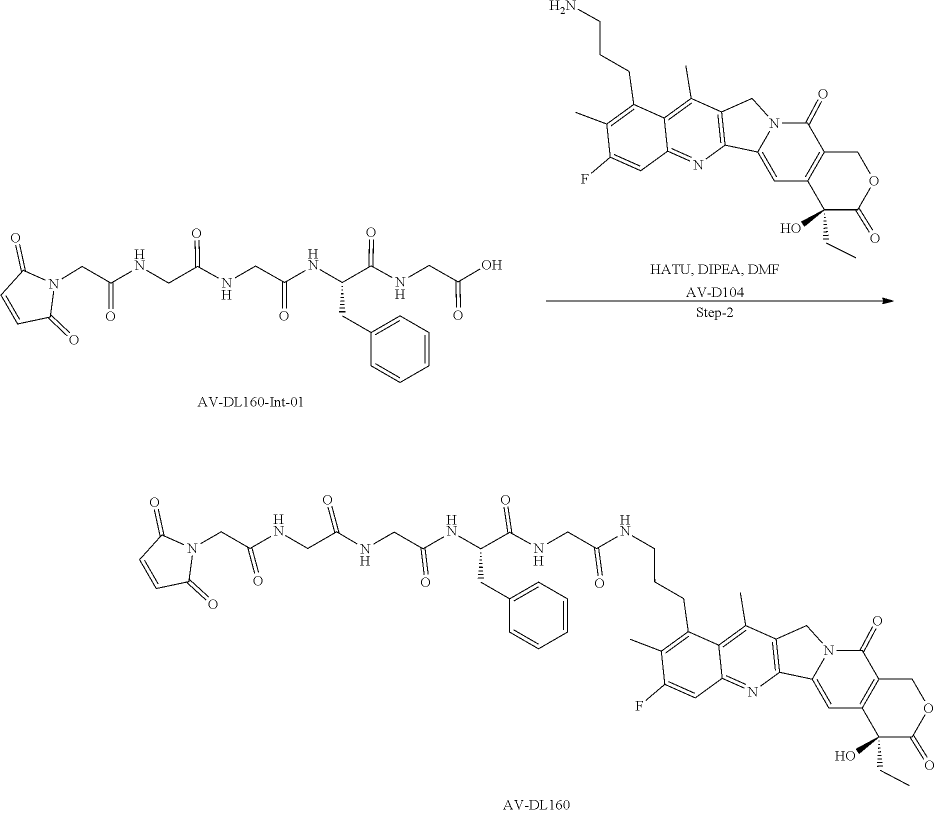 [SMILES: O=C(O)CNC(=O)[C@H](Cc1ccccc1)NC(=O)CNC(=O)CNC(=O)CN1C(=O)C=CC1=O ; CC[C@@]1(O)C(=O)OCc2c1cc1n(c2=O)Cc2c-1nc1cc(F)c(C)c(CCCN)c1c2C ; "AV-D104" ; CC[C@@]1(O)C(=O)OCc2c1cc1n(c2=O)Cc2c-1nc1cc(F)c(C)c(CCCNC(=O)CNC(=O)[C@H](Cc3ccccc3)NC(=O)CNC(=O)CNC(=O)CN3C(=O)C=CC3=O)c1c2C]

To a stirred solution of (2-(2,5-dioxo-2,5-dihydro-1H-pyrrol-1-yl) acetyl)glycylglycyl-L-phenylalanylglycine (AV-DL160-Int-01)(78.6 mg, 0.166 mmol, 1.5 eq) in DMF (0.5 mL) were added HATU (63.2, 0.166 mmol, 1.5 eq) followed by DIPEA (58 μL, 0.332 mmol, 3 eq) at room temperature and stirred for 5 min. Then, was added(S)-10-(3-aminopropyl)-4-ethyl-8 -fluoro-4-hydroxy-9,11-dimethyl-1,12-dihydro-14H-pyrano[3′,4′:6,7]indolizino[1,2-b]quinoline-3,14(4H)-dione (AV-D104, 50 mg, 0.11 mmol, 1 eq) in DMF (0.5 mL) at room temperature and stirred for 4 h (monitored by LCMS). The reaction mixture was diluted with water (10 mL), washed with diether ether (2×5 mL) and the aqueous layer was extracted with 10% MeOH in DCM (2×10 mL). The 10% MeOH in DCM layers were combined, dried over anhydrous sodium sulphate, filtered and concentrated to obtain crude compound which was purified by prep HPLC. The pure fractions were lyophilized to afford(S)-2-(2-(2-(2-(2,5-dioxo-2,5-dihydro-1H-pyrrol-1-yl)acetamido)acetamido)acetamido)-N-(2-((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)amino)-2-oxoethyl)-3-phenylpropanamide (AV-DL160, 6.9 mg, 0.008 mmol, 7.2% yield) as a pale yellow solid.

LCMS (ESI, m/z): 907.3 [M+H]⁺:

Prep-HPLC conditions: Column: GEMINI C18 (250*21.2 mm) 5μ; Buffer A: 0.1% TFA in water, ACN:H₂O (70:30); Gradient (T/% B): 0/35,12/60, 18/960,18.1/98; Flow Rate: 10 mL/min.; Solubility: ACN+H₂O.

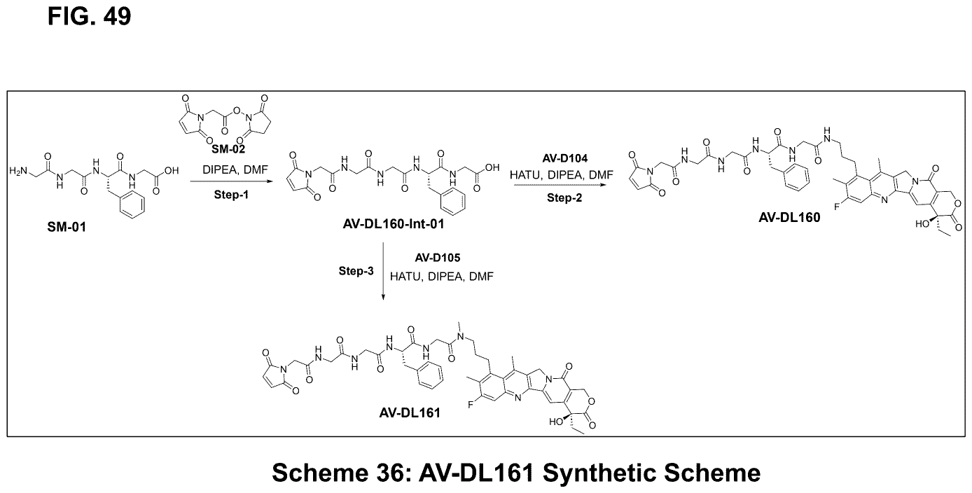

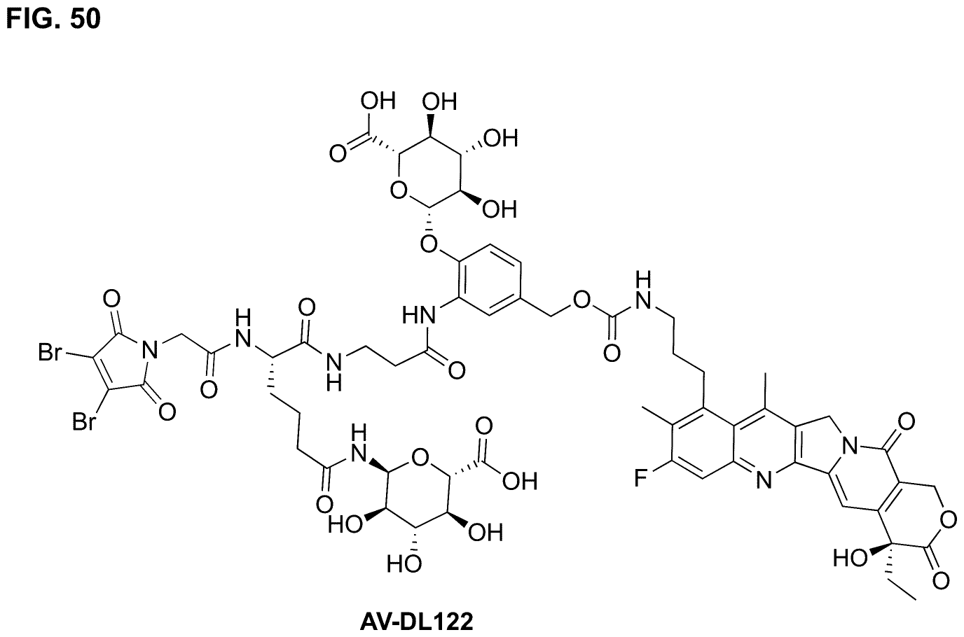

### XXXI. Synthetic Scheme for AV-DL161 (Scheme 36, FIG. 49)

### a. Synthesis of(S)-2-(2-(2-(2-(2,5-dioxo-2,5-dihydro-1H-pyrrol-1-yl)acetamido)acetamido)acetamido)-N-(2-((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)amino)-2-oxoethyl)-3-phenylpropanamide (AV-DL161)

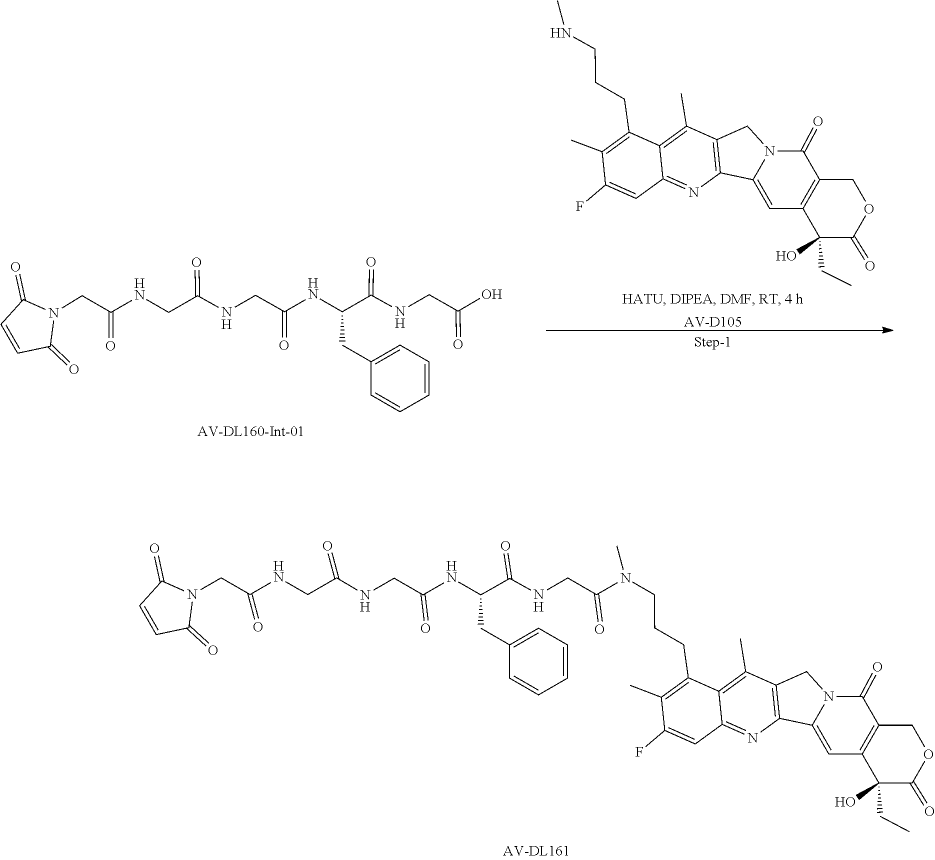 [SMILES: O=C(O)CNC(=O)[C@H](Cc1ccccc1)NC(=O)CNC(=O)CNC(=O)CN1C(=O)C=CC1=O ; CC[C@@]1(O)C(=O)OCc2c1cc1n(c2=O)Cc2c-1nc1cc(F)c(C)c(CCCNC)c1c2C ; "AV-D105" ; CC[C@@]1(O)C(=O)OCc2c1cc1n(c2=O)Cc2c-1nc1cc(F)c(C)c(CCCN(C)C(=O)CNC(=O)[C@H](Cc3ccccc3)NC(=O)CNC(=O)CNC(=O)CN3C(=O)C=CC3=O)c1c2C]

To a stirred solution of (2-(2,5-dioxo-2,5-dihydro-1H-pyrrol-1-yl) acetyl)glycylglycyl-L-phenylalanylglycine (AV-DL160-Int-01)(76.3 mg, 0.161 mmol, 1.5 eq) in DMF (0.5 mL) were added HATU (61.2, 0.161 mmol, 1.5 eq) followed by DIPEA (56.2 μL, 0.322 mmol, 3 eq) at room temperature and stirred for 5 min. Then, was added(S)-4-ethyl-8-fluoro-4-hydroxy-9,11-dimethyl-10-(3-(methylamino)propyl)-1,12-dihydro-14H-pyrano[3′,4′:6,7]indolizino[1,2 -b]quinoline-3,14(4H)-dione (AV-D105, 50 mg, 0.107 mmol, 1 eq) in DMF (0.5 mL) at room temperature and stirred for 4 h (monitored by LCMS). The reaction mixture was diluted with water (10 mL), washed with diether ether (2×5 mL) and the aqueous layer was extracted with 10% MeOH in DCM (2×10 mL). The 10% MeOH in DCM layers were combined, dried over anhydrous sodium sulphate, filtered and concentrated to obtain crude compound which was purified by prep HPLC. The pure fractions were lyophilized to afford(S)-2-(2-(2-(2-(2,5-dioxo-2,5-dihydro-1H-pyrrol-1-yl)acetamido)acetamido)acetamido)-N-(2-((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)(methyl)amino)-2-oxoethyl)-3-phenylpropanamide (AV-DL161, 6.2 mg, 0.007 mmol, 6.5% yield) as a pale yellow solid.

LCMS (ESI, m/z): 921.13 [M+H]⁺:

Prep-HPLC conditions: Column: GEMINI C18 (250*21.2 mm) 5μ; Buffer A: 0.1% TFA in water, ACN:H₂O (70:30); Gradient (T/% B): 0/35,12/60,18/960,18.1/98; Flow Rate: 10 mL/min.; Solubility: ACN+H₂O.

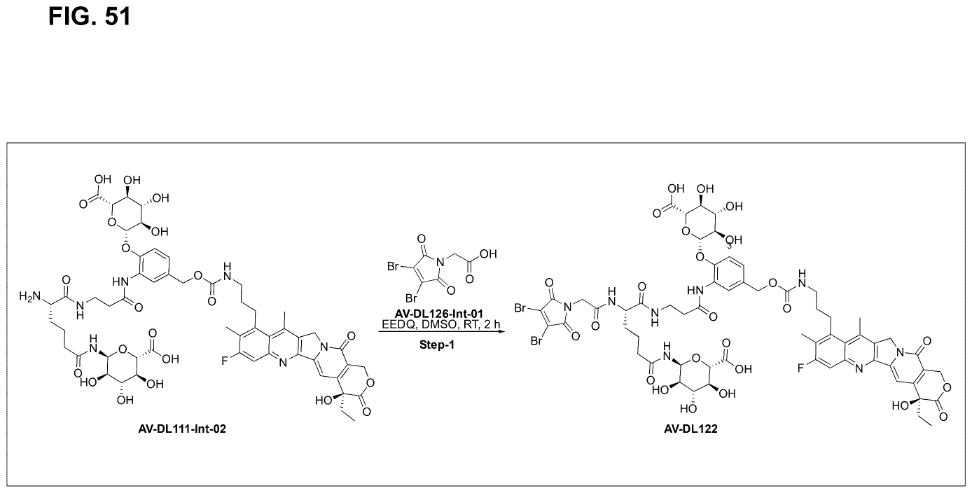

### XXXI. Synthetic Scheme for AV-DL122 (Scheme 37, FIG. 51)

### a. Synthesis of (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic Acid (AV-DL122)

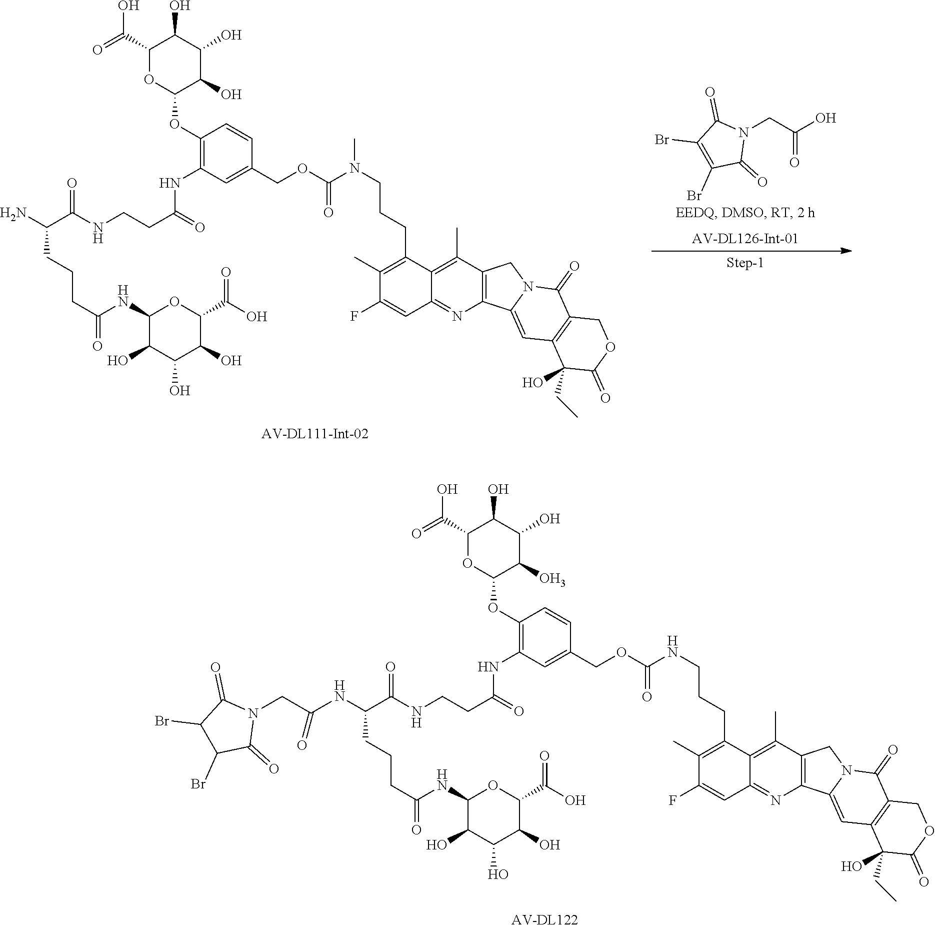 [SMILES: CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@@H](N)CCCC(=O)N[C@H]4O[C@H](C(=O)O)[C@@H](O)[C@H](O)[C@H]4O)c3)c1c2C ; O=C(O)CN1C(=O)C(Br)=C(Br)C1=O ; CC[C@@]1(O)C(=O)OCc2c1cc1n(c2=O)Cc2c-1nc1cc(F)c(C)c(CCCNC(=O)OCc3ccc(O[C@@H]4O[C@H](C(=O)O)[C@@H](O)[C@H](O)[C@H]4O)c(NC(=O)CCNC(=O)[C@H](CCCC(=O)N[C@H]4O[C@H](C(=O)O)[C@@H](O)[C@H](O)[C@H]4O)NC(=O)CN4C(=O)C(Br)C(Br)C4=O)c3)c1c2C]

To a stirred solution of 2-(3,4-dibromo-2,5-dioxo-2,5-dihydro-1H-pyrrol-1-yl) acetic acid (AV-DL126-Int-01, 7.8 mg, 0.025 mmol) in DMSO (0.5 mL) was added EEDQ (7.4 mg, 0.03 mmol) at room temperature and stirred for 5 min, was added ((2S,3S,4S,5R,6S)-6-(2-(3-((S)- 2-amino-6-(((2S,3R,4S,5S,6S)-6-carboxy-3,4,5-trihydroxytetrahydro-2H-pyran-2-yl)amino)-6-oxohexanamido)propanamido)-4-((((3-((S)-4-ethyl-8-fluoro-4-hydroxy-9,11-dimethyl-3,14-dioxo-3,4,12,14-tetrahydro-1H-pyrano[3′,4′:6,7]indolizino[1,2-b]quinolin-10-yl)propyl)carbamoyl)oxy)methyl)phenoxy)-3,4,5-trihydroxytetrahydro-2H-pyran-2-carboxylic acid (AV-DL111-Int-02, 30 mg, 0.025 mmol) in DMSO (0.5 mL) at room temperature and stirred for 2 h (monitored by LCMS). The reaction mixture was diluted with water (0.2 mL) and purified on Biotage flash system by reverse phase C18 column (12g), eluted with 60% MeCN in water to afford 15 mg of semi pure compound which was re-purified by RP prep HPLC and fractions were lyophilized to afford (2S,3S,4S,5R,6S)-6-(2-(3-((S)-6-(((2S,3R,4S,5S,6S)-6-carboxy-3,4,5-trihydroxytetrahydro-2H-pyran-2-yl)amino)-2-(2-(3,4-dibromo-2,5-dioxo-2,5-dihydro-1H-pyrrol-1-yl)acetamido)-6-oxohexanamido)propanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy))phenoxy)-3,4,5-trihydroxytetrahydro-2H-pyran-2-carboxylic acid (AV-DL122, 4 mg, 0.002 mmol, 10% yield) as a pale yellow solid.

LCMS (ESI, m/z): 1490.31 [M+H]⁺: ¹H NMR (400 MHz, DMSO-d₆): δ 12.78 (brs, 2H), 9.11-9.06 (m, 1H), 8.40 (d, J=8.8 Hz, 2H), 8.22 (brs, 1H), 8.08 (brs, 1H), 7.77 (d, J=10.4 Hz, 1H), 7.28 (s, 1H), 7.08-7.03 (m, 2H), 6.50 (brs, 1H), 5.82 (brs, 1H), 5.44 (s, 3H), 5.40-5.12 (m, 6H), 5.01 (s, 2H), 4.85 (brs, 1H), 4.75 (t, J=8.8 Hz, 1H), 4.21-4.18 (m, 3H), 3.88 (d, J=9.2 Hz, 1H), 3.59 (d, J=10.2 Hz, 1H), 3.46-3.40 (m, 4H), 3.10 (t, J=8.8 Hz, 1H), 2.96-2.90 (m, 6H), 2.82 (brs, 2H), 2.60-2.53 (m, 2H), 2.44-2.37 (m, 3H), 2.20-2.07 (m, 3H), 1.89-1.75 (m, 4H), 1.64-1.57 (m, 1H), 1.52-1.40 (m, 4H), 0.88 (t, J=7.2 Hz, 3H).

### Prep-HPLC Conditions: Column: GEMINI C18 (250*21.2 mm) 5μ; Buffer-A: 0.1% TFA in Water, ACN:H₂O (80:20); Gradient (T/% B): 0/35,12/70,20.01/98; Flow Rate: 10 mL/min.; Solubility: ACN+H₂O. Preparation of Conjugates of this Disclosure

The compounds of Formula V or conjugates of the present disclosure were prepared out of the compounds of Formula I provided in Example 1 with a linker selected from the compounds provided in Example 2.

In some embodiments, the payload is attached to the targeting moiety, e.g., an antibody, through conjugation through cysteine lysine, tyrosine or arginine residues in the targeting moiety. In some embodiments, conjugation involves covalent binding through a linker.

a. Cysteine Residue Conjugation

Cysteine residue conjugation involves targeting the thiol (—SH) group of cysteine residues in an antibody. This can be achieved, for example, by reducing existing disulfide bonds to expose free thiols or by engineering additional cysteine residues into the antibody. The exposed thiol groups are reactive and can be conjugated with, for example, maleimide-functionalized drugs or labels, forming stable thioether bonds.

b. Lysine Residue Conjugation

Lysine residue conjugation involves targeting the amine groups (—NH₂) on the side chains of lysine residues. This can be done, for example, by using N-hydroxysuccinimide (NHS) esters, which react with the amine groups to form stable amide bonds.

c. Other Conjugation Methods

Linkers also can be conjugated through a non-natural amino acid, disulfide re-bridging, a peptide tag, glycan modification, or an enzymatic modification method used to generate site-specific immunoconjugates.

### Assessment of Cytotoxicity of the Compounds of this Disclosure

The cytotoxicity of the fluorocamptothecin compounds of Formula I was assessed in vitro with the following cancer cell lines: SKBR3 (breast cancer), JIMT1 (breast tumour model) and SKOV-3 (ovarian cancer).

Cytotoxicity of the compounds of Formula I against said cancer cells was determined by assessing growth inhibition by CellTiter-Glo® (Promega Corporation, Madison, WI) and by measuring luminescence on a Synergy™ H₁ plate reader (BioTek Instruments, Winooski, VT). IC₅₀ values were determined by GraphPad Prism (GraphPad Software, San Diego, CA). Dilutions of compounds of Formula I shown in Table 1 were prepared in RPMI 1640+10% FBS, and 20 μL of each dilution was added to 384-well plates. Cells cultured in log-phase growth were detached by brief incubation in 0.05% Trypsin and resuspended in respective culturing media at 20,000 cells/mL. 50 μL of cell suspension was then added to the plates containing test articles. Cells were incubated with test articles for 4 d at 37° C. and the growth inhibition and corresponding IC₅₀ were measured and is shown in Table 1 below.

TABLE 1
In vitro potency of Compounds of Formula I (IC₅₀)

| | | IC₅₀ nM | | |
|---|---|---|---|---|
| Sl. No. | Payload | SKBR3 | JIMT1 | SKOV3 |
| 1 | Dxd (deruxtecan, | 0.4 | 5.2 | 7.5 |
| | Comparative) | | | |
| 2 | AV-D021 | 0.52 | 0.51 | 0.18 |
| 3 | AV-D042 | 1.32 | 0.51 | 2.71 |
| 4 | AV-D042A | 0.09 | 0.03 | 0.08 |
| 5 | AV-D043 | 1.23 | 1.02 | 5.04 |
| 6 | AV-D043A | 0.55 | 1.7 | 1.3 |
| 7 | AV-D050 | 1.24 | 10.10 | |
| 8 | AV-D050A | 1.28 | 28.64 | |
| 9 | AV-D077 | 0.27 | 14.20 | 5.38 |

The compounds of Formula I showed appreciable potency against the tumour cells. In comparison to the conventionally used deruxtecan (DXd) camptothecin analogue, the compounds of Formula I of the present disclosure exhibited comparable cytotoxicity against the cancer cell lines: SKBR3 (breast cancer), JIMT1 (breast tumour model) and SKOV-3 (ovarian cancer).

The properties of the fluorocamptothecin compounds of Formula IV were assessed.

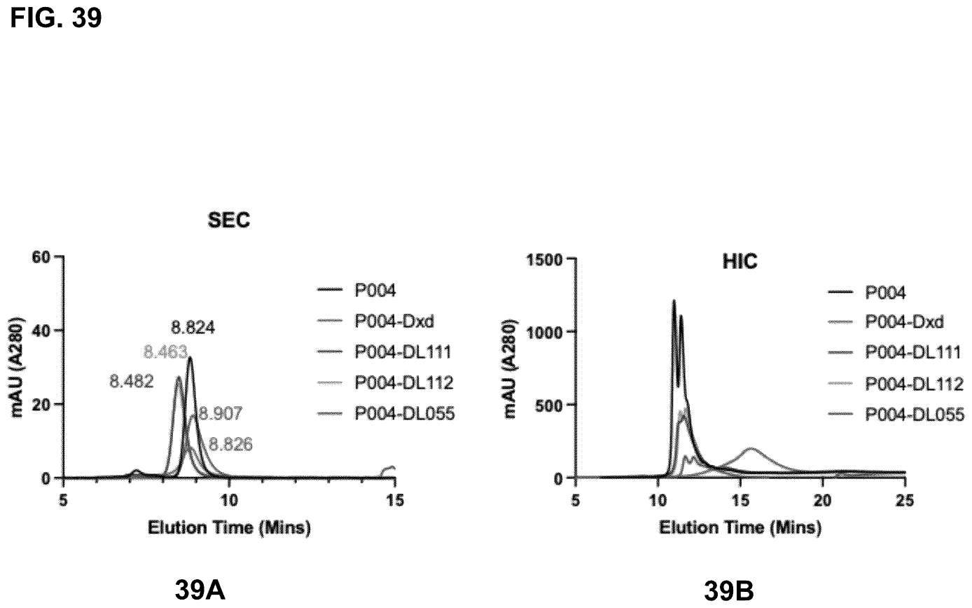

Referring to FIG. 39A, antibody or ADC samples are analyzed on a SEC column-TSKgel QC-PAK-GFC 300 for 20 min at 0.5 mL/min flow, 25° C. column temperature using the mobile phase (1×PBS, 10% Isopropanol, pH 7.4). Protein retention was monitored over an isocratic elution of 1.5 CV in length by monitoring absorbance at either 280 or 215 nm. As shown in the graph, all ADC samples showed a single SEC peak confirming their homogeneity on SEC.

Referring to FIG. 39B (HIC), ADC samples were analyzed by hydrophobic interaction chromatography (HIC) analysis on TSKgel Butyl-NPR for 30 min at 0.6 mL/min flow, 25° C. column temperature using following buffers and buffer gradients: Buffer A: 50 mM Sodium phosphate, 1.5 M Ammonium Sulfate, pH 7.0; Buffer B: 50 mM Sodium phosphate, 20% Isopropanol, pH 7.0. The column mobile phase was used for 0-3 min with Buffer A only and 3-18 min with Buffe B at 0-100% gradient and 18-24 min with Buffer B (100%) and 24-30 min with Buffer A (100%) only. As shown in the HIC graph, P004 (antibody only) showed the elution time of 11 min, while P004-DXd displayed the broadest peak from 11 min to 18 min. This is results of hydrophobicity of the P004-DXd ADC increased upon conjugation to DXd linker-payload. Surprisingly, other three ADCs (P004-DL055, P004-DL111 and P004-DAR112) displayed similar elution profile as P004 indicating that their hydrophobicity did not change upon conjugation to their respective linker-payloads. Please note that all ADCs have same DAR8 (8 linker-payloads per antibody).

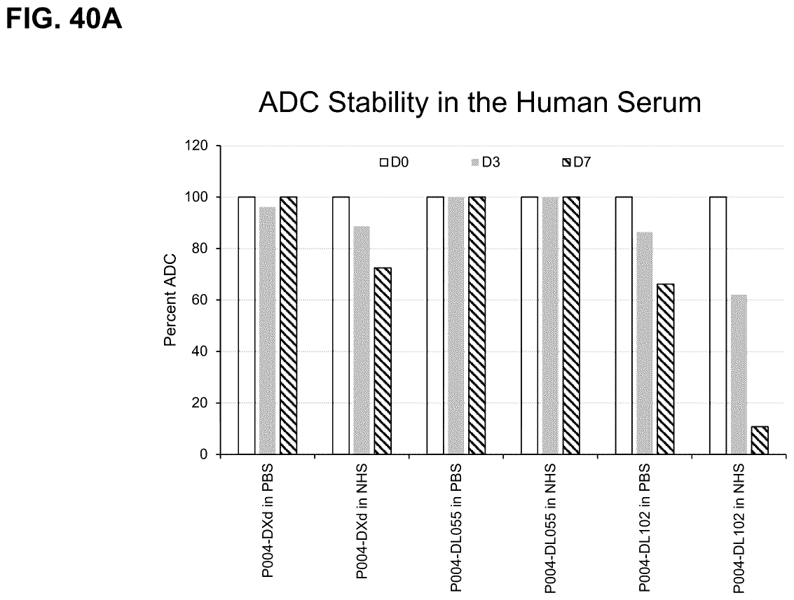

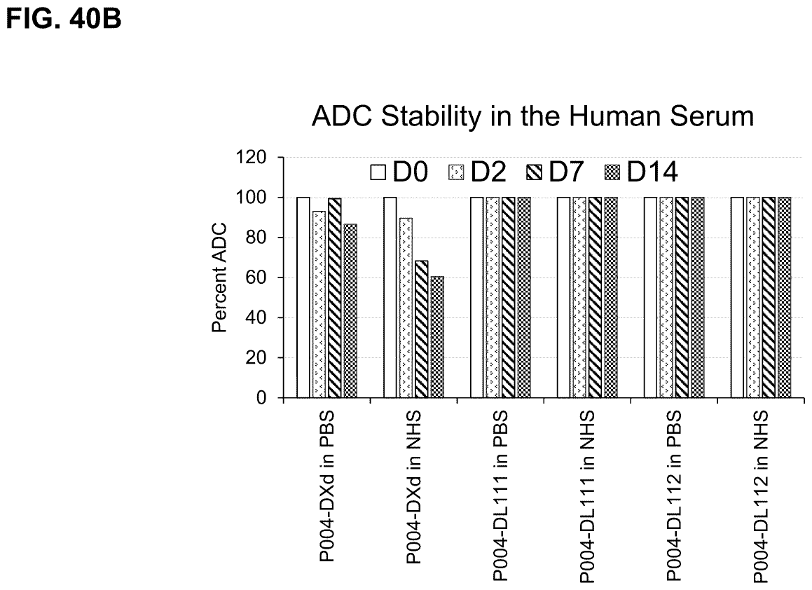

Referring to FIGS. 40A and 40B (Serum Stability), ADC samples were incubated with normal human serum (NHS) samples at a final concentration of 100 μg/ml. PBS with 0.5% BSA was used as a control. Aliquots of 100 μL from each mixture were transferred into silated microcentrifuge tubes and incubated at 37° C. in a CO₂ incubator. This allowed us to keep the plasma pH levels close to the physiological pH of 7.2 throughout the incubation period. To stop the reaction, samples were transferred to a −80° C. freezer at predetermined time points (0, 2 or 3 days, 7 days and 14 days).

ADC samples were purified using biotinylated HER2-ecd/Streptavidin-Dynabeads M-Mix Dynabeads. Samples were then deglycosylated with PNGase F, followed by elution with 30% acetonitrile/0.1% formic acid and analyzed on RP-LC/MS for DAR analysis. As shown in FIGS. 40A and 40B, P004-DXd was relatively stable in PBS buffer but lost 40% DXd linker-payload over 14 days. P004-DL102 ADC was very unstable both in PBS and in the human serum. In contrast, liker-payload attachment in the P004-DL055, P004-DL111 and P004-DL112 ADCs were stable till Day 7 or Day 14 in both PBS and normal human serum.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims.

All publications, patents, and patent applications mentioned in this specification are herein incorporated by reference to the same extent as if each individual publication, patent, or patent application was specifically and individually indicated to be incorporated by reference

### Advantages of the Present Disclosure

The present disclosure provides fluorocamptothecin compounds of Formula I, which shows IC₅₀ and hence better potency against cancer cells having lower IC₅₀. Further, the present disclosure provides a conjugate comprising the compounds of Formula I along with a linker. The present disclosure also provides a convenient yet economic process of preparing the compounds of Formula I.

1. A compound of the formula below, and its a pharmaceutically acceptable salt or derivative thereof:

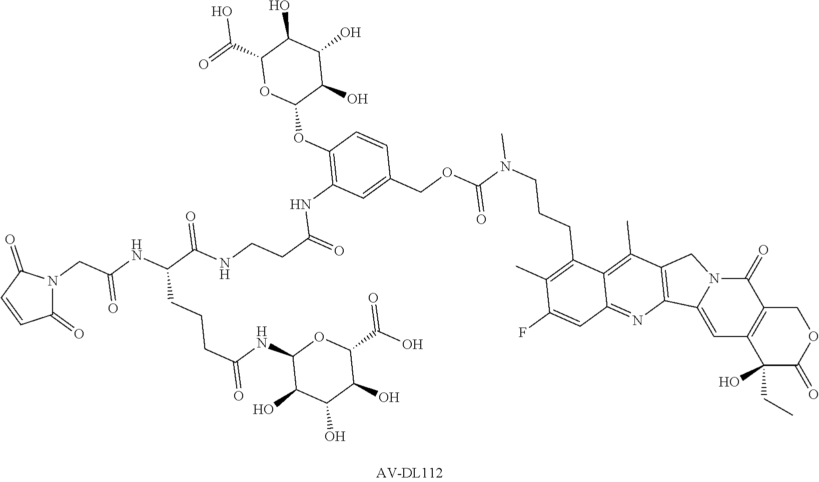 [SMILES: CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@H](CCCC(=O)N[C@H]4O[C@H](C(=O)O)[C@@H](O)[C@H](O)[C@H]4O)NC(=O)CN4C(=O)C=CC4=O)c3)c1c2C]

2. A conjugate comprising a cytotoxic compound attached through a linker to a targeting moiety, wherein the cytotoxic compound and linker have the formula:

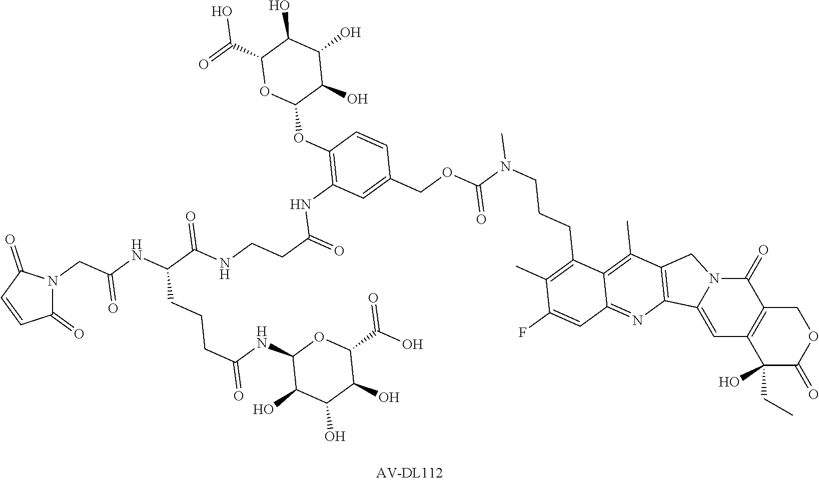 [SMILES: CC[C@@]1(O)C(=O)OCc2c1cc1n(c2=O)Cc2c-1nc1cc(F)c(C)c(CCCN(C)C(=O)OCc3ccc(O[C@@H]4O[C@H](C(=O)O)[C@@H](O)[C@H](O)[C@H]4O)c(NC(=O)CCNC(=O)[C@H](CCCC(=O)N[C@H]4O[C@H](C(=O)O)[C@@H](O)[C@H](O)[C@H]4O)NC(=O)CN4C(=O)C=CC4=O)c3)c1c2C]

3. The conjugate as claimed in claim 2, wherein the targeting moiety comprises an antibody.

4. The conjugate as claimed in claim 3, wherein the antibody binds to a cancer cell.

5. The conjugate as claimed in claim 4, wherein the cancer cell is affected with a cancer selected from adenocarcinoma of the lung, squamous carcinoma of the lung, bladder cancer, blastoma, breast cancer, cancer of the peritoneum, cervical cancer, colon cancer, colorectal cancer, endometrial or uterine carcinoma, esophageal cancer, gastric cancer, glioblastoma, head & neck cancer, hepatic carcinoma, hepatocellular cancer, hepatoma, renal cancer, leukemia, liver cancer, low-grade gliomas (LGG), lung cancer including small-cell lung cancer, lymphoma, mesothelioma, melanoma, multiple myeloma, non-small cell lung cancer (NSCLC), ovarian cancer, pancreatic cancer, prostate cancer, rectal cancer, salivary gland carcinoma, sarcoma, squamous cell cancer, stomach cancer, thyroid cancer, and vulval cancer.

6. The conjugate as claimed in claim 3 or 4, wherein the antibody binds to an antigen selected from: 0772P (CA125, MUC16), 5T4, ADAM9, AG-7, Alkaline phosphatase (ALPP), ASCT2, ASLG659, Axl, BAFF-R (B cell-activating factor receptor), BLyS receptor 3, BR3, BMPR1B (bone morphogenetic protein receptor-type IB), Brevican, B7-H₃, B7-H₄, BCMA, C4.4a, CA6, CA9, CanAg, CCR7, CD19, CD20, CD21 (CR2 (Complement receptor 2), CD22 (B-cell receptor CD22-B isoform), CD30, CD33, CD37, CD38, CD44v6, CD46, CD47, CD48, CD51, CD56, CD70, CD71, CD72 (B-cell differentiation antigen CD72, Lyb-2), CD74, CD79a, CD79b, CD123, CD138, CD142, CD166, CD205, CD228, CD352, CDH6, CEACAM5, CEACAM6, cKIT, CLDN18.2, CLDN6, CLDN9, CLL-1, c-MET, CRIPTO (EGF-CFC), CXCR5 (Burkitt's lymphoma receptor 1), EGFR, EGFRviii, Cripto, CSP-1, DLK-1, DLL3, DPEP3, DR5, EFNA4, ENPP3, EphA2, EphA5, EphB2R, ETBR (Endothelin type B receptor), E16 (LAT1, SLC7A₅), FAP, FcRH1 (Fc receptor-like protein 1), FcRH2, FCRH5, FGFR2, FGFR3, FGFR1-IIIc, FGFR2-IIIc, FGFR3-IIIc, Fibronectin extra-domain B, FLT3, Folate Receptor Alpha, GCC, GD3, Globo H, GPC3, GEDA, GFRA1, gpNMB, GPR20, GPRC5D, HER-2, HER-3, HLA-DOB, IGF-1R, IL1RAP, IL20Rα, IRTA2 (Immunoglobulin superfamily receptor translocation associated 2), Integrin beta-6, KAAG-1, LAMP-1, Lewis Y antigen, LIV-1, LRRC15, Ly6E, LY64 (Lymphocyte antigen 64), MDP, MPF (megakaryocyte potentiating factor), Mesothelin (MSLN), MT1-MMP (MMP14), MUC-1, MUC18, NaPi2b, Napi3b, NCA, Nectin-4, NOTCH3, P2X5 (Purinergic receptor P2X ligand-gated ion channel 5), P-Cadherin, PDGFR, PD-L1, Prolactin Receptor (PRLR), PSCA, PSCA homolog, PSMA, PTK7, RET, RNF43, ROR1, ROR2, Sema 5b, SEZ6, SLAMF7, SLC44A₄, SLITRK6, STEAP-1, STEAP2, STn, TAA, TIM-1, Tissue factor, TNF-alpha, TENB2, TrpM4, and TROP-2.

7. The conjugate as claimed in claim 3 or 4, wherein the antibody binds to an antigen selected from: ALPV/ALPPL2; CCR2; CD25; CDH17; TAG-72; and TM4SF1.

8. The conjugate as claimed in claim 3, wherein the antibody comprises between 1 and 20 cytotoxic compounds attached thereto.

9. The conjugate as claimed in claim 3, wherein the antibody 8 cytotoxic compounds attached thereto.

10. A pharmaceutical composition comprising a compound or conjugate as claimed in any one of the claims 1 to 9, and a pharmaceutically-acceptable excipient.

11. The composition as claimed in claim 10, wherein the composition is in the form of a tablet, capsule, liquid, or solution.

12. The composition as claimed in claim 10, wherein the composition is formulated for intravenous or subcutaneous injection.

13. The composition as claimed in claim 10, wherein the composition is in the form of a sterile solution.

14. The composition as claimed in claim 13, wherein the composition comprises a saline solution.

15. A conjugate having Formula V
T-[(W—Rₘ-L-D)]ₙ Formula V
wherein:
T— is a targeting moiety selected from a protein, a portion of a protein, a peptide or a nucleic acid;
—W—Rₘ— is a moiety formed by the reaction of maleimide and a functional group of T;
-L-D is a moiety having the formula: 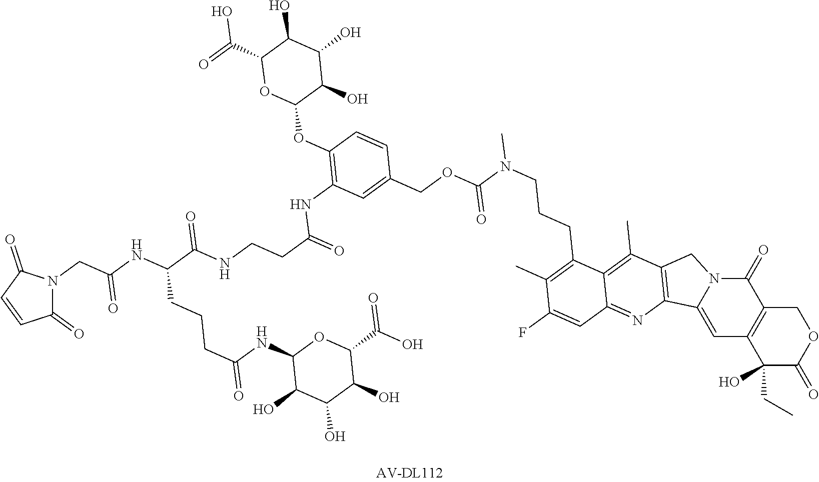
wherein n is an integer between 1 and 20.

16. The conjugate of claim 15, wherein the targeting moiety comprises an antibody.
